(19) United States
(12) Patent Application Publication
(10) Pub. No.: US 2024/0316051 A1
(43) Pub. Date: Sep. 26, 2024

(54) A PHARMACEUTICAL COMBINATION AND USE THEREOF

(71) Applicants: Ascentage Pharma (Suzhou) Co., Ltd., Suzhou, Jiangsu (CN); Ascentage Pharma Group Corp Limited, Hong Kong (CN)

(72) Inventors: Yifan ZHAI, Suzhou, Jiangsu (CN); Dajun YANG, Suzhou, Jiangsu (CN); Douglas Dong FANG, Suzhou, Jiangsu (CN); Saijie ZHU, Suzhou, Jiangsu (CN); Hengrui ZHU, Suzhou, Jiangsu (CN); Ran TAO, Suzhou, Jiangsu (CN)

(21) Appl. No.: 18/551,910

(22) PCT Filed: Apr. 19, 2022
(86) PCT No.: PCT/CN2022/087769

§ 371 Date: Sep. 22, 2023

(30) Foreign Application Priority Data
Apr. 19, 2021 (WO) PCT/CN2021/088213
Nov. 3, 2021 (WO) PCT/CN2021/128495

(51) Int. Cl. A61K 31/519; A61K 31/404; A61K 31/496; A61K 31/506; A61P 35/00

(52) U.S. Cl. CPC A61K 31/519; A61K 31/404; A61K 31/496; A61K 31/506; A61P 35/00

(57) ABSTRACT

Provided herein are the pharmaceutical combination comprising an embryonic ectoderm development (EED) inhibitor and one or more anticancer reagents, and the use of the combination in the treatment of a disease. Provided also is a pharmaceutical composition or a kit comprising the combination.

### TECHNICAL FIELD

The present invention pertains to the pharmaceutical field, and particularly relates to a pharmaceutical combination comprising an embryonic ectoderm development (EED) inhibitor and one or more anticancer reagents, and the use of the combination to treat a disease. The invention also relates to a pharmaceutical composition or kit comprising the combination.

### BACKGROUND OF THE INVENTION

Proliferative diseases represent a serious threat to modern society. Cancerous growths pose serious challenges for modern medicine due to their unique characteristics, including uncontrollable cell proliferation, an ability to invade local and even remote tissues, lack of differentiation, lack of detectable symptoms and lack of effective therapy and prevention. Worldwide, more than 10 million people are diagnosed with cancer every year, and cancer causes six million deaths every year or 12% of the deaths worldwide.

Many drugs targeting different targets for the treatment of cancer have been developed, including targeted-drugs (e.g., drugs targeting embryonic ectoderm development (EED), ALK, CDK4/6, Mek, MDM2, HDAC, PD-1, PARP, VEGF, BCR-ABL, Bcl-2 inhibitor etc.), however, there are still large uncertainties about which specific target drugs and which specific structure compounds are more effective in cancer treatment. Further, some drugs used alone tend to have problems of insufficient efficacy, excessive doses, and problems of drug-resistance. As compared with drugs used alone, drug combinations have many potential advantages. In particular, drug combinations targeting multiple different targets may reduce the dosage of each drug, avoid the emergence of single-drug resistance, and carefully screened combinations may further produce synergistic effects, thus improving the effectiveness of cancer treatment.

Accordingly, overcoming the drug resistance of the targeted drugs as well as other anticancer reagents and improving the efficacy are some of the main objectives in drug research and development, and there is a continuous and urgent need for individual drugs and pharmaceutical combinations with improved therapeutic efficacy and/or reduced drug resistance in the field of cancer treatment.

### SUMMARY OF THE INVENTION

In one aspect, the invention provides a pharmaceutical combination comprising an embryonic ectoderm development (EED) inhibitor and one or more anticancer

In another aspect, the invention provides a pharmaceutical composition comprising the pharmaceutical combination of the present invention, and optionally a pharmaceutically acceptable carrier.

In another aspect, the invention provides a method for treating or suppressing a cancer, reducing its severity, lowering its risk or inhibiting its metastasis in an individual, comprising administering to the individual a therapeutically effective amount of an EED inhibitor, and optionally a therapeutically effective amount of one or more anticancer reagents.

In another aspect, the invention provides the use of an EED inhibitor alone or in combination with one or more anticancer reagents for treating or suppressing a cancer, reducing its severity, lowering its risk or inhibiting its metastasis in an individual.

In another aspect, the invention provides the use of an EED inhibitor alone or in combination with one or more anticancer reagents in the manufacture of a medicament for treating or suppressing a cancer, reducing its severity, lowering its risk or inhibiting its metastasis in an individual.

In another aspect, the invention provides a kit, comprising:
(a) a first component in a first container, the first component comprising an EED inhibitor, and optionally a pharmaceutically acceptable carrier;
(b) a second component in a second container, the second component comprising an anticancer reagent, and optionally a pharmaceutically acceptable carrier; and
(c) an optional specification.

Preferably, the one or more anticancer reagents in the above aspects are selected from the group consisting of an ALK inhibitor, a BCR-ABL inhibitor, a MDM2 inhibitor, a Bcl-2 inhibitor and other inhibitors.

### BRIEF DESCRIPTION OF THE DRAWINGS

FIG. 1 shows that Compound 73 showed antitumor activity in BAP1 mut mesothelioma PDX model.

FIG. 2 shows the synergistic anti-tumor effect of Compound 73 in combination with Compound A-1 in BAP1 mut mesothelioma PDX model.

FIG. 3 shows the synergistic anti-tumor effect of Compound 73 in combination with Compound 2-5 in BAP1 mut mesothelioma PDX model.

FIG. 4 shows that Compound 73 exhibits antitumor activity in NCI-H226 (BAP1mut, CDKN2Adel, FGFR high) xenograft.

FIG. 5 shows the synergistic anti-tumor effect of Compound 73 in combination with Compound 2-5 in NCI-H226 (BAP1^{mut}, CDKN2^{Adel}, FGFR^{high}) xenograft.

FIG. 6 shows the synergistic anti-tumor effect of Compound 73 in combination with Compound C in subcutaneous malignant pleural mesothelioma (MPM) PDX model (FGFR1^{high}, BAP1^{mut}).

FIG. 7 shows body weight change (g) of mice under the treatment of Compound 73 and Compound C in subcutaneous malignant pleural mesothelioma (MPM) PDX model (FGFR1^{high}, BAP1^{mut}).

FIG. 8 shows the synergistic anti-tumor effect of Compound 73 in combination with Compound C in subcutaneous BAP1 mut mesothelioma PDX model.

FIG. 9 shows body weight change (%) of mice under the treatment of Compound 73 and Compound C in subcutaneous BAP1 mut mesothelioma PDX model.

FIG. 10 shows In vivo antitumor activity of Compound 73 and Compound C in H226 (BAP1mut, UHRF1high, TP53WT CDKN2Adel, FGFRhigh) subcutaneous mesothelioma xenograft model.

FIG. 11 shows body weight change (%) of mice under the treatment of Compound 73 and Compound C in H226 (BAP1mut, UHRF1high, TP53WT, CDKN2Adel, FGFRhigh) subcutaneous mesothelioma xenograft model.

FIG. 12 shows In vivo antitumor activity of Compound C and Compound 73 in NCI-H28(X1) (BAP1^{mut}, UHRF1^{high}, TP53^{WT}, CDKN2A^{del}, FGFR^{low}, NF2^{WT}) subcutaneous mesothelioma xenograft model.

FIG. 13 shows In vivo antitumor activity of Compound C and Compound 73 in subcutaneous 22RV1 prostate xenograft model.

FIG. 14 shows body weight change (%) of mice under the treatment of Compound C and Compound 73 in subcutaneous 22RV1 prostate xenograft model.

FIG. 15 shows In vivo antitumor activity of Compound C and Compound 73 in subcutaneous LnCap prostate xenograft model.

FIG. 16 shows In vitro anti-proliferative activity of Compound 73 and Compound 4 in EZH2 mut BCL2 translocation DLBCL cells lines (KARPAS-422)

FIG. 17 shows In vitro anti-proliferative activity of Compound 73 and Compound 4 in EZH2 mut BCL2 translocation DLBCL cells lines (SU-DHL-6)

FIG. 18 shows In vivo antitumor activity of Compound 73 and Compound 4 in KARPAS-422 subcutaneous DLBCL xenograft model

### DETAILED DESCRIPTION OF THE INVENTION

Unless otherwise defined below, all technical and scientific terms used herein have the same meanings as commonly understood by one of ordinary skill in the art. References to techniques used herein are intended to refer to techniques that are generally understood in the art, including those obvious changes or equivalent replacements of the techniques for those skilled in the art. While it is believed that the following terms are well understood by those skilled in the art, the following definitions are set forth to better explain the invention.

As used herein, the terms “including”, “comprising”, “having”, “containing” or “comprising”, and other variants thereof, are inclusive or open, and do not exclude other unlisted elements or method steps.

The use of the terms “a”, “an”, “the”, and similar referents in the context of describing the invention (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

As used herein, “EED” refers to embryonic ectoderm development protein, which is overexpressed in many cancers including, but not limited to, breast cancer, prostate cancer, and hepatocellular carcinoma. See Moritz and Trievel, J. Biol. Chem. 293(36): 13805-13814 (2018).

As used herein, “ALK” refers to anaplastic lymphoma kinase, and “ALK inhibitor” refers to an agent having an inhibitory effect on ALK. In some embodiments, the ALK inhibitor also has an inhibitory effect on one or more other targets (e.g., FAK (focal adhesion kinase) and/or ROS1 (a tyrosine protein kinase encoded by ROS1 proto-oncogene in human)).

As used herein, “BCR-ABL inhibitor” refers to an agent that targets the fusion gene of abelson murine leukemia (Abl) and breakpoint cluster region (Bcr).

As used herein, “MDM2 inhibitor” refers to an agent that targets the murine double minute (MDM2) protein.

As used herein, “Bcl-2” is the founding member of the Bcl-2 family of regulator proteins that regulate cell death (apoptosis), by either inducing (pro-apoptotic) or inhibiting (anti-apoptotic) apoptosis.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “synergistic effect” as used herein refers to action of two therapeutic agents, for example, slowing the symptomatic progression of a proliferative disease, particularly cancer, or symptoms thereof, which is greater than the simple addition of the effects of each drug administered by themselves. A synergistic effect can be calculated, for example, using various methods and equations well known in the art, such as those listed in the Examples of the present invention.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as used herein by itself or as part of another group refers to —OH.

The term “alkyl” as used herein, alone or as part of another group, refers to an unsubstituted straight or branched aliphatic hydrocarbon containing from 1 to 12 carbon atoms (ie, C₁₋₁₂ alkyl) or an indicated number of carbon atoms, for example, C₁ alkyl such as methyl, C₂ alkyl such as ethyl, C₃ alkyl such as n-propyl or isopropyl, C₁₋₃ alkyl such as methyl, ethyl, n-propyl or isopropyl, or the like. In one embodiment, the alkyl is C₁₋₄ alkyl. Non-limiting examples of C₁₋₁₂ alkyl include methyl, ethyl, n-propyl, isopropyl, n-butyl, sec-butyl, tert-butyl, isobutyl, 3-pentyl, hexyl, heptyl, octyl, nonyl and decyl. Examples of C₁₋₄ alkyl include methyl, ethyl, n-propyl, isopropyl, n-butyl, sec-butyl, tert-butyl, and isobutyl.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “alkynyl” as used herein by itself or as part of another group refers to an alkyl group containing one, two, or three carbon-to-carbon triple bonds. In one embodiment, the alkynyl is a C₂-C₆ alkynyl. In another embodiment, the alkynyl is a C₂-C₄ alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group. Non-limiting exemplary alkoxy groups include methoxy, ethoxy, and tert-butoxy.

The term “cycloalkyl” as used herein, alone or as part of another group, refers to a saturated or partially unsaturated (containing one or two double bonds) cyclic aliphatic hydrocarbon, which comprises 1 or 2 rings having 3 to 12 carbon atoms or an indicated number of carbon atoms (i.e., C₃₋₁₂ cycloalkyl). In one embodiment, the cycloalkyl has two rings. In one embodiment, the cycloalkyl has one ring. In another embodiment, the cycloalkyl group is selected from the group consisting of C₃₋₈ cycloalkyl groups. In another embodiment, the cycloalkyl group is selected from the group consisting of C₃₋₆ cycloalkyl groups. Non-limiting examples of cycloalkyl include cyclopropyl, cyclobutyl, cyclopentyl, cyclohexyl, cycloheptyl, cyclooctyl, norbornyl, decahydronaphthyl, adamantyl, cyclohexenyl, and cyclopentenyl.

The term “heterocycle” or “heterocyclyl” as used herein, alone or as part of another group, refers to a saturated or partially unsaturated (e.g., comprising one or two double bonds) cyclic group, which comprises 1, 2 or 3 rings having 3 to 14 ring members (i.e., 3- to 14-membered heterocyclyl), wherein at least one carbon atom of one of the rings is replaced by a heteroatom. Each heteroatom is independently selected from the group consisting of atoms of oxygen, sulfur (including sulfoxide and sulfone) and/or nitrogen (which may be oxidized or quaternized). The term “heterocyclyl” is intended to include a group wherein —CH₂— in the ring is replaced by —C(═O)—, for example, cyclic ureido (such as 2-imidazolidinone) and cyclic amido (such as β-lactam, γ-lactam, δ-lactam, ε-lactam) and piperazin-2-one. In one embodiment, the heterocyclyl is a 3- to 8-membered cyclic group comprising 1 ring and 1 or 2 oxygen and/or nitrogen atoms. In one embodiment, the heterocyclyl is a 4-, 5- or 6-membered cyclic group comprising 1 ring and 1 or 2 oxygen and/or nitrogen atoms. In one embodiment, the heterocyclyl is a 4- or 6-membered cyclic group comprising 1 ring and 1 or 2 oxygen and/or nitrogen atoms. The heterocyclyl can be attached to the remainder of molecule via any available carbon or nitrogen atom. Non-limiting examples of the heterocyclyl include dioxanyl, tetrahydropyranyl, 2-oxopyrrolidin-3-yl, piperazin-2-one, piperazin-2,6-dione, 2-imidazolidinone, piperidinyl, morpholinyl, piperazinyl, pyrrolidinyl and dihydroindolyl.

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “heteroaryl” as used herein by itself or as part of another group refers to monocyclic and bicyclic aromatic ring systems having five to fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “carboxy” as used by itself or as part of another group refers to a radical of the formula —C(═O)OH.

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

The combination of the present invention encompasses any of the compounds of the invention being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a compound wherein substantially all of the atoms at a position within the compound of the invention are replaced by an atom having a different atomic mass or mass number. In another embodiment, provided is a compound wherein substantially all of the atoms at a position within the compound of the invention are replaced by deuterium atoms, e.g., all of the hydrogen atoms of a —CH₃ group are replaced by deuterium atoms to give a —CD₃ group. In another embodiment, provided is a compound wherein a portion of the atoms at a position within the compound of the invention are replaced, i.e., the compound of the invention is enriched at a position with an atom having a different atomic mass or mass number. In another embodiment, provided is a compound wherein none of the atoms of the compound of the invention are replaced by an atom having a different atomic mass or mass number. Isotopically-labelled compounds of the invention can be prepared by methods known in the art.

The compounds in the combination of the invention may contain one or more asymmetric centers and may thus give rise to enantiomers, diastereomers, and other stereoisomeric forms. The present invention encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present invention.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “pharmaceutically acceptable salt”, as used herein, includes both acid addition salts and base addition salts of a compound.

Suitable acid addition salts are formed from acids which form non-toxic salts. Examples include acetate, adipate, aspartate, benzoate, besylate, bicarbonate/carbonate, bisulfate/sulfate, borate, camphorsulfonate, citrate, cyclohexylaminosulfonate, ethanedisulfonate, ethanesulfonate, formate, fumarate, glucoheptonate, gluconate, glucuronate, hexafluorophosphate, hibenzate, hydrochloride/chloride, hydrobromide/bromide, hydroiodide/iodide, isethionate, lactate, malate, maleate, malonate, methanesulfonate, methylsulfate, naphthylate, 2-naphthalenesulfonate, nicotinate, nitrate, orotate, oxalate, palmitate, pamoate, phosphate/hydrogen phosphate/dihydrogen phosphate, pyroglutamate, aldarate, stearate, succinate, tannate, tartrate, tosylate, trifluoroacetate and xinofoate.

Suitable base addition salts are formed from bases which form non-toxic salts. Examples include aluminum salts, arginine salts, benzathine benzylpenicillin salts, calcium salts, choline salts, diethylamine salts, diethanolamine salts, glycine salts, lysine salts, magnesium salts, meglumine salts, ethanolamine salts, potassium salts, sodium salts, tromethamine salts and zinc salts.

For a review of suitable salts, see “Handbook of Pharmaceutical Salts: Properties, Selection, and Use” by Stahl and Wermuth (Wiley-VCH, 2002). Methods for preparing the pharmaceutically acceptable salts of the compounds of the invention are known to those skilled in the art.

The term “solvate” as used herein is a substance formed by combination, physical binding and/or solvation of a compound of the invention with a solvent molecule, such as a disolvate, a monosolvate or a hemisolvate, wherein the ratio of the solvent molecule to the compound of the invention is about 2:1, about 1:1 or about 1:2, respectively. This kind of physical bonding involves ionization and covalent bonding (including hydrogen bonding) in different degrees. In some cases (e.g., when one or more solvent molecules are incorporated into crystal lattice of crystalline solid), the solvate can be isolated. Thus, the solvate comprises both solution phase and isolatable solvates. The compounds of the invention may be in solvated forms with pharmaceutically acceptable solvents (such as water, methanol and ethanol), and the present application is intended to encompass both solvated and unsolvated forms of the compounds of the invention.

One type of solvate is a hydrate. “Hydrate” relates to a specific subset of solvates wherein the solvent molecule is water. Solvates generally function in the form of pharmacological equivalents. The preparation of solvates is known in the art, see for example, M. Caira et al, J. Pharmaceut. Sci., 93(3): 601-611 (2004), which describes the preparation of a solvate of fluconazole with ethyl acetate and water. Similar methods for the preparation of solvates, hemisolvates, hydrates and the like are described by van Tonder et al, AAPS Pharm. Sci. Tech., 5(1): Article 12 (2004) and A. L. Bingham et al, Chem. Commun. 603-604 (2001). A representative and non-limiting method for the preparation of solvate involves dissolving a compound of the invention in a desired solvent (organic solvent, water or a mixture thereof) at a temperature above 20° C. to about 25° C., and then the solution is cooled at a rate sufficient to form a crystal, and the crystal is separated by a known method such as filtration. Analytical techniques such as infrared spectroscopy can be used to confirm the presence of the solvent in the crystal of the solvate.

“Pharmaceutically acceptable carrier” in the context of the present invention refers to a diluent, adjuvant, excipient or vehicle together with which the therapeutic agent is administered, and which is suitable for contacting a tissue of human and/or other animals within the scope of reasonable medical judgment, and without excessive toxicity, irritation, allergic reactions, or other problems or complications corresponding to a reasonable benefit/risk ratio.

The pharmaceutically acceptable carriers that can be used in the pharmaceutical compositions or kits of the invention include, but are not limited to, sterile liquids such as water and oils, including those oils derived from petroleum, animals, vegetables or synthetic origins, for example, peanut oil, soybean oil, mineral oil, sesame oil, etc. Water is an exemplary carrier when the pharmaceutical composition is administered intravenously. It is also possible to use physiological saline and an aqueous solution of glucose and glycerin as a liquid carrier, particularly for injection. Suitable pharmaceutical excipients include starch, glucose, lactose, sucrose, gelatin, maltose, chalk, silica gel, sodium stearate, glyceryl monostearate, talc, sodium chloride, skimmed milk powder, glycerin, propylene glycol, water, ethanol and the like. The pharmaceutical composition may further contain a small amount of a wetting agent, an emulsifier or a pH buffering agent as needed. Oral formulations may contain standard carriers such as pharmaceutical grades of mannitol, lactose, starch, magnesium stearate, sodium saccharin, cellulose, magnesium carbonate, and the like. Examples of suitable pharmaceutically acceptable carriers are as described in Remington's Pharmaceutical Sciences (1990).

The pharmaceutical compositions and the components of the kit of the invention may act systemically and/or locally. For this purpose, they may be administered via a suitable route, for example by injection (e.g., intravenous, intraarterial, subcutaneous, intraperitoneal, intramuscular administration, including instillation) or transdermal administration; or by oral, buccal, nasal, transmucosal, topical administration, in form of ophthalmic preparation or by inhalation.

For these routes of administration, the pharmaceutical compositions and the components of the kit of the invention may be administered in a suitable dosage form.

The dosage forms include, but are not limited to, tablets, capsules, troches, hard candy, pulvis, sprays, creams, ointments, suppositories, gels, pastes, lotions, ointments, aqueous suspensions, injectable solutions, elixirs, syrups.

The term “container” as used herein refers to a container for holding a pharmaceutical component. This container can be used for preparation, storage, transportation and/or stand-alone/bulk sale, which is intended to include bottles, cans, vials, flasks, syringes, tubes (e.g., those used in cream products), or any other containers for preparation, containment, storage or distribution of a drug product.

The term “specification/instruction” as used herein refers to an insert, a tag, a label, etc., which records information about a pharmaceutical component located in the container. The information as recorded is typically determined by the regulatory agency (e.g., the United States Food and Drug Administration) that governs the area in which the product is to be sold. Preferably, the package leaflet specifically lists an indication for which the use of the pharmaceutical component is approved. The package leaflet can be made of any material from which information contained therein or thereon can be read. Preferably, the package leaflet is a printable material (e.g., paper, plastic, cardboard, foil, adhesive paper or plastic, etc.) on which the desired information can be formed (e.g., printed or applied).

The term “effective amount” as used herein refers to an amount of active ingredient that, after administration, will relieve to some extent one or more symptoms of the condition being treated.

As used herein, “individual” includes a human or a non-human animal. Exemplary human individual includes a human individual (referred to as a patient) suffering from a disease (such as the disease described herein) or a normal individual. “Non-human animal” in the present invention includes all vertebrates, such as non-mammals (e.g., birds, amphibians, reptiles) and mammals, such as non-human primates, domestic animals, and/or domesticated animals (e.g., sheep, dogs, cats, cows, pigs, etc.).

As used herein, “cancer metastasis” refers to a cancer that spreads (metastasizes) from its original site to another area of the body. Almost all cancers have the potential to metastasize. Whether metastasis will occur depends on complex interactions between multiple tumor cell factors (including type of cancer, degree of maturation (differentiation) of tumor cells, location and age of cancer, and other factors that are not fully understood). There are three ways of metastasis: local expansion from a tumor to a surrounding tissue, arrival through bloodstream to a distant site, or arrival through lymphatic system to an adjacent or distant lymph node. Each cancer can have a representative diffusion route. Tumors are named according to their primary sites (for example, breast cancer that has metastasized to the brain is called metastatic breast cancer that metastasizes to the brain).

As used herein, “resistance” refers to that a cancer cell is resistant to chemotherapy. Cancer cells may acquire resistance to chemotherapy through a range of mechanisms, including mutation or overexpression of drug targets, inactivation of drugs, or elimination of drugs from cells.

The term “about,” as used herein, includes the recited number±10%. Thus, “about 10” means 9 to 11.

### Pharmaceutical Combinations and Uses

In one embodiment, the invention provides a method for treating or suppressing a cancer, reducing its severity, lowering its risk or inhibiting its metastasis in an individual, comprising administering to the individual a therapeutically effective amount of an EED inhibitor.

In one embodiment, the invention provides the use of an EED inhibitor for treating or suppressing a cancer, reducing its severity, lowering its risk or inhibiting its metastasis in an individual.

In one embodiment, the invention provides the use of an EED inhibitor in the manufacture of a medicament for treating or suppressing a cancer, reducing its severity, lowering its risk or inhibiting its metastasis in an individual.

In one embodiment, the invention provides a pharmaceutical combination comprising an embryonic ectoderm development (EED) inhibitor and one or more anticancer reagents.

In a preferred embodiment, the one or more anticancer reagents are selected from the group consisting of an ALK inhibitor, a BCR-ABL inhibitor, a MDM2 inhibitor, a Bcl-2 inhibitor and other inhibitors.

In a preferred embodiment, the EED inhibitor is a compound of Formula I:

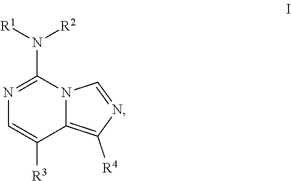

wherein:
R¹ is aralkyl;
R² is selected from the group consisting of hydrogen and C₁-C₄ alkyl;
R³ and R⁴ taken together with the carbon atoms to which they are attached form a radical of Formula I-A, I-B, or I-C:

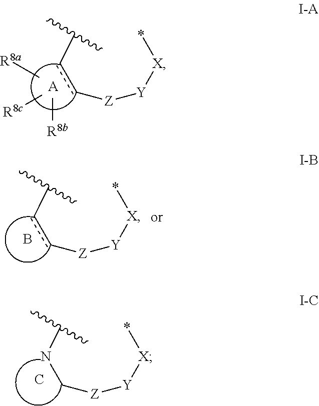

X is selected from the group consisting of —C(R^{5a})(R^{5b})—, —C(═O)—, and —S(═O)₂—;
R^{5a} and R^{5b} are independently selected from the group consisting of hydrogen and C₁-C₄ alkyl;

Y is selected from the group consisting of —C(R^{6a})(R^{6b})—, —S—, —O—, and —N(R⁷)—; or
X and Y taken together form a 5-membered heteroarylenyl;
Z is —C(R^{6c})(R^{6d})ₘ—;
R^{6a} and R^{6b} are independently selected from the group consisting of hydrogen and C₁-C₄ alkyl;
each R^{6c} and R^{6d} is independently selected from the group consisting of hydrogen and C₁-C₄ alkyl;
m is 0, 1, or 2;
R⁷ is selected from the group consisting of hydrogen, C₁-C₆ alkyl, C₁-C₆ haloalkyl, optionally substituted C₃-C₈ cycloalkyl, optionally substituted C₄-C₈ heterocyclo, hydroxyalkyl, (alkoxy)alkyl, (cycloalkyl)alkyl, and (heterocyclo)alkyl;
R^{8a}, R^{8b}, and R^{8c} are independently selected from the group consisting of hydrogen, halo, C₁-C₄ alkyl, C₁-C₄ haloalkyl, C₁-C₄ alkoxy, carboxamido, optionally substituted C₃-C₈ cycloalkyl, optionally substituted 4- to 8-membered heterocyclo, (heterocyclo)C₁-C₄ alkyl, and alkylsulfonyl;

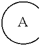

is a fused phenyl, fused 5-membered heteroaryl, or fused 6-membered heteroaryl;

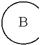

is an optionally substituted fused 3- to 8-membered cycloalkyl or optionally substituted fused 4- to 8-membered heterocyclo;

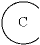

is an optionally substituted fused 4- to 8-membered heterocyclo; and the bond designated with a “ [symbol] ” is attached at the R³ position of Formula I and the bond designated with an “*” is attached at the R⁴ position of Formula I; and
[symbol] is a single or double bond, or a pharmaceutically acceptable salt or solvate thereof.

In another embodiment, The EED inhibitors are compounds of Formula I, wherein:
X is selected from the group consisting of —C(R^{5a})(R^{5b})—, —C(═O)—, and —S(═O)₂—;
Y is selected from the group consisting of —C(R^{6a})(R^{6b})—, —S—, —O—, and —N(R⁷)—; and
R^{8a}, R^{8b}, and R^{8c} are independently selected from the group consisting of hydrogen, halo, C₁-C₄ alkyl, C₁-C₄ haloalkyl, C₁-C₄ alkoxy, and alkylsulfonyl, or a pharmaceutically acceptable salt or solvate thereof.

In another embodiment, The EED inhibitors are compounds of Formula I, wherein R³ and R4 taken together with the carbon atoms to which they are attached form a radical of Formula I-A, I-B, or I-C, or a pharmaceutically acceptable salt or solvate thereof.

In another embodiment, The EED inhibitors are compounds of Formula II:

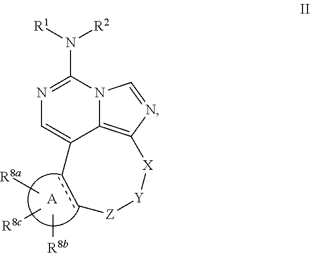

wherein R¹, R², R^{8a}, R^{8b}, R^{8c}, X, Y, Z,

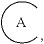

and [symbol] are as defined in connection with Formula I, or a pharmaceutically acceptable salt or solvate thereof.

In another embodiment, The EED inhibitors are compounds of Formula III:

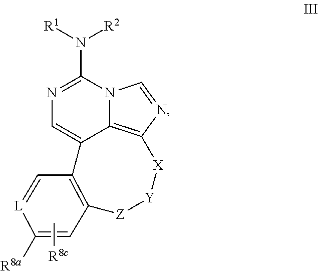

wherein L is selected from the group consisting of —C(R^{8b})═ and —N═; and R¹, R², R^{8a}, R^{8b}, R^{8c}, X, Y, and Z are as defined in connection with Formula I, or a pharmaceutically acceptable salt or solvate thereof.

In another embodiment, The EED inhibitors are compounds of Formula IV:

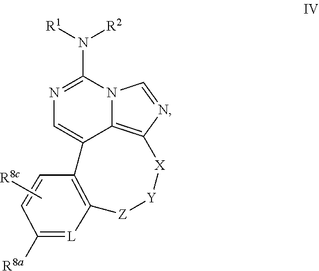

wherein L is selected from the group consisting of —C(R^{8b})═ and —N═; and R¹, R², R^{8a}, R^{8b}, R^{8c}, X, Y, and Z are as defined in connection with Formula I, or a pharmaceutically acceptable salt or solvate thereof.

In another embodiment, The EED inhibitors are compounds of Formula V:

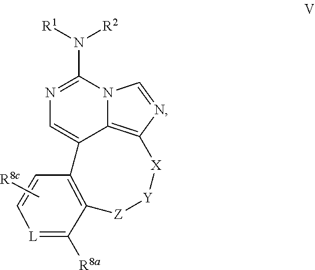

wherein L is selected from the group consisting of —C(R^{8b})═ and —N═; and R¹, R², R^{8a}, R^{8b}, R^{8c}, X, Y, and Z are as defined in connection with Formula I, or a pharmaceutically acceptable salt or solvate thereof.

In another embodiment, The EED inhibitors are compounds of Formula VI:

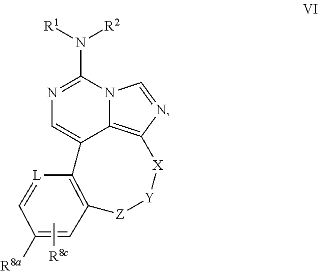

wherein L is selected from the group consisting of —C(R^{8b})═ and —N═; and R¹, R², R^{8a}, R^{8b}, R^{8c}, X, Y, and Z are as defined in connection with Formula I, or a pharmaceutically acceptable salt or solvate thereof.

In another embodiment, The EED inhibitors are compounds of any one of Formulae III-VI, wherein L is —C(R^{8b})—, or a pharmaceutically acceptable salt or solvate thereof.

In another embodiment, The EED inhibitors are compounds of any one of Formulae III-VI, wherein L is —N═, or a pharmaceutically acceptable salt or solvate thereof.

In another embodiment, The EED inhibitors are compounds of any one of Formulae I-VI, wherein R^{8a}, R^{8b}, and R^{8c} are independently selected from the group consisting of hydrogen, C₁-C₄ alkyl, C₁-C₄ haloalkyl, and C₃-C₆ cycloalkyl, or a pharmaceutically acceptable salt or solvate thereof. In another embodiment, R^{8a} is selected from the group consisting of —CHF₂, —CF₃, —CH₃, —CD₃, and cyclopropyl; and R^{8b} and R^{8c} are hydrogen. In another embodiment, R^{8a} is selected from the group consisting of —CF₃ or —CH₃; and R^{8b} and R^{8c} are hydrogen.

In another embodiment The EED inhibitors are compounds of any one of Formulae I-VI, wherein, R^{8a} is selected from the group consisting of C₁-C₄ alkyl, 4- to 8-membered heterocyclo, and (heterocyclo)C₁-C₄ alkyl; and R^{8b} and R^{8c} are hydrogen, or a pharmaceutically acceptable salt or solvate thereof. In another embodiment, R^{8a} is C₁-C₄ alkyl. In another embodiment, R^{8a} is 4- to 8-membered heterocyclo. In another embodiment, R^{8a} is (heterocyclo)C₁-C₄ alkyl. In another embodiment, R^{8a} is selected from the group consisting of:

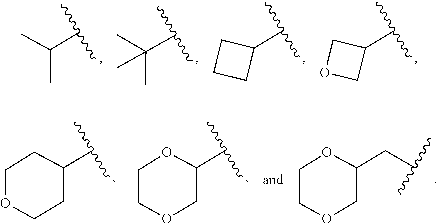

In another embodiment, The EED inhibitors are compounds of Formula VII:

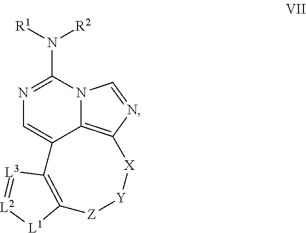

wherein:
L¹ is selected from the group consisting of —S—, —O—, and —N(R^{8a})—;
L² is selected from the group consisting of —C(R^{8b})═ and —N═;
L³ is selected from the group consisting of —C(R^{8c})═ and —N═;
R^{8a} is selected from the group consisting of hydrogen and C₁-C₄ alkyl;
R^{8b} is selected from the group consisting of hydrogen, C₁-C₄ alkyl, and C₁-C₄ haloalkyl;

R^{8c} is selected from the group consisting of hydrogen, C₁-C₄ alkyl, and C₁-C₄ haloalkyl; and

R¹, R², X, Y, and Z are as defined in connection with Formula I, or a pharmaceutically acceptable salt or solvate thereof.

In another embodiment, The EED inhibitors are compounds of Formula VIII:

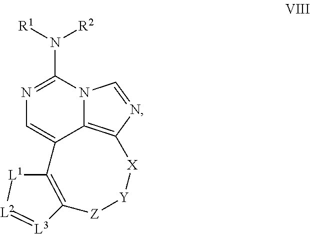

wherein:
L¹ is selected from the group consisting of —S—, —O—, and —N(R^{8a})—;
L² is selected from the group consisting of —C(R^{8b})═ and —N═;
L³ is selected from the group consisting of —C(R^{8c})═ and —N═;
R^{8a} is selected from the group consisting of hydrogen and C₁-C₄ alkyl;
R^{8b} is selected from the group consisting of hydrogen, C₁-C₄ alkyl, and C₁-C₄ haloalkyl;
R^{8c} is selected from the group consisting of hydrogen, C₁-C₄ alkyl, and C₁-C₄ haloalkyl; and
R¹, R², X, Y, and Z are as defined in connection with Formula I, or a pharmaceutically acceptable salt or solvate thereof.

In another embodiment, The EED inhibitors are compounds of Formula IX:

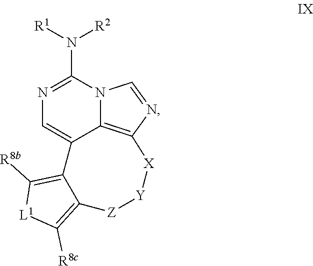

wherein:
L¹ is selected from the group consisting of —S—, —O—, and —N(R^{8a})—;
R^{8a} is selected from the group consisting of hydrogen and C₁-C₄ alkyl;
R^{8b} is selected from the group consisting of hydrogen, C₁-C₄ alkyl, and C₁-C₄ haloalkyl;
R^{8c} is selected from the group consisting of hydrogen, C₁-C₄ alkyl, and C₁-C₄ haloalkyl; and
R¹, R², X, Y, and Z are as defined in connection with Formula I, or a pharmaceutically acceptable salt or solvate thereof.

In another embodiment, The EED inhibitors are compounds of Formula X:

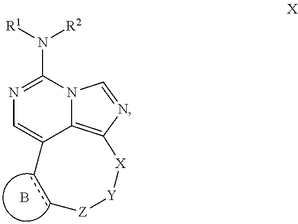

wherein R¹, R², X, Y, Z,

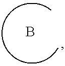

and [symbol] are as defined in connection with Formula I, or a pharmaceutically acceptable salt or solvate thereof.

In another embodiment, The EED inhibitors are compounds of Formula XI:

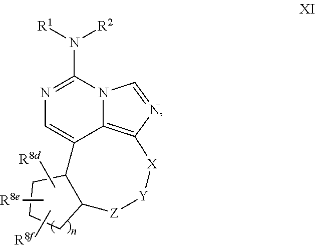

wherein:
R^{8d}, R^{8e}, and R^{8f} are independently selected from the group consisting of hydrogen, halo, and C₁-C₄ alkyl;
n is 1, 2, or 3; and
R¹, R², X, Y, and Z are as defined in connection with Formula I, or a pharmaceutically acceptable salt or solvate thereof.

In another embodiment, The EED inhibitors are compounds of Formula XI-A:

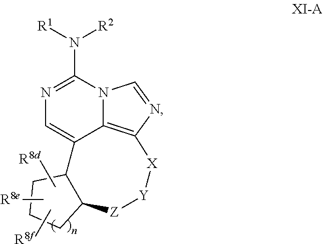

wherein R¹, R², R^{8d}, R^{8e}, R^{8f}, n, X, Y, and Z are as defined in connection with Formula XI, or a pharmaceutically acceptable salt or solvate thereof.

In another embodiment, The EED inhibitors are compounds of Formula XI-B:

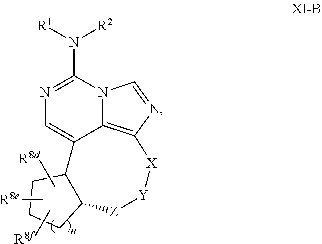

wherein R¹, R², R^{8d}, R^{8e}, R^{8f}, n, X, Y, and Z are as defined in connection with Formula XI, or a pharmaceutically acceptable salt or solvate thereof.

In another embodiment, The EED inhibitors are compounds of Formula XII:

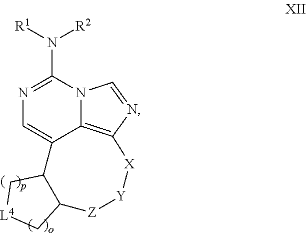

wherein:
L⁴ is selected from the group consisting of —S—, —O—, and —N(R^{8g})—;
R^{8g} is selected from the group consisting of hydrogen, C₁-C₄ alkyl, optionally substituted C₃-C₆ cycloalkyl, and optionally substituted 4- to 8-membered heterocyclo;
o is 0, 1, 2, or 3;
p is 0, 1, 2, or 3;
wherein the sum of o and p is 1, 2, 3, 4, or 5; and
R¹, R², X, Y, and Z are as defined in connection with Formula I, or a pharmaceutically acceptable salt or solvate thereof.

In another embodiment, The EED inhibitors are compounds of Formula XII-A:

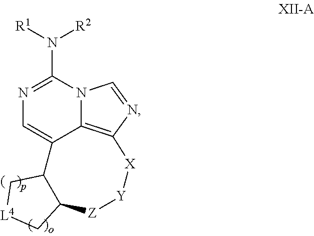

wherein R¹, R², L⁴, o, p, X, Y, and Z are as defined in connection with Formula XII, or a pharmaceutically acceptable salt or solvate thereof.

In another embodiment, The EED inhibitors are compounds of Formula XII-B:

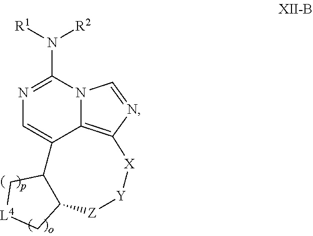

wherein R¹, R², L⁴, o, p, X, Y, and Z are as defined in connection with Formula XII, or a pharmaceutically acceptable salt or solvate thereof.

In another embodiment, The EED inhibitors are compounds of Formula XIII:

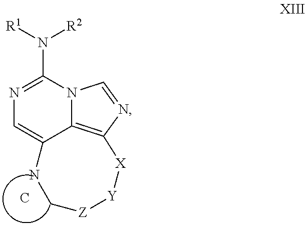

wherein R¹, R², X, Y, Z, and

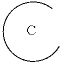

are as defined in connection with Formula I, or a pharmaceutically acceptable salt or solvate thereof.

In another embodiment, The EED inhibitors are compounds of Formula XIII-A:

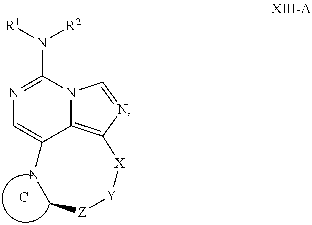

wherein R¹, R², X, Y, Z, and

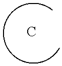

are as defined in connection with Formula I, or a pharmaceutically acceptable salt or solvate thereof.

In another embodiment, The EED inhibitors are compounds of Formula XIII-B:

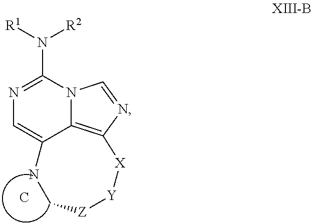

wherein R¹, R², X, Y, Z, and

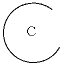

are as defined in connection with Formula I, or a pharmaceutically acceptable salt or solvate thereof.

In another embodiment, The EED inhibitors are compounds of Formula XIV:

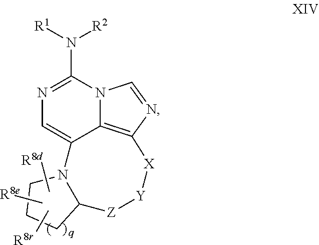

wherein:
R^{8d}, R^{8e}, and R^{8f} are independently selected from the group consisting of hydrogen and C₁-C₄ alkyl;
q is 1, 2, or 3; and
R¹, R², X, Y, and Z are as defined in connection with Formula I, or a pharmaceutically acceptable salt or solvate thereof.

In another embodiment, The EED inhibitors are compounds of Formula XIV-A:

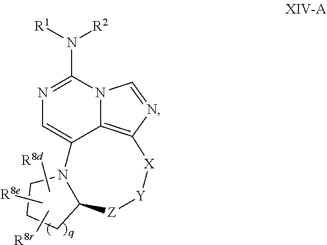

wherein R¹, R², R^{8d}, R^{8e}, R^{8f}, q, X, Y, and Z are as defined in connection with Formula XIV, or a pharmaceutically acceptable salt or solvate thereof.

In another embodiment, The EED inhibitors are compounds of Formula XIV-B:

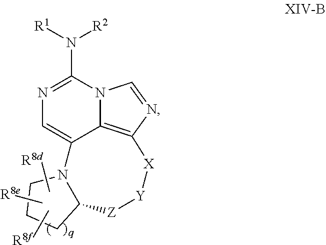

wherein R¹, R², R^{8d}, R^{8e}, R^{8f}, q, X, Y, and Z are as defined in connection with Formula XIV, or a pharmaceutically acceptable salt or solvate thereof.

In another embodiment, The EED inhibitors are compounds of Formula XV:

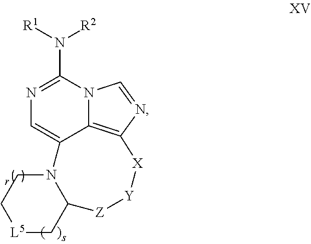

wherein:
L⁵ is selected from the group consisting of —S—, —O—, and —N(R^{8h})—;
R^{8h} is selected from the group consisting of hydrogen, C₁-C₄ alkyl, optionally substituted C₃-C₆ cycloalkyl, optionally substituted 4- to 8-membered heterocyclo, —C(═O)R^{14a}, and —S(═O)₂R^{14b};
R^{14a} and R^{14b} are independently selected from the group consisting of C₁-C₆ alkyl and optionally substituted C₃-C₈ cycloalkyl;
r is 1, 2, or 3;
s is 1, 2, or 3; and
R¹, R², X, Y, and Z are as defined in connection with Formula I, or a pharmaceutically acceptable salt or solvate thereof.

In another embodiment, The EED inhibitors are compounds of Formula XV-A:

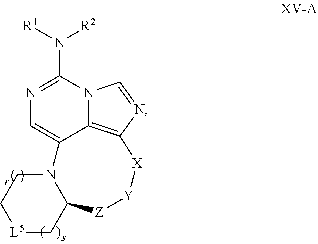

wherein R¹, R², L⁵, r, s, X, Y, and Z are as defined in connection with Formula XV, or a pharmaceutically acceptable salt or solvate thereof.

In another embodiment, The EED inhibitors are compounds of Formula XV-B:

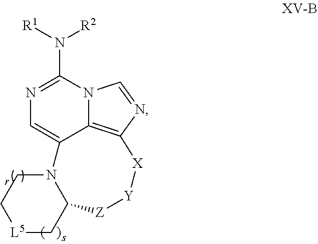

wherein R¹, R², L⁵, r, s, X, Y, and Z are as defined in connection with Formula XV, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein Z is —CH₂—, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X is —CH₂—, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X is —C(═O)—, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein X is-S(═O)₂—, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein Y is-O—, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein Y is —N(R⁷)—, or a pharmaceutically acceptable salt or solvate thereof. In another embodiment, R⁷ is selected from the group consisting of C₁-C₆ alkyl, C₁-C₆ haloalkyl, and optionally substituted C₃-C₈ cycloalkyl, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein Z is —CH₂—, X is —C(═O)—, and Y is —N(R⁷)—, or a pharmaceutically acceptable salt or solvate thereof. In another embodiment, R⁷ is selected from the group consisting of C₁-C₆ alkyl, C₁-C₆ haloalkyl, and optionally substituted C₃-C₈ cycloalkyl. In another embodiment, R⁷ is C₁-C₄ alkyl. In another embodiment, R⁷ is selected from the group consisting of methyl, ethyl, propyl, or isopropyl.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein R² is hydrogen, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein:
R¹ is R¹-1:

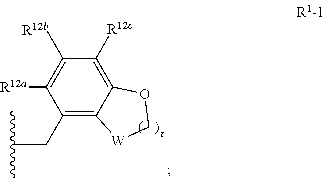

R^{12a}, R^{12b}, and R^{12c} are each independently selected from the group consisting of hydrogen, halo, C₁-C₄ alkyl, C₁-C₄ haloalkyl, and C₁-C₄ alkoxy;
W is selected from the group consisting of —CH₂— and —C(═O)—; and
t is 1 or 2, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein R¹ is R¹-1, R^{12a} is fluoro; and R^{12b} and R^{12c} are independently selected from the group consisting of hydrogen and fluoro. In another embodiment, R^{12a} is fluoro; and R^{12b} and R^{12c} are hydrogen.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein:
R¹ is R¹-2:

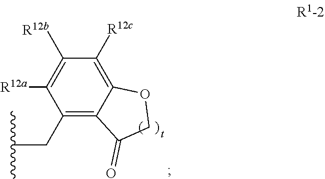

R^{12a}, R^{12b}, and R^{12c} are each independently selected from the group consisting of hydrogen, halo, C₁-C₄ alkyl, C₁-C₄ haloalkyl, and C₁-C₄ alkoxy; and
t is 1 or 2, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein R¹ is R¹-2, R^{12a} is fluoro; and R^{12b} and R^{12c} are independently selected from the group consisting of hydrogen and fluoro. In another embodiment, R^{12a} is fluoro; and R^{12b} and R^{12c} are hydrogen.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein:
R¹ is R¹-3:

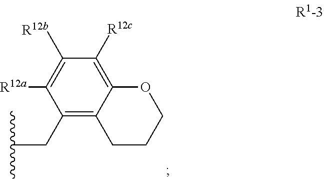

and
R^{12a}, R^{12b}, and R^{12c} are each independently selected from the group consisting of hydrogen, halo, C₁-C₄ alkyl, C₁-C₄ haloalkyl, and C₁-C₄ alkoxy, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein R¹ is R¹-3, R^{12a} is fluoro; and R^{12b} and R^{12c} are independently selected from the group consisting of hydrogen and fluoro. In another embodiment, R^{12a} is fluoro; and R^{12b} and R^{12c} are hydrogen.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein:
R¹ is R¹-4:

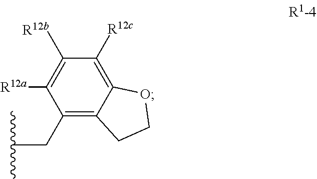

and
R^{12a}, R^{12b}, and R^{12c} are each independently selected from the group consisting of hydrogen, halo, C₁-C₄ alkyl, C₁-C₄ haloalkyl, and C₁-C₄ alkoxy, or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein R¹ is R¹-4, R^{12a} is fluoro; and R^{12b} and R^{12c} are independently selected from the group consisting of hydrogen and fluoro. In another embodiment, R^{12a} is fluoro; and R^{12b} and R^{12c} are hydrogen.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein R¹ is selected from the group consisting of:

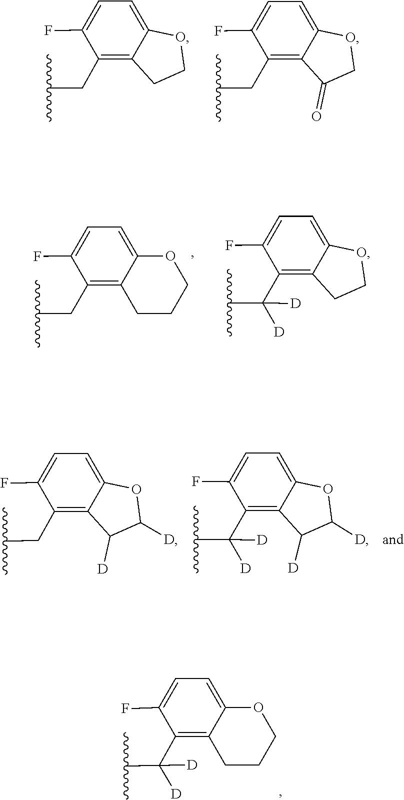

or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein R¹ is selected from the group consisting of:

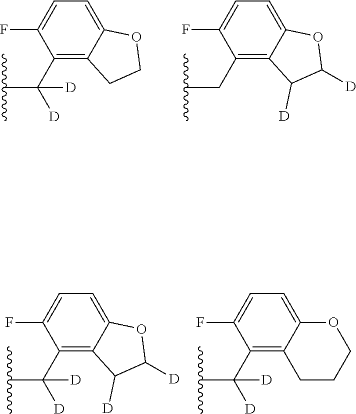

or a pharmaceutically acceptable salt or solvate thereof.

In another embodiment, The EED inhibitors are compounds of any one of Formulae I-XI, XI-A, XI-B, XII, XII-A, XII-B, XIII, XIII-A, XIII-B, XIV, XIV-A, XIV-B, XV, XV-A, or XV-B, wherein R¹ is selected from the group consisting of:

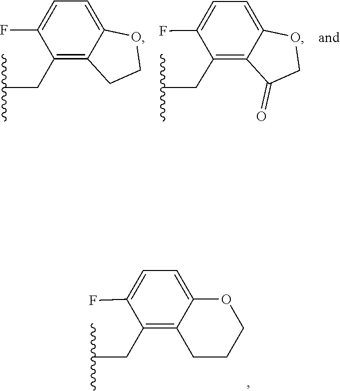

or a pharmaceutically acceptable salt or solvate thereof.

In another embodiment, The EED inhibitors are any one or more of the compounds listed in Table 1, or a pharmaceutically acceptable salt or solvate thereof.

TABLE 1

| Cpd. | | |
|---|---|---|
| No. | Structure | Name |
| 1 | 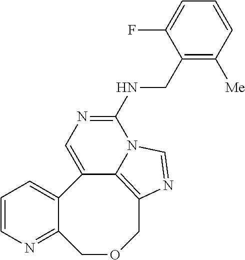 [SMILES: Cc1cccc(F)c1CNc1ncc2c3c(ncn13)COCc1ncccc1-2] | N-(2-fluoro-6-methylbenzyl)-3H,5H-4-oxa-2,6,11, 12a-tetraazabenzo[4,5]cycloocta[1,2,3-cd]inden-1 2-amine |
| 2 | 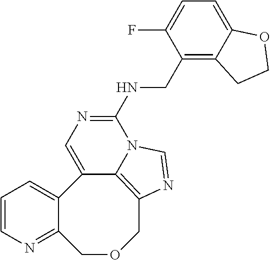 [SMILES: Fc1ccc2c(c1CNc1ncc3c4c(ncn14)COCc1ncccc1-3)CCO2] | N-((5-fluoro-2,3-dihydrobenzofuran-4-yl)methyl)- 3H,5H-4-oxa-2,6,11,12a-tetraazabenzo[4,5] cycloocta[1,2,3-cd]inden-12-amine |
| 3 | 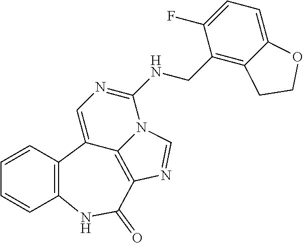 [SMILES: O=C1Nc2ccccc2-c2cnc(NCc3c(F)ccc4c3CCO4)n3cnc1c23] | 11-(((5-fluoro-2,3-dihydrobenzofuran-4-yl)methyl) amino)-2,4,10,11a-tetraazadibenzo[cd,f]azulen-3 (4H)-one |
| 4 | 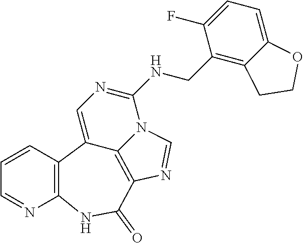 [SMILES: O=C1Nc2ncccc2-c2cnc(NCc3c(F)ccc4c3CCO4)n3cnc1c23] | 11-(((5-fluoro-2,3-dihydrobenzofuran-4-yl)methyl) amino)-2,4,5,10,11a-pentaazadibenzo[cd,f]azulen- 3(4H)-one |
| 5 | 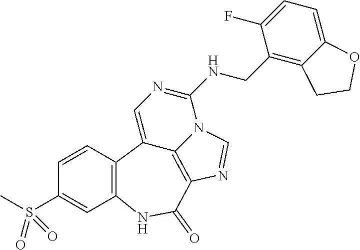 [SMILES: CS(=O)(=O)c1ccc2c(c1)NC(=O)c1ncn3c(NCc4c(F)ccc5c4CCO5)ncc-2c13] | 11-(((5-fluoro-2,3-dihydrobenzofuran-4-yl)methyl) amino)-6-(methylsulfonyl)-2,4,10,11a- tetraazadibenzo[cd,f]azulen-3(4H)-one |
| 6 | 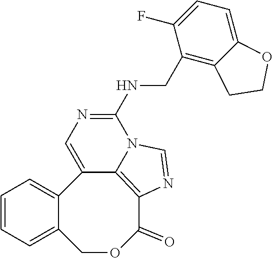 [SMILES: O=C1OCc2ccccc2-c2cnc(NCc3c(F)ccc4c3CCO4)n3cnc1c23] | 12-(((5-fluoro-2,3-dihydrobenzofuran-4-yl)methyl) amino)-3H,5H-4-oxa-2,11,12a-triazabenzo[4,5] cycloocta[1,2,3-cd]inden-3-one |
| 7 | 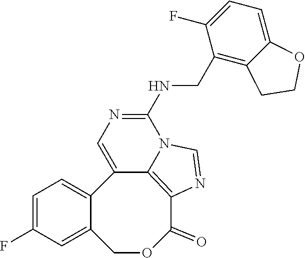 [SMILES: O=C1OCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-3H,5H-4-oxa-2,11,12a-triazabenzo [4,5]cycloocta[1,2,3-cd]inden-3-one |
| 8 | 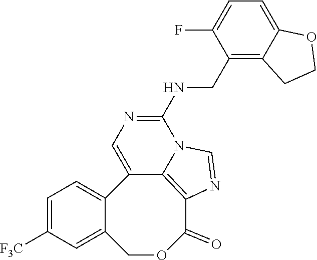 [SMILES: O=C1OCc2cc(C(F)(F)F)ccc2-c2cnc(NCc3c(F)ccc4c3CCO4)n3cnc1c23] | 12-(((5-fluoro-2,3-dihydrobenzofuran-4-yl)methyl) amino)-7-(trifluoromethyl)-3H,5H-4-oxa-2,11,12a- triazabenzo[4,5]cycloocta[1,2,3-cd]inden-3-one |
| 9 | 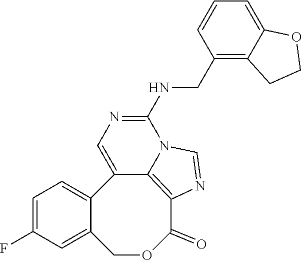 [SMILES: O=C1OCc2cc(F)ccc2-c2cnc(NCc3cccc4c3CCO4)n3cnc1c23] | 12-(((2,3-dihydrobenzofuran-4-yl)methyl)amino)- 7-fluoro-3H,5H-4-oxa-2,11,12a-triazabenzo[4,5] cycloocta[1,2,3-cd]inden-3-one |
| 10 | 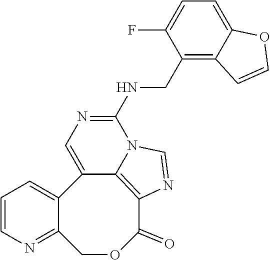 [SMILES: O=C1OCc2ncccc2-c2cnc(NCc3c(F)ccc4occc34)n3cnc1c23] | 12-(((5-fluorobenzofuran-4-yl)methyl)amino)-3H, 5H-4-oxa-2,6,11,12a-tetraazabenzo[4,5]cycloocta [1,2,3-cd]inden-3-one |
| 11 | 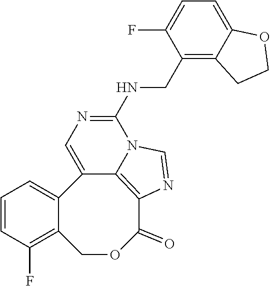 [SMILES: O=C1OCc2c(F)cccc2-c2cnc(NCc3c(F)ccc4c3CCO4)n3cnc1c23] | 6-fluoro-12-(((5-fluoro-2,3-dihydrobenzofuran-4- yl)methyl)amino)-3H,5H-4-oxa-2,11,12a-triazabenzo [4,5]cycloocta[1,2,3-cd]inden-3-one |
| 12 | 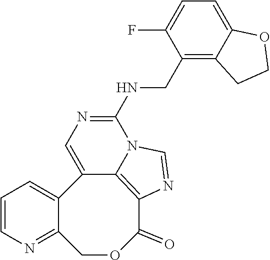 [SMILES: O=C1OCc2ncccc2-c2cnc(NCc3c(F)ccc4c3CCO4)n3cnc1c23] | 12-(((5-fluoro-2,3-dihydrobenzofuran-4-yl)methyl) amino)-3H,5H-4-oxa-2,6,11,12a-tetraazabenzo [4,5]cycloocta[1,2,3-cd]inden-3-one |
| 13 | 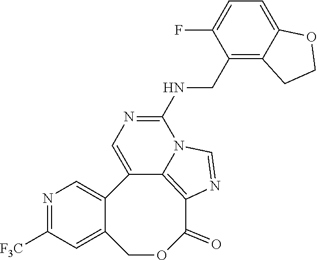 [SMILES: O=C1OCc2cc(C(F)(F)F)ncc2-c2cnc(NCc3c(F)ccc4c3CCO4)n3cnc1c23] | 12-(((5-fluoro-2,3-dihydrobenzofuran-4-yl)methyl) amino)-7-(trifluoromethyl)-3H,5H-4-oxa-2,8,11, 12a-tetraazabenzo[4,5]cycloocta[1,2,3-cd]inden-3- one |
| 14 | 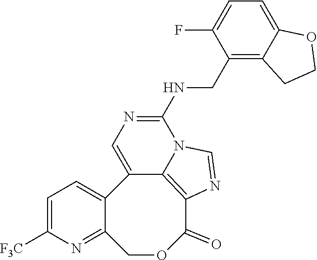 [SMILES: O=C1OCc2nc(C(F)(F)F)ccc2-c2cnc(NCc3c(F)ccc4c3CCO4)n3cnc1c23] | 12-(((5-fluoro-2,3-dihydrobenzofuran-4-yl)methyl) amino)-7-(trifluoromethyl)-3H,5H-4-oxa-2,6,11, 12a-tetraazabenzo[4,5]cycloocta[1,2,3-cd]inden-3- one |
| 15 | 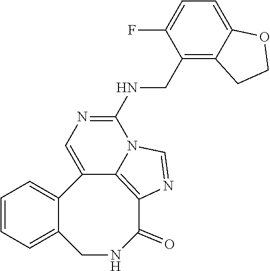 [SMILES: O=C1NCc2ccccc2-c2cnc(NCc3c(F)ccc4c3CCO4)n3cnc1c23] | 12-(((5-fluoro-2,3-dihydrobenzofuran-4-yl)methyl) amino)-4,5-dihydro-3H-2,4,11,12a-tetraazabenzo [4,5]cycloocta[1,2,3-cd]inden-3-one |
| 16 | 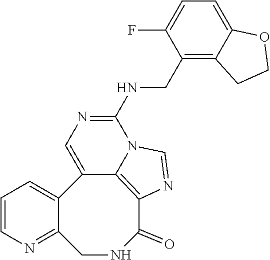 [SMILES: O=C1NCc2ncccc2-c2cnc(NCc3c(F)ccc4c3CCO4)n3cnc1c23] | 12-(((5-fluoro-2,3-dihydrobenzofuran-4-yl)methyl) amino)-4,5-dihydro-3H-2,4,6,11,12a-pentaazabenzo [4,5]cycloocta[1,2,3-cd]inden-3-one |
| 17 | 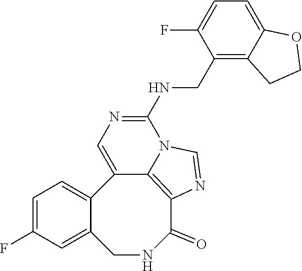 [SMILES: O=C1NCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-4,5-dihydro-3H-2,4,11,12a-tetra azabenzo[4,5]cycloocta[1,2,3-cd]inden-3-one |
| 18 | 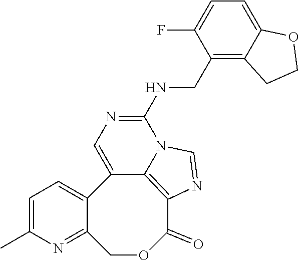 [SMILES: Cc1ccc2c(n1)COC(=O)c1ncn3c(NCc4c(F)ccc5c4CCO5)ncc-2c13] | 12-(((5-fluoro-2,3-dihydrobenzofuran-4-yl)methyl) amino)-7-methyl-3H,5H-4-oxa-2,6,11,12a- tetraazabenzo[4,5]cycloocta[1,2,3-cd]inden-3-one |
| 19 | 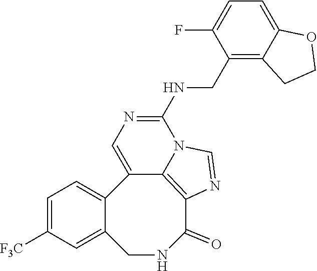 [SMILES: O=C1NCc2cc(C(F)(F)F)ccc2-c2cnc(NCc3c(F)ccc4c3CCO4)n3cnc1c23] | 12-(((5-fluoro-2,3-dihydrobenzofuran-4-yl)methyl) amino)-7-(trifluoromethyl)-4,5-dihydro-3H-2,4,11, 12a-tetraazabenzo[4,5]cycloocta[1,2,3-cd]inden- 3-one |
| 20 | 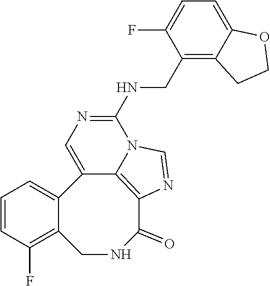 [SMILES: O=C1NCc2c(F)cccc2-c2cnc(NCc3c(F)ccc4c3CCO4)n3cnc1c23] | 6-fluoro-12-(((5-fluoro-2,3-dihydrobenzofuran-4- yl)methyl)amino)-4,5-dihydro-3H-2,4,11,12a-tetra azabenzo[4,5]cycloocta[1,2,3-cd]inden-3-one |
| 21 | 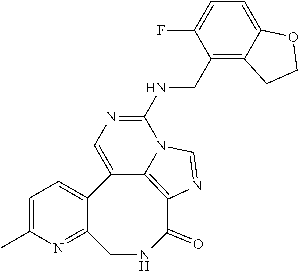 [SMILES: Cc1ccc2c(n1)CNC(=O)c1ncn3c(NCc4c(F)ccc5c4CCO5)ncc-2c13] | 12-(((5-fluoro-2,3-dihydrobenzofuran-4-yl)methyl) amino)-7-methyl-4,5-dihydro-3H-2,4,6,11,12a- pentaazabenzo[4,5]cycloocta[1,2,3-cd]inden-3-one |
| 22 | 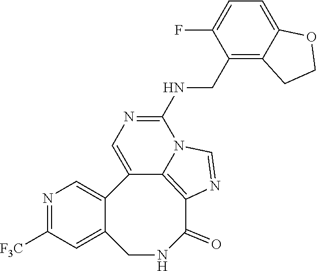 [SMILES: O=C1NCc2cc(C(F)(F)F)ncc2-c2cnc(NCc3c(F)ccc4c3CCO4)n3cnc1c23] | 12-(((5-fluoro-2,3-dihydrobenzofuran-4-yl)methyl) amino)-7-(trifluoromethyl)-4,5-dihydro-3H-2,4,8, 11,12a-pentaazabenzo[4,5]cycloocta[1,2,3-cd] inden-3-one |
| 23 | 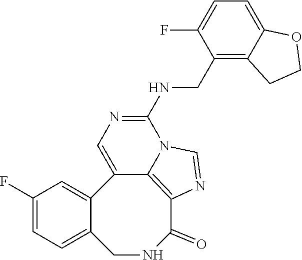 [SMILES: O=C1NCc2ccc(F)cc2-c2cnc(NCc3c(F)ccc4c3CCO4)n3cnc1c23] | 8-fluoro-12-(((5-fluoro-2,3-dihydrobenzofuran-4- yl)methyl)amino)-4,5-dihydro-3H-2,4,11,12a-tetra azabenzo[4,5]cycloocta[1,2,3-cd]inden-3-one |
| 24 | 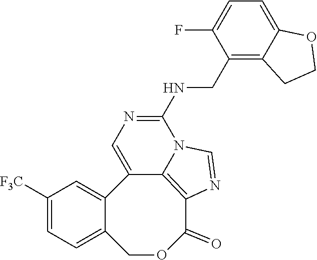 [SMILES: O=C1OCc2ccc(C(F)(F)F)cc2-c2cnc(NCc3c(F)ccc4c3CCO4)n3cnc1c23] | 12-(((5-fluoro-2,3-dihydrobenzofuran-4-yl)methyl) amino)-8-(trifluoromethyl)-3H,5H-4-oxa-2,11,12a- triazabenzo[4,5]cycloocta[1,2,3-cd]inden-3-one |
| 25 | 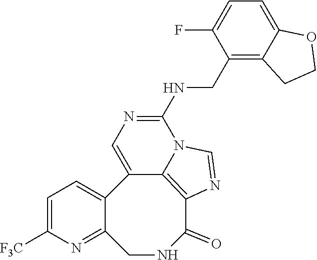 [SMILES: O=C1NCc2nc(C(F)(F)F)ccc2-c2cnc(NCc3c(F)ccc4c3CCO4)n3cnc1c23] | 12-(((5-fluoro-2,3-dihydrobenzofuran-4-yl)methyl) amino)-7-(trifluoromethyl)-4,5-dihydro-3H-2,4,6, 11,12a-pentaazabenzo[4,5]cycloocta[1,2,3-cd] inden-3-one |
| 26 | 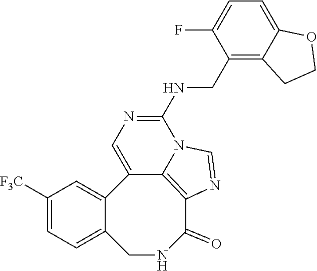 [SMILES: O=C1NCc2ccc(C(F)(F)F)cc2-c2cnc(NCc3c(F)ccc4c3CCO4)n3cnc1c23] | 12-(((5-fluoro-2,3-dihydrobenzofuran-4-yl)methyl) amino)-8-(trifluoromethyl)-4,5-dihydro-3H-2,4,11, 12a-tetraazabenzo[4,5]cycloocta[1,2,3-cd]inden- 3-one |
| 27 | 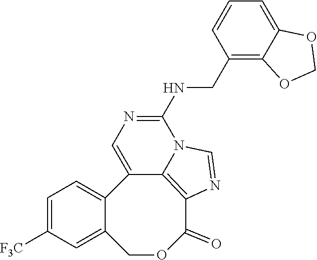 [SMILES: O=C1OCc2cc(C(F)(F)F)ccc2-c2cnc(NCc3cccc4c3OCO4)n3cnc1c23] | 12-((benzo[d][1,3]dioxol-4-ylmethyl)amino)-7- (trifluoromethyl)-3H,5H-4-oxa-2,11,12a-triazabenzo [4,5]cycloocta[1,2,3-cd]inden-3-one |
| 28 | 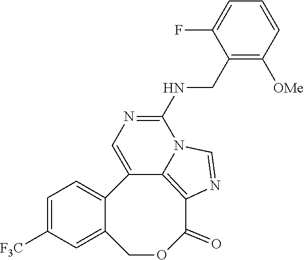 [SMILES: COc1cccc(F)c1CNc1ncc2c3c(ncn13)C(=O)OCc1cc(C(F)(F)F)ccc1-2] | 12-((2-fluoro-6-methoxybenzyl)amino)-7-(trifluoro- methyl)-3H,5H-4-oxa-2,11,12a-triazabenzo[4,5] cycloocta[1,2,3-cd]inden-3-one |
| 29 | 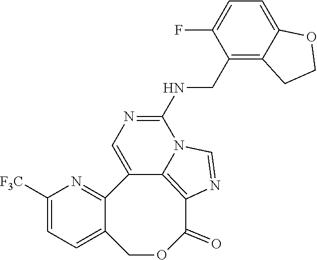 [SMILES: O=C1OCc2ccc(C(F)(F)F)nc2-c2cnc(NCc3c(F)ccc4c3CCO4)n3cnc1c23] | 12-(((5-fluoro-2,3-dihydrobenzofuran-4-yl)methyl) amino)-8-(trifluoromethyl)-3H,5H-4-oxa-2,9,11, 12a-tetraazabenzo[4,5]cycloocta[1,2,3-cd]inden-3- one |
| 30 | 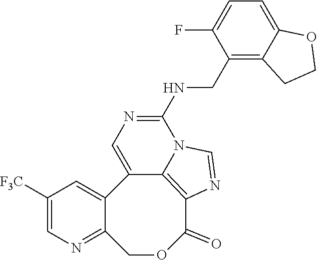 [SMILES: O=C1OCc2ncc(C(F)(F)F)cc2-c2cnc(NCc3c(F)ccc4c3CCO4)n3cnc1c23] | 12-(((5-fluoro-2,3-dihydrobenzofuran-4-yl)methyl) amino)-8-(trifluoromethyl)-3H,5H-4-oxa-2,6,11, 12a-tetraazabenzo[4,5]cycloocta[1,2,3-cd]inden-3- one |
| 31 | 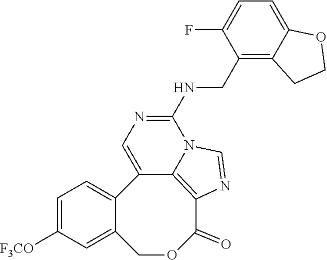 [SMILES: O=C1OCc2cc(OC(F)(F)F)ccc2-c2cnc(NCc3c(F)ccc4c3CCO4)n3cnc1c23] | 12-(((5-fluoro-2,3-dihydrobenzofuran-4-yl)methyl) amino)-7-(trifluoromethoxy)-3H,5H-4-oxa-2,11, 12a-triazabenzo[4,5]cycloocta[1,2,3-cd]inden-3-one |
| 32 | 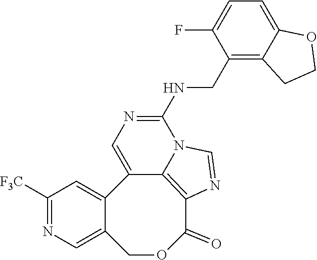 [SMILES: O=C1OCc2cnc(C(F)(F)F)cc2-c2cnc(NCc3c(F)ccc4c3CCO4)n3cnc1c23] | 12-(((5-fluoro-2,3-dihydrobenzofuran-4-yl)methyl) amino)-8-(trifluoromethyl)-3H,5H-4-oxa-2,7,11, 12a-tetraazabenzo[4,5]cycloocta[1,2,3-cd]inden-3- one |
| 33 | 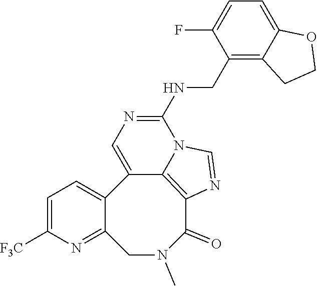 [SMILES: CN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4-yl)methyl) amino)-4-methyl-7-(trifluoromethyl)-4,5-dihydro- 3H-2,4,6,11,12a-pentaazabenzo[4,5]cycloocta [1,2,3-cd]inden-3-one |
| 34 | 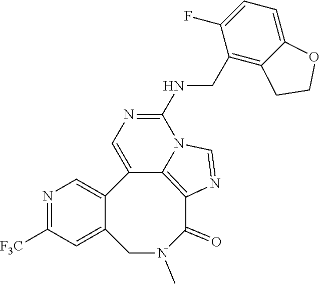 [SMILES: CN1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4-yl)methyl) amino)-4-methyl-7-(trifluoromethyl)-4,5-dihydro- 3H-2,4,8,11,12a-pentaazabenzo[4,5]cycloocta [1,2,3-cd]inden-3-one |
| 35 | 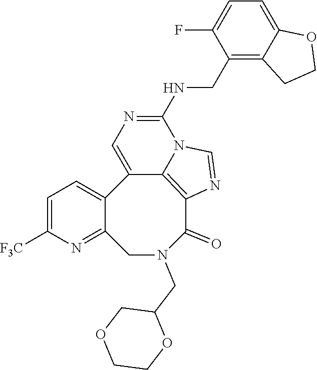 [SMILES: O=C1c2ncn3c(NCc4c(F)ccc5c4CCO5)ncc(c23)-c2ccc(C(F)(F)F)nc2CN1CC1COCCO1] | 4-((1,4-dioxan-2-yl)methyl)-12-(((5-fluoro-2,3- dihydrobenzofuran-4-yl)methyl)amino)-7-(trifluoro- methyl)-4,5-dihydro-3H-2,4,6,11,12a-pentaazabenzo [4,5]cycloocta[1,2,3-cd]inden-3-one |
| 36 | 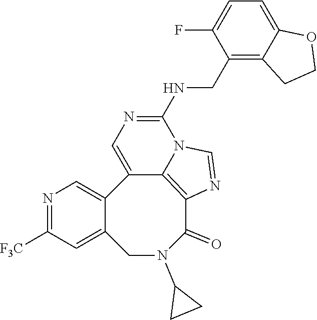 [SMILES: O=C1c2ncn3c(NCc4c(F)ccc5c4CCO5)ncc(c23)-c2cnc(C(F)(F)F)cc2CN1C1CC1] | 4-cyclopropyl-12-(((5-fluoro-2,3-dihydrobenzo- furan-4-yl)methyl)amino)-7-(trifluoromethyl)-4,5- dihydro-3H-2,4,8,11,12a-pentaazabenzo[4,5] cycloocta[1,2,3-cd]inden-3-one |
| 37 | 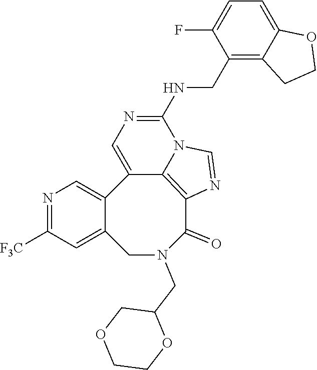 [SMILES: O=C1c2ncn3c(NCc4c(F)ccc5c4CCO5)ncc(c23)-c2cnc(C(F)(F)F)cc2CN1CC1COCCO1] | 4-((1,4-dioxan-2-yl)methyl)-12-(((5-fluoro-2,3- dihydrobenzofuran-4-yl)methyl)amino)-7-(trifluoro- methyl)-4,5-dihydro-3H-2,4,8,11,12a-pentaazabenzo [4,5]cycloocta[1,2,3-cd]inden-3-one |
| 38 | 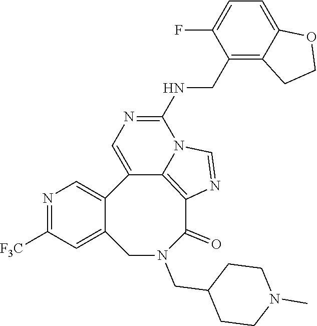 [SMILES: CN1CCC(CN2Cc3cc(C(F)(F)F)ncc3-c3cnc(NCc4c(F)ccc5c4CCO5)n4cnc(c34)C2=O)CC1] | 12-(((5-fluoro-2,3-dihydrobenzofuran-4-yl)methyl) amino)-4-((1-methylpiperidin-4-yl)methyl)-7- (trifluoromethyl)-4,5-dihydro-3H-2,4,8,11,12a- pentaazabenzo[4,5]cycloocta[1,2,3-cd]inden-3-one |
| 39 | 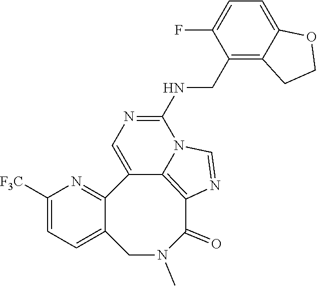 [SMILES: CN1Cc2ccc(C(F)(F)F)nc2-c2cnc(NCc3c(F)ccc4c3CCO4)n3cnc(c23)C1=O] | 12-(((5-fluoro-2,3-dihydrobenzofuran-4-yl)methyl) amino)-4-methyl-8-(trifluoromethyl)-4,5-dihydro- 3H-2,4,9,11,12a-pentaazabenzo[4,5]cycloocta [1,2,3-cd]inden-3-one |
| 40 | 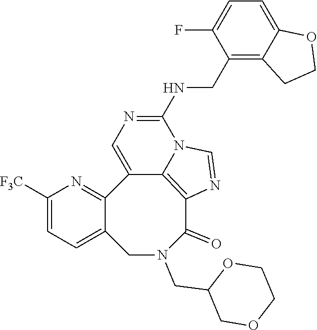 [SMILES: O=C1c2ncn3c(NCc4c(F)ccc5c4CCO5)ncc(c23)-c2nc(C(F)(F)F)ccc2CN1CC1COCCO1] | 4-((1,4-dioxan-2-yl)methyl)-12-(((5-fluoro-2,3- dihydrobenzofuran-4-yl)methyl)amino)-8-(trifluoro- methyl)-4,5-dihydro-3H-2,4,9,11,12a-pentaazabenzo [4,5]cycloocta[1,2,3-cd]inden-3-one |
| 41 | 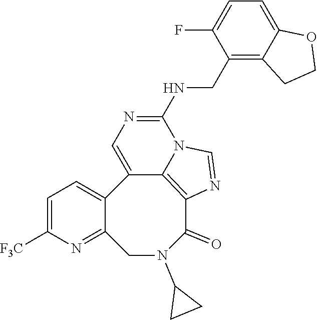 [SMILES: O=C1c2ncn3c(NCc4c(F)ccc5c4CCO5)ncc(c23)-c2ccc(C(F)(F)F)nc2CN1C1CC1] | 4-cyclopropyl-12-(((5-fluoro-2,3-dihydrobenzofuran- 4-yl)methyl)amino)-7-(trifluoromethyl)-4,5- dihydro-3H-2,4,6,11,12a-pentaazabenzo[4,5] cycloocta[1,2,3-cd]inden-3-one |
| 42 | 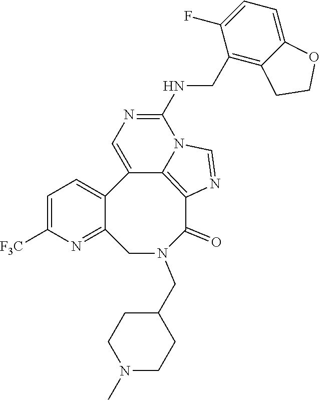 [SMILES: CN1CCC(CN2Cc3nc(C(F)(F)F)ccc3-c3cnc(NCc4c(F)ccc5c4CCO5)n4cnc(c34)C2=O)CC1] | 12-(((5-fluoro-2,3-dihydrobenzofuran-4-yl)methyl) amino)-4-((1-methylpiperidin-4-yl)methyl)-7- (trifluoromethyl)-4,5-dihydro-3H-2,4,6,11,12a- pentaazabenzo[4,5]cycloocta[1,2,3-cd]inden-3-one |
| 43 | 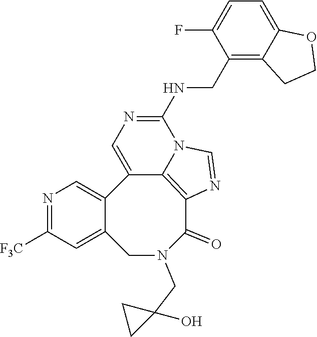 [SMILES: O=C1c2ncn3c(NCc4c(F)ccc5c4CCO5)ncc(c23)-c2cnc(C(F)(F)F)cc2CN1CC1(O)CC1] | 12-(((5-fluoro-2,3-dihydrobenzofuran-4-yl)methyl) amino)-4-((1-hydroxycyclopropyl)methyl)-7- (trifluoromethyl)-4,5-dihydro-3H-2,4,8,11,12a- pentaazabenzo[4,5]cycloocta[1,2,3-cd]inden-3-one |
| 44 | 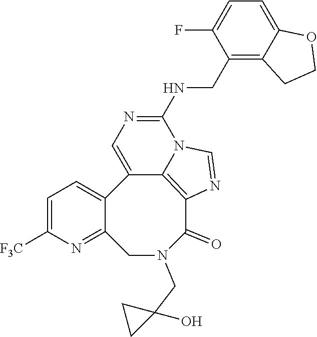 [SMILES: O=C1c2ncn3c(NCc4c(F)ccc5c4CCO5)ncc(c23)-c2ccc(C(F)(F)F)nc2CN1CC1(O)CC1] | 12-(((5-fluoro-2,3-dihydrobenzofuran-4-yl)methyl) amino)-4-((1-hydroxycyclopropyl)methyl)-7- (trifluoromethyl)-4,5-dihydro-3H-2,4,6,11,12a- pentaazabenzo[4,5]cycloocta[1,2,3-cd]inden-3-one |
| 45 | 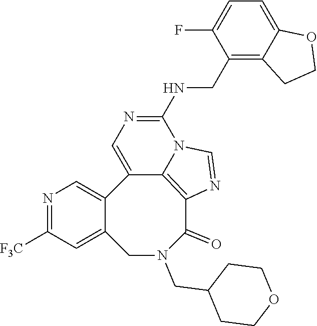 [SMILES: O=C1c2ncn3c(NCc4c(F)ccc5c4CCO5)ncc(c23)-c2cnc(C(F)(F)F)cc2CN1CC1CCOCC1] | 12-(((5-fluoro-2,3-dihydrobenzofuran-4-yl)methyl) amino)-4-((tetrahydro-2H-pyran-4-yl)methyl)-7- (trifluoromethyl)-4,5-dihydro-3H-2,4,8,11,12a- pentaazabenzo[4,5]cycloocta[1,2,3-cd]inden-3-one |
| 46 | 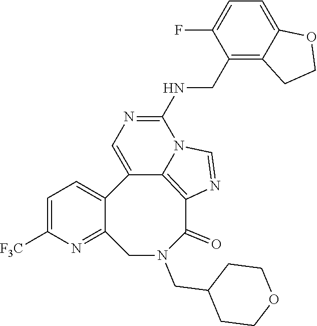 [SMILES: O=C1c2ncn3c(NCc4c(F)ccc5c4CCO5)ncc(c23)-c2ccc(C(F)(F)F)nc2CN1CC1CCOCC1] | 12-(((5-fluoro-2,3-dihydrobenzofuran-4-yl)methyl) amino)-4-((tetrahydro-2H-pyran-4-yl)methyl)-7- (trifluoromethyl)-4,5-dihydro-3H-2,4,6,11, 12a- pentaazabenzo[4,5]cycloocta[1,2,3-cd]inden-3-one |
| 47 | 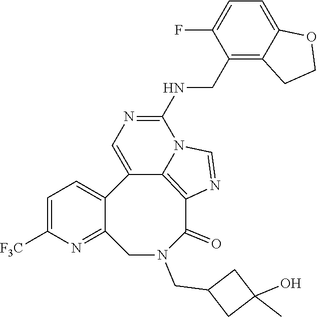 [SMILES: CC1(O)CC(CN2Cc3nc(C(F)(F)F)ccc3-c3cnc(NCc4c(F)ccc5c4CCO5)n4cnc(c34)C2=O)C1] | 12-(((5-fluoro-2,3-dihydrobenzofuran-4-yl)methyl) amino)-4-((3-hydroxy-3-methylcyclobutyl)methyl)- 7-(trifluoromethyl)-4,5-dihydro-3H-2,4,6,11,12a- pentaazabenzo[4,5]cycloocta[1,2,3-cd]inden-3-one |
| 48 | 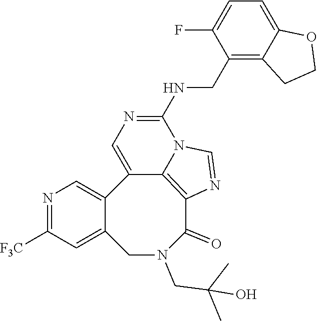 [SMILES: CC(C)(O)CN1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4-yl)methyl) amino)-4-(2-hydroxy-2-methylpropyl)-7-(trifluoro- methyl)-4,5-dihydro-3H-2,4,8,11,12a-pentaaza- benzo[4,5]cycloocta[1,2,3-cd]inden-3-one |
| 49 | 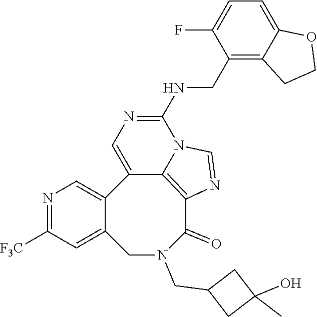 [SMILES: CC1(O)CC(CN2Cc3cc(C(F)(F)F)ncc3-c3cnc(NCc4c(F)ccc5c4CCO5)n4cnc(c34)C2=O)C1] | 12-(((5-fluoro-2,3-dihydrobenzofuran-4-yl)methyl) amino)-4-((3-hydroxy-3-methylcyclobutyl)methyl)- 7-(trifluoromethyl)-4,5-dihydro-3H-2,4,8,11,12a- pentaazabenzo[4,5]cycloocta[1,2,3-cd]inden-3-one |
| 50 | 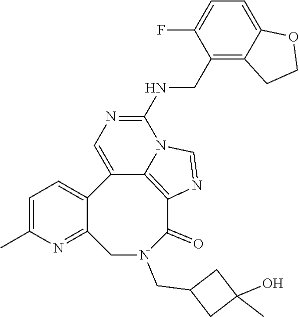 [SMILES: Cc1ccc2c(n1)CN(CC1CC(C)(O)C1)C(=O)c1ncn3c(NCc4c(F)ccc5c4CCO5)ncc-2c13] | 12-(((5-fluoro-2,3-dihydrobenzofuran-4-yl)methyl) amino)-4-((3-hydroxy-3-methylcyclobutyl)methyl)- 7-methyl-4,5-dihydro-3H-2,4,6,11,12a-pentaaza benzo[4,5]cycloocta[1,2,3-cd]inden-3-one |
| 51 | 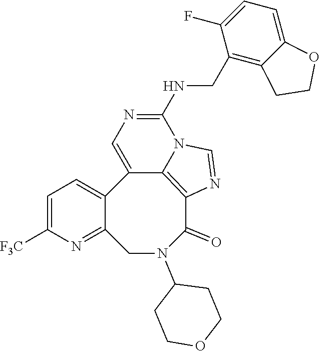 [SMILES: O=C1c2ncn3c(NCc4c(F)ccc5c4CCO5)ncc(c23)-c2ccc(C(F)(F)F)nc2CN1C1CCOCC1] | 12-(((5-fluoro-2,3-dihydrobenzofuran-4-yl)methyl) amino)-4-(tetrahydro-2H-pyran-4-yl)-7-(trifluoro methyl)-4,5-dihydro-3H-2,4,6,11, 12a-pentaazabenzo [4,5]cycloocta[1,2,3-cd]inden-3-one |
| 52 | 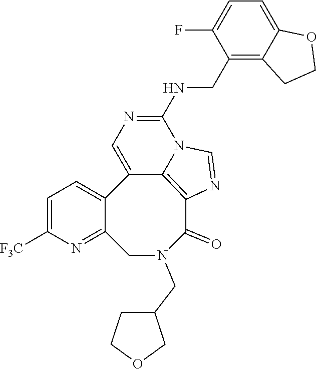 [SMILES: O=C1c2ncn3c(NCc4c(F)ccc5c4CCO5)ncc(c23)-c2ccc(C(F)(F)F)nc2CN1CC1CCOC1] | 12-(((5-fluoro-2,3-dihydrobenzofuran-4-yl)methyl) amino)-4-((tetrahydrofuran-3-yl)methyl)-7-(trifluoro- methyl)-4,5-dihydro-3H-2,4,6,11,12a-pentaaza benzo[4,5]cycloocta[1,2,3-cd]inden-3-one |
| 53 | 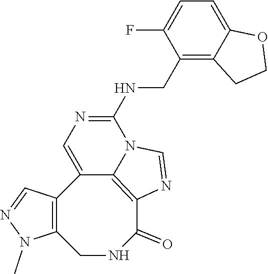 [SMILES: Cn1ncc2c1CNC(=O)c1ncn3c(NCc4c(F)ccc5c4CCO5)ncc-2c13] | 11-(((5-fluoro-2,3-dihydrobenzofuran-4-yl)methyl) amino)-6-methyl-5,6-dihydro-2,4,6,7,10,11a- hexaazacyclopenta[4,5]cycloocta[1,2,3-cd]inden- 3(4H)-one |
| 54 | 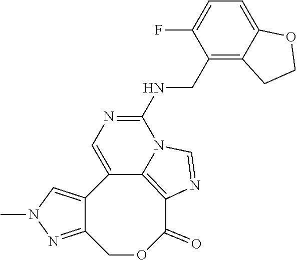 [SMILES: Cn1cc2c(n1)COC(=O)c1ncn3c(NCc4c(F)ccc5c4CCO5)ncc-2c13] | 11-(((5-fluoro-2,3-dihydrobenzofuran-4-yl)methyl) amino)-7-methyl-5,7-dihydro-3H-4-oxa-2,6,7,10, 11a-pentaazacyclopenta[4,5]cycloocta[1,2,3-cd] inden-3-one |
| 55 | 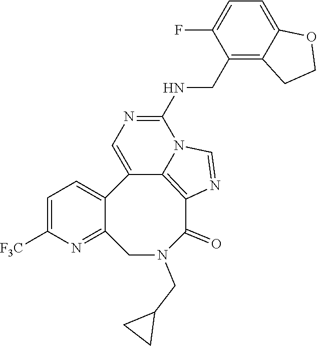 [SMILES: O=C1c2ncn3c(NCc4c(F)ccc5c4CCO5)ncc(c23)-c2ccc(C(F)(F)F)nc2CN1CC1CC1] | 4-(cyclopropylmethyl)-12-(((5-fluoro-2,3-dihydro benzofuran-4-yl)methyl)amino)-7-(trifluoromethyl)- 4,5-dihydro-3H-2,4,6,11,12a-pentaazabenzo[4,5] cycloocta[1,2,3-cd]inden-3-one |
| 56 | 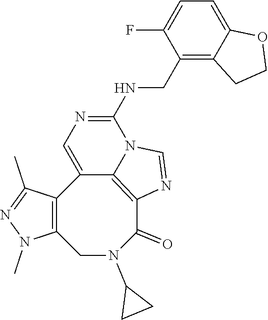 [SMILES: Cc1nn(C)c2c1-c1cnc(NCc3c(F)ccc4c3CCO4)n3cnc(c13)C(=O)N(C1CC1)C2] | 4-cyclopropyl-11-(((5-fluoro-2,3-dihydrobenzo- furan-4-yl)methyl)amino)-6,8-dimethyl-5,6-dihydro- 2,4,6,7,10,11a-hexaazacyclopenta[4,5]cycloocta [1,2,3-cd]inden-3(4H)-one |
| 57 | 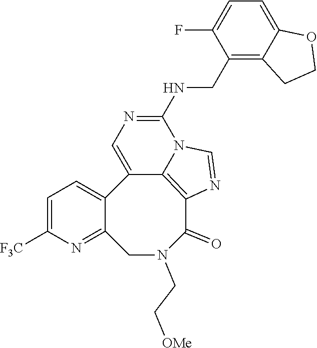 [SMILES: COCCN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4-yl)methyl) amino)-4-(2-methoxyethyl)-7-(trifluoromethyl)-4,5- dihydro-3H-2,4,6,11,12a-pentaazabenzo[4,5] cycloocta[1,2,3-cd]inden-3-one |
| 58 | 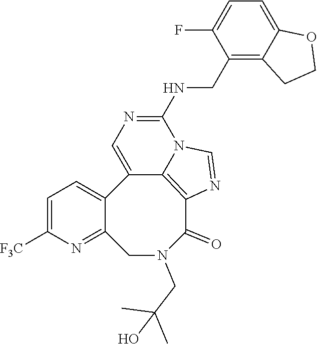 [SMILES: CC(C)(O)CN1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4-yl)methyl) amino)-4-(2-hydroxy-2-methylpropyl)-7-(trifluoro- methyl)-4,5-dihydro-3H-2,4,6,11,12a-pentaazabenzo [4,5]cycloocta[1,2,3-cd]inden-3-one |
| 59 | 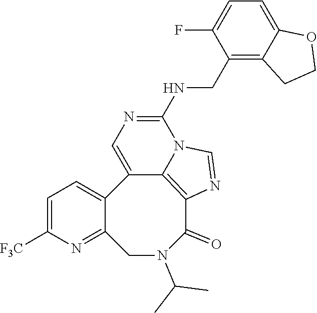 [SMILES: CC(C)N1Cc2nc(C(F)(F)F)ccc2-c2cnc(NCc3c(F)ccc4c3CCO4)n3cnc(c23)C1=O] | 12-(((5-fluoro-2,3-dihydrobenzofuran-4-yl)methyl) amino)-4-isopropyl-7-(trifluoromethyl)-4,5-dihydro- 3H-2,4,6,11,12a-pentaazabenzo[4,5]cycloocta [1,2,3-cd]inden-3-one |
| 60 | 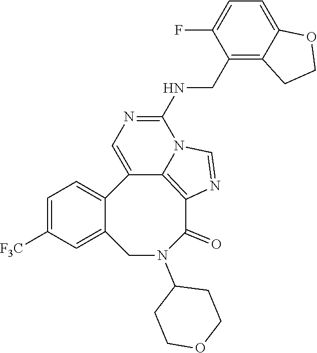 [SMILES: O=C1c2ncn3c(NCc4c(F)ccc5c4CCO5)ncc(c23)-c2ccc(C(F)(F)F)cc2CN1C1CCOCC1] | 12-(((5-fluoro-2,3-dihydrobenzofuran-4-yl)methyl) amino)-4-(tetrahydro-2H-pyran-4-yl)-7-(trifluoro methyl)-4,5-dihydro-3H-2,4,11,12a-tetraazabenzo [4,5]cycloocta[1,2,3-cd]inden-3-one |
| 61 | 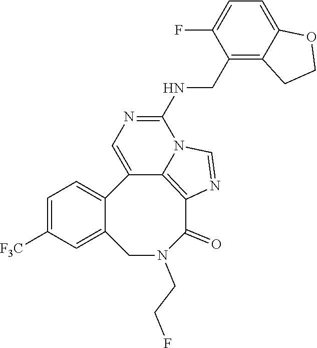 [SMILES: O=C1c2ncn3c(NCc4c(F)ccc5c4CCO5)ncc(c23)-c2ccc(C(F)(F)F)cc2CN1CCF] | 12-(((5-fluoro-2,3-dihydrobenzofuran-4-yl)methyl) amino)-4-(2-fluoroethyl)-7-(trifluoromethyl)-4,5- dihydro-3H-2,4,11,12a-tetraazabenzo[4,5] cycloocta[1,2,3-cd]inden-3-one |
| 62 | 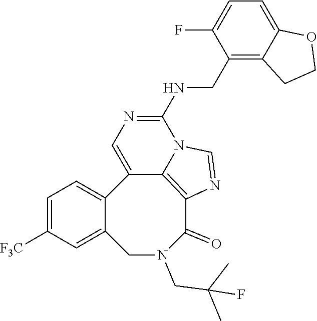 [SMILES: CC(C)(F)CN1Cc2cc(C(F)(F)F)ccc2-c2cnc(NCc3c(F)ccc4c3CCO4)n3cnc(c23)C1=O] | 12-(((5-fluoro-2,3-dihydrobenzofuran-4-yl)methyl) amino)-4-(2-fluoro-2-methylpropyl)-7-(trifluoro methyl)-4,5-dihydro-3H-2,4, 11,12a-tetraazabenzo [4,5]cycloocta[1,2,3-cd]inden-3-one |
| 63 | 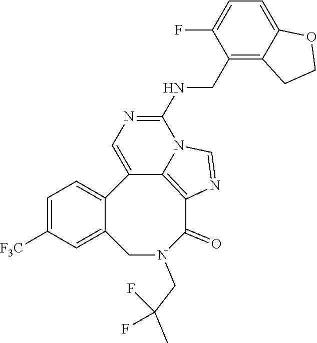 [SMILES: CC(F)(F)CN1Cc2cc(C(F)(F)F)ccc2-c2cnc(NCc3c(F)ccc4c3CCO4)n3cnc(c23)C1=O] | 4-(2,2-difluoropropyl)-12-(((5-fluoro-2,3-dihydro benzofuran-4-yl)methyl)amino)-7-(trifluoromethyl)- 4,5-dihydro-3H-2,4,11,12a-tetraazabenzo[4,5] cycloocta[1,2,3-cd]inden-3-one |
| 64 | 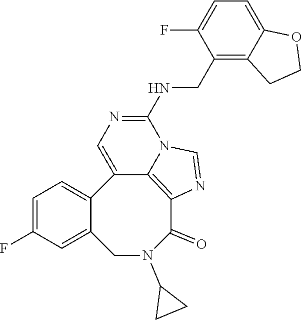 [SMILES: O=C1c2ncn3c(NCc4c(F)ccc5c4CCO5)ncc(c23)-c2ccc(F)cc2CN1C1CC1] | 4-cyclopropyl-7-fluoro-12-(((5-fluoro-2,3-dihydro benzofuran-4-yl)methyl)amino)-4,5-dihydro-3H- 2,4,11,12a-tetraazabenzo[4,5]cycloocta[1,2,3-cd] inden-3-one |
| 65 | 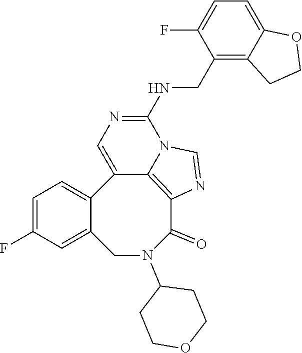 [SMILES: O=C1c2ncn3c(NCc4c(F)ccc5c4CCO5)ncc(c23)-c2ccc(F)cc2CN1C1CCOCC1] | 7-fluoro-12-(((5-fluoro-2,3-dihydrobenzofuran-4- yl)methyl)amino)-4-(tetrahydro-2H-pyran-4-yl)- 4,5-dihydro-3H-2,4,11,12a-tetraazabenzo[4,5] cycloocta[1,2,3-cd]inden-3-one |
| 66 | 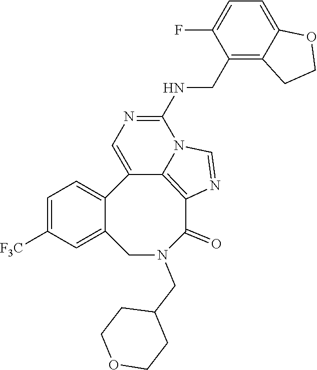 [SMILES: O=C1c2ncn3c(NCc4c(F)ccc5c4CCO5)ncc(c23)-c2ccc(C(F)(F)F)cc2CN1CC1CCOCC1] | 12-(((5-fluoro-2,3-dihydrobenzofuran-4-yl)methyl) amino)-4-((tetrahydro-2H-pyran-4-yl)methyl)-7- (trifluoromethyl)-4,5-dihydro-3H-2,4,11,12a-tetra- azabenzo[4,5]cycloocta[1,2,3-cd]inden-3-one |
| 67 | 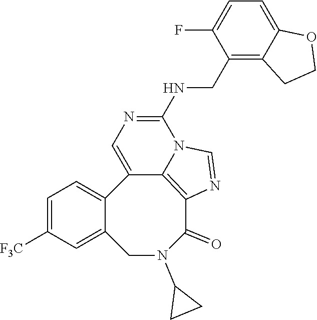 [SMILES: O=C1c2ncn3c(NCc4c(F)ccc5c4CCO5)ncc(c23)-c2ccc(C(F)(F)F)cc2CN1C1CC1] | 4-cyclopropyl-12-(((5-fluoro-2,3-dihydrobenzo- furan-4-yl)methyl)amino)-7-(trifluoromethyl)-4,5- dihydro-3H-2,4,11,12a-tetraazabenzo[4,5]cycloocta [1,2,3-cd]inden-3-one |
| 68 | 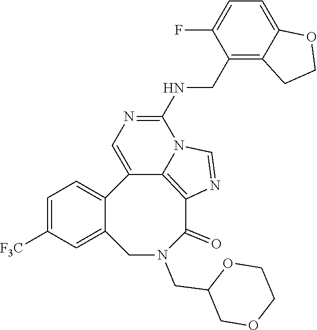 [SMILES: O=C1c2ncn3c(NCc4c(F)ccc5c4CCO5)ncc(c23)-c2ccc(C(F)(F)F)cc2CN1CC1COCCO1] | 4-((1,4-dioxan-2-yl)methyl)-12-(((5-fluoro-2,3- dihydrobenzofuran-4-yl)methyl)amino)-7-(trifluoro- methyl)-4,5-dihydro-3H-2,4,11,12a-tetraazabenzo [4,5]cycloocta[1,2,3-cd]inden-3-one |
| 69 | 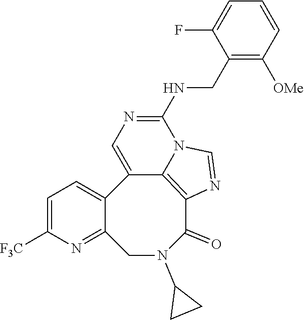 [SMILES: COc1cccc(F)c1CNc1ncc2c3c(ncn13)C(=O)N(C1CC1)Cc1nc(C(F)(F)F)ccc1-2] | 4-cyclopropyl-12-((2-fluoro-6-methoxybenzyl) amino)-7-(trifluoromethyl)-4,5-dihydro-3H-2,4,6, 11,12a-pentaazabenzo[4,5]cycloocta[1,2,3-cd] inden-3-one |
| 70 | 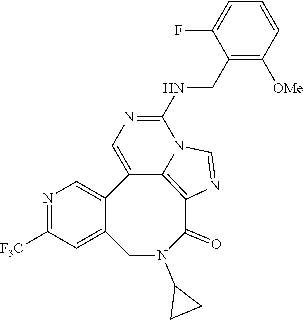 [SMILES: COc1cccc(F)c1CNc1ncc2c3c(ncn13)C(=O)N(C1CC1)Cc1cc(C(F)(F)F)ncc1-2] | 4-cyclopropyl-12-((2-fluoro-6-methoxybenzyl) amino)-7-(trifluoromethyl)-4,5-dihydro-3H-2,4,8, 11,12a-pentaazabenzo[4,5]cycloocta[1,2,3-cd] inden-3-one |
| 71 | 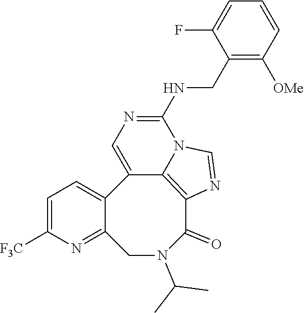 [SMILES: COc1cccc(F)c1CNc1ncc2c3c(ncn13)C(=O)N(C(C)C)Cc1nc(C(F)(F)F)ccc1-2] | 12-((2-fluoro-6-methoxybenzyl)amino)-4-isopropyl- 7-(trifluoromethyl)-4,5-dihydro-3H-2,4,6,11,12a- pentaazabenzo[4,5]cycloocta[1,2,3-cd]inden-3-one |
| 72 | 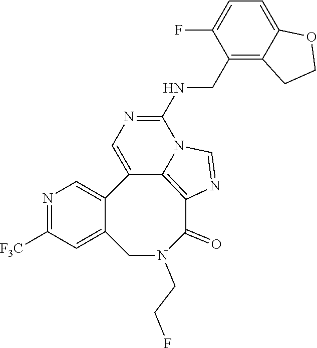 [SMILES: O=C1c2ncn3c(NCc4c(F)ccc5c4CCO5)ncc(c23)-c2cnc(C(F)(F)F)cc2CN1CCF] | 12-(((5-fluoro-2,3-dihydrobenzofuran-4-yl)methyl) amino)-4-(2-fluoroethyl)-7-(trifluoromethyl)-4,5- dihydro-3H-2,4,8,11,12a-pentaazabenzo[4,5] cycloocta[1,2,3-cd]inden-3-one |
| 73 | 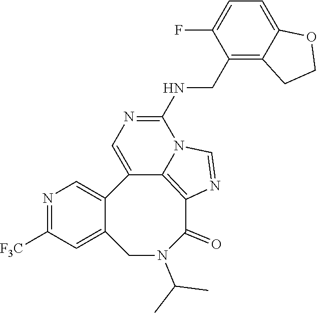 [SMILES: CC(C)N1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4-yl)methyl) amino)-4-isopropyl-7-(trifluoromethyl)-4,5-dihydro- 3H-2,4,8,11,12a-pentaazabenzo[4,5]cycloocta [1,2,3-cd]inden-3-one |
| 74 | 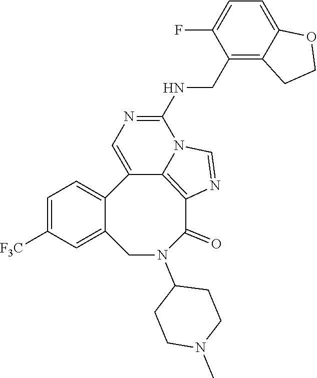 [SMILES: CN1CCC(N2Cc3cc(C(F)(F)F)ccc3-c3cnc(NCc4c(F)ccc5c4CCO5)n4cnc(c34)C2=O)CC1] | 12-(((5-fluoro-2,3-dihydrobenzofuran-4-yl)methyl) amino)-4-(1-methylpiperidin-4-yl)-7-(trifluoro- methyl)-4,5-dihydro-3H-2,4,11,12a-tetraazabenzo [4,5]cycloocta[1,2,3-cd]inden-3-one |
| 75 | 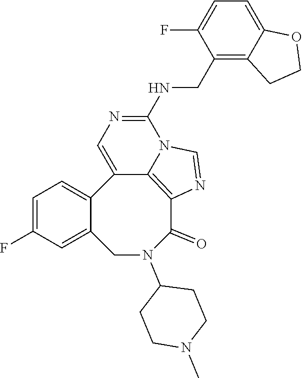 [SMILES: CN1CCC(N2Cc3cc(F)ccc3-c3cnc(NCc4c(F)ccc5c4CCO5)n4cnc(c34)C2=O)CC1] | 7-fluoro-12-(((5-fluoro-2,3-dihydrobenzofuran-4- yl)methyl)amino)-4-(1-methylpiperidin-4-yl)-4,5- dihydro-3H-2,4,11,12a-tetraazabenzo[4,5] cycloocta[1,2,3-cd]inden-3-one |
| 76 | 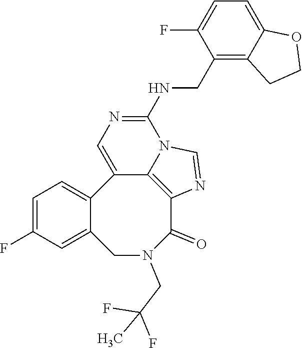 [SMILES: CC(F)(F)CN1Cc2cc(F)ccc2-c2cnc(NCc3c(F)ccc4c3CCO4)n3cnc(c23)C1=O] | 4-(2,2-difluoropropyl)-7-fluoro-12-(((5-fluoro-2,3- dihydrobenzofuran-4-yl)methyl)amino)-4,5- dihydro-3H-2,4,11,12a-tetraazabenzo[4,5]cycloocta [1,2,3-cd]inden-3-one |
| 77 | 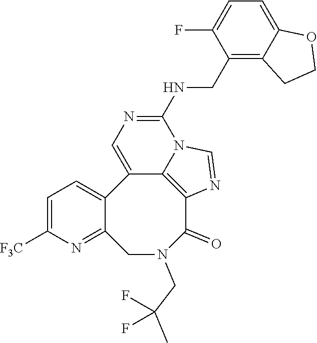 [SMILES: CC(F)(F)CN1Cc2nc(C(F)(F)F)ccc2-c2cnc(NCc3c(F)ccc4c3CCO4)n3cnc(c23)C1=O] | 4-(2,2-difluoropropyl)-12-(((5-fluoro-2,3-dihydro benzofuran-4-yl)methyl)amino)-7-(trifluoromethyl)- 4,5-dihydro-3H-2,4,6,11,12a-pentaazabenzo[4,5] cycloocta[1,2,3-cd]inden-3-one |
| 78 | 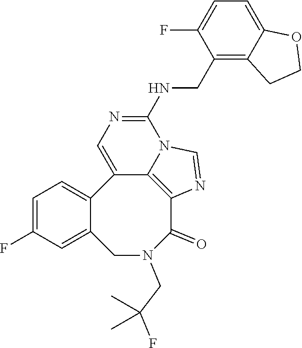 [SMILES: CC(C)(F)CN1Cc2cc(F)ccc2-c2cnc(NCc3c(F)ccc4c3CCO4)n3cnc(c23)C1=O] | 7-fluoro-12-(((5-fluoro-2,3-dihydrobenzofuran-4- yl)methyl)amino)-4-(2-fluoro-2-methylpropyl)-4,5- dihydro-3H-2,4,11,12a-tetraazabenzo[4,5] cycloocta[1,2,3-cd]inden-3-one |
| 79 | 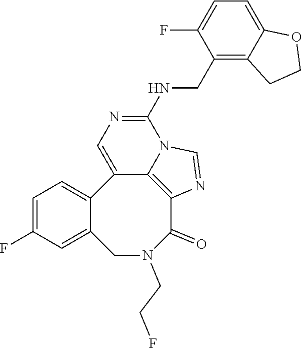 [SMILES: O=C1c2ncn3c(NCc4c(F)ccc5c4CCO5)ncc(c23)-c2ccc(F)cc2CN1CCF] | 7-fluoro-12-(((5-fluoro-2,3-dihydrobenzofuran-4- yl)methyl)amino)-4-(2-fluoroethyl)-4,5-dihydro- 3H-2,4,11,12a-tetraazabenzo[4,5]cycloocta [1,2,3-cd]inden-3-one |
| 80 | 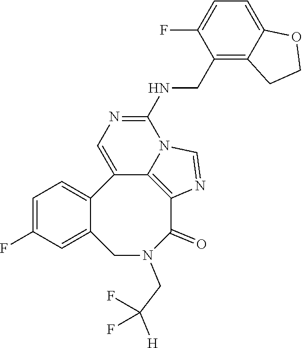 [SMILES: O=C1c2ncn3c(NCc4c(F)ccc5c4CCO5)ncc(c23)-c2ccc(F)cc2CN1CC(F)F] | 4-(2,2-difluoroethyl)-7-fluoro-12-(((5-fluoro-2,3- dihydrobenzofuran-4-yl)methyl)amino)-4,5-dihydro- 3H-2,4,11,12a-tetraazabenzo[4,5]cycloocta[1,2, 3-cd]inden-3-one |
| 81 | 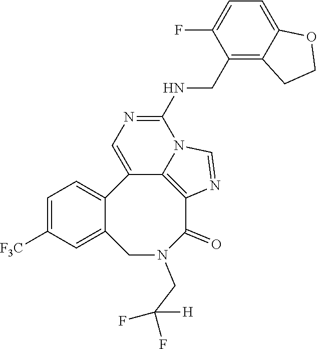 [SMILES: O=C1c2ncn3c(NCc4c(F)ccc5c4CCO5)ncc(c23)-c2ccc(C(F)(F)F)cc2CN1CC(F)F] | 4-(2,2-difluoroethyl)-12-(((5-fluoro-2,3-dihydro- benzofuran-4-yl)methyl)amino)-7-(trifluoromethyl)- 4,5-dihydro-3H-2,4,11,12a-tetraazabenzo[4,5] cycloocta[1,2,3-cd]inden-3-one |
| 82 | 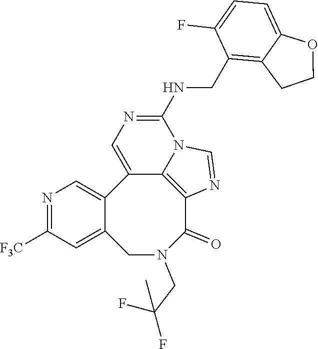 [SMILES: CC(F)(F)CN1Cc2cc(C(F)(F)F)ncc2-c2cnc(NCc3c(F)ccc4c3CCO4)n3cnc(c23)C1=O] | 4-(2,2-difluoropropyl)-12-(((5-fluoro-2,3-dihydro benzofuran-4-yl)methyl)amino)-7-(trifluoromethyl)- 4,5-dihydro-3H-2,4,8,11,12a-pentaazabenzo[4,5] cycloocta[1,2,3-cd]inden-3-one |
| 83 | 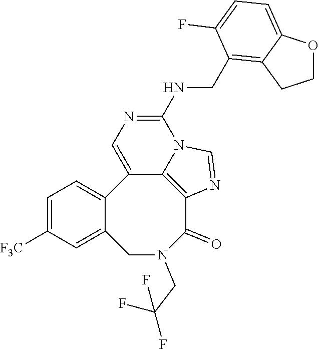 [SMILES: O=C1c2ncn3c(NCc4c(F)ccc5c4CCO5)ncc(c23)-c2ccc(C(F)(F)F)cc2CN1CC(F)(F)F] | 12-(((5-fluoro-2,3-dihydrobenzofuran-4-yl)methyl) amino)-4-(2,2,2-trifluoroethyl)-7-(trifluoromethyl)- 4,5-dihydro-3H-2,4,11,12a-tetraazabenzo[4,5] cycloocta[1,2,3-cd]inden-3-one |
| 84 | 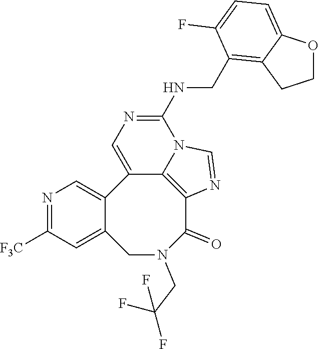 [SMILES: O=C1c2ncn3c(NCc4c(F)ccc5c4CCO5)ncc(c23)-c2cnc(C(F)(F)F)cc2CN1CC(F)(F)F] | 12-(((5-fluoro-2,3-dihydrobenzofuran-4-yl)methyl) amino)-4-(2,2,2-trifluoroethyl)-7-(trifluoromethyl)- 4,5-dihydro-3H-2,4,8,11,12a-pentaazabenzo[4,5] cycloocta[1,2,3-cd]inden-3-one |
| 85 | 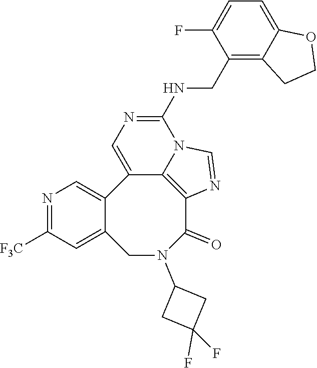 [SMILES: O=C1c2ncn3c(NCc4c(F)ccc5c4CCO5)ncc(c23)-c2cnc(C(F)(F)F)cc2CN1C1CC(F)(F)C1] | 4-(3,3-difluorocyclobutyl)-12-(((5-fluoro-2,3- dihydrobenzofuran-4-yl)methyl)amino)-7-(trifluoro- methyl)-4,5-dihydro-3H-2,4,8,11,12a-pentaazabenzo [4,5]cycloocta[1,2,3-cd]inden-3-one |
| 86 | 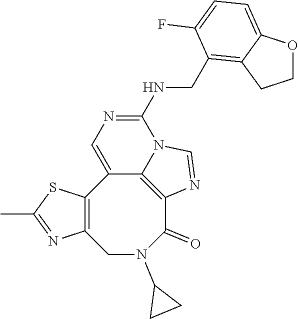 [SMILES: Cc1nc2c(s1)-c1cnc(NCc3c(F)ccc4c3CCO4)n3cnc(c13)C(=O)N(C1CC1)C2] | 4-cyclopropyl-11-(((5-fluoro-2,3-dihydrobenzofuran- 4-yl)methyl)amino)-7-methyl-4,5-dihydro-3H- 8-thia-2,4,6,10,11a-pentaazacyclopenta[4,5] cycloocta[1,2,3-cd]inden-3-one |
| 87 | 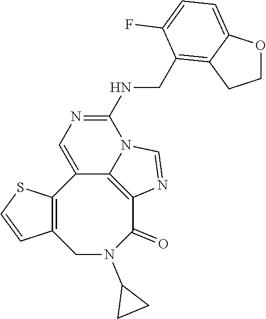 [SMILES: O=C1c2ncn3c(NCc4c(F)ccc5c4CCO5)ncc(c23)-c2sccc2CN1C1CC1] | 4-cyclopropyl-11-(((5-fluoro-2,3-dihydrobenzofuran- 4-yl)methyl)amino)-4,5-dihydro-3H-8-thia-2,4, 10,11a-tetraazacyclopenta[4,5]cycloocta[1,2,3-cd] inden-3-one |
| 88 | 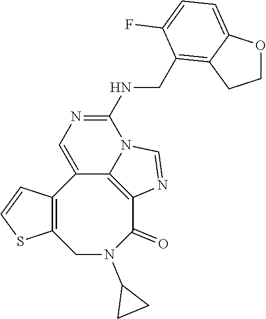 [SMILES: O=C1c2ncn3c(NCc4c(F)ccc5c4CCO5)ncc(c23)-c2ccsc2CN1C1CC1] | 4-cyclopropyl-11-(((5-fluoro-2,3-dihydrobenzofuran- 4-yl)methyl)amino)-4,5-dihydro-3H-6-thia-2,4, 10,11a-tetraazacyclopenta[4,5]cycloocta[1,2,3-cd] inden-3-one |
| 89 | 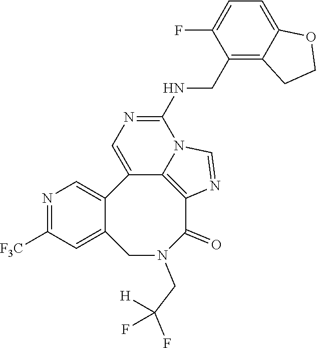 [SMILES: O=C1c2ncn3c(NCc4c(F)ccc5c4CCO5)ncc(c23)-c2cnc(C(F)(F)F)cc2CN1CC(F)F] | 4-(2,2-difluoroethyl)-12-(((5-fluoro-2,3-dihydro- benzofuran-4-yl)methyl)amino)-7-(trifluoromethyl)- 4,5-dihydro-3H-2,4,8,11,12a-pentaazabenzo[4,5] cycloocta[1,2,3-cd]inden-3-one |
| 90 | 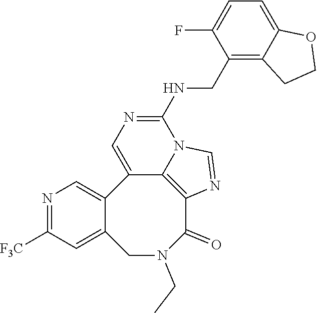 [SMILES: CCN1Cc2cc(C(F)(F)F)ncc2-c2cnc(NCc3c(F)ccc4c3CCO4)n3cnc(c23)C1=O] | 4-ethyl-12-(((5-fluoro-2,3-dihydrobenzofuran-4-yl) methyl)amino)-7-(trifluoromethyl)-4,5-dihydro-3H- 2,4,8,11,12a-pentaazabenzo[4,5]cycloocta [1,2,3-cd]inden-3-one |
| 91 | 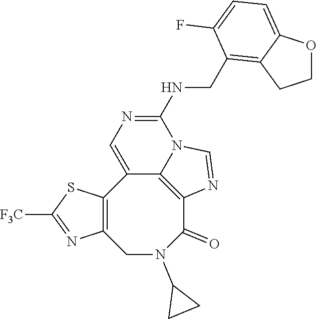 [SMILES: O=C1c2ncn3c(NCc4c(F)ccc5c4CCO5)ncc(c23)-c2sc(C(F)(F)F)nc2CN1C1CC1] | 4-cyclopropyl-11-(((5-fluoro-2,3-dihydrobenzofuran- 4-yl)methyl)amino)-7-(trifluoromethyl)-4,5- dihydro-3H-8-thia-2,4,6,10,11a-pentaazacyclopenta [4,5]cycloocta[1,2,3-cd]inden-3-one |
| 92 | 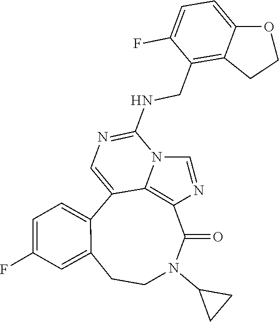 [SMILES: O=C1c2ncn3c(NCc4c(F)ccc5c4CCO5)ncc(c23)-c2ccc(F)cc2CCN1C1CC1] | 4-cyclopropyl-8-fluoro-13-(((5-fluoro-2,3-dihydro benzofuran-4-yl)methyl)amino)-5,6-dihydro-2,4, 12,13a-tetraazabenzo[4,5]cyclonona[1,2,3-cd] inden-3(4H)-one |
| 93 | 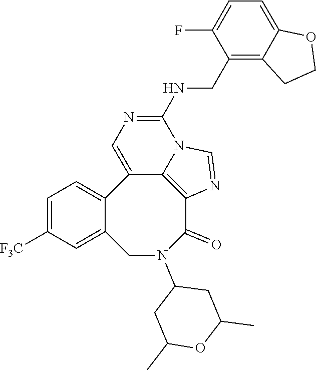 [SMILES: CC1CC(N2Cc3cc(C(F)(F)F)ccc3-c3cnc(NCc4c(F)ccc5c4CCO5)n4cnc(c34)C2=O)CC(C)O1] | 4-(2,6-dimethyltetrahydro-2H-pyran-4-yl)-12-(((5- fluoro-2,3-dihydrobenzofuran-4-yl)methyl)amino)- 7-(trifluoromethyl)-4,5-dihydro-3H-2,4,11,12a- tetraazabenzo[4,5]cycloocta[1,2,3-cd]inden-3-one |
| 94 | 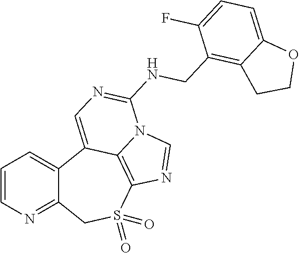 [SMILES: O=S1(=O)Cc2ncccc2-c2cnc(NCc3c(F)ccc4c3CCO4)n3cnc1c23] | 11-(((5-fluoro-2,3-dihydrobenzofuran-4-yl)methyl) amino)-4H-3-thia-2,5,10,11a-tetraazadibenzo [cd,f]azulene 3,3-dioxide |
| 95 | 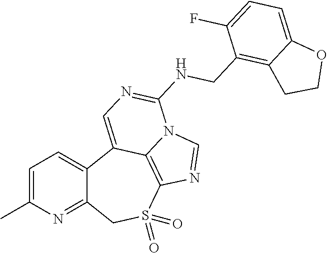 [SMILES: Cc1ccc2c(n1)CS(=O)(=O)c1ncn3c(NCc4c(F)ccc5c4CCO5)ncc-2c13] | 11-(((5-fluoro-2,3-dihydrobenzofuran-4-yl)methyl) amino)-6-methyl-4H-3-thia-2,5,10,11a-tetraaza- dibenzo[cd,f]azulene 3,3-dioxide |
| 96 | 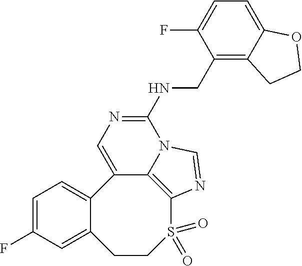 [SMILES: O=S1(=O)CCc2cc(F)ccc2-c2cnc(NCc3c(F)ccc4c3CCO4)n3cnc1c23] | 7-fluoro-12-(((5-fluoro-2,3-dihydrobenzofuran-4- yl)methyl)amino)-4,5-dihydro-3-thia-2,11,12a- triazabenzo[4,5]cycloocta[1,2,3-cd]indene 3,3-dioxide |
| 97 | 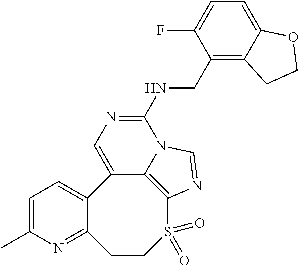 [SMILES: Cc1ccc2c(n1)CCS(=O)(=O)c1ncn3c(NCc4c(F)ccc5c4CCO5)ncc-2c13] | 12-(((5-fluoro-2,3-dihydrobenzofuran-4-yl)methyl) amino)-7-methyl-4,5-dihydro-3-thia-2,6,11,12a- tetraazabenzo[4,5]cycloocta[1,2,3-cd]indene 3,3-dioxide |
| 98 | 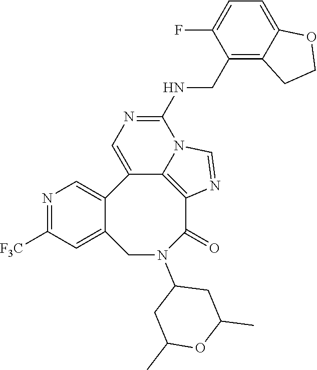 [SMILES: CC1CC(N2Cc3cc(C(F)(F)F)ncc3-c3cnc(NCc4c(F)ccc5c4CCO5)n4cnc(c34)C2=O)CC(C)O1] | 4-(2,6-dimethyltetrahydro-2H-pyran-4-yl)-12-(((5- fluoro-2,3-dihydrobenzofuran-4-yl)methyl)amino)- 7-(trifluoromethyl)-4,5-dihydro-3H-2,4,8,11,12a- pentaazabenzo[4,5]cycloocta[1,2,3-cd]inden-3-one |
| E1 | 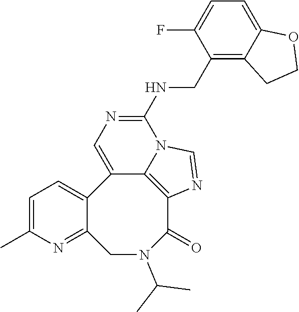 [SMILES: Cc1ccc2c(n1)CN(C(C)C)C(=O)c1ncn3c(NCc4c(F)ccc5c4CCO5)ncc-2c13] | 12-(((5-fluoro-2,3-dihydrobenzofuran-4-yl)methyl) amino)-4-isopropyl-7-methyl-4,5-dihydro-3H-2,4, 6,11,12a-pentaazabenzo[4,5]cycloocta[1,2,3-cd] inden-3-one |
| E2 | 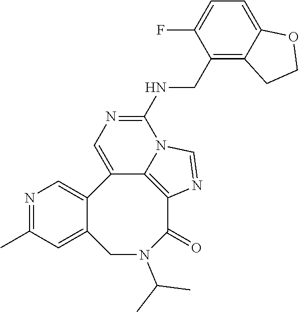 [SMILES: Cc1cc2c(cn1)-c1cnc(NCc3c(F)ccc4c3CCO4)n3cnc(c13)C(=O)N(C(C)C)C2] | 12-(((5-fluoro-2,3-dihydrobenzofuran-4-yl)methyl) amino)-4-isopropyl-7-methyl-4,5-dihydro-3H-2,4, 8,11,12a-pentaazabenzo[4,5]cycloocta[1,2,3-cd] inden-3-one |
| E3 | 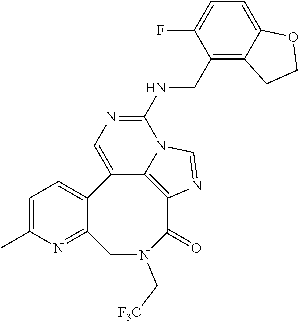 [SMILES: Cc1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4-yl)methyl) amino)-7-methyl-4-(2,2,2-trifluoroethyl)-4,5- dihydro-3H-2,4,6,11, 12a-pentaazabenzo[4,5] cycloocta[1,2,3-cd]inden-3-one |
| E4 | 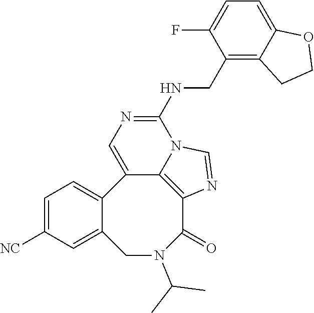 [SMILES: CC(C)N1Cc2cc(C#N)ccc2-c2cnc(NCc3c(F)ccc4c3CCO4)n3cnc(c23)C1=O] | 12-(((5-fluoro-2,3-dihydrobenzofuran-4-yl)methyl) amino)-4-isopropyl-3-oxo-4,5-dihydro-3H-2,4,11, 12a-tetraazabenzo[4,5]cycloocta[1,2,3-cd]indene- 7-carbonitrile |
| E5 | 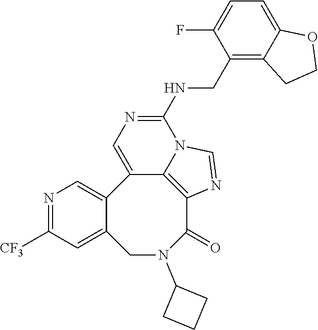 [SMILES: O=C1c2ncn3c(NCc4c(F)ccc5c4CCO5)ncc(c23)-c2cnc(C(F)(F)F)cc2CN1C1CCC1] | 4-cyclobutyl-12-(((5-fluoro-2,3-dihydrobenzofuran- 4-yl)methyl)amino)-7-(trifluoromethyl)-4,5- dihydro-3H-2,4,8,11,12a-pentaazabenzo[4,5] cycloocta[1,2,3-cd]inden-3-one |
| E6 | 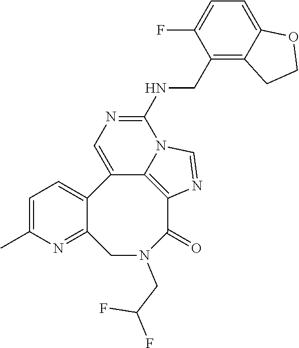 [SMILES: Cc1ccc2c(n1)CN(CC(F)F)C(=O)c1ncn3c(NCc4c(F)ccc5c4CCO5)ncc-2c13] | 4-(2,2-difluoroethyl)-12-(((5-fluoro-2,3-dihydro- benzofuran-4-yl)methyl)amino)-7-methyl-4,5- dihydro-3H-2,4,6,11,12a-pentaazabenzo[4,5] cycloocta[1,2,3-cd]inden-3-one |
| E7 | 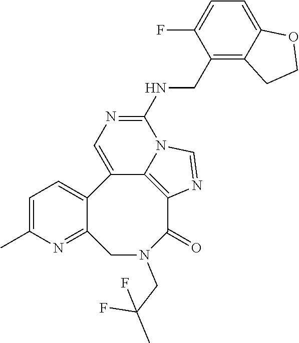 [SMILES: Cc1ccc2c(n1)CN(CC(C)(F)F)C(=O)c1ncn3c(NCc4c(F)ccc5c4CCO5)ncc-2c13] | 4-(2,2-difluoropropyl)-12-(((5-fluoro-2,3-dihydro benzofuran-4-yl)methyl)amino)-7-methyl-4,5- dihydro-3H-2,4,6,11,12a-pentaazabenzo[4,5] cycloocta[1,2,3-cd]inden-3-one |
| E8 | 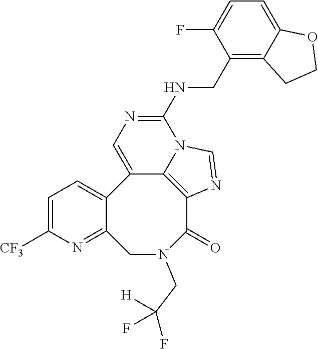 [SMILES: O=C1c2ncn3c(NCc4c(F)ccc5c4CCO5)ncc(c23)-c2ccc(C(F)(F)F)nc2CN1CC(F)F] | 4-(2,2-difluoroethyl)-12-(((5-fluoro-2,3-dihydro- benzofuran-4-yl)methyl)amino)-7-(trifluoromethyl)- 4,5-dihydro-3H-2,4,6, 11,12a-pentaazabenzo[4,5] cycloocta[1,2,3-cd]inden-3-one |
| E9 | 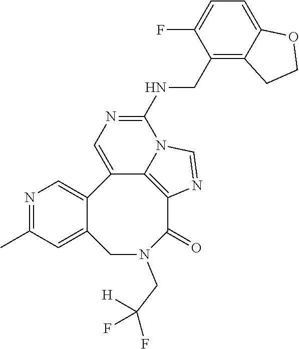 [SMILES: Cc1cc2c(cn1)-c1cnc(NCc3c(F)ccc4c3CCO4)n3cnc(c13)C(=O)N(CC(F)F)C2] | 4-(2,2-difluoroethyl)-12-(((5-fluoro-2,3-dihydro- benzofuran-4-yl)methyl)amino)-7-methyl-4,5- dihydro-3H-2,4,8,11,12a-pentaazabenzo[4,5] cycloocta[1,2,3-cd]inden-3-one |
| E10 | 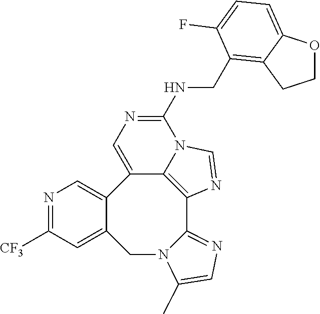 [SMILES: Cc1cnc2n1Cc1cc(C(F)(F)F)ncc1-c1cnc(NCc3c(F)ccc4c3CCO4)n3cnc-2c13] | N-((5-fluoro-2,3-dihydrobenzofuran-4-yl)methyl)- 5-methyl-8-(trifluoromethyl)-6H-2,3,5a,9,12,13a- hexaazabenzo[4,5]cyclopenta[7,8]cycloocta[1,2,3- cd]inden-13-amine |
| E11 | 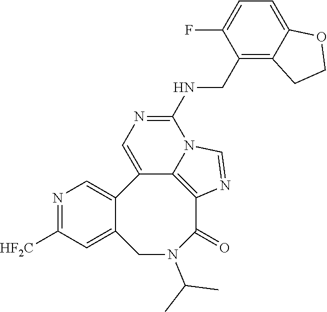 | 7-(difluoromethyl)-12-(((5-fluoro-2,3-dihydro- benzofuran-4-yl)methyl)amino)-4-isopropyl-4,5- dihydro-3H-2,4,8,11,12a-pentaazabenzo[4,5] cycloocta[1,2,3-cd]inden-3-one |
| E12 | 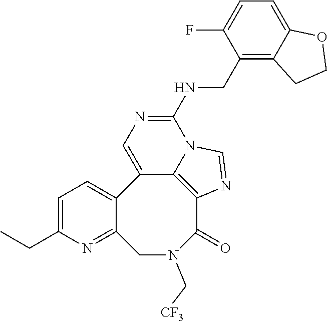 [SMILES: CCc1ccc2c(n1)CN(CC(F)(F)F)C(=O)c1ncn3c(NCc4c(F)ccc5c4CCO5)ncc-2c13] | 7-ethyl-12-(((5-fluoro-2,3-dihydrobenzofuran-4-yl) methyl)amino)-4-(2,2,2-trifluoroethyl)-4,5-dihydro- 3H-2,4,6,11,12a-pentaazabenzo[4,5]cycloocta [1,2,3-cd]inden-3-one |
| E13 | 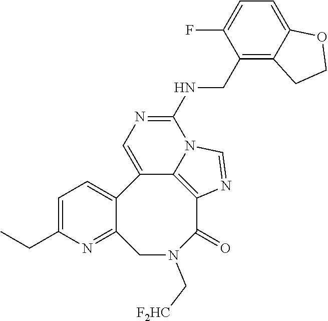 [SMILES: CCc1ccc2c(n1)CN(CC(F)F)C(=O)c1ncn3c(NCc4c(F)ccc5c4CCO5)ncc-2c13] | 4-(2,2-difluoroethyl)-7-ethyl-12-(((5-fluoro-2,3- dihydrobenzofuran-4-yl)methyl)amino)-4,5-dihydro- 3H-2,4,6,11,12a-pentaazabenzo[4,5]cycloocta [1,2,3-cd]inden-3-one |
| E14 | 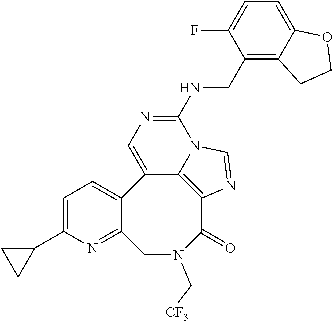 [SMILES: O=C1c2ncn3c(NCc4c(F)ccc5c4CCO5)ncc(c23)-c2ccc(C3CC3)nc2CN1CC(F)(F)F] | 7-cyclopropyl-12-(((5-fluoro-2,3-dihydrobenzo- furan-4-yl)methyl)amino)-4-(2,2,2-trifluoroethyl)- 4,5-dihydro-3H-2,4,6,11,12a-pentaazabenzo[4,5] cycloocta[1,2,3-cd]inden-3-one |
| E15 | 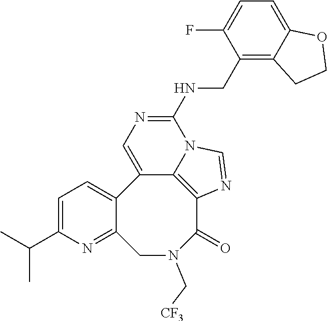 [SMILES: CC(C)c1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4-yl)methyl) amino)-7-isopropyl-4-(2,2,2-trifluoroethyl)-4,5- dihydro-3H-2,4,6,11,12a-pentaazabenzo[4,5] cycloocta[1,2,3-cd]inden-3-one |
| E16 | 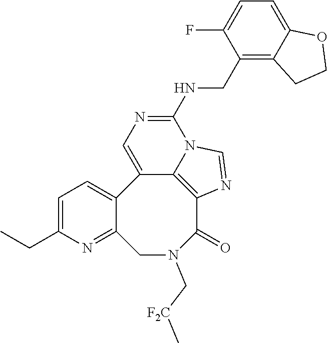 [SMILES: CCc1ccc2c(n1)CN(CC(C)(F)F)C(=O)c1ncn3c(NCc4c(F)ccc5c4CCO5)ncc-2c13] | 4-(2,2-difluoropropyl)-7-ethyl-12-(((5-fluoro-2,3- dihydrobenzofuran-4-yl)methyl)amino)-4,5-dihydro- 3H-2,4,6,11,12a-pentaazabenzo[4,5]cycloocta [1,2,3-cd]inden-3-one |
| E17 | 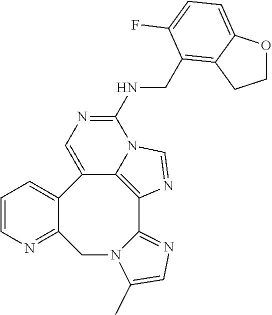 [SMILES: Cc1cnc2n1Cc1ncccc1-c1cnc(NCc3c(F)ccc4c3CCO4)n3cnc-2c13] | N-((5-fluoro-2,3-dihydrobenzofuran-4-yl)methyl)- 5-methyl-6H-2,3,5a,7,12,13a-hexaazabenzo[4,5] cyclopenta[7,8]cycloocta[1,2,3-cd]inden-13-amine |
| E18 | 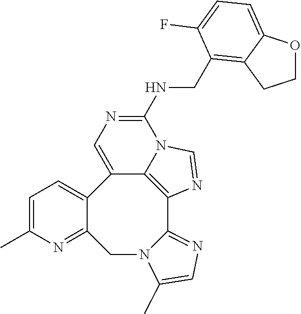 [SMILES: Cc1ccc2c(n1)Cn1c(C)cnc1-c1ncn3c(NCc4c(F)ccc5c4CCO5)ncc-2c13] | N-((5-fluoro-2,3-dihydrobenzofuran-4-yl)methyl)- 5,8-dimethyl-6H-2,3,5a,7,12,13a-hexaazabenzo[4, 5]cyclopenta[7,8]cycloocta[1,2,3-cd]inden-13- amine |
| E19 | 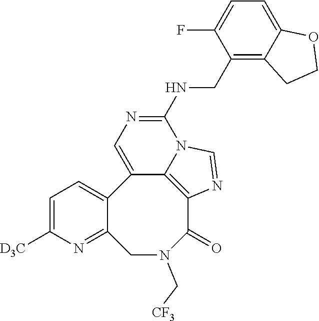 [SMILES: [2H]C([2H])([2H])c1ccc2c(n1)CN(CC(F)(F)F)C(=O)c1ncn3c(NCc4c(F)ccc5c4CCO5)ncc-2c13] | 12-(((5-fluoro-2,3-dihydrobenzofuran-4-yl)methyl) amino)-7-(methyl-d3)-4-(2,2,2-trifluoroethyl)-4,5- dihydro-3H-2,4,6,11,12a-pentaazabenzo[4,5] cycloocta[1,2,3-cd]inden-3-one |
| E20 | 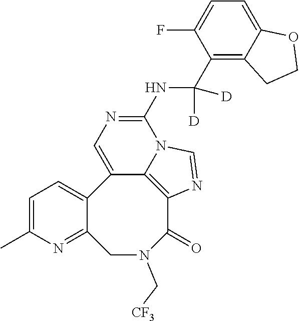 [SMILES: [2H]C([2H])(Nc1ncc2c3c(ncn13)C(=O)N(CC(F)(F)F)Cc1nc(C)ccc1-2)c1c(F)ccc2c1CCO2] | 12-(((5-fluoro-2,3-dihydrobenzofuran-4-yl)methyl- d2)amino)-7-methyl-4-(2,2,2-trifluoroethyl)-4,5- dihydro-3H-2,4,6,11,12a-pentaazabenzo[4,5] cycloocta[1,2,3-cd]inden-3-one |
| E21 | 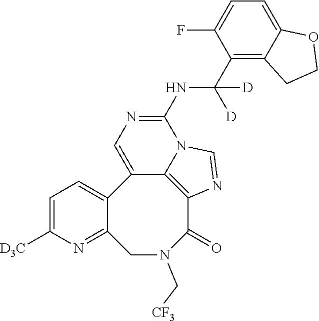 [SMILES: [2H]C([2H])([2H])c1ccc2c(n1)CN(CC(F)(F)F)C(=O)c1ncn3c(NC([2H])([2H])c4c(F)ccc5c4CCO5)ncc-2c13] | 12-(((5-fluoro-2,3-dihydrobenzofuran-4-yl)methyl- d2)amino)-7-(methyl-d3)-4-(2,2,2-trifluoroethyl)- 4,5-dihydro-3H-2,4,6, 11,12a-pentaazabenzo[4,5] cycloocta[1,2,3-cd]inden-3-one |
| E22 | 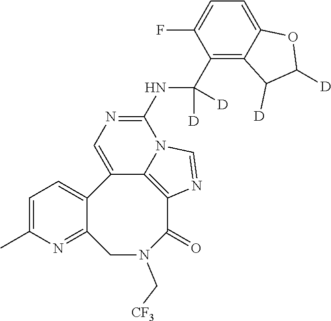 [SMILES: [2H]C1Oc2ccc(F)c(C([2H])([2H])Nc3ncc4c5c(ncn35)C(=O)N(CC(F)(F)F)Cc3nc(C)ccc3-4)c2C1[2H]] | 12-(((5-fluoro-2,3-dihydrobenzofuran-4-yl-2,3-d2) methyl-d2)amino)-7-methyl-4-(2,2,2-trifluoroethyl)- 4,5-dihydro-3H-2,4,6, 11,12a-pentaazabenzo[4,5] cycloocta[1,2,3-cd]inden-3-one |
| E23 | 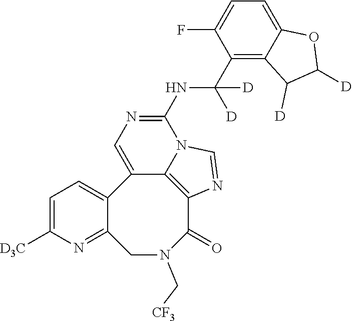 [SMILES: [2H]C1Oc2ccc(F)c(C([2H])([2H])Nc3ncc4c5c(ncn35)C(=O)N(CC(F)(F)F)Cc3nc(C([2H])([2H])[2H])ccc3-4)c2C1[2H]] | 12-(((5-fluoro-2,3-dihydrobenzofuran-4-yl-2,3-d2) methyl-d2)amino)-7-(methyl-d3)-4-(2,2,2-trifluoro- ethyl)-4,5-dihydro-3H-2,4,6,11,12a-pentaaza- benzo[4,5]cycloocta[1,2,3-cd]inden-3-one |
| E24 | 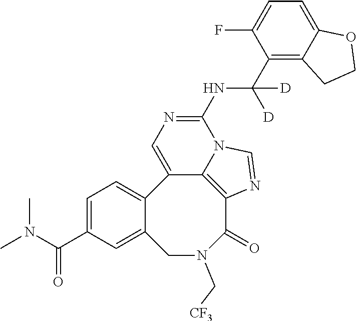 [SMILES: [2H]C([2H])(Nc1ncc2c3c(ncn13)C(=O)N(CC(F)(F)F)Cc1cc(C(=O)N(C)C)ccc1-2)c1c(F)ccc2c1CCO2] | 12-(((5-fluoro-2,3-dihydrobenzofuran-4-yl)methyl- d2)amino)-N,N-dimethyl-3-oxo-4-(2,2,2-trifluoro ethyl)-4,5-dihydro-3H-2,4,11,12a-tetraazabenzo[4, 5]cycloocta[1,2,3-cd]indene-7-carboxamide |
| E25 | 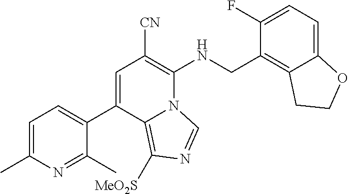 [SMILES: Cc1ccc(-c2cc(C#N)c(NCc3c(F)ccc4c3CCO4)n3cnc(S(C)(=O)=O)c23)c(C)n1] | 8-(2,6-dimethylpyridin-3-yl)-5-(((5-fluoro-2,3- dihydrobenzofuran-4-yl)methyl)amino)-1- (methylsulfonyl)imidazo[1,5-a]pyridine-6- carbonitrile |
| E26 | 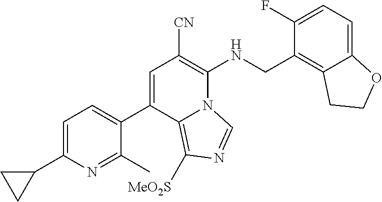 [SMILES: Cc1nc(C2CC2)ccc1-c1cc(C#N)c(NCc2c(F)ccc3c2CCO3)n2cnc(S(C)(=O)=O)c12] | 8-(6-cyclopropylpyridin-3-yl)-5-(((5-fluoro-2,3- dihydrobenzofuran-4-yl)methyl)amino)-1- (methylsulfonyl)imidazo[1,5-a]pyridine-6- carbonitrile |
| E27 | 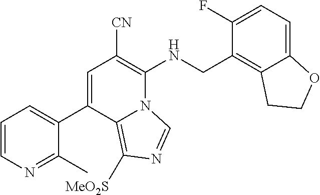 [SMILES: Cc1ncccc1-c1cc(C#N)c(NCc2c(F)ccc3c2CCO3)n2cnc(S(C)(=O)=O)c12] | 5-(((5-fluoro-2,3-dihydrobenzofuran-4-yl)methyl) amino)-8-(2-methylpyridin-3-yl)-1-(methylsulfonyl) imidazo[1,5-a]pyridine-6-carbonitrile |
| E28 | 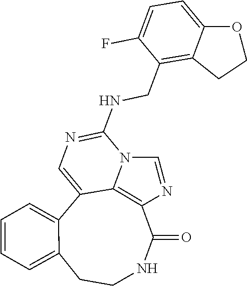 [SMILES: O=C1NCCc2ccccc2-c2cnc(NCc3c(F)ccc4c3CCO4)n3cnc1c23] | 13-(((5-fluoro-2,3-dihydrobenzofuran-4-yl)methyl) amino)-5,6-dihydro-2,4, 12, 13a-tetraazabenzo[4,5] cyclonona[1,2,3-cd]inden-3(4H)-one |
| E29 | 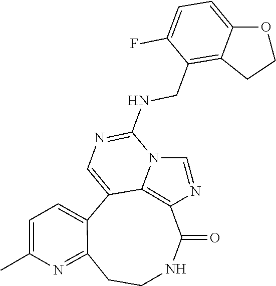 [SMILES: Cc1ccc2c(n1)CCNC(=O)c1ncn3c(NCc4c(F)ccc5c4CCO5)ncc-2c13] | 13-(((5-fluoro-2,3-dihydrobenzofuran-4-yl)methyl) amino)-8-methyl-5,6-dihydro-2,4,7,12,13a- pentaazabenzo[4,5]cyclonona[1,2,3-cd]inden- 3(4H)-one |
| E30 | 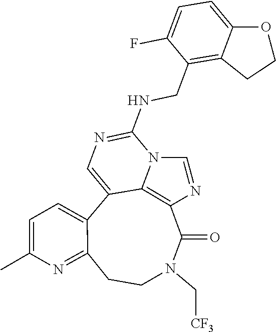 [SMILES: Cc1ccc2c(n1)CCN(CC(F)(F)F)C(=O)c1ncn3c(NCc4c(F)ccc5c4CCO5)ncc-2c13] | 13-(((5-fluoro-2,3-dihydrobenzofuran-4-yl)methyl) amino)-8-methyl-4-(2,2,2-trifluoroethyl)-5,6- dihydro-2,4,7,12,13a-pentaazabenzo[4,5] cyclonona[1,2,3-cd]inden-3(4H)-one |
| E31 | 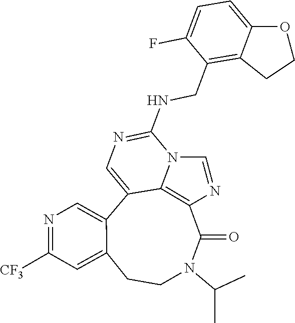 [SMILES: CC(C)N1CCc2cc(C(F)(F)F)ncc2-c2cnc(NCc3c(F)ccc4c3CCO4)n3cnc(c23)C1=O] | 13-(((5-fluoro-2,3-dihydrobenzofuran-4-yl)methyl) amino)-4-isopropyl-8-(trifluoromethyl)-5,6-dihydro- 2,4,9,12,13a-pentaazabenzo[4,5]cyclonona[1,2, 3-cd]inden-3(4H)-one |
| E32 | 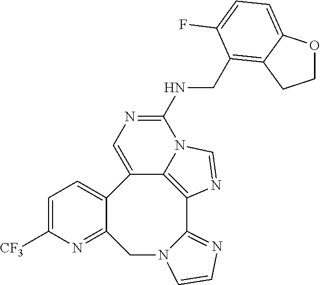 [SMILES: Fc1ccc2c(c1CNc1ncc3c4c(ncn14)-c1nccn1Cc1nc(C(F)(F)F)ccc1-3)CCO2] | N-((5-fluoro-2,3-dihydrobenzofuran-4-yl)methyl)- 8-(trifluoromethyl)-6H-2,3,5a,7,12,13a-hexaaza- benzo[4,5]cyclopenta[7,8]cycloocta[1,2,3-cd]inden- 13-amine |
| E33 | 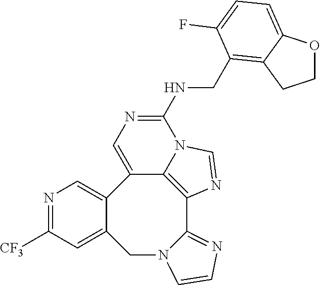 [SMILES: Fc1ccc2c(c1CNc1ncc3c4c(ncn14)-c1nccn1Cc1cc(C(F)(F)F)ncc1-3)CCO2] | N-((5-fluoro-2,3-dihydrobenzofuran-4-yl)methyl)- 8-(trifluoromethyl)-6H-2,3,5a,9,12,13a-hexaaza- benzo[4,5]cyclopenta[7,8]cycloocta[1,2,3-cd]inden- 13-amine |
| E34 | 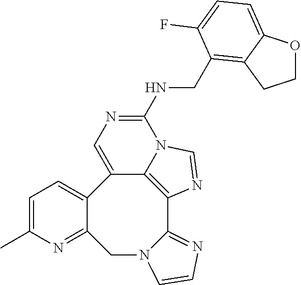 [SMILES: Cc1ccc2c(n1)Cn1ccnc1-c1ncn3c(NCc4c(F)ccc5c4CCO5)ncc-2c13] | N-((5-fluoro-2,3-dihydrobenzofuran-4-yl)methyl)- 8-methyl-6H-2,3,5a,7,12,13a-hexaazabenzo[4,5] cyclopenta[7,8]cycloocta[1,2,3-cd]inden-13-amine |
| E35 | 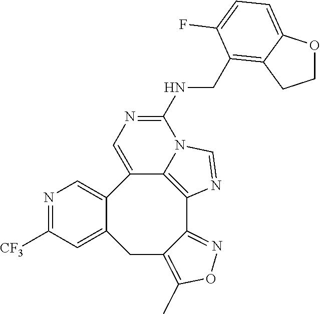 [SMILES: Cc1onc2c1Cc1cc(C(F)(F)F)ncc1-c1cnc(NCc3c(F)ccc4c3CCO4)n3cnc-2c13] | N-((5-fluoro-2,3-dihydrobenzofuran-4-yl)methyl)- 5-methyl-8-(trifluoromethyl)-6H-4-oxa-2,3,9,12, 13a-pentaazabenzo[4,5]cyclopenta[7,8]cycloocta [1,2,3-cd]inden-13-amine |
| E36 | 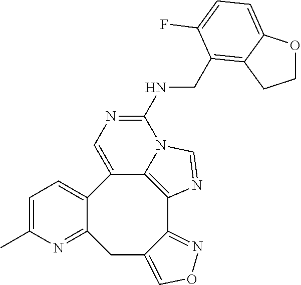 [SMILES: Cc1ccc2c(n1)Cc1conc1-c1ncn3c(NCc4c(F)ccc5c4CCO5)ncc-2c13] | N-((5-fluoro-2,3-dihydrobenzofuran-4-yl)methyl)- 8-methyl-6H-4-oxa-2,3,7,12,13a-pentaazabenzo [4,5]cyclopenta[7,8]cycloocta[1,2,3-cd]inden-13- amine |
| E37 | 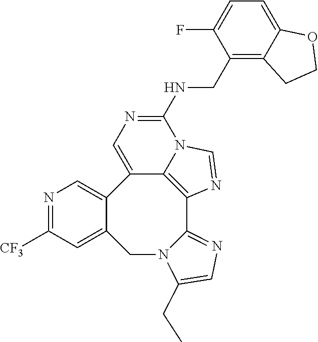 [SMILES: CCc1cnc2n1Cc1cc(C(F)(F)F)ncc1-c1cnc(NCc3c(F)ccc4c3CCO4)n3cnc-2c13] | 5-ethyl-N-((5-fluoro-2,3-dihydrobenzofuran-4-yl) methyl)-8-(trifluoromethyl)-6H-2,3,5a,9,12,13a- hexaazabenzo[4,5]cyclopenta[7,8]cycloocta[1,2,3- cd]inden-13-amine |
| E38 | 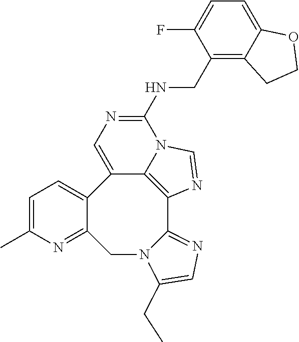 [SMILES: CCc1cnc2n1Cc1nc(C)ccc1-c1cnc(NCc3c(F)ccc4c3CCO4)n3cnc-2c13] | 5-ethyl-N-((5-fluoro-2,3-dihydrobenzofuran-4-yl) methyl)-8-methyl-6H-2,3,5a,7,12,13a-hexaazabenzo [4,5]cyclopenta[7,8]cycloocta[1,2,3-cd]inden-1 3-amine |
| E39 | 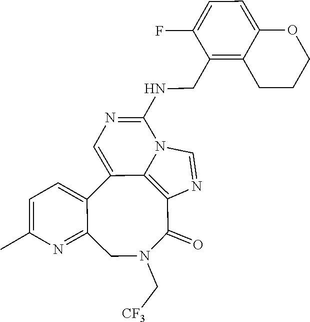 [SMILES: Cc1ccc2c(n1)CN(CC(F)(F)F)C(=O)c1ncn3c(NCc4c(F)ccc5c4CCCO5)ncc-2c13] | 12-(((6-fluorochroman-5-yl)methyl)amino)-7- methyl-4-(2,2,2-trifluoroethyl)-4,5-dihydro-3H-2,4,6, 11,12a-pentaazabenzo[4,5]cycloocta[1,2,3-cd] inden-3-one |
| E40 | 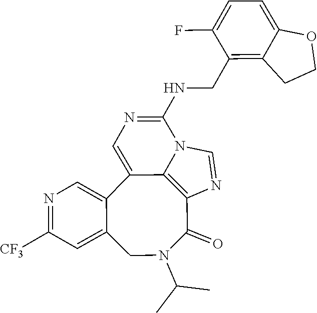 [SMILES: CC(C)N1Cc2cc(C(F)(F)F)ncc2-c2cnc(NCc3c(F)ccc4c3CCO4)n3cnc(c23)C1=O] | 12-(((5-fluoro-2,3-dihydrobenzofuran-4-yl)methyl) amino)-4-isopropyl-7-(trifluoromethyl)-4,5-dihydro- 3H-2,4,8,11,12a-pentaazabenzo[4,5]cycloocta [1,2,3-cd]inden-3-one |
| E41 | 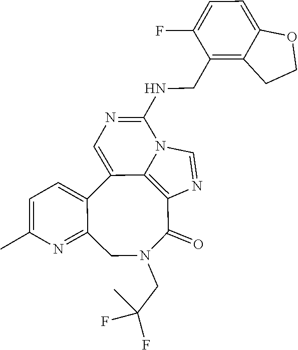 [SMILES: Cc1ccc2c(n1)CN(CC(C)(F)F)C(=O)c1ncn3c(NCc4c(F)ccc5c4CCO5)ncc-2c13] | 4-(2,2-difluoropropyl)-12-(((5-fluoro-2,3-dihydro benzofuran-4-yl)methyl)amino)-7-methyl-4,5- dihydro-3H-2,4,6,11,12a-pentaazabenzo[4,5] cycloocta[1,2,3-cd]inden-3-one |
| E42 | 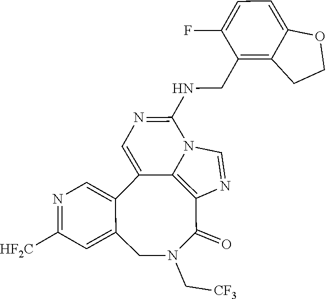 | 7-(difluoromethyl)-12-(((5-fluoro-2,3-dihydrobenzo- furan-4-yl)methyl)amino)-4-(2,2,2-trifluoroethyl)- 4,5-dihydro-3H-2,4,8,11,12a-pentaazabenzo[4,5] cycloocta[1,2,3-cd]inden-3-one |
| E43 | 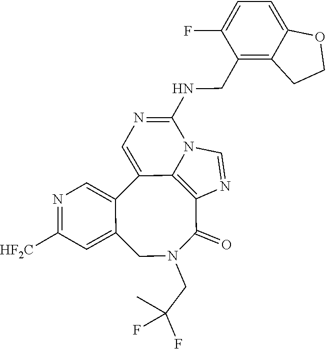 | 7-(difluoromethyl)-4-(2,2-difluoropropyl)-12-(((5- fluoro-2,3-dihydrobenzofuran-4-yl)methyl)amino)- 4,5-dihydro-3H-2,4,8,11,12a-pentaazabenzo[4,5] cycloocta[1,2,3-cd]inden-3-one |
| E44 | 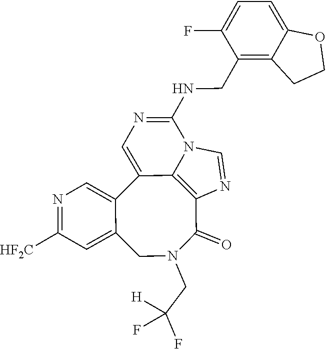 | 4-(2,2-difluoroethyl)-7-(difluoromethyl)-12-(((5- fluoro-2,3-dihydrobenzofuran-4-yl)methyl)amino)- 4,5-dihydro-3H-2,4,8,11,12a-pentaazabenzo[4,5] cycloocta[1,2,3-cd]inden-3-one |
| E45 | 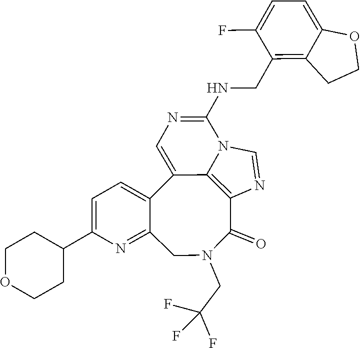 [SMILES: O=C1c2ncn3c(NCc4c(F)ccc5c4CCO5)ncc(c23)-c2ccc(C3CCOCC3)nc2CN1CC(F)(F)F] | 12-(((5-fluoro-2,3-dihydrobenzofuran-4-yl)methyl) amino)-7-(tetrahydro-2H-pyran-4-yl)-4-(2,2,2- trifluoroethyl)-4,5-dihydro-3H-2,4,6,11,12a- pentaazabenzo[4,5]cycloocta[1,2,3-cd] inden-3-one |
| E46 | 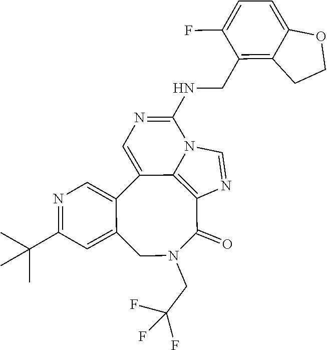 [SMILES: CC(C)(C)c1cc2c(cn1)-c1cnc(NCc3c(F)ccc4c3CCO4)n3cnc(c13)C(=O)N(CC(F)(F)F)C2] | 7-(tert-butyl)-12-(((5-fluoro-2,3-dihydrobenzofuran- 4-yl)methyl)amino)-4-(2,2,2-trifluoroethyl)-4,5- dihydro-3H-2,4,8,11,12a-pentaazabenzo[4,5] cycloocta[1,2,3-cd]inden-3-one |
| E47 | 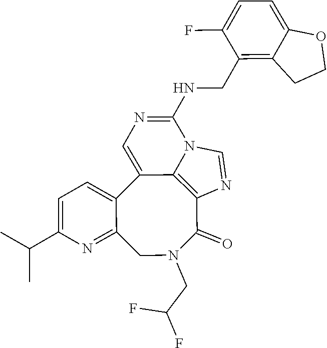 [SMILES: CC(C)c1ccc2c(n1)CN(CC(F)F)C(=O)c1ncn3c(NCc4c(F)ccc5c4CCO5)ncc-2c13] | 4-(2,2-difluoroethyl)-12-(((5-fluoro-2,3-dihydro- benzofuran-4-yl)methyl)amino)-7-isopropyl-4,5- dihydro-3H-2,4,6,11,12a-pentaazabenzo[4,5] cycloocta[1,2,3-cd]inden-3-one |
| E48 | 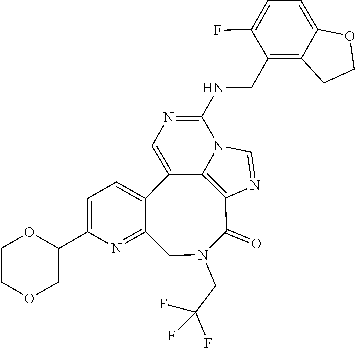 [SMILES: O=C1c2ncn3c(NCc4c(F)ccc5c4CCO5)ncc(c23)-c2ccc(C3COCCO3)nc2CN1CC(F)(F)F] | 7-(1,4-dioxan-2-yl)-12-(((5-fluoro-2,3-dihydrobenzo- furan-4-yl)methyl)amino)-4-(2,2,2-trifluoroethyl)- 4,5-dihydro-3H-2,4,6, 11,12a-pentaazabenzo[4,5] cycloocta[1,2,3-cd]inden-3-one |
| E49 | 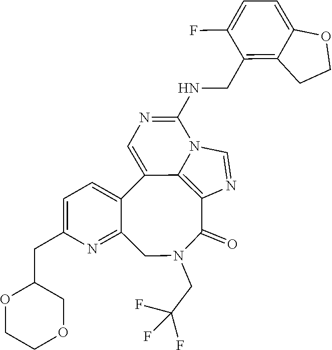 [SMILES: O=C1c2ncn3c(NCc4c(F)ccc5c4CCO5)ncc(c23)-c2ccc(CC3COCCO3)nc2CN1CC(F)(F)F] | 7-((1,4-dioxan-2-yl)methyl)-12-(((5-fluoro-2,3- dihydrobenzofuran-4-yl)methyl)amino)-4-(2,2,2- trifluoroethyl)-4,5-dihydro-3H-2,4,6,11,12a- pentaazabenzo[4,5]cycloocta[1,2,3-cd] inden-3-one |
| E50 | 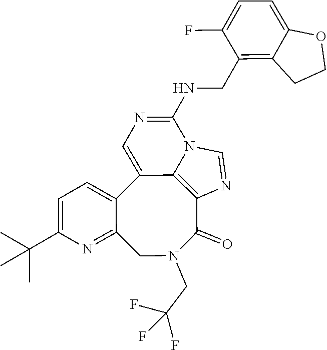 [SMILES: CC(C)(C)c1ccc2c(n1)CN(CC(F)(F)F)C(=O)c1ncn3c(NCc4c(F)ccc5c4CCO5)ncc-2c13] | 7-(tert-butyl)-12-(((5-fluoro-2,3-dihydrobenzofuran- 4-yl)methyl)amino)-4-(2,2,2-trifluoroethyl)-4,5- dihydro-3H-2,4,6,11,12a-pentaazabenzo[4,5] cycloocta[1,2,3-cd]inden-3-one |
| E51 | 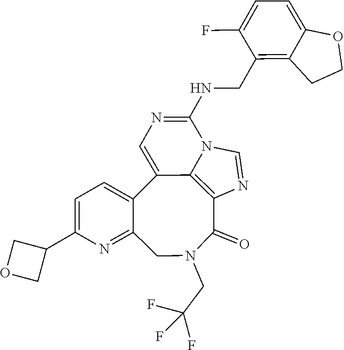 [SMILES: O=C1c2ncn3c(NCc4c(F)ccc5c4CCO5)ncc(c23)-c2ccc(C3COC3)nc2CN1CC(F)(F)F] | 12-(((5-fluoro-2,3-dihydrobenzofuran-4-yl)methyl) amino)-7-(oxetan-3-yl)-4-(2,2,2-trifluoroethyl)-4, 5-dihydro-3H-2,4,6,11,12a-pentaazabenzo[4,5] cycloocta[1,2,3-cd]inden-3-one |
| E52 | 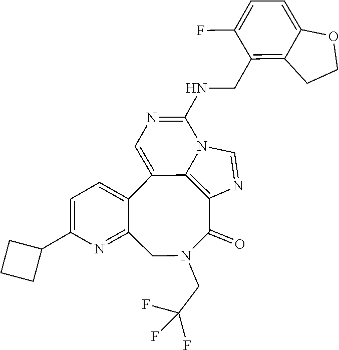 [SMILES: O=C1c2ncn3c(NCc4c(F)ccc5c4CCO5)ncc(c23)-c2ccc(C3CCC3)nc2CN1CC(F)(F)F] | 7-cyclobutyl-12-(((5-fluoro-2,3-dihydrobenzofuran- 4-yl)methyl)amino)-4-(2,2,2-trifluoroethyl)-4,5- dihydro-3H-2,4,6,11,12a-pentaazabenzo[4,5] cycloocta[1,2,3-cd]inden-3-one |

In another embodiment, The EED inhibitors are compounds of Formula I selected from group consisting of:
4-ethyl-12-(((5-fluoro-2,3-dihydrobenzofuran-4-yl)methyl)amino)-7-(trifluoromethyl)-4,5-dihydro-3H-2,4,8,11,12a-pentaazabenzo[4,5]cycloocta[1,2,3-cd]inden-3-on e;
12-(((5-fluoro-2,3-dihydrobenzofuran-4-yl)methyl)amino)-4-(2,2,2-trifluoroethyl)-7-(trifluoromethyl)-4,5-dihydro-3H-2,4,8,11,12a-pentaazabenzo[4,5]cycloocta[1,2,3-cd]inden-3-one;
4-cyclopropyl-12-(((5-fluoro-2,3-dihydrobenzofuran-4-yl)methyl)amino)-7-(trifluoromethyl)-4,5-dihydro-3H-2,4,8,11,12a-pentaazabenzo[4,5]cycloocta[1,2,3-cd]inden-3-one;
12-(((5-fluoro-2,3-dihydrobenzofuran-4-yl)methyl)amino)-4-isopropyl-7-(trifluoromethyl)-4,5-dihydro-3H-2,4,8,11,12a-pentaazabenzo[4,5]cycloocta[1,2,3-cd]inden-3-one; and
11-(((5-fluoro-2,3-dihydrobenzofuran-4-yl)methyl)amino)-6-methyl-4H-3-thia-2,5,10,11a-tetraazadibenzo[cd,f]azulene 3,3-dioxide,

or a pharmaceutically acceptable salt or solvate thereof.

In another embodiment, the EED inhibitor is Compound 73 as follows:

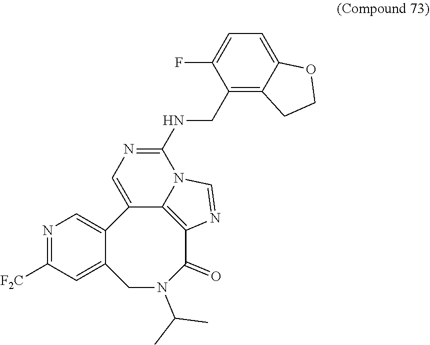

i.e., 12-(((5-fluoro-2,3-dihydrobenzofuran-4-yl)methyl)amino)-4-isopropyl-7-(trifluoromethyl)-4,5-dihydro-3H-2,4,8,11,12a-pentaazabenzo[4,5]cycloocta[1,2,3-cd]inden-3-one, or a pharmaceutically acceptable salt or solvate thereof.

In a preferred embodiment, the ALK inhibitor is a compound of Formula (1) or a pharmaceutically acceptable salt or solvate thereof:

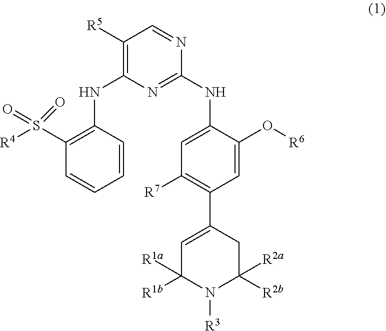

wherein:
R^{1a} and R^{1b} are independently selected from the group consisting of hydrogen, C₁₋₆ alkyl, and C₃₋₈ cycloalkyl;
R^{2a} and R^{2b} are independently selected from the group consisting of hydrogen, C₁₋₆ alkyl, and C₃₋₈ cycloalkyl;
R³ is selected from the group consisting of hydrogen, C₁₋₆ alkyl, C₃₋₆ cycloalkyl, and 4- to 8-membered heterocyclyl,
R⁴ is selected from the group consisting of C₁₋₄ alkyl and C₃₋₆ cycloalkyl;
R⁵ is halo;
R⁶ is selected from the group consisting of C₁₋₄ alkyl and C₃₋₆ cycloalkyl; and
R⁷ is selected from the group consisting of hydrogen, C₁₋₄ alkyl, and C₃₋₆ cycloalkyl,
with proviso that when R^{1a}, R^{1b}, R^{2a}, and R^{2b} are each hydrogen, then R³ is selected from the group consisting of C₃₋₆ cycloalkyl and 4- to 8-membered heterocyclyl.

In a preferred embodiment, the ALK inhibitor is a compound in the following Table 2 or a pharmaceutically acceptable salt or solvate thereof:

TABLE 2

| No. | Structure | Name |
|---|---|---|
| 2-1 | 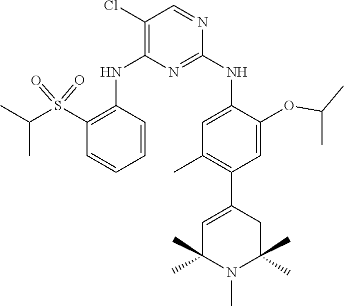 | 5-chloro-N²-(2-isopropoxy-5-methyl-4-(1,2,2,6,6-penta- methyl-1,2,3,6-tetrahydropyridin-4-yl)phenyl)-N⁴-(2- (isopropylsulfonyl)phenyl)pyrimidine-2,4-diamine; |
| 2-2 | 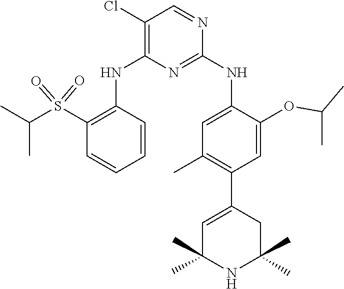 | 5-chloro-N²-(2-isopropoxy-5-methyl-4-(2,2,6,6-tetra- methyl-1,2,3,6-tetrahydropyridin-4-yl)phenyl)-N⁴-(2- (isopropylsulfonyl)phenyl)pyrimidine-2,4-diamine; |
| 2-3 | 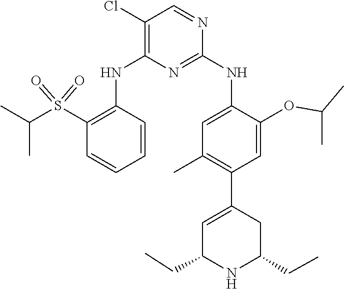 [SMILES: CC[C@H]1CC(c2cc(OC(C)C)c(Nc3ncc(Cl)c(Nc4ccccc4S(=O)(=O)C(C)C)n3)cc2C)=C[C@@H](CC)N1] | 5-chloro-N²-(4-((cis)-2,6-diethyl-1,2,3,6-tetrahydro- pyridin-4-yl)-2-isopropoxy-5-methylphenyl)-N⁴-(2- (isopropylsulfonyl)phenyl)pyrimidine-2,4-diamine; |
| 2-4 | 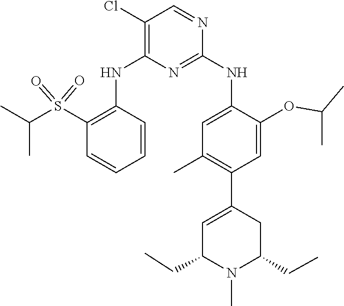 [SMILES: CC[C@H]1CC(c2cc(OC(C)C)c(Nc3ncc(Cl)c(Nc4ccccc4S(=O)(=O)C(C)C)n3)cc2C)=C[C@@H](CC)N1C] | 5-chloro-N²-(4-((cis)-2,6-diethyl-1-methyl-1,2,3,6-tetra- hydropyridin-4-yl)-2-isopropoxy-5-methylphenyl)-N⁴- (2-(isopropylsulfonyl)phenyl)pyrimidine-2,4-diamine; |
| 2-5 | 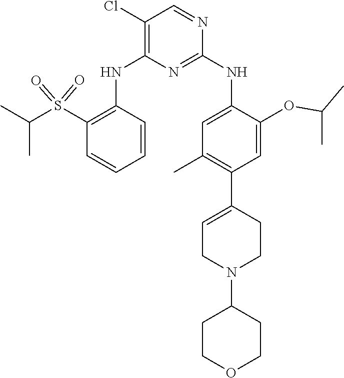 [SMILES: Cc1cc(Nc2ncc(Cl)c(Nc3ccccc3S(=O)(=O)C(C)C)n2)c(OC(C)C)cc1C1=CCN(C2CCOCC2)CC1] | 5-chloro-N²-(2-isopropoxy-5-methyl-4-(1-(tetrahydro- 2H-pyran-4-yl)-1,2,3,6-tetrahydropyridin-4-yl)phenyl)- N⁴-(2-(isopropylsulfonyl)phenyl)pyrimidine-2,4- diamine; |
| 2-6 | 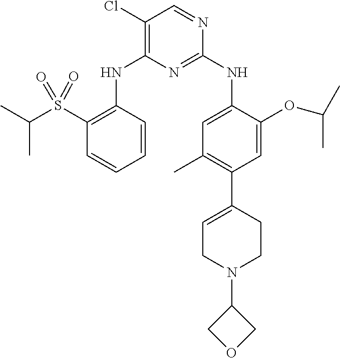 [SMILES: Cc1cc(Nc2ncc(Cl)c(Nc3ccccc3S(=O)(=O)C(C)C)n2)c(OC(C)C)cc1C1=CCN(C2COC2)CC1] | 5-chloro-N²-(2-isopropoxy-5-methyl-4-(1-(oxetan-3- yl)-1,2,3,6-tetrahydropyridin-4-yl)phenyl)-N⁴-(2- (isopropylsulfonyl)phenyl)pyrimidine-2,4-diamine; |
| 2-7 | 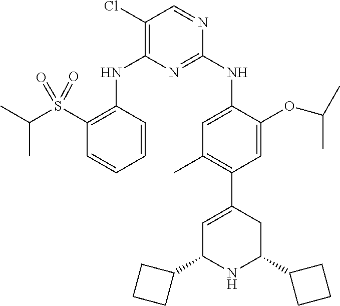 [SMILES: Cc1cc(Nc2ncc(Cl)c(Nc3ccccc3S(=O)(=O)C(C)C)n2)c(OC(C)C)cc1C1=C[C@@H](C2CCC2)N[C@@H](C2CCC2)C1] | 5-chloro-N²-(4-((cis)-2,6-dicyclobutyl-1,2,3,6-tetra- hydropyridin-4-yl)-2-isopropoxy-5-methylphenyl)-N⁴- (2-(isopropylsulfonyl)phenyl)pyrimidine-2,4-diamine; |
| 2-8 | 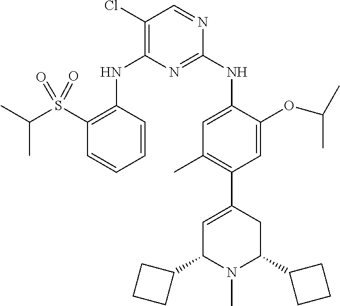 [SMILES: Cc1cc(Nc2ncc(Cl)c(Nc3ccccc3S(=O)(=O)C(C)C)n2)c(OC(C)C)cc1C1=C[C@@H](C2CCC2)N(C)[C@@H](C2CCC2)C1] | 5-chloro-N²-(4-((cis)-2,6-dicyclobutyl-1-methyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 2-9 | 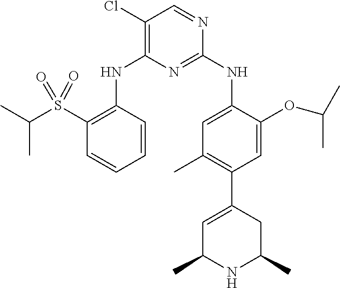 [SMILES: Cc1cc(Nc2ncc(Cl)c(Nc3ccccc3S(=O)(=O)C(C)C)n2)c(OC(C)C)cc1C1=C[C@H](C)N[C@H](C)C1] | 5-chloro-N²-(4-((cis)-2,6-dimethyl-1,2,3,6-tetrahydro- pyridin-4-yl)-2-isopropoxy-5-methylphenyl)-N⁴-(2- (isopropylsulfonyl)phenyl)pyrimidine-2,4-diamine; |
| 2-10 | 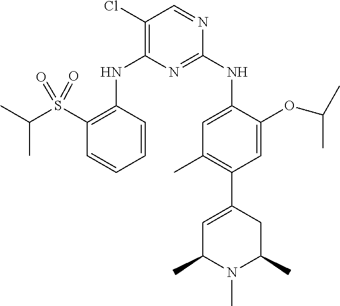 [SMILES: Cc1cc(Nc2ncc(Cl)c(Nc3ccccc3S(=O)(=O)C(C)C)n2)c(OC(C)C)cc1C1=C[C@H](C)N(C)[C@H](C)C1] | 5-chloro-N²-(2-isopropoxy-5-methyl-4-((cis)-1,2,6-tri- methyl-1,2,3,6-tetrahydropyridin-4-yl)phenyl)-N⁴-(2- (isopropylsulfonyl)phenyl)pyrimidine-2,4-diamine; |
| 2-11 | 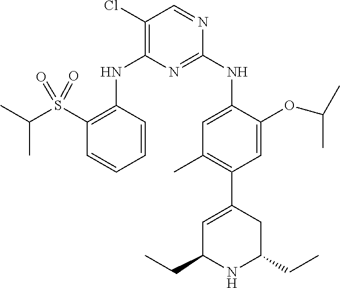 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1] | 5-chloro-N²-(4-((trans)-2,6-diethyl-1,2,3,6-tetrahydro- pyridin-4-yl)-2-isopropoxy-5-methylphenyl)-N⁴-(2- (isopropylsulfonyl)phenyl)pyrimidine-2,4-diamine; |
| 2-12 | 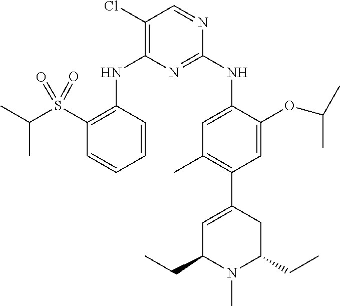 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C] | 5-chloro-N²-(4-((trans)-2,6-diethyl-1-methyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 2-13 | 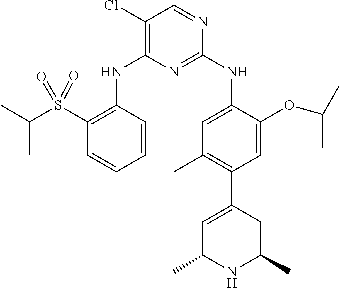 [SMILES: Cc1cc(Nc2ncc(Cl)c(Nc3ccccc3S(=O)(=O)C(C)C)n2)c(OC(C)C)cc1C1=C[C@@H](C)N[C@H](C)C1] | 5-chloro-N²-(4-((trans)-2,6-dimethyl-1,2,3,6-tetrahydro- pyridin-4-yl)-2-isopropoxy-5-methylphenyl)-N⁴-(2-(iso- propylsulfonyl)phenyl)pyrimidine-2,4-diamine; |
| 2-14 | 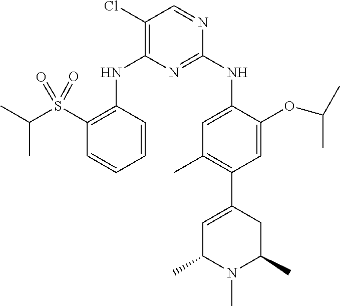 [SMILES: Cc1cc(Nc2ncc(Cl)c(Nc3ccccc3S(=O)(=O)C(C)C)n2)c(OC(C)C)cc1C1=C[C@@H](C)N(C)[C@H](C)C1] | 5-chloro-N²-(2-isopropoxy-5-methyl-4-((trans)-1,2,6- trimethyl-1,2,3,6-tetrahydropyridin-4-yl)phenyl)-N⁴-(2- (isopropylsulfonyl)phenyl)pyrimidine-2,4-diamine; |
| 2-15 | 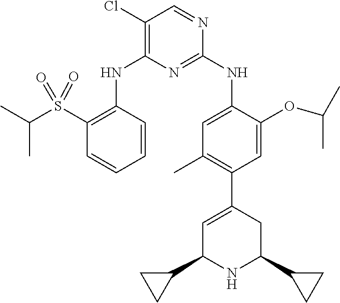 [SMILES: Cc1cc(Nc2ncc(Cl)c(Nc3ccccc3S(=O)(=O)C(C)C)n2)c(OC(C)C)cc1C1=C[C@H](C2CC2)N[C@H](C2CC2)C1] | 5-chloro-N²-(4-((cis)-2,6-dicyclopropyl-1,2,3,6-tetra- hydropyridin-4-yl)-2-isopropoxy-5-methylphenyl)-N⁴- (2-(isopropylsulfonyl)phenyl)pyrimidine-2,4-diamine; |
| 2-16 | 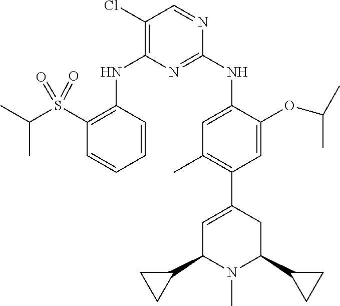 [SMILES: Cc1cc(Nc2ncc(Cl)c(Nc3ccccc3S(=O)(=O)C(C)C)n2)c(OC(C)C)cc1C1=C[C@H](C2CC2)N(C)[C@H](C2CC2)C1] | 5-chloro-N²-(4-((cis)-2,6-dicyclopropyl-1-methyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 2-17 | 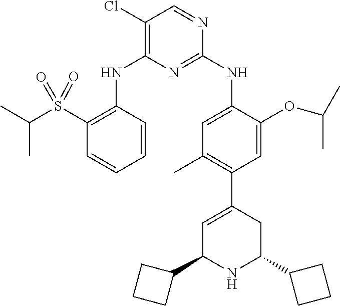 [SMILES: Cc1cc(Nc2ncc(Cl)c(Nc3ccccc3S(=O)(=O)C(C)C)n2)c(OC(C)C)cc1C1=C[C@H](C2CCC2)N[C@@H](C2CCC2)C1] | 5-chloro-N²-(4-((trans)-2,6-dicyclobutyl-1,2,3,6-tetra- hydropyridin-4-yl)-2-isopropoxy-5-methylphenyl)-N⁴- (2-(isopropylsulfonyl)phenyl)pyrimidine-2,4-diamine; |
| 2-18 | 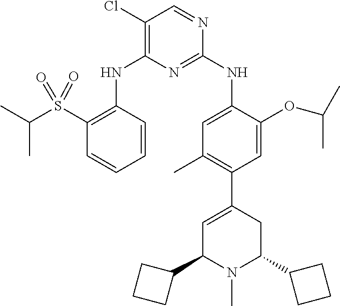 [SMILES: Cc1cc(Nc2ncc(Cl)c(Nc3ccccc3S(=O)(=O)C(C)C)n2)c(OC(C)C)cc1C1=C[C@H](C2CCC2)N(C)[C@@H](C2CCC2)C1] | 5-chloro-N²-(4-((trans)-2,6-dicyclobutyl-1-methyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 2-19 | 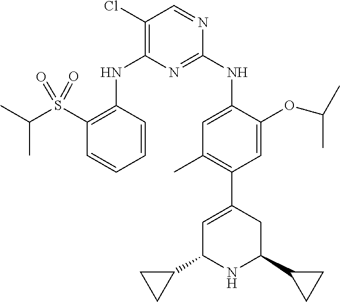 [SMILES: Cc1cc(Nc2ncc(Cl)c(Nc3ccccc3S(=O)(=O)C(C)C)n2)c(OC(C)C)cc1C1=C[C@@H](C2CC2)N[C@H](C2CC2)C1] | 5-chloro-N²-(4-((trans)-2,6-dicyclopropyl-1,2,3,6-tetra- hydropyridin-4-yl)-2-isopropoxy-5-methylphenyl)-N⁴- (2-(isopropylsulfonyl)phenyl)pyrimidine-2,4-diamine; |
| 2-20 | 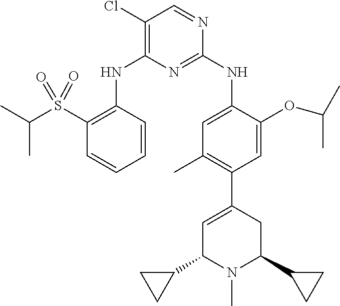 [SMILES: Cc1cc(Nc2ncc(Cl)c(Nc3ccccc3S(=O)(=O)C(C)C)n2)c(OC(C)C)cc1C1=C[C@@H](C2CC2)N(C)[C@H](C2CC2)C1] | 5-chloro-N²-(4-((trans)-2,6-dicyclopropyl-1-methyl-1,2,3,6- tetrahydropyridin-4-yl)-2-isopropoxy-5-methylphenyl)- N⁴-(2-(isopropylsulfonyl)phenyl)pyrimidine-2,4- diamine; |
| 2-21 | 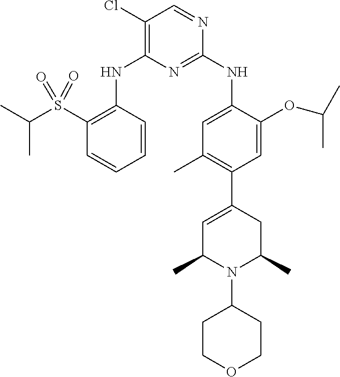 [SMILES: Cc1cc(Nc2ncc(Cl)c(Nc3ccccc3S(=O)(=O)C(C)C)n2)c(OC(C)C)cc1C1=C[C@H](C)N(C2CCOCC2)[C@H](C)C1] | 5-chloro-N²-(4-((cis)-2,6-dimethyl-1-(tetrahydro-2H- pyran-4-yl)-1,2,3,6-tetrahydropyridin-4-yl)-2-isopro- poxy-5-methylphenyl)-N⁴-(2-(isopropylsulfonyl)phenyl) pyrimidine-2,4-diamine; |
| 2-22 | 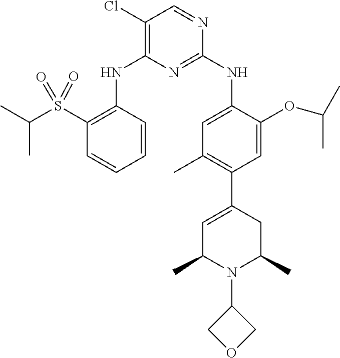 [SMILES: Cc1cc(Nc2ncc(Cl)c(Nc3ccccc3S(=O)(=O)C(C)C)n2)c(OC(C)C)cc1C1=C[C@H](C)N(C2COC2)[C@H](C)C1] | 5-chloro-N²-(4-((cis)-2,6-dimethyl-1-(oxetan-3-yl)-1,2,3,6- tetrahydropyridin-4-yl)-2-isopropoxy-5-methylphenyl)- N⁴-(2-(isopropylsulfonyl)phenyl)pyrimidine-2,4- diamine; |
| 2-23 | 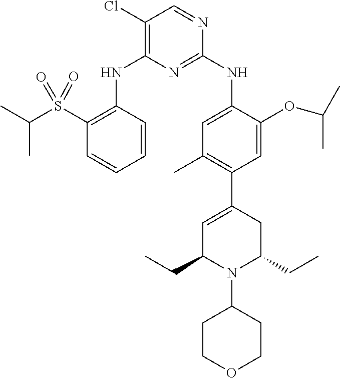 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C1CCOCC1] | 5-chloro-N²-(4-((trans)-2,6-diethyl-1-(tetrahydro-2H- pyran-4-yl)-1,2,3,6-tetrahydropyridin-4-yl)-2-isopro- poxy-5-methylphenyl)-N⁴-(2-(isopropylsulfonyl)phenyl) pyrimidine-2,4-diamine; |
| 2-24 | 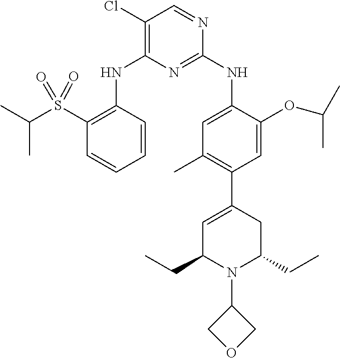 [SMILES: CC[C@H]1CC(c2cc(OC(C)C)c(Nc3ncc(Cl)c(Nc4ccccc4S(=O)(=O)C(C)C)n3)cc2C)=C[C@H](CC)N1C1COC1] | 5-chloro-N²-(4-((2S,6S)-2,6-diethyl-1-(oxetan-3-yl)- 1,2,3,6-tetrahydropyridin-4-yl)-2-isopropoxy-5-methyl- phenyl)-N⁴-(2-(isopropylsulfonyl)phenyl)pyrimidine- 2,4-diamine; |
| 2-25 | 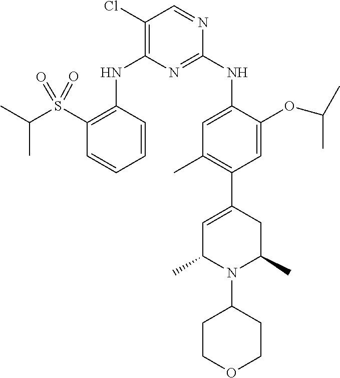 [SMILES: Cc1cc(Nc2ncc(Cl)c(Nc3ccccc3S(=O)(=O)C(C)C)n2)c(OC(C)C)cc1C1=C[C@@H](C)N(C2CCOCC2)[C@H](C)C1] | 5-chloro-N²-(4-((trans)-2,6-dimethyl-1-(tetrahydro-2H- pyran-4-yl)-1,2,3,6-tetrahydropyridin-4-yl)-2-isopro- poxy-5-methylphenyl)-N⁴-(2-(isopropylsulfonyl)phenyl) pyrimidine-2,4-diamine; |
| 2-26 | 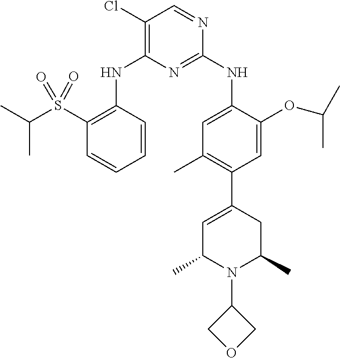 [SMILES: Cc1cc(Nc2ncc(Cl)c(Nc3ccccc3S(=O)(=O)C(C)C)n2)c(OC(C)C)cc1C1=C[C@@H](C)N(C2COC2)[C@H](C)C1] | 5-chloro-N²-(4-((trans)-2,6-dimethyl-1-(oxetan-3-yl)- 1,2,3,6-tetrahydropyridin-4-yl)-2-isopropoxy-5-methyl- phenyl)-N⁴-(2-(isopropylsulfonyl)phenyl)pyrimidine- 2,4-diamine; |
| 2-27 | 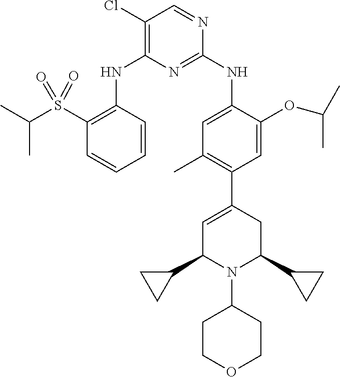 [SMILES: Cc1cc(Nc2ncc(Cl)c(Nc3ccccc3S(=O)(=O)C(C)C)n2)c(OC(C)C)cc1C1=C[C@H](C2CC2)N(C2CCOCC2)[C@H](C2CC2)C1] | 5-chloro-N²-(4-((cis)-2,6-dicyclopropyl-1-(tetrahydro- 2H-pyran-4-yl)-1,2,3,6-tetrahydropyridin-4-yl)-2-iso- propoxy-5-methylphenyl)-N⁴-(2-(isopropylsulfonyl) phenyl)pyrimidine-2,4-diamine; |
| 2-28 | 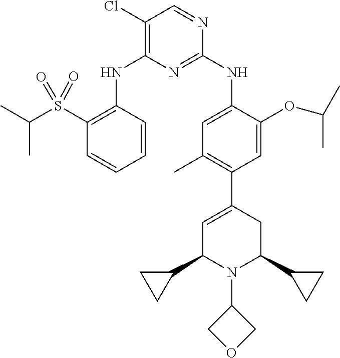 [SMILES: Cc1cc(Nc2ncc(Cl)c(Nc3ccccc3S(=O)(=O)C(C)C)n2)c(OC(C)C)cc1C1=C[C@H](C2CC2)N(C2COC2)[C@H](C2CC2)C1] | 5-chloro-N²-(4-((cis)-2,6-dicyclopropyl-1-(oxetan-3- yl)-1,2,3,6-tetrahydropyridin-4-yl)-2-isopropoxy-5- methylphenyl)-N⁴-(2-(isopropylsulfonyl)phenyl) pyrimidine-2,4-diamine; |
| 2-29 | 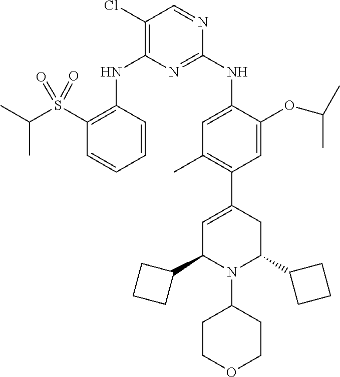 [SMILES: Cc1cc(Nc2ncc(Cl)c(Nc3ccccc3S(=O)(=O)C(C)C)n2)c(OC(C)C)cc1C1=C[C@H](C2CCC2)N(C2CCOCC2)[C@@H](C2CCC2)C1] | 5-chloro-N²-(4-((trans)-2,6-dicyclobutyl-1-(tetrahydro- 2H-pyran-4-yl)-1,2,3,6-tetrahydropyridin-4-yl)-2-iso- propoxy-5-methylphenyl)-N⁴-(2-(isopropylsulfonyl) phenyl)pyrimidine-2,4-diamine; |
| 2-30 | 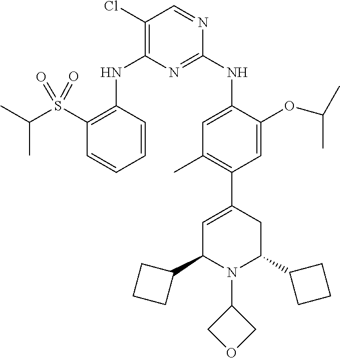 [SMILES: Cc1cc(Nc2ncc(Cl)c(Nc3ccccc3S(=O)(=O)C(C)C)n2)c(OC(C)C)cc1C1=C[C@H](C2CCC2)N(C2COC2)[C@@H](C2CCC2)C1] | 5-chloro-N²-(4-((trans)-2,6-dicyclobutyl-1-(oxetan-3- yl)-1,2,3,6-tetrahydropyridin-4-yl)-2-isopropoxy-5- methylphenyl)-N⁴-(2-(isopropylsulfonyl)phenyl) pyrimidine-2,4-diamine; |
| 2-31 | 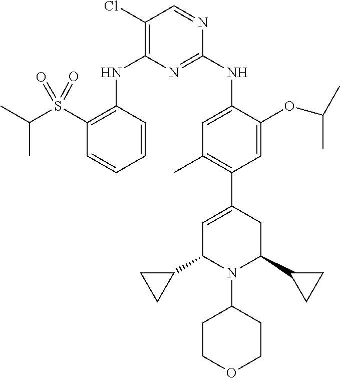 [SMILES: Cc1cc(Nc2ncc(Cl)c(Nc3ccccc3S(=O)(=O)C(C)C)n2)c(OC(C)C)cc1C1=C[C@@H](C2CC2)N(C2CCOCC2)[C@H](C2CC2)C1] | 5-chloro-N²-(4-((trans)-2,6-dicyclopropyl-1-(tetrahydro- 2H-pyran-4-yl)-1,2,3,6-tetrahydropyridin-4-yl)-2-iso- propoxy-5-methylphenyl)-N⁴-(2-(isopropylsulfonyl) phenyl)pyrimidine-2,4-diamine; or |
| 2-32 | 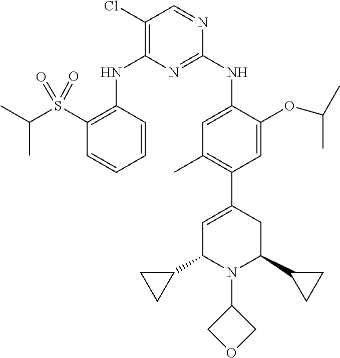 [SMILES: Cc1cc(Nc2ncc(Cl)c(Nc3ccccc3S(=O)(=O)C(C)C)n2)c(OC(C)C)cc1C1=C[C@@H](C2CC2)N(C2COC2)[C@H](C2CC2)C1] | 5-chloro-N²-(4-((trans)-2,6-dicyclopropyl-1-(oxetan-3- yl)-1,2,3,6-tetrahydropyridin-4-yl)-2-isopropoxy-5- methylphenyl)-N⁴-(2-(isopropylsulfonyl)phenyl) pyrimidine-2,4-diamine. |

In a preferred embodiment, the ALK inhibitor is Compound 2-5 as follows:

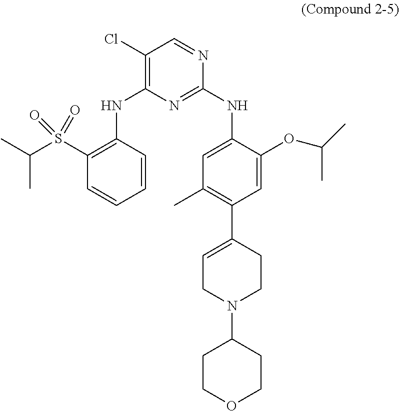 [SMILES: Cc1cc(Nc2ncc(Cl)c(Nc3ccccc3S(=O)(=O)C(C)C)n2)c(OC(C)C)cc1C1=CCN(C2CCOCC2)CC1]

i.e., 5-chloro-N2-(2-isopropoxy-5-methyl-4-(1-(tetrahydro-2H-pyran-4-yl)-1,2,3,6-tetra hydropyridin-4-yl)phenyl)-N1-(2-(isopropylsulfonyl)phenyl)pyrimidine-2,4-diamine, or a pharmaceutically acceptable salt or hydrate thereof.

In a preferred embodiment, the BCR-ABL inhibitor is compound of the formula (A) or pharmaceutically acceptable salt or solvate thereof:

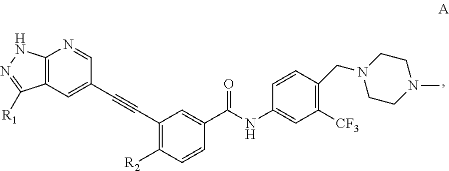

wherein,
R₁ is hydrogen, C₁-C₄ alkyl, C₃-C₆ cycloalkyl, C₁-C₄ alkyloxy, or phenyl; and R₂ is hydrogen, C₁-C₄ alkyl, C₃-C₆ cycloalkyl, or halogen.

In a preferred embodiment, the BCR-ABL inhibitor is Compound A-1 with the following structure including any tautomer forms, or a pharmaceutically acceptable salt or solvate thereof:

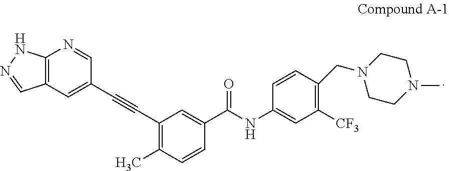 [SMILES: Cc1ccc(C(=O)Nc2ccc(CN3CCN(C)CC3)c(C(F)(F)F)c2)cc1C#Cc1cnc2[nH]ncc2c1]

In a preferred embodiment, the MDM2 inhibitor is a compound of the following formula (VI), or a pharmaceutically acceptable salt thereof:

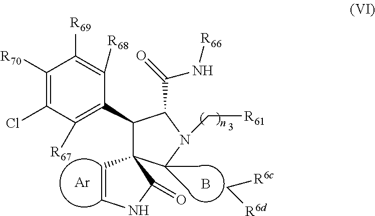

wherein

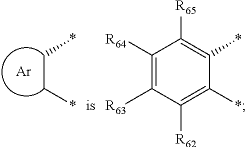

B is

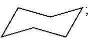

R61 is H, or unsubstituted C1-4 alkyl;
n3 is 0, 1, or 2;
R62, R63, R64, R65, R67, R68, R69, and R70, independently, are selected from the group consisting of H, F, Cl, CH3, and CF3;
R66 is

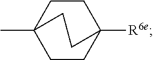

and
R6c and R6d are substituents on one carbon atom of ring B, wherein
R6c is H, C1-3 alkyl, C1-3 alkylene-OR6a, OR6a, or halo;
R6d is H, C1-3 alkyl, C1-3 alkylene-OR6a, OR6a, or halo;
R6e is —C(═O)OR6a, —C(═O)NR6aR6b, or —C(═O)NHSO2CH3;
R6a is hydrogen or unsubstituted C1-4 alkyl; and
R6b is hydrogen or unsubstituted C1-4 alkyl.

In some embodiments, n3 is 0, or 1.

In some embodiments, R61 is H or CH3.

In some embodiments,

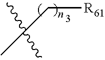

### is H, CH3, or CH2CH3.

In some embodiments, R62 is H, R63 is F or C1, and R64 and R65 are H.

In some embodiments, R67 is fluoro, each of R68, R69, and R70 is H, R6c is H, CH3, OH, or halo, and R6d is H, CH3, OH, or halo.

In some embodiments, the MDM2 inhibitor is selected from the following group of compounds or a pharmaceutically acceptable salt thereof:

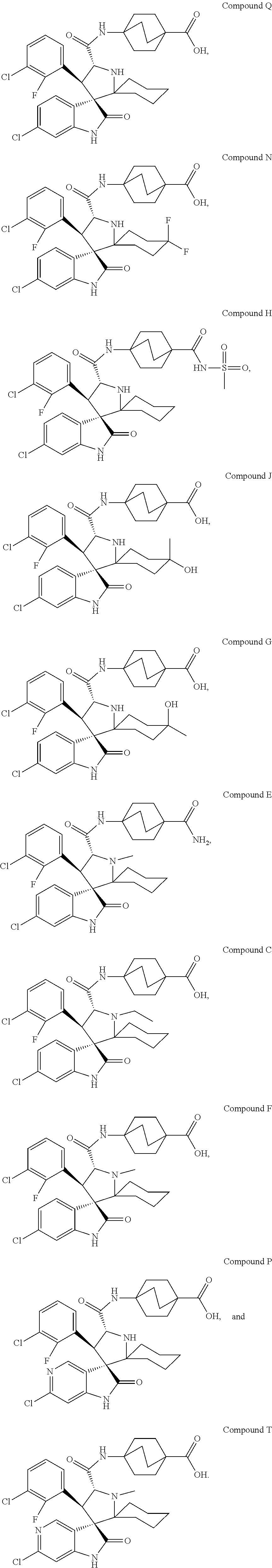

In some embodiments, the MIDM2 inhibitor is the compound having the following formula or a pharmaceutically acceptable salt thereof:

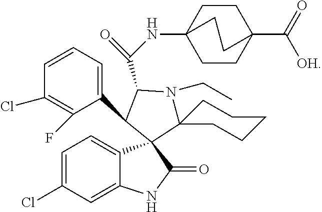 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

In a preferred embodiment, the Bcl-2 inhibitor is a compound of the following formula (V), or a pharmaceutically acceptable salt thereof:

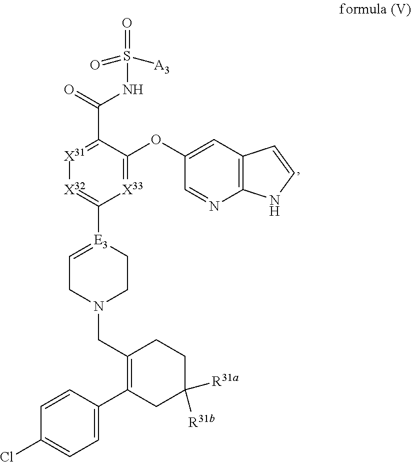

or a pharmaceutically acceptable salt or solvate thereof, wherein:
A₃ is selected from

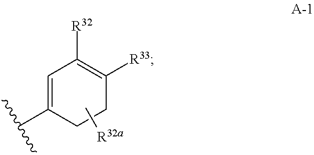

E₃ is a nitrogen atom and [symbol] is a single bond;
X³¹, X³², and X³³ are each independently selected from the group consisting of —CR³⁸═ and —N═;
R^{31a} and R^{31b} taken together with the carbon atom to which they are attached form a 3-, 4-, or 5-membered cycloalkyl;
R³² is selected from the group consisting of —NO₂, —SO₂CH₃, and —SO₂CF₃;
R^{32a} is selected from the group consisting of hydrogen and halogen;
R³³ is selected from —N(R^{34a})(R^{34b});
R^{34a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{34b} is selected from the group consisting of hydrogen and C₁₋₄ alkyl;
R³⁸ is selected from the group consisting of hydrogen and halogen.

In a preferred embodiment, the Bcl-2 inhibitor is selected from the group consisting of:

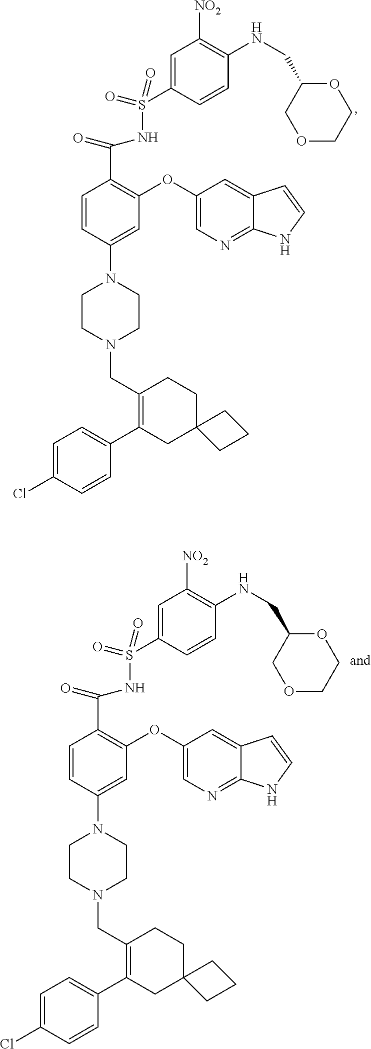 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

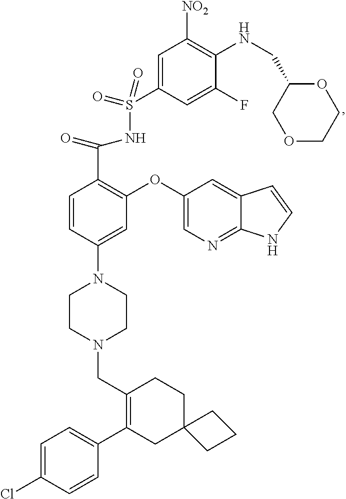 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

or a pharmaceutically acceptable salt or solvate thereof.

In a preferred embodiment, the Bcl-2 inhibitor is Compound 4:

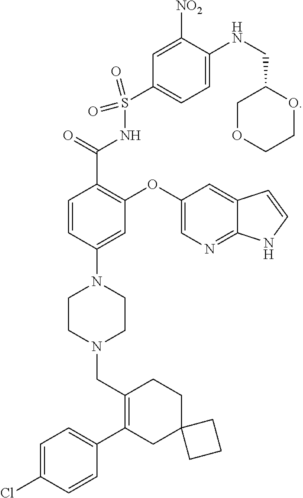 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In a preferred embodiment, the pharmaceutical composition is for use in treating or suppressing a cancer, reducing its severity, lowering its risk or inhibiting its metastasis in an individual, and the cancer is preferably selected from the group consisting of bladder cancer, breast cancer, cervical cancer, colon cancer (including colorectal cancer), esophageal cancer, esophageal squamous cell carcinoma, head and neck cancer, liver cancer, lung cancer (including small cell lung cancer and non-small cell lung cancer), lung squamous cell carcinoma, mesothelial tumor, melanoma, myeloma, rhabdomyosarcoma, inflammatory myofibroblastic tumor, neuroturbo chargeoma, pancreatic cancer, prostate cancer, kidney cancer, renal cell carcinoma, sarcoma (including osteosarcoma), skin cancer, squamous cell carcinoma, spindle cell carcinoma, gastric cancer, testicular cancer, thyroid cancer, uterine cancer, mesothelioma, neuroblastoma, cholangiocarcinoma, leiomyosarcoma, liposarcoma, nasopharyngeal carcinoma, neuroendocrine carcinoma, ovarian cancer, salivary gland cancer, metastasis caused by spindle cell carcinoma, anaplastic large cell lymphoma, thyroid undifferentiated carcinoma, non-Hodgkin's lymphoma, Hodgkin's lymphoma, and hematological malignancies, such as acute myeloid leukemia (AML), acute lymphoblastic leukemia (ALL), diffuse large B-cell lymphoma (DLBCL), follicular lymphoma (FL), chronic lymphocytic leukemia (CLL), chronic myeloid leukemia (CML), uveal melanoma, pleural mesothelioma, peritoneal mesothelioma;

In a preferred embodiment, the cancer is mesothelioma, neuroblastoma, non-small cell lung cancer, lung adenocarcinoma (LUAD), and lung squamous cell carcinoma, ovarian cancer, uveal melanoma, colon cancer, and liver cancer.

In a preferred embodiment, the cancer is mesothelioma (including pleural mesothelioma, peritoneal mesothelioma, mesothelioma with wild type BAP1 and mesothelioma with mutant BAP1), prostate cancer and diffuse large B-cell lymphoma (DLBCL)(including EZH2 mut DLBCL and EZH2 mut Bcl-2 translocation DLBCL).

In a preferred embodiment, the weight ratio between the EED inhibitor and the one or more anticancer reagents is 0.005-5000:0.005-5000, for example, 0.05-1500:0.005-5000, 0.1-6:0.005-4, 100:0.5-400, 100:1-350, 100:2-300, 100:5-200, 100:10-150, 100:10-100, 100:10-90, 100:20-80.

In a preferred embodiment, the molar ratio between the EED inhibitor and the one or more anticancer reagents is 10-1: 1-10, for example, 10:1, 9:1, 8:1, 7:1, 6:1, 5:1, 4:1, 3:1, 2:1, 1:1:1:2, 1:3, 1:4, 1:5, 1:6, 1:7, 1:8, 1:9, 1:10, and the ranges between any of the aforementioned values are also included.

In a preferred embodiment, the EED inhibitor is Compound 73 as follows:
12-(((5-fluoro-2,3-dihydrobenzofuran-4-yl)methyl)amino)-4-isopropyl-7-(trifluoromethyl)-4,5-dihydro-3H-2,4,8,11,12a-pentaazabenzo[4,5]cycloocta[1,2,3-cd]inden-3-one, or a pharmaceutically acceptable salt or solvate thereof;
the ALK inhibitor is Compound 2-5 as follows:
5-chloro-N²-(2-isopropoxy-5-methyl-4-(1-(tetrahydro-2H-pyran-4-yl)-1,2,3,6-tetrahydropyridin-4-yl)phenyl)-N-(2-(isopropylsulfonyl)phenyl)pyrimidine-2,4-diamine, or a pharmaceutically acceptable salt or hydrate thereof; and/or
   the BCR-ABL inhibitor is the compound A-1 with the following structure or a pharmaceutically acceptable salt or solvate thereof:

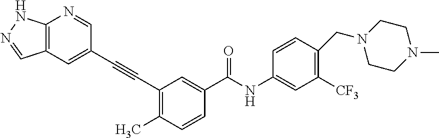 [SMILES: Cc1ccc(C(=O)Nc2ccc(CN3CCN(C)CC3)c(C(F)(F)F)c2)cc1C#Cc1cnc2[nH]ncc2c1]

the MDM2 inhibitor is the compound C having the following formula or a pharmaceutically acceptable salt thereof:

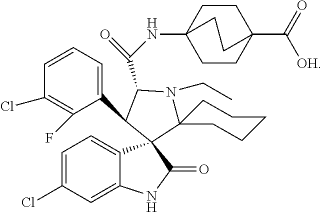 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

the Bcl-2 inhibitor is the compound 4 having the following formula or a pharmaceutically acceptable salt thereof:

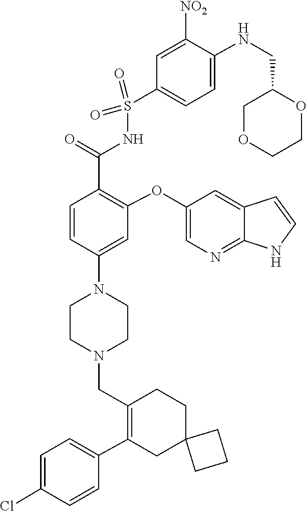 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In another aspect, the present invention provides a pharmaceutical composition comprising any pharmaceutical combination of the present invention, and optionally a pharmaceutically acceptable carrier.

In a preferred embodiment, the pharmaceutical composition may be in any form, for example, the composition is in the form of a tablet, a capsule, a granule, a syrup, a powder, a lozenge, a sachet, a cachet, an elixir, a suspension, an emulsion, a solution, an aerosol, an ointment, a cream and an injection.

The pharmaceutical combinations typically are administered in admixture with a pharmaceutical carrier to give a pharmaceutical composition selected with regard to the intended route of administration and standard pharmaceutical practice. Pharmaceutical compositions for use in accordance with the present invention are formulated in a conventional manner using one or more physiologically acceptable carriers comprising excipients and/or auxiliaries that facilitate processing of the pharmaceutical combinations.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the pharmaceutical combination is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of the pharmaceutical combination. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of the pharmaceutical combination.

When a therapeutically effective amount of pharmaceutical combination is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

The pharmaceutical combination can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding the pharmaceutical combination to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The pharmaceutical combination can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of the pharmaceutical combination can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

The pharmaceutical combination also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the pharmaceutical combination can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The pharmaceutical combination also can be injected parenterally, for example, intravenously, intramuscularly, subcutaneously, or intracoronarily. For parenteral administration, the pharmaceutical combination are typically used in the form of a sterile aqueous solution which can contain other substances, for example, salts or monosaccharides, such as mannitol or glucose, to make the solution isotonic with blood.

In a preferred embodiment, the invention provides a method for treating or suppressing a cancer, reducing its severity, lowering its risk or inhibiting its metastasis in an individual, comprising administering to the individual a therapeutically effective amount of an EED inhibitor, and optionally a therapeutically effective amount of one or more anticancer reagents.

In a preferred embodiment, the one or more anticancer reagents are selected from the group consisting of an ALK inhibitor, a BCR-ABL inhibitor, MDM2 inhibitor, Bcl-2 inhibitor and other inhibitors.

In a preferred embodiment, the EED, ALK, BCR-ABL, MDM2 and/or Bcl-2 inhibitor is as defined above and the cancer is as defined above.

In a preferred embodiment, the EED inhibitor is administrated in an amount of from about 0.005 mg/day to about 5000 mg/day, such as an amount of about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

In a preferred embodiment, the EED inhibitor is administrated in an amount of from about 1 ng/kg to about 200 mg/kg, about 1 μg/kg to about 100 mg/kg, or about 1 mg/kg to about 50 mg/kg per unit dose, for example, administrated in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg per unit dose, and administrated with one or more (e.g., 1, 2, 3, 4, 5, 6, 7, 8, 9 or 10) unit doses per day.

In a preferred embodiment, the one or more anticancer reagents are administrated in an amount of from 0.005 mg/day to about 5000 mg/day, for example, about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

In a preferred embodiment, the one or more anticancer reagents are administrated in an amount of from about 1 ng/kg to about 200 mg/kg, from about 1 μg/kg to about 100 mg/kg, or from about 1 mg/kg to about 50 mg/kg per unit dose, for example, administrated in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg per unit dose, and administered with one or more (e.g., 1, 2, 3, 4, 5, 6, 7, 8, 9, or 10) unit doses per day.

In a preferred embodiment, the EED inhibitor, and the one or more anticancer reagents are administered together, simultaneously, sequentially or alternately.

In a preferred embodiment, the EED inhibitor, and the one or more anticancer reagents are administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days.

In a preferred embodiment, the EED inhibitor, and the one or more anticancer reagents are administered for one or more (e.g., 1, 2, 3, 4, 5, 6, 7, 8, 9 or 10) courses of treatment, in which each of the courses lasts at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days; and there is an interval of 0, 1, 2, 3, 4, 5, 6, 7, 8, 9, 10 days, two weeks, three weeks or four weeks between every two courses of treatment.

In a preferred embodiment, when there are a plurality of courses of treatment, the amount of the EED inhibitor and/or anticancer reagents administered in each course of treatment is same or different. In a more preferred embodiment, the amount of the ALK inhibitor and/or anticancer reagents administered during the previous course of treatment is 1-10 times, preferably 1-5 times, such as 1.5, 2, 2.5, 3, 3.5, 4, 4.5 or 5 times, the amount administered during the subsequent course of treatment.

In a preferred embodiment, the EED inhibitor, and the one or more anticancer reagents are administrated via the same (e.g., oral) or different routes (e.g., oral and parenteral (e.g., injection), respectively).

In a preferred embodiment, the anticancer reagent is administrated in a lower dose in comparison with the dose of the anticancer reagent that is administered alone or when the one or more EED inhibitors are not administered.

In a preferred embodiment, the EED inhibitor enhances the therapeutic efficacy of the anticancer reagent in treatment of a cancer and/or reducing a side-effect of the anticancer reagent in treatment of a cancer.

In a preferred embodiment, the EED inhibitor and one or more anticancer reagents (e.g., those selected from the group consisting of an ALK inhibitor, a BCR-ABL inhibitor, a MDM2 inhibitor, a Bcl-2 inhibitor and other inhibitors) achieve synergistic effect (synergy) in treatment of a cancer and/or reducing a side-effect of the anticancer reagent in treatment of a cancer.

In a preferred embodiment, the invention provides the use of an EED inhibitor alone or in combination with one or more anticancer reagents (e.g., those selected from the group consisting of an ALK inhibitor, a BCR-ABL inhibitor, a MDM2 inhibitor, a Bcl-2 inhibitor and other inhibitors) for treating or suppressing a cancer, reducing its severity, lowering its risk or inhibiting its metastasis in an individual.

In a preferred embodiment, the EED, ALK, BCR-ABL, MDM2 and/or Bcl-2 inhibitor is as defined above and the cancer is as defined above.

In a preferred embodiment, the individual suffers from an advanced cancer.

In a preferred embodiment, the individual suffers from a refractory cancer, a recurrent cancer or a drug-resistant cancer.

In a preferred embodiment, the invention provides the use of an EED inhibitor alone or in combination with one or more anticancer reagents (e.g., those selected from the group consisting of an ALK inhibitor, a BCR-ABL inhibitor, a MDM2 inhibitor, a Bcl-2 inhibitor and other inhibitors) in the manufacture of a medicament for treating or suppressing a cancer, reducing its severity, lowering its risk or inhibiting its metastasis in an individual.

In a preferred embodiment, the EED, ALK, BCR-ABL MDM2 and/or Bcl-2 inhibitor is as defined above and the cancer is as defined above.

In a preferred embodiment, the individual suffers from an advanced cancer.

In a preferred embodiment, the individual suffers from a refractory cancer, a recurrent cancer or a drug-resistant cancer.

In another embodiment, the invention provides a kit, comprising:
(a) a first component in a first container, the first component comprising an EED inhibitor, and optionally a pharmaceutically acceptable carrier;
(b) a second component in a second container, the second component comprising an anticancer reagent, and optionally a pharmaceutically acceptable carrier; and
(c) an optional specification.

### EXAMPLES

In order to make the objects and technical solutions of the present invention clearer, the present invention will be further described below in conjunction with specific example. It should be understood that the examples are not intended to limit the scope of the invention. Further, specific experimental methods not mentioned in the following examples were carried out in accordance with a conventional experimental method.

Specifically, the below Examples 1-13 illustrate the synthesis and characterization of the EED inhibitor of the present invention, and Examples 14-27 illustrate the biological Assays of the EED inhibitor and the combination of the present invention.

### Example 1

### Synthesis of 12-(((5-fluoro-2,3-dihydrobenzofuran-4-yl)methyl)amino)-3H,5H-4-oxa-2,6,11,12a-tetraazabenzo[4,5]cycloocta[1,2,3-cd]inden-3-one (Cpd. No. 12)

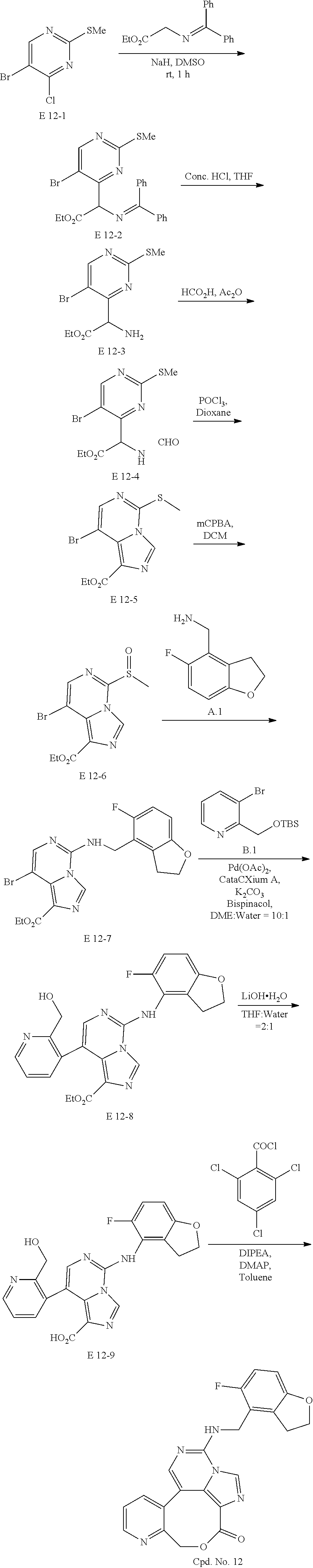

A solution of ethyl 2-(diphenylmethyleneamino)acetate (18.4 g, 69 mmol) in DMSO (50 ml) was added dropwise at 0° C. to a suspension of NaH (60%) (5.0 g, 125.5 mmol) in 70 ml of anhydrous DMSO. The reaction mixture turned orange immediately. After 5 min ethyl 2-((diphenylmethylene)amino)acetate (15 g, 62.7 mmol) in 50 ml DMSO was added dropwise. The mixture was stirred at room temperature for 2 h. After that the reaction mixture was quenched by careful addition of aq. NH₄Cl solution. The mixture was extracted then with ethyl acetate, washed with brine, dried and concentrated and used as crude for the next step. LC-MS: [M+H]+=470.01.

To a solution of compound E 12-2 (crude, 5.0 g, 10.6 mmol) in THF (50 ml) was added 10 ml 3 N HCl in water at 0° C. The mixture was stirred at room temperature for 1 h and the reaction mixture was concentrated followed by basification to pH 8˜9 with aq. Na₂CO₃ solution. The mixture was extracted with DCM, washed with brine. Concentration under reduced pressure followed by purification by flash chromatography (0-100% EtOAc/Hexane) gave the desired compound E 12-3 in 80% overall yield. LC-MS: [M+H]+=305.95.

A mixture of HCOOH (4 ml) and Ac₂O (4 ml) was heated at 50° C. for 1 h. The reaction mixture was cooled to room temperature and added to a solution of ethyl 2-amino-2-(methylthio) pyrimidin-4-yl) acetate (compound E 12-3, 2.0 g, 6.55 mmol) in 20 ml of DCM. The mixture was stirred at room temperature for 2 h. After completion of the reaction, the mixture was concentrated. The mixture was extracted with DCM (2×50 ml), washed successively with water (20 ml), and brine (10 ml). The organic phase was dried (Na₂SO₄), filtered and concentrated to afford the crude title compound E 12-4 as an oil, which was used for the next steps without further purification. LC-MS: [M+H]+=334.05.

To a solution of compound E 12-4 (2.0 g, crude) in dioxane (20 ml) was added POCl₃ (1.5 ml) dropwise. The reaction mixture was heated under reflux for 4 h. The mixture was cooled to rt and concentrated. Ice cooled water (50 ml) was added, and the mixture was adjusted to pH 8 with satd. aq. NaHCO₃. The mixture was extracted with DCM (2×50 ml), washed with brine (10 ml), dried (Na₂SO₄) and filtered. The filtrate was concentrated, and the residue was purified by silica gel column chromatography (eluted with 50-100% EtOAc/Hexane) to afford the title compound E 12-5 as a white solid (1.42 g, 4.59 mmol) in 70% overall yield in two steps. LC-MS: [M+H]+=315.70. ¹H NMR (400 MHz, DMSO d₆): 8.67 (s, 1H), 7.99 (s, 1H), 4.33 (q, 2H), 2.76 (s, 3H), 1.34 (t, 3H).

To a solution of compound E 12-5 (567 mg, 1.8 mmol, 1.0 eq.) in DCM (18 ml) was added m-CPBA (464 mg, 2.7 mmol, ≤77%, 1.5 eq.) at 0° C. After 45 min, Et₃N (1 ml, 7.6 mmol, 4 eq.) was added at 0° C. and stirred for 2 min, followed by addition of compound A.1 (300 mg, 1.8 mmol). The reaction mixture was then stirred at room temperature for 3 h. After that the reaction mixture was concentrated and the residue was purified by silica gel column chromatography (eluted with 50-100% EtOAc/Hexane) to afford E 12-7 (429 mg, 0.99 mmol) in 55% yield. LC-MS: [M+H]+=434.03. ¹H NMR (400 MHz, DMSO-d₆) δ 8.75 (s, 1H), 8.65 (t, J=5.1 Hz, 1H), 7.68 (s, 1H), 6.94 (t, J=9.5 Hz, 1H), 6.70 (dd, J=8.7, 3.9 Hz, 1H), 4.68 (d, J=5.0 Hz, 2H), 4.54 (t, J=8.7 Hz, 2H), 4.29 (q, J=7.1 Hz, 2H), 3.27 (t, J=8.8 Hz, 2H), 1.32 (t, J=7.1 Hz, 3H).

Palladium (II) acetate (70 mg, 0.31 mmol, 0.1 eq.) and cataCXium A (221 mg, 0.62 mmol, 0.2 eq.) were mixed together in DME (2.0 ml, degassed) and resulting solution was added via pipette to a stirred solution of compound E 12-7 (1.34 g, 3.1 mmol, 1.0 eq.), compound B.1 (1.86 g, 6.2 mmol, 2.0 eq.), bis-pinacolatediboron (1.6 g, 6.2 mmol, 2.0 eq.) and K₂CO₃ (1.71 g, 12.4 mmol, 4.0 eq.) in DME/H₂O (10:1, 22 ml, degassed) at 70° C. The reaction mixture was the stirred for 12 h. After that the reaction mixture was concentrated and extracted with ethyl acetate (2×50 ml), washed with water and brine, and then dried over Na₂SO₄. The mixture was concentrated, and residue was purified by HPLC to afford the desired compound, which on treatment of TFA/DCM afforded the desired compound E 12-8 (719 mg, 1.55 mmol) as a white solid in 50% yield. LC-MS: [M+H]+=464.16.

A mixture of E 12-8 (40 mg, 0.090 mmol, 1 equiv.) and LiOH (20 mg, 0.90 mmol, 10 eq.) in THF (4 ml) and water (1.0 ml) was heated at 70° C. for overnight. 3 N aq. HCl was added drop wise at 0° C. until pH 2-3. The mixture was concentrated, and residue was purified by HPLC to afford the title compound E 12-9 (35 mg, 0.081 mmol) as a white solid in 90% yield. LC-MS: [M+H]+=436.13.

To a cloudy mixture of 2,4,6-trichlorobenzoyl chloride (24 mg, 0.01 mmol, 1.5 eq.), DIPEA (85 mg, 0.66 mmol, 10.0 eq.), and DMAP (4 mg, 0.033 mmol, 0.5 eq.) in toluene (2 ml) was added a clear solution of secoacid E 12-9 (31 mg, 0.066 mmol) in toluene (1 ml) slowly via cannula. After 2 h, the reaction mixture was concentrated. The crude product was extracted with ethyl acetate (2×10 ml), washed in brine, and dried over Na₂SO₄. The mixture was concentrated, and residue was purified by HPLC to afford Cpd. No. 12 (16 mg, 0.039 mmol) as a white solid in 60% yield. LC-MS: [M+H]+=418.12. ¹H NMR (400 MHz, Acetone-d₆) δ 8.77 (s, 1H), 8.66 (d, J=4.7 Hz, 1H), 8.20 (s, 1H), 8.04 (d, J=7.9 Hz, 1H), 7.75 (s, 1H), 7.56 (dd, J=8.0, 4.6 Hz, 1H), 6.90 (t, J=9.4 Hz, 1H), 6.67 (dd, J=8.6, 3.8 Hz, 1H), 6.04 (s, 1H), 5.08 (s, 1H), 4.90 (s, 2H), 4.59 (t, J=8.6 Hz, 2H), 3.49 (t, J=8.6 Hz, 2H).

### Example 2

### 12-(((5-fluoro-2,3-dihydrobenzofuran-4-yl)methyl)amino)-4,5-dihydro-3H-2,4,6,11,12a-pentaazabenzo[4,5]cycloocta[1,2,3-cd]inden-3-one (Cpd. No. 16)

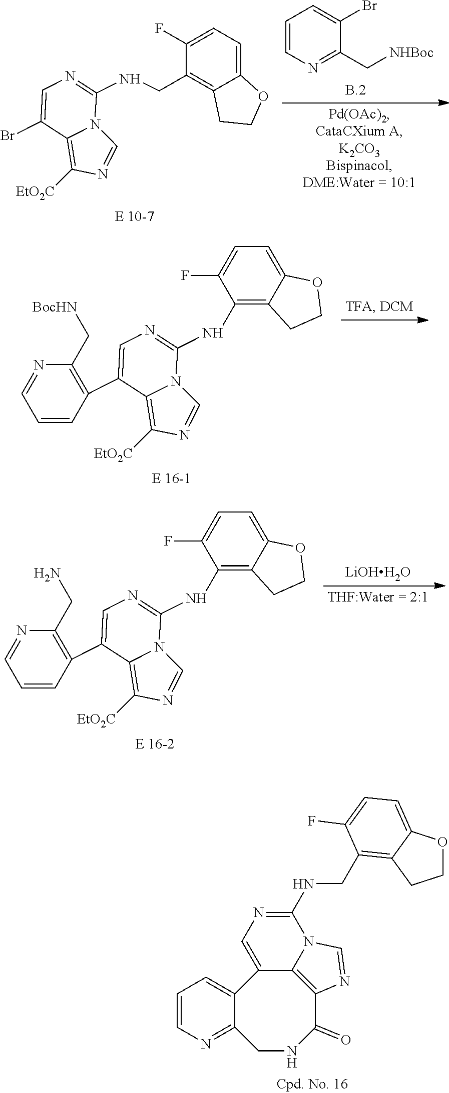

Palladium (II) acetate (70 mg, 0.31 mmol, 0.1 eq.) and cataCXium A (221 mg, 0.62 mmol, 0.2 eq.) were mixed together in DME (2.0 ml, degassed) and resulting solution was added via pipette to a stirred solution of compound E 10-7 (1.34 g, 3.1 mmol, 1.0 eq.), compound B.2 (1.77 g, 6.2 mmol, 2.0 eq.), bis-pinacolatediboron (1.6 g, 6.2 mmol, 2.0 eq.) and K₂CO₃ (1.71 g, 12.4 mmol, 4.0 eq.) in DME/H₂O (10:1, 22 ml, degassed) at 70° C. The reaction mixture was the stirred for 12 h. After that the reaction mixture was concentrated and extracted with ethyl acetate (2×50 ml), washed with water and brine, and then dried over Na₂SO₄. The mixture was concentrated, and residue was purified by HPLC to afford the title compound E 16-1 (871 mg, 1.55 mmol) as a white solid in 50% yield. LC-MS: [M+H]+=563.16.

Compound E 16-1 was treated with 25% TFA/DCM at room temperature for 1 h, after that the volatiles were removed in vacuo. The crude was diluted with ethyl acetate, washed with satd. aq. Na₂CO₃, then brine. The organic layer was over Na₂SO₄ and concentrated in vacuo to provide the compound E 16-2, which was used as crude for the next step.

A mixture of E 16-2 (40 mg, 0.09 mmol, 1 eq.) and LiOH (20 mg, 0.90 mmol, 10 eq.) in THF (4 ml) and water (1.0 ml) was heated at 70° C. for overnight. 3 N aq. HCl was added drop wise at 0° C. until pH 2-3. The mixture was concentrated, and residue was purified by HPLC to afford Cpd. No. 16 as a white solid in 90% yield. LC-MS: [M+H]+=416.14. ¹H NMR (400 MHz, DMSO-d₆) δ 8.82 (s, 1H), 8.65 (t, J=5.1 Hz, 1H), 8.54 (dd, J=4.8, 1.6 Hz, 1H), 8.47 (s, 1H), 7.92 (dd, J=7.8, 1.6 Hz, 1H), 7.53-7.48 (m, 2H), 6.96 (dd, J=10.3, 8.7 Hz, 1H), 6.72 (dd, J=8.7, 3.9 Hz, 1H), 4.98-4.94 (m, 1H), 4.75 (s, 2H), 4.57-4.53 (m, 2H), 4.03 (m, 1H), 3.35 (t, J=8.7 Hz, 2H).

### Example 3

### Synthesis of 11-(((5-fluoro-2,3-dihydrobenzofuran-4-yl)methyl)amino)-2,4,10,11a-tetraazadibenzo[cd,f]azulen-3(4H)-one (Cpd. No. 3)

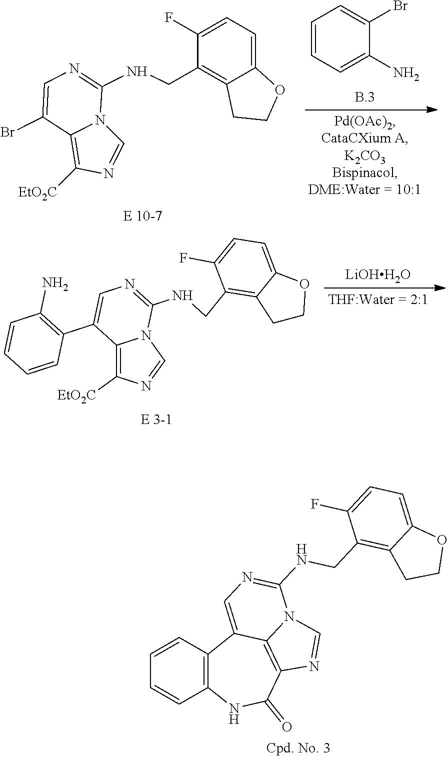

Palladium (II) acetate (70 mg, 0.31 mmol, 0.1 eq.) and cataCXium A (221 mg, 0.62 mmol, 0.2 eq.) were mixed together in DME (2.0 ml, degassed) and resulting solution was added via pipette to a stirred solution of compound E 10-7 (1.34 g, 3.1 mmol, 1.0 eq.), compound B.3 (1.05 g, 6.2 mmol, 2.0 eq.), bis-pinacolatediboron (1.6 g, 6.2 mmol, 2.0 eq.) and K₂CO₃ (1.71 g, 12.4 mmol, 4.0 eq.) in DME/H₂O (10:1, 22 ml, degassed) at 70° C. The reaction mixture was the stirred for 12 h. After that the reaction mixture was concentrated and extracted with ethyl acetate (2×50 ml), washed with water and brine, and then dried over Na₂SO₄. The mixture was concentrated, and residue was purified by HPLC to afford the title compound E 3-1 (692 mg, 1.55 mmol) as a white solid in 50% yield. LC-MS: [M+H]+=563.16.

A mixture of E 3-1 (40 mg, 0.090 mmol, 1 eq.) and LiOH (20 mg, 0.90 mmol, 10 eq.) in THF (4 ml) and water (1.0 ml) was heated at 70° C. for overnight. 3 N aq. HCl was added drop wise at 0° C. until pH 2-3. The mixture was concentrated, and residue was purified by HPLC to afford the Cpd. No. 3 (32 mg, 0.081 mmol) as a white solid in 90% yield. LC-MS: [M+H]+=402.13. ¹H NMR (400 MHz, DMSO-d₆) δ 9.50 (s, 1H), 8.67 (s, 1H), 8.53 (t, J=5.1 Hz, 1H), 7.99 (s, 1H), 7.88-7.80 (m, 1H), 7.19 (dd, J=6.1, 1.6 Hz, 2H), 7.02 (ddd, J=8.3, 6.1, 2.4 Hz, 1H), 6.96 (dd, J=10.3, 8.7 Hz, 1H), 6.71 (dd, J=8.6, 3.9 Hz, 1H), 4.73 (d, J=4.8 Hz, 2H), 4.56 (t, J=8.7 Hz, 2H), 3.31 (d, J=8.5 Hz, 2H).

### Example 4

### Synthesis of 4-cyclopropyl-12-(((5-fluoro-2,3-dihydrobenzofuran-4-yl)methyl)amino)-7-(trifluoromethyl)-4,5-dihydro-3H-2,4,8,11,12a-pentaazabenzo[4,5]cycloocta[1,2,3-cd]inden-3-one (Cpd. No. 36)

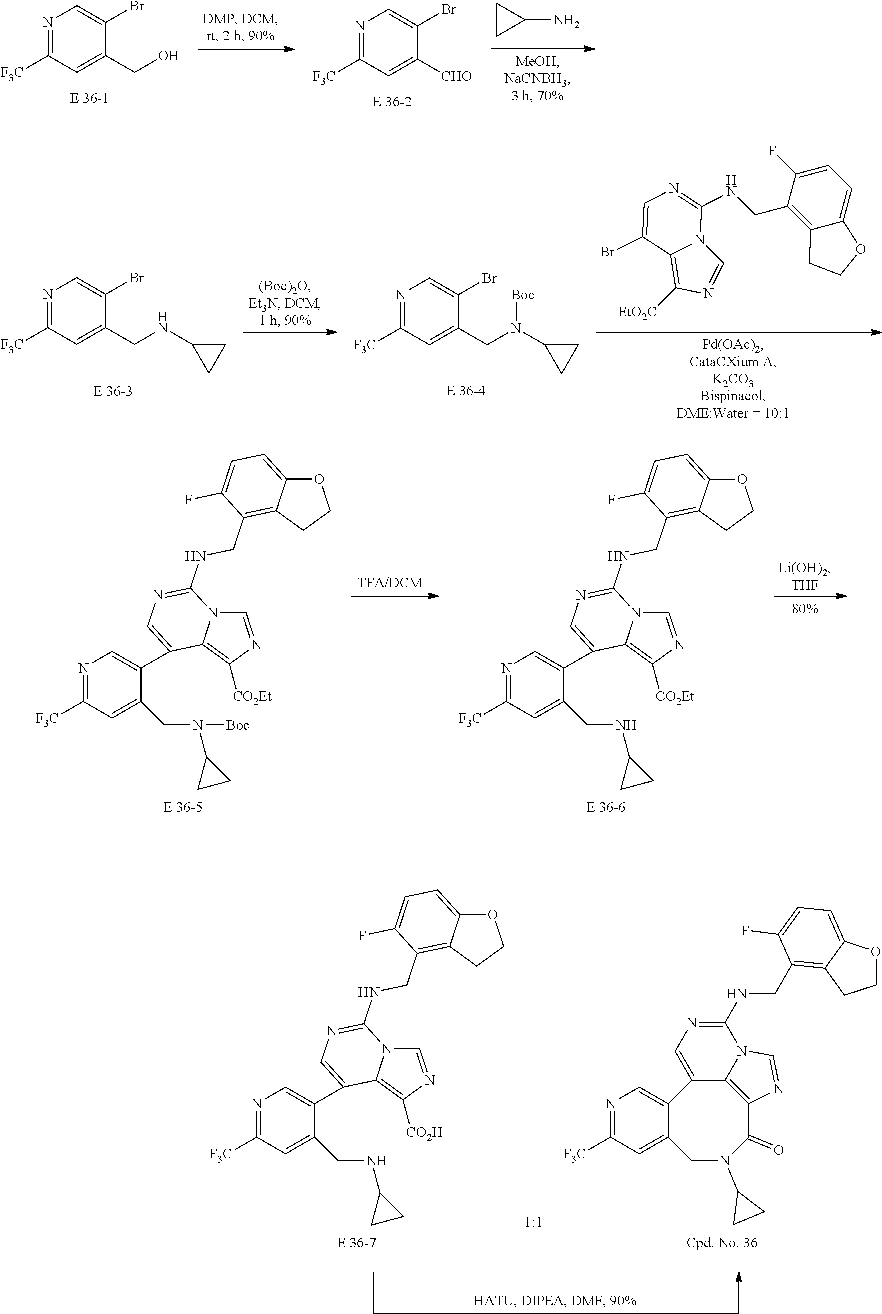

An aliquot of (5-bromo-2-(trifluoromethyl)pyridin-4-yl)methanol (E 36-1) was dissolved in dry DCM (˜0.2 M), then to this solution 1.5 eq. of Dess-Martin periodinane was added and the reaction mixture is allowed to stirring for 1 h, monitored via TLC. Upon completion quenched with saturated NH₄Cl solution, then extracted with DCM and washed with water and brine. The organic layers were collected and combined, washed with brine, dried over anhydrous Na₂SO₄, and concentrated in vacuo. Purification was performed on silica gel normal phase column chromatography with increasing amounts of ethyl acetate in hexanes to afford the desired aldehyde E 36-2 (yield ˜90%).

To the obtained aldehyde was added methanol (˜0.2 M), followed by 2.2 eq. of cyclopropanamine, 2 eq. of Na(CN)BH₃ and 2 eq. of acetic acid under ice bath. Then remove the ice bath and reaction mixture is allowed to stir for 3 h, monitored via TLC. Upon completion, the reaction mixture was concentrated, and residue was purified by HPLC to afford the title compound E 36-3 in 70% yield. LC-MS: [M+H]+=294.99.

To the obtained secondary amine was added 1.5 eq. of (Boc)₂O, dissolved by dry DCM (˜0.2 M), then followed by 3 eq of TEA, the reaction mixture is allowed to stir for 1 h, monitored via TLC. Upon completion it was quenched with saturated NH₄Cl solution, then extracted with DCM and washed with brine. The organic layers were collected and combined, dried over anhydrous Na₂SO₄, and concentrated in vacuo. Purification was performed on silica gel normal phase column chromatography with increasing amounts of ethyl acetate in hexanes to afford the Boc protected secondary amine E 36-4 (yield ˜90%). LC-MS: [M+H]+=395.10.

Palladium (II) acetate (0.1 eq.) and cataCXium A (0.2 eq.) were mixed together in DME (0.5 ml, degassed) and resulting solution was added via pipette to a mixture of ethyl 8-bromo-5-(((5-fluoro-2,3-dihydrobenzofuran-4-yl)methyl)amino) imidazo[1,5-c]pyrimidine-1-carboxylate (1 eq.), the Boc protected secondary amine E 36-4 (2 eq.), bis-pinacolatediboron (2.0 eq.) and K₂CO₃ (4.0 eq.) in DME/H₂O (10:1, 10 ml, degassed) at 70° C. The reaction mixture was the stirred for 12 h. After that the reaction mixture was concentrated and extracted with ethyl acetate (2×50 ml), washed with water and brine, and then dried over Na₂SO₄. The mixture was concentrated, and residue was purified by HPLC to afford the title compound E 36-5 in 40% yield. LC-MS: [M+H]+=671.25.

Compound E 36-5 was treated with 25% TFA/DCM at 0° C. for 1 h, after that the volatiles were removed in vacuo, which was used as crude (E 36-6) for the next step. LC-MS: [M+H]+=571.25.

A mixture of compound E 36-6 (1 eq.) and LiOH (10 eq.) in THF (10 ml/mmol) and water (5 ml/mmol) was heated at 70° C. for overnight. The mixture was concentrated, and residue was then purified by prep-HPLC to afford E 36 and compound E 36-7 in ˜1:1 ratio.

To a mixture of compound E 36-7 (1 eq.) and HATU (2 eq.) in DMF (5 ml/mmol) was added DIPEA (5 eq.). The reaction mixture is allowed to stir overnight. Then it was concentrated, and the residue was purified by prep-HPLC to Cpd. No. 36. The combined yield for both compounds is ˜90%. LC-MS: [M+H]+=525.15. ¹H NMR (400 MHz, methanol-d₄) δ 8.84 (s, 1H), 8.74 (s, 1H), 7.86 (s, 1H), 7.70 (s, 1H), 6.86 (t, J=9.6 Hz, 1H), 6.65 (dd, J=8.7, 4.0 Hz, 1H), 5.42 (d, J=14.7 Hz, 1H), 4.81 (d, J=6.1 Hz, 2H), 4.59 (t, J=8.9 Hz, 2H), 4.37 (d, J=14.9 Hz, 1H), 3.42 (t, J=8.7 Hz, 2H), 2.55 (s, 1H), 1.16 (s, 1H), 1.00 (d, J=5.5 Hz, 2H), 0.94-0.77 (m, 1H).

### Example 5

### Synthesis of N-((5-fluoro-2,3-dihydrobenzofuran-4-yl)methyl)-3H,5H-4-oxa-2,6,11,12a-tetraazabenzo[4,5]cycloocta[1,2,3-cd]inden-12-amine (Cpd. No. 2)

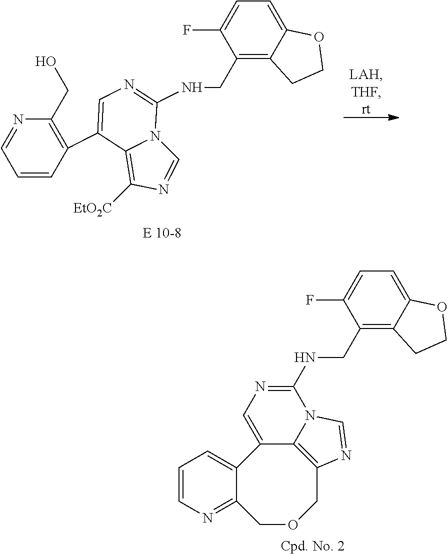 [SMILES: CCOC(=O)c1ncn2c(NCc3c(F)ccc4c3CCO4)ncc(-c3cccnc3CO)c12 ; "LAH,THF,rt" ; Fc1ccc2c(c1CNc1ncc3c4c(ncn14)COCc1ncccc1-3)CCO2]

Compound E 10-8 (25 mg, 0.053 mmol) in 2 ml of THF was treated with LAH (0.2 ml 1M solution of LAH in THF) at 0° C. After that temperature was increased to 50° C. and stirred overnight. After cooling to room temperature, the reaction was slowly quenched with satd. Na₂SO₄ at 0° C. It was then filtered and washed several times with ethyl acetate. Purification by flash chromatography (0-10% MeOH in DCM) gives Cpd. No. 2 (10 mg, 0.024 mmol) in 50% yield. LC-MS: [M+H]+=404.14.

### Example 6

### Synthesis of 11-(((5-fluoro-2,3-dihydrobenzofuran-4-yl)methyl)amino)-6-methyl-4H-3-thia-2,5,10,11a-tetraazadibenzo[cd,f]azulene 3,3-dioxide (Cpd. No. 95)

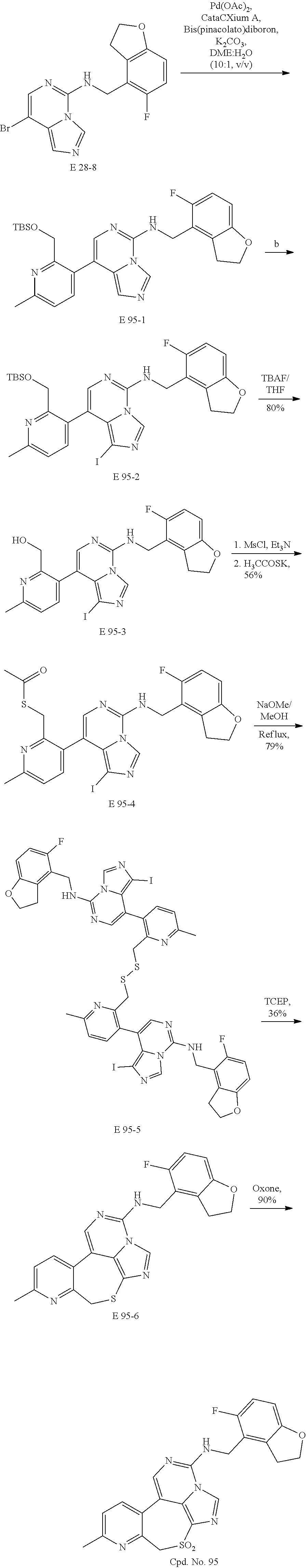

### Synthesis of 8-(2-(((tert-butyldimethylsilyl)oxy)methyl)-6-methylpyridin-3-yl)-N-((5-fluoro-2,3-dihydrobenzofuran-4-yl)methyl)imidazo[1,5-c]pyrimidin-5-amine (E 95-1)

8-bromo-N-((5-fluoro-2,3-dihydrobenzofuran-4-yl)methyl)imidazo[1,5-c]pyrimidin-5-amine (795 mg, 2.19 mmol), 3-bromo-2-(((tert-butyldimethylsilyl)oxy)methyl)-6-methylpyridine (1.393 g, 4.38 mmol), palladium (II) acetate (0.1 eq.), cataCXium A (0.2 eq.), bis-pinacolatediboron (2.0 eq.) and K2CO3 (5.0 eq.) were mixed together in DME:water (10:1, 17.4 ml, degassed) under N2 atmosphere. The reaction mixture was stirred for 12 h at 70° C. After that the reaction mixture was concentrated and extracted with ethyl acetate (2×200 ml), washed with water and brine, and then dried over Na2SO4. The mixture was concentrated, and the residue was purified by HPLC to afford the title compound E 95-1 (304 mg, 0.585 mmol) in 38% yield. LC-MS: [M+H]+=520.30.

### Synthesis of 8-(2-(((tert-butyldimethylsilyl)oxy)methyl)-6-methylpyridin-3-yl)-N-((5-fluoro-2,3-dihydrobenzofuran-4-yl)methyl)-1-iodoimidazo[1,5-c]pyrimidin-5-amine (E 95-2)

To a solution of 8-(2-(((tert-butyldimethylsilyl)oxy)methyl)-6-methylpyridin-3-yl)-N-((5-fluoro-2,3-dihydrobenzofuran-4-yl)methyl)imidazo[1,5-c]pyrimidin-5-a mine (304 mg, 0.585 mmol) in DMF (5 ml) at 0° C. was added NIS (125 mg, 0.95 mmol), and stirred at room temperature for 15 mins. The mixture was extracted with EA (4×50 ml), washed with brine (30 ml), dried (Na₂SO₄) and filtered. The filtrate was concentrated, and the residue was purified by column chromatography (silica gel, eluted with 20-80% EtOAc/Hexane) to afford E 95-2 as a yellow solid (190 mg, 0.29 mmol, 50%). LC-MS: [M+H]+=646.21.

### Synthesis of (3-(5-(((5-fluoro-2,3-dihydrobenzofuran-4-yl)methyl)amino)-1-iodoimidazo[1,5-c]pyrimidin-8-yl)-6-methylpyridin-2-yl)methanol (E 95-3)

To a solution of 8-(2-(((tert-butyldimethylsilyl)oxy)methyl)-6-methylpyridin-3-yl)-N-((5-fluoro-2,3-dihydrobenzofuran-4-yl)methyl)-1-iodoimidazo[1,5-c]pyrimidin-5-amine (190 mg, 0.29 mmol) in THF (6 ml) was added TBAF (3 ml), and stirred at room temperature overnight. Upon completion the mixture was extracted with EA (3×60 ml), washed with brine (30 ml), dried over Na₂SO₄ and filtered. The filtrate was concentrated, and the residue was purified by column chromatography (silica gel, eluted with 0-15% MeOH/DCM) to afford E 95-3 (125 mg, 0.24 mmol, 80%). LC-MS: [M+H]+=532.19.

### Synthesis of S-((3-(5-(((5-fluoro-2,3-dihydrobenzofuran-4-yl)methyl)amino)-1-iodoimidazo[1,5-c]pyrimidin-8-yl)-6-methylpyridin-2-yl)methyl) ethanethioate (E 95-4)

MsCl (41 mg, 0.352 mmol) in THF (0.5 ml) was added dropwise to a solution of (3-(5-(((5-fluoro-2,3-dihydrobenzofuran-4-yl)methyl)amino)-1-iodoimidazo[1,5-c]pyrimidin-8-yl)-6-methylpyridin-2-yl)methanol (125 mg, 0.24 mmol) and Et₃N (36 mg, 0.352 mmol) in THF (2 ml) at 0° C. A white ppt of Et₃N hydrochloride formed immediately. The reaction mixture was stirred for 2-3 hrs, upon completion potassium thioacetate (81 mg, 0.704 mmol) in DMF (1.0 ml) was added, resulting in an orange solution which tuned red after several hours. The reaction was monitored via UPLC, upon completion the reaction mixture was stopped and concentrated, then dissolved in DCM. This mixture solution was washed with saturated LiCl twice, followed by water and brine. The saturated LiCl, brine and water washes were combined and separately back-extracted with ethyl acetate. All organic layers are combined, dried over Na₂SO₄, filtered and concentrated to dark oil. Flash chromatography (silica gel, eluted with 20-100% EtOAc/Hexane) to afford E 95-4 as a yellow solid (78 mg, 0.13 mmol, 56%). LC-MS: [M+H]+=590.03.

### Synthesis of 8,8′-((disulfanediylbis(methylene))bis(6-methylpyridine-2,3-diyl))bis(N-((5-fluoro-2,3-dihydrobenzofuran-4-yl)methyl)-1-iodoimidazo[1,5-c]pyrimidin-5-amine) (E 95-5)

To a solution of S-((3-(5-(((5-fluoro-2,3-dihydrobenzofuran-4-yl)methyl)amino)-1-iodoimidazo[1,5-c]pyrimidin-8-yl)-6-methylpyridin-2-yl)methyl) ethanethioate (78 mg, 0.13 mmol) in methanol (5.0 ml, degassed) was added 0.9 eq. of NaOMe (7 mg, 0.12 mmol) under N₂ atmosphere. The reaction mixture was refluxed at 80° C. for an hour. Flash chromatography (silica gel, eluted with 20-100% EtOAc/Hexane) to afford E 95-5 as a yellow solid (57 mg, 0.052 mmol, 79%). LC-MS: [M/2+H]+=547.14.

### Synthesis of N-((5-fluoro-2,3-dihydrobenzofuran-4-yl)methyl)-6-methyl-4H-3-thia-2,5,10,11a-tetraazadibenzo[cd,f]azulen-11-amine (E 95-6)

To a solution of 8,8′-((disulfanediylbis(methylene))bis(6-methylpyridine-2,3-diyl))bis(N-((5-fluoro-2,3-dihydrobenzofuran-4-yl)methyl)-1-iodoimidazo[1,5-c]pyrimidin-5-amine) (57 mg, 0.052 mmol) in DMF (3.0 ml, degassed) was added 1.2 eq. of TCEP (18 mg, 0.0626 mmol) under N₂ atmosphere. The reaction mixture was stirred at room temperature for 24 hrs. The reaction was monitored by UPLC. Upon completion it was purified by reverse phase HPLC to afford E 95-6 as a pale yellow solid (38 mg, 0.091 mmol, 87%). LC-MS: [M+H]+=420.15.

### Synthesis of 11-(((5-fluoro-2,3-dihydrobenzofuran-4-yl)methyl)amino)-6-methyl-4H-3-thia-2,5,10,11a-tetraazadibenzo[cd,f]azulene 3,3-dioxide (E 95)

To a solution of N-((5-fluoro-2,3-dihydrobenzofuran-4-yl)methyl)-6-methyl-4H-3-thia-2,5,10,11a-tetraazadibenzo[cd,f]azulen-11-amine (38 mg, 0.091 mmol) in a mixed solvent of MeOH:H2O:THF (4.0 mL, 5:5:10) was added 5 eq. of oxone (279 mg, 0.45 mmol). The reaction mixture was stirred for 5 hours. Upon completion reverse phase HPLC afforded Cpd. No. 95 as a pale yellow solid (17 mg, 0.038 mmol, 41%). LC-MS: [M+H]+=452.25.

### Example 7

### Synthesis of 12-(((5-fluoro-2,3-dihydrobenzofuran-4-yl)methyl)amino)-7-(methyl-d3)-4-(2,2,2-trifluoroethyl)-4,5-dihydro-3H-2,4,6,11,12a-pentaazabenzo[4,5]cycloocta[1,2,3-cd]inden-3-one (Cpd. No. E 19)

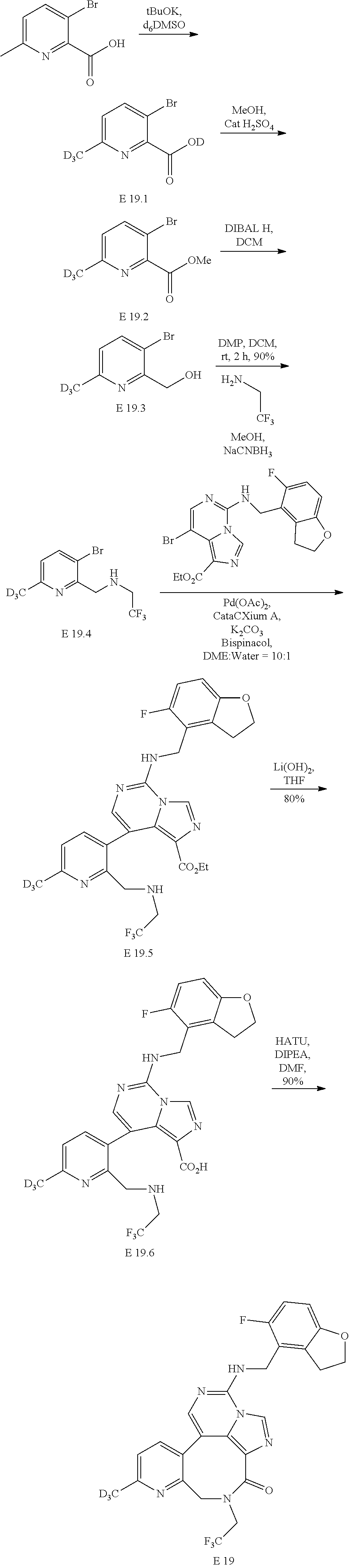

To a flame-dried flask was added 3-bromo-6-methylpicolinic acid (1 gm, 4.67 mmol), followed by potassium tert-butoxide (1.0 gm, 9.34 mmol) and DMSO-d6 (12 mL), and the mixture was stirred at room temperature under argon for 12 h. The reaction mixture was diluted with cold water (10 mL) and extracted with ethyl acetate (20 mL). The ethyl acetate layer was washed with brine (20 mL×2), dried over anhydrous sodium sulfate, and concentrated to yield crude product (E 19.1) in 90% yield. LC-MS: [M+H]+=215.95.

H₂SO₄ (1.0 eq.) was added to a solution of 3-bromo-6-(methyl-d3)picolinic acid-d (E 19.1, 5.0 gm, 23.3 mmol, 1.0 eq.) in MeOH (50 ml). The resulting solution was stirred for 14 h while the temperature was maintained at reflux in an oil bath. The mixture was cooled to room temperature and concentrated under vacuum. The residue was dissolved in ethyl acetate (50 ml) and washed with water and satd. aq. NaCl (2×100 ml), dried over anhydrous Na₂SO₄ and concentrated under vacuum. The residue was purified by silica gel column chromatography and eluted with EtOAc/Hexane (1:5) to yield methyl 3-bromo-6-methylpicolinate (E 19.2, 4.7 gm) in 90% yield. LC-MS [M+H]⁺=232.99.

To a solution of methyl 3-bromo-6-(methyl-d3)picolinate (E 19.2, 520 mg, 2.15 mmol) in DCM (15 ml) at −60° C., DIBAL-H (4.6 ml, 4.60 mmol, 1 M in cyclohexane) was added dropwise. The reaction mixture was maintained at −60° C. to −15° C. for 30 min, allowed to rise to room temperature, and stirred for another 12 h. The reaction mixture was cooled to 0° C. again, and was quenched with satd. aq. NH₄Cl (50 ml). The resulting mixture was extracted with DCM (3×100 ml), washed with brine (50 ml), dried (Na₂SO₄), filtered, and concentrated. The residue was purified by silica gel column chromatography and eluted with EtOAc/Hexane (1:3) to afford E 19.3 as a colorless liquid (1.36 mmol, 273 mg, 60%). LC-MS [M+H]+=204.99.

An aliquot of (3-bromo-6-(methyl-d3)pyridin-2-yl)methanol (E 19.3) was dissolved in dry DCM (˜0.2 M). To this solution, 1.5 eq. of DMP was added, and the reaction mixture was allowed to stir for 1 h (monitored by TLC). Upon completion, the reaction was quenched with saturated NH₄Cl solution, extracted with DCM, and washed with water and brine. The organic layers were collected and combined, washed with brine, dried over anhydrous Na₂SO₄, and concentrated in vacuo. Purification was performed on silica gel normal phase column chromatography with increasing amounts of ethyl acetate in hexanes to afford the desired aldehyde.

To the aldehyde was added methanol (˜0.2 M), followed by 2.2 eq. of c trifluoroethan-1-amine, 2 eq. of Na(CN)BH₃, and 2 eq. of acetic acid at 0° C. The ice bath was removed and reaction mixture was allowed to stir for 3 h. Upon completion, the reaction mixture was concentrated and residue was purified by HPLC to afford E 19.4 in 70% yield. LC-MS: [M+H]+=286.01.

Palladium (II) acetate (0.1 eq.) and cataCXium A (0.2 eq.) were mixed together in DME (0.5 ml, degassed), and resulting solution was added via pipette to a mixture of ethyl 8-bromo-5-(((5-fluoro-2,3-dihydrobenzofuran-4-yl)methyl)amino) imidazo[1,5-c]pyrimidine-1-carboxylate (1 eq.), the secondary amine E 19.4 (2 eq.), bis-pinacolatediboron (2.0 eq.) and K₂CO₃ (4.0 eq.) in DME/H₂O (10:1, 10 ml, degassed) at 70° C. The reaction mixture was the stirred for 12 h. The reaction mixture was concentrated and extracted with ethyl acetate (2×50 ml), washed with water and brine, and dried over Na₂SO₄. The mixture was concentrated and residue was purified by HPLC to afford E19.5 in 50% yield. LC-MS: [M+H]+=561.21.

A mixture of compound E 19.5 (1 eq.) and LiOH (10 eq.) in THF (10 ml/mmol) and water (5 ml/mmol) was heated at 70° C. overnight. The mixture was concentrated, and residue was then purified by prep-HPLC to afford E 19.6 in 80% yield. LC-MS: [M+H]+=534.18.

To a mixture of compound E 19.6 (1 eq.) and HATU (2 eq.) in DMF (5 ml/mmol) was added DIPEA (5 eq.). The reaction mixture was allowed to stir overnight, and concentrated. The residue was purified by prep-HPLC to afford Cpd. No. E 19 in 90% yields. LC-MS: [M+H]⁺=516.17. ¹H NMR (400 MHz, DMSO-d⁶) δ 8.85 (s, 1H), 8.71 (t, J=5.1 Hz, 1H), 7.93 (d, J=8.0 Hz, 1H), 7.54 (s, 1H), 7.44 (d, J=8.1 Hz, 1H), 6.96 (dd, J=10.3, 8.6 Hz, 1H), 6.71 (dd, J=8.7, 3.9 Hz, 1H), 5.46 (d, J=15.0 Hz, 1H), 4.75 (d, J=4.5 Hz, 2H), 4.71-4.61 (m, 1H), 4.56 (t, J=8.8 Hz, 2H), 4.19 (d, J=15.0 Hz, 1H), 4.08 (dq, J=15.2, 9.0 Hz, 1H), 3.34 (t, J=8.7 Hz, 2H), 2.57 (s, 3H).

### Example 8

### Synthesis of 8-(6-cyclopropylpyridin-3-yl)-5-(((5-fluoro-2,3-dihydrobenzofuran-4-yl)methyl)amino)-1-(methylsulfonyl)imidazo[1,5-a]pyridine-6-carbonitrile (Cpd. No. E 26)

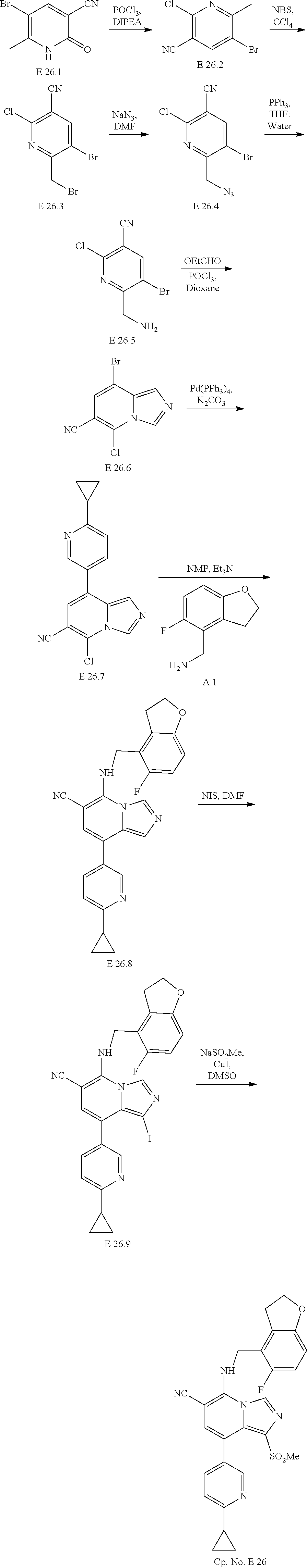

A mixture of 5-bromo-6-methyl-2-oxo-1,2-dihydropyridine-3-carbonitrile (E 26.1, 120 mg, 0.55 mmol) and DIPEA (145 mg, 1.14 mmol) in POCl₃ (2.0 ml) was refluxed for 3 h. The resulting brown mixture was evaporated in vacuo, and 10 ml of EtOAc and 5 ml aq. NaHCO₃ solution were added. The mixture was extracted with EtOAc (20 ml×3), dried (Na₂SO₄), filtered and concentrated. The residue was purified by flash chromatography (silica gel, eluted with PE/EA=1/3˜1/5) to afford E 26.2 (70 mg, 54% yield) as a white solid. LC-MS: [M+H]+=230.1.

To a solution of 5-bromo-2-chloro-6-methylnicotinonitrile (4.46 gm, 19.4 mmol) and NBS (3.79 mg, 21.3 mmol) in CCl₄ (80 ml) was added BPO (469 mg, 1.94 mmol) at room temperature. The resulting mixture was degassed and stirred at 80° C. for 4 h under nitrogen. The reaction mixture was filtered, washed with brine, dried (Na₂SO₄), filtered, and concentrated. The crude product was purified by column chromatography on silica gel (eluted with 10% EtOAc/petroleum ether) to give 5-bromo-6-(bromomethyl)-2-chloronicotinonitrile (E 26.3) as yellow oil (4.1 gm, 74%). LC-MS: [M+H]+=308.83.

To a solution of 5-bromo-6-(bromomethyl)-2-chloronicotinonitrile (4.46 gm, 14.5 mmol) in DMF (50 ml) was added NaN₃ (1.89 gm, 29 mmol). The mixture was stirred room temperature for 2 h, poured into water, and extracted with EtOAc (30 ml×3). The combined organic layers were washed with brine, dried (Na₂SO₄), filtered, and concentrated to give 6-(azidomethyl)-5-bromo-2-chloronicotinonitrile as yellow oil (3.4 gm, yield: 87%), which was used directly to next step. LC-MS: [M+H]+=271.92.

To a solution of 6-(azidomethyl)-5-bromo-2-chloronicotinonitrile (3.4 gm, 12.6 mmol) in THF (50 ml) and H₂O (5 ml) was added PPh₃ (4.93 gm, 18.9 mmol). The resulting mixture was heated at 50° C. for 1 h, and concentrated. The residue was dissolved in 50 ml of aq. HCl and washed with DCM (20 ml×2). The aqueous layer was basified by addition of aq. NaOH to pH˜8 and extracted with EtOAc (30 ml×3). The organic layers were dried (Na₂SO₄), filtered, and concentrated to give the desired product 6-(aminomethyl)-5-bromo-2-chloronicotinonitrile as yellow oil (2.28 gm, 74%). LC-MS: [M+H]+=245.93.

To a solution of 6-(aminomethyl)-5-bromo-2-chloronicotinonitrile (2.25 gm, 9.3 mmol) in ethyl formate (40 ml) was added NaHCO₃ (391 mg, 4.6 mmol). The mixture was stirred at room temperature for 24 h and filtered. The filtrate was concentrated to give desired compound as brown oil (2.1 gm, 90%), which was used directly to next step. LC-MS: [M+H]+=273.93.

To a solution of the crude compound in dioxane (30 ml) was added POCl₃ (2.59 gm, 16.8 mmol). The mixture was refluxed for 3 h. The reaction mixture was quenched with aq. NaHCO₃ and extracted with EtOAc (30×3). The organic layers were washed with brine, dried (Na₂SO₄), filtered, and concentrated. The crude was purified by column chromatography on silica gel (10% EtOAc/petroleum) to give the desired compound (E.26.6) as light yellow solid (1.6 gm, 83%). LC-MS: [M+H]+=255.91.

A mixture of 8-bromo-5-chloroimidazo[1,5-a]pyridine-6-carbonitrile (60 mg, 0.24 mmol), (6-cyclopropylpyridin-3-yl)boronic acid (50 mg, 0.31 mmol), Pd(dppf)Cl₂ (12 mg, 0.015 mmol) and Na₂CO₃ (81 mg, 0.77 mmol) in H₂O (0.5 ml) and dioxane (1.5 ml) was heated at 110° C. for 1 h under N₂. The reaction mixture was filtered and concentrated. The crude product was purified by prep-HPLC to give 5-chloro-8-(6-cyclopropylpyridin-3-yl)imidazo[1,5-a]pyridine-6-carbonitrile (E 26.7, 74 mg, yield: 90%). LC-MS: [M+H]+=295.06.

To a solution of 5-chloro-8-(6-cyclopropylpyridin-3-yl)imidazo[1,5-a]pyridine-6-carbonitrile (38 mg, 0.13 mmol) and (5-fluoro-2,3-dihydrobenzofuran-4-yl)methanamine (65 mg, 0.39 mmol) in NMP (0.5 ml) was added triethylamine (39 mg, 0.39 mmol) at room temperature The resulting solution was heated at 130° C. by microwave for 1 h. The reaction mixture was concentrated and purified by column chromatography on silica gel (eluted with PE:EA=1:1) to give a yellow solid (E 26.8, 66%). LC-MS (m/z): 426.16 [M+H]+.

To a solution of N-((5-fluoro-2,3-dihydrobenzofuran-4-yl)methyl)-8-phenylimidazo[1,5-c]pyrimidin-5-amine (500 mg, 1.38 mmol) in DMF (10 ml) at 0° C. was added NIS (278 mg, 1.24 mmol). The mixture was stirred at room temperature for 15 min. The mixture was extracted with DCM (4×50 ml), washed with brine (30 ml), dried (Na₂SO₄), and filtered. The filtrate was concentrated, and the residue was purified by column chromatography (silica gel, eluted with 20-50% EtOAc/Hexane) to afford the title compound (E 26.9) as a yellow solid (610 mg, 1.26 mmol, 70%). LC-MS: [M+H]+=552.06.

A mixture of compound E 26.9 (50 mg, 0.09 mmol), MeSO₂Na (30 mg. 0.3 mmol), and CuI (57 mg, 0.3 mmol) in DMSO (2 ml) was bubbled with N₂ for 5 mins, and the sealed tube was then heated in a microwave reactor at 120° C. for 20 min, and then at 100° C. for 3 h. The mixture was concentrated, and residue was purified by HPLC to afford Cpd. No. E 26 (25 mg, 0.05 mmol) in 50% yield. LC-MS: [M+H]+=504.14.

### Example 9

### Synthesis of N-((5-fluoro-2,3-dihydrobenzofuran-4-yl)methyl)-5-methyl-8-(trifluoromethyl)-6H-2,3,5a,9,12,13a-hexaazabenzo[4,5]cyclopenta[7,8]cycloocta[1,2,3-cd]inden-13-amine (Cpd. No. E 10)

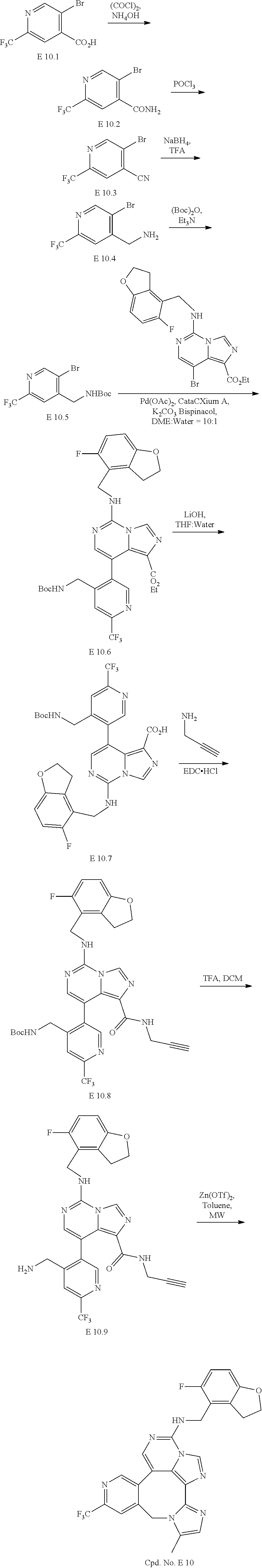

To a 500 ml round bottom flask equipped with a stir bar, condenser, and nitrogen inlet was charged 38.9 gm (144 mmol) of 5-bromo-2-trifluoromethyl-isonicotinic acid (E 10.1). To the solid was added 250 ml of anhydrous DCM followed by 13.2 ml (151 mmol, 1.05 eq.) of oxalyl chloride. To the mixture was added 0.5 ml of anhydrous DMF and the mixture was stirred at ambient temperature for 2 h. The solvent was removed under vacuum. To a 1 liter Erlenmeyer flask equipped with a stir bar in an ice bath was charged 500 ml of aq. NH₄OH. To the chilled solution was added dropwise the crude acid chloride. The residue was transferred with a small amount of acetonitrile. The mixture was stirred for 20 minutes following the addition. The resulting precipitate was collected by filtration and washed with water. The filter cake was dried in vacuo at 45 affording 5-bromo-2-trifluoromethyl-isonicotinamide (E 10.2, 118 mmol, 31.52 gm, 82% yield) as an off-white solid. LC-MS [M+H]+=269.95.

To a 100 mL round bottom equipped with a stir bar, condenser, and nitrogen inlet was charged 5.2 gm (19.3 mmol) of 5-bromo-2-trifluoromethylisonicotinamide (E 10.2). The solid was diluted with 12 ml of POCl₃. The mixture was heated at 70° C. for 3 h. The mixture was cooled to room temperature and poured onto ice. The mixture was neutralized with the careful addition of 50% sodium hydroxide. The resulting off-white solid was collected by filtration, washed with water and dried in vacuo for at 50° C. 18 h. This afforded 4.5 gm of 5-bromo-2-trifluoromethyl-isonicotinonitrile (E 10.3, 4.53 gm, 18.1 mmol) as an off-white solid in a 94% yield. LC-MS [M+H]+=250.95. ¹H NMR (CDCl₃): δ, 9.03 (s, 1H), 7.91 (s, 1H).

NaBH₄ (0.66 g, 14.81 mmol) was charged to a 100 ml flask followed by anhydrous THF 20 ml. The mixture was cooled in an ice-water bath. TFA (1.5 ml) was added to THF (4 ml) at that temperature for 0.5 h. The ice-water bath was removed and the resulting mixture was stirred at room temperature for 2 h. 5-bromo-2-(trifluoromethyl)isonicotinonitrile (E 10.3, 2 gm, 8.0 mmol) was dissolved in THF (10 ml). The TFA/NaBH₄ mixture was again cooled in an ice-water bath and the nitrile solution was added over 0.5 h. The mixture was allowed to reach ambient temperature while stirring for 16 h. LC analysis of an aliquot revealed completion of the reaction. The mixture was cooled in an ice bath and 10 ml methanol was added slowly. Volatiles were removed under vacuum and ethyl acetate (50 ml) was added. This mixture was washed with water (10 ml). The aqueous layer was washed with ethyl acetate (10 ml) and the combined organic layers were washed with brine (10 ml), dried over Na₂SO₄, filtered, and concentrated. The residue was purified by purified by reverse phase combi flash (eluted with 1-20% acetonitrile/H₂O) to afford the title compound (E 10.4, 1.6 gm, 80%) as a colorless liquid. LC-MS [M+H]+=254.96.

Compound (E 10.4, 512 mg, 2 mmol) is stirred at room temperature for 3 h with (Boc)₂O (0.51 gm, 2.4 mmol, 1.2 eq.) and Et₃N (2 eq., 4 mmol, 380 mg) in 20 ml DCM. The residue was purified by a column chromatography using 0-50% EtOAc/Hexane to give desired compound (E 10.5, 560 mg) 80% overall yield. LC-MS [M+H]+=355.16.

Palladium (II) acetate (0.1 eq.) and cataCXium A (0.2 eq.) were mixed together in DME (0.5 ml, degassed) and resulting solution was added via pipette to a mixture of ethyl 8-bromo-5-(((5-fluoro-2,3-dihydrobenzofuran-4-yl)methyl)amino) imidazo[1,5-c]pyrimidine-1-carboxylate (1 eq.), the secondary amine E 10.5 (2 eq.), bis-pinacolatediboron (2.0 eq.) and K₂CO₃ (4.0 eq.) in DME/H₂O (10:1, 10 ml, degassed) at 70° C. The reaction mixture was stirred for 12 h. The reaction mixture was concentrated and extracted with ethyl acetate (2×50 ml), washed with water and brine, and then dried over Na₂SO₄. The mixture was concentrated, and residue was purified by HPLC to afford E 10.6 in 50% yield. LC-MS: [M+H]+=631.22

A mixture of compound E 10.6 (1 eq.) and LiOH (10 eq.) in THF (10 ml/mmol) and water (5 ml/mmol) was heated at 70° C. for overnight. The mixture was concentrated, and residue was then purified by prep-HPLC to afford E 10.7 in 80% yield. LC-MS: [M+H]+=603.19.

In a 250 ml round bottom flask, a stirred solution of E 10.7 (3.29 gm, 5.47 mmol) and prop-2-yn-1-amine (0.601 gm, 10.94 mmol) in DMF (15 mL) was treated sequentially with EDCI·HCl (2.28 gm, 11.94 mmol), HOBt (1.61 gm, 11.93 mmol) and Et₃N (2.03 ml, 14.92 mmol) at room temperature under nitrogen atmosphere. The reaction mixture was stirred at room temperature for 12 h under nitrogen atmosphere. Upon completion of reaction (TLC), the reaction mixture was diluted with ice cold water. The mixture was concentrated, and residue was then purified by prep-HPLC to afford E 10.8 in 80% yield. LC-MS: [M+H]+=640.22

Compound E 10.8 was treated with 25% TFA/DCM at room temperature for 1 h, and the volatiles were removed in vacuo. The crude product was diluted with ethyl acetate, washed with satd. aq. Na₂CO₃, and brine. The organic layer was dried over Na₂SO₄ and concentrated under vacuum to provide the compound E 10.9, which was used as crude for the next step. LC-MS: [M+H]+=540.22.

In a 20 ml microwave vial, a solution of compound E 10.9 (0.22 g, 0.42 mmol) and (2-methoxyphenyl)methanamine (0.086 g, 0.63 mmol) in toluene (5 mL) was treated with Zn(OTf)₂ (0.009 g, 0.021 mmol) at room temperature under nitrogen atmosphere. The reaction mixture was subjected to microwave irradiation at 140° C. for 1 h. Upon completion of reaction (TLC), the reaction mixture was diluted with water and extracted with EtOAc (30 mL). The organic extract was washed with saturated NaHCO₃ and brine, and dried over anhydrous Na₂SO₄. The solution was concentrated under reduced pressure, and residue obtained was purified by prep-HPLC to afford Cpd. No. E 10 in 40% yields. LC-MS: [M+H]+=522.15.

### Example 10

### Synthesis of 12-(((5-fluoro-2,3-dihydrobenzofuran-4-yl)methyl)amino)-7-(tetrahydro-2H-pyran-4-yl)-4-(2,2,2-trifluoroethyl)-4,5-dihydro-3H-2,4,6,11,12a-pentaazabenzo[4,5]cycloocta[1,2,3-cd]inden-3-one (Cpd. No. E 45)

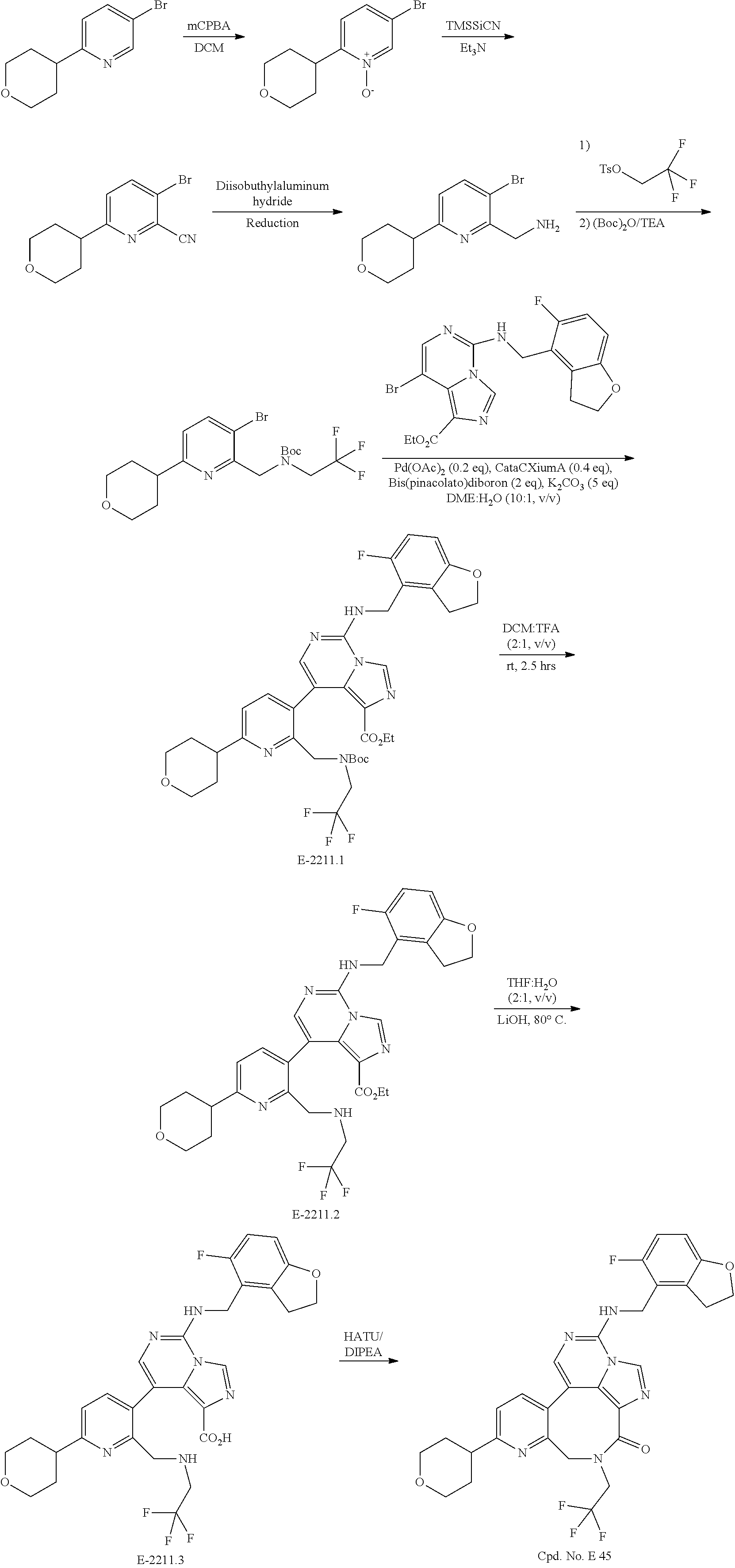

To a solution of 5-bromo-2-(tetrahydro-2H-pyran-4-yl)pyridine (5.00 g, 20.65 mmol) in DCM (100 ml) was added 1.5 eq of mCPBA (5.35 g, 30.97 mmol) slowly. After 4 h the reaction mixture was quenched with 2.0 eq of Ca(OH)₂ (3.90 g, 41.3 mmol), and the resulting precipitate was stirred for 30 minutes. The precipitate was filtered and washed with 3:1 DCM/methanol. The filtrate was concentrated in vacuo to give 5-bromo-2-(tetrahydro-2H-pyran-4-yl)pyridine 1-oxide as a crude solid, which was used for the subsequent reaction without further purification.

To the solution of crude 5-bromo-2-(tetrahydro-2H-pyran-4-yl)pyridine 1-oxide (4.00 g, 15.63 mmol) obtained from the previous step in acetonitrile (78 ml, 0.2 M) was added 6.0 eq of trimethylsilyl cyanide (TMSCN) (9.48 g, 94.00 mmol) and 4.5 eq of triethylamine (5.26 g, 70.34 mmol). The reaction was heated at 100° C. overnight. After cooling to room temperature, the solvent was concentrated in vacuo, and the residue was purified by HPLC (Acetonitrile/H₂O, started from 25% ACN, obtained compound at 42% ACN in H₂O) to give 3-bromo-6-(tetrahydro-2H-pyran-4-yl) picolinonitrile (1.60 g, 5.97 mmol, 29% in yield for 2 steps). LC-MS [M+H]+=266.97/268.96. ¹H NMR (400 MHz, DMSO-d₆) δ 8.30 (d, J=8.4 Hz, 1H), 7.64 (d, J=8.4 Hz, 1H), 3.96-3.93 (m, 2H), 3.46-3.40 (m, 2H), 3.05-2.98 (m, 1H), 1.79-1.68 (m, 4H).

3-bromo-6-(tetrahydro-2H-pyran-4-yl)picolinonitrile (1.60 g, 5.97 mmol) was dissolved in 50 ml of dry DCM and cooled to −78° C. While stirring the solution, 2 eq of DIBAL-H solution in toluene (12 ml, 11.94 mmol) was added dropwise. The mixture was stirred 5 hr, quenched by slow addition of saturated aqueous Rochelle's salt (sodium potassium tartrate), warmed to room temperature, diluted with ethyl acetate, and stirred until two easily separable clear layers were formed. HPLC purification gave (3-bromo-6-(tetrahydro-2H-pyran-4-yl)pyridin-2-yl)methanamine (1.12 g, 4.12 mmol, 69%) as a liquid. LC-MS [M+H]+=271.04/273.03. ¹H NMR (400 MHz, DMSO-d₆) δ 8.29 (s, broad, 2H), 8.07 (d, J=8.4 Hz, 1H), 7.29 (d, J=8.4 Hz, 1H), 4.53-4.22 (m, 2H), 3.48-3.42 (m, 2H), 3.00-2.93 (m, 1H), 1.91-1.69 (m, 4H).

To a solution of (3-bromo-6-(tetrahydro-2H-pyran-4-yl)pyridin-2-yl) methanamine (181 mg, 0.67 mmol) in DCM (10 ml) was added 2.0 eq of 2,2,2-trifluoroethyl 4-methylbenzenesulfonate (311 mg, 1.34 mmol) and 2.0 eq of DIPEA (173 mg, 1.34 mmol). After 3 hrs the reaction mixture was quenched with TFA and H₂O, followed by HPLC purification gave N-((3-bromo-6-(tetrahydro-2H-pyran-4-yl)pyridin-2-yl)methyl)-2,2,2-trifluoroethan-1-amine. LC-MS: [M+H]+=354.01.

After freeze drying via lyophilization, the compound was stirred at room temperature for 5 h with 2 eq of (Boc)₂O (292 mg, 1.34 mmol) and 3 eq of Et₃N (2.01 mmol, 203 mg) in 4 ml DCM. Upon completion the residue was purified by combi-flash column chromatography using 0-100% EtOAc/Hexane to give compound tert-butyl ((3-bromo-6-(tetrahydro-2H-pyran-4-yl)pyridin-2-yl)methyl)(2,2,2-trifluoroethyl) carbamate (85 mg, 0.19 mmol, 28% in yield for 2 steps). LC-MS [M+H]+=454.07.

Palladium (II) acetate (0.2 eq.) and cataCXium A (0.4 eq.) were mixed together in DME (0.5 ml, degassed), and resulting solution was added via pipette to a mixture of ethyl 8-bromo-5-(((5-fluoro-2,3-dihydrobenzofuran-4-yl)methyl)amino) imidazo[1,5-c]pyrimidine-1-carboxylate (1 eq.), tert-butyl ((3-bromo-6-(tetrahydro-2H-pyran-4-yl)pyridin-2-yl)methyl)(2,2,2-trifluoroethyl) carbamate (83 mg, 0.18 mmol, 1.2 eq.), bis-pinacolatediboron (2 eq.) and K₂CO₃ (5 eq.) in DME/H₂O (10:1, 5.0 ml, degassed) at 70° C. The reaction mixture was stirred overnight. Then it was concentrated and extracted with ethyl acetate (2×30 ml), washed with water and brine, and dried over anhydrous Na₂SO₄. The mixture was concentrated followed by preparative HPLC purification afforded E-2211.1. LC-MS: [M+H]+=729.29. Removal of the Boc protecting group of E-2211.1 afforded E-2211.2. LC-MS: [M+H]+=629.21.

A mixture of compound E-2211.2 (1 eq.) and LiOH (10 eq.) in THF (10 ml/mmol) and water (5 ml/mmol) was heated at 80° C. overnight. The mixture was concentrated, and residue was then purified by prep-HPLC to afford E-2211.3 in 50% yield for the 3 steps.

To a mixture of compound E-2211.3 (1 eq.) and HATU (2 eq.) in DMF (5 ml/mmol) was added DIPEA (5 eq.). The reaction mixture was allowed to stir 2 h, and concentrated. The residue was purified by prep-HPLC to afford Cpd. No. E 45 in quantitative yield. LC-MS: [M+H]+=583.09. ¹H NMR (400 MHz, DMSO-d₆) δ 8.83 (s, 1H), 8.68 (t, J=4.8 Hz, 1H), 7.90 (d, J=8.4 Hz, 1H), 7.54 (s, 1H), 7.43 (d, J=8.4 Hz, 1H), 6.95 (dd, J=9.6, 8.8 Hz, 1H), 6.72 (dd, J=8.8, 4.0 Hz, 1H), 5.47 (d, J=14.8 Hz, 1H), 4.74 (d, J=4.8 Hz, 2H), 4.70-4.63 (m, 1H), 4.55 (t, J=8.8 Hz, 2H), 4.16 (d, J=14.8 Hz, 1H), 4.07-4.03 (m, 4H), 3.45-3.51 (m, 2H), 3.33 (t, J=8.4 Hz, 2H), 2.97-3.05 (m, 1H), 1.75-1.84 (m, 4H).

### Example 11

### Synthesis of 7-(tert-butyl)-12-(((5-fluoro-2,3-dihydrobenzofuran-4-yl)methyl)amino)-4-(2,2,2-trifluoroethyl)-4,5-dihydro-3H-2,4,8,11,12a-pentaazabenzo[4,5]cyclo octa[1,2,3-cd]inden-3-one (Cpd. No. E 46)

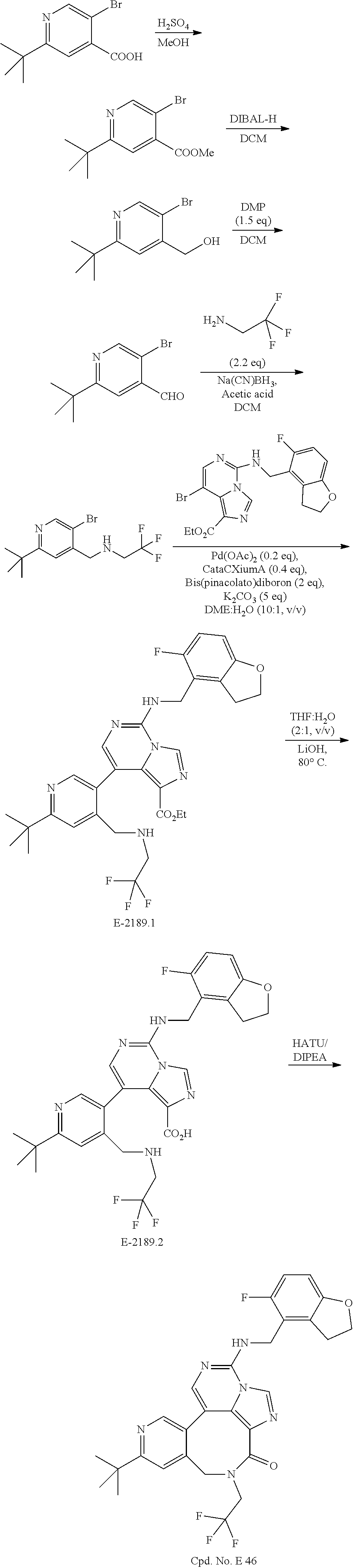

H₂SO₄ (0.5 mL, 9.69 mmol, 1.0 eq.) was added to a solution of 5-bromo-2-(tert-butyl)isonicotinic acid (2.5 g, 9.69 mmol, 1.0 eq.) in MeOH (25 ml). The resulting solution was stirred for 14 h at reflux. The mixture was cooled to room temperature and concentrated under vacuum. The residue was dissolved in ethyl acetate (50 ml), washed with water and satd. aq. NaCl (2×50 ml), dried over anhydrous Na₂SO₄, and concentrated under vacuum. The residue was purified by silica gel column chromatography eluted with EtOAc/hexane to yield methyl 5-bromo-2-(tert-butyl)isonicotinate.

To a solution of methyl 5-bromo-2-(tert-butyl)isonicotinate in DCM (50 ml) at −78° C., DIBAL-H (18 ml, 18.90 mmol, 1.05 M in toluene) was added dropwise. The reaction mixture was maintained at −78° C. to −15° C. for 30 min, then was allowed to warm to room temperature and stirred for another 12 h. The reaction mixture was cooled to 0° C. and quenched with satd. aq. NH₄Cl (50 ml). The resulting mixture was extracted with DCM (3×250 ml), washed with brine (150 ml), dried over anhydrous (Na₂SO₄), filtered, and concentrated. The residue was purified by silica gel column chromatography eluted with EtOAc/hexane to afford (5-bromo-2-(tert-butyl)pyridine-4-yl)methanol (1.78 g, 7.28 mmol, 75% for 2 steps). LC-MS [M+H]+=243.98/245.99.

An aliquot of (5-bromo-2-(tert-butyl)pyridin-4-yl)methanol (1.78 g, 7.28 mmol) was dissolved in dry DCM (˜0.2 M), then to this solution 1.3 eq. of Dess-Martin periodinane (4.11 g, 9.46 mmol) was added and the reaction mixture is allowed to stir for 1 h, monitored via TLC. Upon completion quenched with saturated NH₄Cl solution, then extracted with DCM. The organic layers were collected and combined, washed with brine, dried over anhydrous Na₂SO₄, and concentrated in vacuo. Purification was performed on silica gel normal phase column chromatography with increasing amounts of ethyl acetate in hexanes to afford the desired 5-bromo-2-(tert-butyl)isonicotinaldehyde.

To 5-bromo-2-(tert-butyl)isonicotinaldehyde was added methanol (˜0.2 M), followed by 2.2 eq. of 2,2,2-trifluoroethan-1-amine, 2 eq. of Na(CN)BH₃ and 2 eq. of acetic acid under ice bath. The ice bath was removed and reaction mixture was allowed to stir for 3 h, monitored via TLC. Upon completion, the reaction mixture was concentrated, and residue was purified by HPLC to afford N-((5-bromo-2-(tert-butyl)pyridin-4-yl)methyl)-2,2,2-trifluoroethan-1-amine (800 mg, 2.27 mmol, 31% for 2 steps). LC-MS: [M+H]+=324.89/326.86.

Palladium (II) acetate (0.1 eq.) and cataCXium A (0.2 eq.) were mixed together in DME (0.5 ml, degassed) and the resulting solution was added via pipette to a mixture of ethyl 8-bromo-5-(((5-fluoro-2,3-dihydrobenzofuran-4-yl)methyl)amino) imidazo[1,5-c]pyrimidine-1-carboxylate (1 eq.), N-((5-bromo-2-(tert-butyl)pyridin-4-yl)methyl)-2,2,2-trifluoroethan-1-amine (2 eq.), bis-pinacolatediboron (2 eq.) and K₂CO₃ (5 eq.) in DME/H₂O (10:1, 22 ml, degassed) at 70° C. The reaction mixture was stirred overnight. The reaction mixture was concentrated and extracted with ethyl acetate (2×50 ml), washed with water and brine, and dried over Na₂SO₄. The mixture was concentrated, and residue was purified by HPLC to afford E-2189.1 in ˜15% yield. LC-MS: [M+H]+=601.20.

A mixture of compound E-2189.1 (1 eq.) and LiOH (10 eq.) in THF (10 ml/mmol) and water (5 ml/mmol) was heated at 80° C. for overnight. The mixture was concentrated, and residue was purified by prep-HPLC to afford E-2189.2 and Cpd. No. E 46 in ˜5:1 ratio. LC-MS: [M+H]+=573.15.

To a mixture of compound E-2189.2 (1 eq.) and HATU (2 eq.) in DMF (5 ml/mmol) was added DIPEA (5 eq.). The reaction mixture was allowed to stir 2 h. The reaction mixture was concentrated, and the residue was purified by prep-HPLC to give Cpd. No. E 46 in a combined yield of ˜90%. LC-MS: [M+H]+=555.10. ¹H NMR (400 MHz, DMSO-d₆) δ 8.85 (s, 1H), 8.72 (t, 1H), 8.65 (d, J=2.4, 1H), 7.91 (d, J=7.8 Hz, 1H), 7.58 (d, J=1.6 Hz, 1H), 6.96 (t, J=8.8 Hz, 1H), 6.71 (dd, J=8.8, 4.0 Hz, 1H), 5.34 (d, J=14.8 Hz, 1H), 4.75 (d, J=4.0 Hz, 2H), 4.56 (t, J=8.8 Hz, 2H), 4.29 (d, J=14.8 Hz, 1H), 4.06-4.12 (m, 1H), 3.34 (t, J=8.8 Hz, 2H), 1.40 (s, 9H).

### Example 12

### Synthesis of 4-(2,2-difluoroethyl)-12-(((5-fluoro-2,3-dihydrobenzofuran-4-yl)methyl)amino)-7-isopropyl-4,5-dihydro-3H-2,4,6,11,12a-pentaazabenzo[4,5]cycloocta[1,2,3-cd]inden-3-one (Cpd. No. E 47)

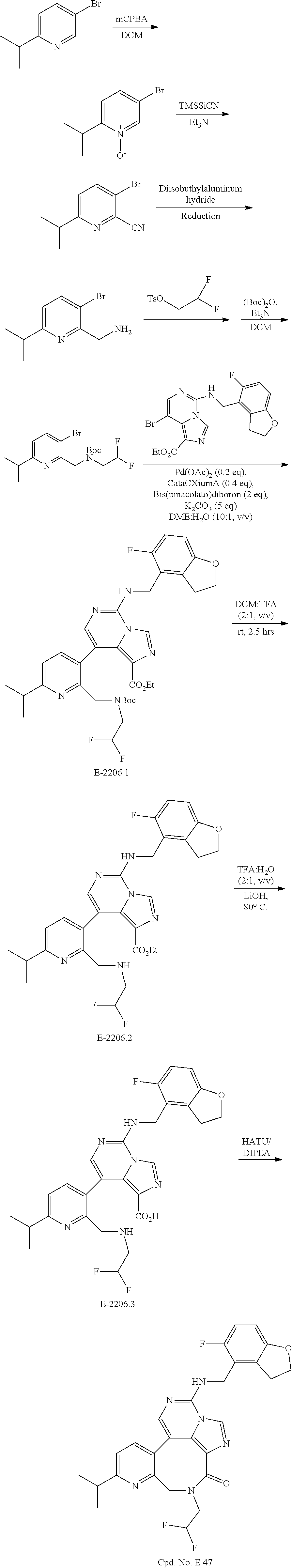

To a solution of 5-bromo-2-isopropylpyridine (1.00 g, 5.00 mmol) in DCM (20 ml) was added 1.5 eq of mCPBA. After 4 h the reaction mixture was quenched with 2.0 eq of Ca(OH)₂, and the resulting precipitate was stirred for 30 minutes. The precipitate was filtered and washed with 3:1 DCM/methanol. The filtrate was concentrated in vacuo to give 5-bromo-2-isopropylpyridine 1-oxide as a crude solid, which was used for the subsequent reaction without further purification.

To the solution of crude 5-bromo-2-isopropylpyridine 1-oxide obtained from the previous step in acetonitrile (20 ml, 0.2 M) was added 6.0 eq of trimethylsilyl cyanide (TMSCN) and 4.5 eq of triethylamine. The reaction was heated at 100° C. overnight. After cooling to room temperature, the solvent was concentrated in vacuo, and the residue was purified by pre-HPLC to give 3-bromo-6-isopropylpicolinonitrile (443 mg, 1.97 mmol, 39% in yield for 2 steps). LC-MS [M+H]+=225.01/227.03.

3-bromo-6-isopropylpicolinonitrile (443 mg, 1.97 mmol) was dissolved in 10 ml of dry DCM and cooled to −78° C. While stirring the solution, 2 eq of DIBAL-H solution in toluene was added dropwise. The mixture was stirred 5 h, then quenched by slow addition of saturated aqueous Rochelle's salt (sodium potassium tartrate). The reaction mixture was allowed to warm, diluted with ethyl acetate, and stirred until two easily separable clear layers formed. HPLC purification gave (3-bromo-6-isopropylpyridine-2-yl)methanamine as a liquid. LC-MS [M+H]+=229.01/230.97. ¹H NMR (400 MHz, DMSO-d₆) δ 8.34 (s, broad, 2H), 8.04 (d, J=8.4 Hz, 1H), 7.28 (d, J=8.4 Hz, 1H), 4.24-4.23 (m, 2H), 3.33-3.02 (m, 1H), 1.26 (d, J=6.8 Hz, 6H).

To a solution of (3-bromo-6-isopropylpyridine-2-yl)methanamine (107 mg, 0.47 mmol) in DCM (10 ml) was added 2.0 eq of 2,2-difluoroethyl 4-methylbenzenesulfonate (200 mg, 0.94 mmol) and 2.0 eq of DIPEA. After 3 h the reaction mixture was quenched with TFA and H₂O. HPLC purification gave N-((3-bromo-6-isopropylpyridine-2-yl)methyl)-2,2-difluoroethane-1-amine. LC-MS [M+H]+=293.04/285.09.

After freeze drying via lyophilization, the N-((3-bromo-6-isopropylpyridine-2-yl)methyl)-2,2-difluoroethane-1-amine (103 mg, 0.35 mmol) was stirred at room temperature for 5 h with 2 eq of (Boc)₂O (153 mg, 0.70 mmol) and 3 eq of Et₃N (203 mg, 1.05 mmol) in 3 ml dry DCM. Upon completion the residue was purified by combi-flush column chromatography using 0-100% EtOAc/Hexane to give tert-butyl ((3-bromo-6-isopropylpyridine-2-yl)methyl)(2,2-difluoroethyl)carbamate (52 mg, 0.13 mmol, 28% in yield for 2 steps).

Palladium (II) acetate (0.2 eq.) and cataCXium A (0.4 eq.) were mixed together in DME (0.5 ml, degassed), and the resulting solution was added via pipette to a mixture of ethyl 8-bromo-5-(((5-fluoro-2,3-dihydrobenzofuran-4-yl)methyl)amino) imidazo[1,5-c]pyrimidine-1-carboxylate (1 eq.), tert-butyl ((3-bromo-6-isopropylpyridine-2-yl)methyl)(2,2-difluoroethyl)carbamate (52 mg, 0.13 mmol, 1.2 eq.), bis-pinacolatediboron (2.0 eq.) and K₂CO₃ (5.0 eq.) in DME/H₂O (10:1, 4.0 ml, degassed) at 70° C. The reaction mixture was stirred overnight. Then it was concentrated and extracted with ethyl acetate (2×30 ml), washed with water and brine, and dried over Na₂SO₄. The mixture was concentrated and purified by HPLC to afford E-2206.1. LC-MS: [M+H]+=669.33. The Boc protecting group of E-2206.1 was removed to afford E-2206.2. LC-MS: [M+H]+=569.17.

A mixture of E-2206.2 (1 eq.) and LiOH (10 eq.) in THF (10 ml/mmol) and water (5 ml/mmol) was heated at 80° C. overnight. The mixture was concentrated, and residue was then purified by prep-HPLC to afford E-2206.3. LC-MS: [M+H]+=541.10.

To a mixture of E-2206.3 (1 eq.) and HATU (2 eq.) in DMF (3 ml/mmol) was added DIPEA (5 eq.). The reaction mixture was allowed to stir 2 h, and concentrated. The residue was purified by prep-HPLC to afford Cpd. No. E 47 in quantitative yield. LC-MS: [M+H]+=523.15. ¹H NMR (400 MHz, DMSO-d₆) δ 8.80 (s, 1H), 8.63 (t, 1H), 7.85 (d, J=8.0 Hz, 1H), 7.49 (s, 1H), 7.37 (d, J=8.0 Hz, 1H), 6.94 (dd, J=9.6, 8.8 Hz, 1H), 6.70 (dd, J=8.8, 4.0 Hz, 1H), 6.29 (t, J=57.2 Hz, 1H), 5.40 (d, J=8.4 Hz, 1H), 4.72 (d, J=3.6 Hz, 2H), 4.54 (t, J=8.8 Hz, 2H), 4.14 (d, J=14.8 Hz, 1H), 4.09-4.03 (m, 1H), 3.75-3.33 (m, 1H), 3.33 (t, J=8.8 Hz, 2H), 3.10-3.03 (m, 2H), 1.29-1.22 (m, 6H).

### Example 13

### Compound Characterization

The compounds of Table 3 were prepared using methodology described in EXAMPLES 1-12, see, e.g., “Synthetic method” column, and known in the art. All compounds were characterized by mass spectroscopy and/or ¹H NMR as the TFA salt.

TABLE 3

| | | Synthetic |
|---|---|---|
| Cpd. No. | ¹H NMR (400 MHz)/LC-MS data | method |
| 1 | LC-MS: [M + H]+ = 376.14 | Example 5 |
| 2 | LC-MS: [M + H]+ = 404.14 | Example 5 |
| 3 | ¹H NMR (400 MHz, DMSO-d₆) δ 9.50 (s, 1H), 8.67 | Example 3 |
| | (s, 1H), 8.53 (t, J = 5.1 Hz, 1H), 7.99 (s, 1H), 7.88- | |
| | 7.80 (m, 1H), 7.19 (dd, J = 6.1, 1.6 Hz, 2H), 7.02 | |
| | (ddd, J = 8.3, 6.1, 2.4 Hz, 1H), 6.96 (dd, J = 10.3, 8.7 | |
| | Hz, 1H), 6.71 (dd, J = 8.6, 3.9 Hz, 1H), 4.73 (d, J = | |
| | 4.8 Hz, 2H), 4.56 (t, J = 8.7 Hz, 2H), 3.31 (d, J = 8.5 | |
| | Hz, 2H). | |
| | LC-MS: [M + H]+ = 402.12 | |
| 4 | ¹H NMR (400 MHz, DMSO-d₆) δ 9.26 (s, 1H), 8.69 | Example 3 |
| | (s, 1H), 8.66 (t, J = 5.1 Hz, 1H), 8.32 (dd, J = 8.1, 1.7 | |
| | Hz, 1H), 8.19 (dd, J = 4.5, 1.6 Hz, 1H), 8.06 (s, 1H), | |
| | 7.09 (dd, J = 8.0, 4.6 Hz, 1H), 6.96 (dd, J = 10.4, 8.7 | |
| | Hz, 1H), 6.71 (dd, J = 8.6, 3.9 Hz, 1H), 4.73 (d, J = | |
| | 4.9 Hz, 2H), 4.56 (t, J = 8.7 Hz, 2H), 3.31 (d, J = 8.5 | |
| | Hz, 2H). | |
| | LC-MS: [M + H]+ = 403.12 | |
| 5 | ¹H NMR (400 MHz, DMSO-d₆) δ 9.77 (s, 1H), 8.76 | Example 3 |
| | (t, J = 5.1 Hz, 1H), 8.70 (s, 1H), 8.15-8.01 (m, 2H), | |
| | 7.75 (d, J = 2.0 Hz, 1H), 7.47 (dd, J = 8.5, 2.0 Hz, | |
| | 1H), 6.96 (dd, J = 10.3, 8.7 Hz, 1H), 6.71 (dd, J = 8.6, | |
| | 3.9 Hz, 1H), 4.74 (d, J = 5.0 Hz, 2H), 4.56 (t, J = 8.7 | |
| | Hz, 2H), 3.36 (t, J = 8.7 Hz, 2H), 3.21 (s, 3H). | |
| | LC-MS: [M + H]+ = 480.11 | |
| 6 | ¹H NMR (400 MHz, DMSO-d₆) δ 8.87 (s, 1H), 8.73 | Example 1 |
| | (t, J = 5.1 Hz, 1H), 7.68 (s, 1H), 7.60-7.51 (m, 2H), | |
| | 7.48 (qd, J = 7.5, 1.7 Hz, 1H), 6.97 (dd, J = 10.3, 8.7 | |
| | Hz, 1H), 6.72 (dd, J = 8.7, 3.9 Hz, 1H), 5.78 (d, J = | |
| | 12.4 Hz, 1H), 5.04 (d, J = 12.5 Hz, 1H), 4.76 (d, J = | |
| | 4.8 Hz, 2H), 4.57 (t, J = 8.7 Hz, 2H), 3.36 (t, J = 8.8 | |
| | Hz, 2H). | |
| | LC-MS: [M + H]+ = 417.13 | |
| 7 | ¹H NMR (400 MHz, DMSO-d₆) δ 8.87 (s, 1H), 8.74 | Example 1 |
| | (t, J = 5.1 Hz, 1H), 7.66 (s, 1H), 7.62 (dd, J = 8.7, 5.6 | |
| | Hz, 1H), 7.48 (dd, J = 9.2, 2.8 Hz, 1H), 7.38 (td, J = | |
| | 8.6, 2.8 Hz, 1H), 7.02-6.91 (m, 1H), 6.72 (dd, J = | |
| | 8.6, 3.9 Hz, 1H), 5.76 (d, J = 12.5 Hz, 1H), 5.03 (d, J = | |
| | 12.5 Hz, 1H), 4.76 (d, J = 4.8 Hz, 2H), 4.57 (t, J = | |
| | 8.8 Hz, 2H), 3.35 (t, J = 8.7 Hz, 2H). | |
| | LC-MS: [M + H]+ = 435.12 | |
| 8 | ¹H NMR (400 MHz, DMSO-d₆) δ 8.88 (d, J = 9.5 Hz, | Example 1 |
| | 2H), 7.98 (d, J = 1.8 Hz, 1H), 7.92-7.84 (m, 1H), | |
| | 7.79 (d, J = 14.4 Hz, 2H), 6.97 (t, J = 9.5 Hz, 1H), | |
| | 6.72 (dd, J = 8.7, 3.9 Hz, 1H), 5.87 (d, J = 12.5 Hz, | |
| | 1H), 5.20 (d, J = 12.7 Hz, 1H), 4.78 (d, J = 4.8 Hz, | |
| | 2H), 4.57 (t, J = 8.7 Hz, 2H), 3.36 (t, J = 8.8 Hz, 2H). | |
| | LC-MS: [M + H]+ = 485.12 | |
| 9 | LC-MS: [M + H]+ = 417.12 | Example 1 |
| 10 | LC-MS: [M + H]+ = 416.10 | Example 1 |
| 11 | ¹H NMR (400 MHz, DMSO-d₆) δ 8.89 (s, 1H), 8.81 | Example 1 |
| | (t, J = 5.1 Hz, 1H), 7.75 (s, 1H), 7.57 (td, J = 8.1, 6.0 | |
| | Hz, 1H), 7.42 (dd, J = 8.0, 1.1 Hz, 1H), 7.35 (ddd, J = | |
| | 9.4, 8.2, 1.1 Hz, 1H), 6.97 (dd, J = 10.3, 8.7 Hz, 1H), | |
| | 6.72 (dd, J = 8.7, 3.9 Hz, 1H), 5.67 (dd, J = 12.9, 2.6 | |
| | Hz, 1H), 5.29 (d, J = 13.1 Hz, 1H), 4.86-4.66 (m, | |
| | 2H), 4.57 (t, J = 8.8 Hz, 2H), 3.36 (t, J = 8.7 Hz, 2H). | |
| | LC-MS: [M + H]+ = 435.12 | |
| 13 | ¹H NMR (400 MHz, DMSO-d₆) δ 8.98 (d, J = 2.7 Hz, | Example 1 |
| | 2H), 8.92 (s, 1H), 8.16 (s, 1H), 7.92 (s, 1H), 6.97 (dd, | |
| | J = 10.3, 8.7 Hz, 1H), 6.72 (dd, J = 8.6, 3.9 Hz, 1H), | |
| | 5.91 (brs, 1H), 5.22 (brs, 1H), 4.79 (d, J = 4.8 Hz, | |
| | 2H), 4.57 (t, J = 8.7 Hz, 2H), 3.36 (t, J = 8.7 Hz, 2H). | |
| | LC-MS: [M + H]+ = 486.11 | |
| 14 | ¹H NMR (400 MHz, DMSO-d₆) δ 9.00 (d, J = 5.1 Hz, | Example 1 |
| | 1H), 8.92 (s, 1H), 8.32 (d, J = 8.1 Hz, 1H), 8.05 (d, J = | |
| | 8.3 Hz, 1H), 7.83 (s, 1H), 6.97 (dd, J = 10.3, 8.7 Hz, | |
| | 1H), 6.72 (dd, J = 8.7, 3.9 Hz, 1H), 6.12-5.96 (m, | |
| | 1H), 5.06 (d, J = 12.6 Hz, 1H), 4.78 (d, J = 4.9 Hz, | |
| | 2H), 4.57 (t, J = 8.8 Hz, 2H), 3.36 (t, J = 8.7 Hz, 2H). | |
| | LC-MS: [M + H]+ = 486.11 | |
| 15 | ¹H NMR (400 MHz, DMSO-d₆) | Example 2 |
| | δ 8.79 (s, 1H), 8.53 (t, J = 5.1 Hz, 1H), 8.24 (t, J = 7.4 | |
| | Hz, 1H), 7.53-7.35 (m, 4H), 7.33-7.24 (m, 1H), | |
| | 7.03-6.90 (m, 1H), 6.72 (dd, J = 8.7, 3.9 Hz, 1H), | |
| | 4.83-4.70 (m, 3H), 4.57 (t, J = 8.8 Hz, 2H), 4.03 (dd, | |
| | J = 14.2, 5.7 Hz, 1H), 3.35 (t, J = 8.7 Hz, 2H). | |
| | LC-MS: [M + H]+ = 416.14 | |
| 17 | ¹H NMR (400 MHz, DMSO-d₆) δ 8.79 (s, 1H), 8.57 | Example 2 |
| | (t, J = 5.1 Hz, 1H), 8.22 (d, J = 7.6 Hz, 1H), 7.50 (dd, | |
| | J = 8.6, 5.7 Hz, 1H), 7.41 (s, 1H), 7.24 (td, J = 8.6, 2.7 | |
| | Hz, 1H), 7.11 (dd, J = 9.2, 2.8 Hz, 1H), 6.96 (dd, J = | |
| | 10.3, 8.7 Hz, 1H), 6.71 (dd, J = 8.7, 3.8 Hz, 1H), 4.77- | |
| | 4.33 (m, 3H), 4.56 (t, J = 8.7 Hz, 2H), 3.97 (dd, J = | |
| | 14.6, 5.4 Hz, 1H), 3.35 (t, J = 8.7 Hz, 2H). | |
| | LC-MS: [M + H]+ = 434.14 | |
| 18 | ¹H NMR (400 MHz, DMSO-d₆) δ 8.90 (s, 1H), 8.85 | Example 1 |
| | (t, J = 5.1 Hz, 1H), 7.96 (d, J = 8.0 Hz, 1H), 7.71 (s, | |
| | 1H), 7.46 (d, J = 8.1 Hz, 1H), 6.96 (t, J = 9.5 Hz, 1H), | |
| | 6.72 (dd, J = 8.7, 3.8 Hz, 1H), 5.91 (d, J = 12.2 Hz, | |
| | 1H), 4.95 (d, J = 12.1 Hz, 1H), 4.76 (d, J = 4.2 Hz, | |
| | 2H), 4.56 (t, J = 8.7 Hz, 2H), 3.35 (t, J = 8.8 Hz, 2H), | |
| | 2.57 (s, 3H). | |
| | LC-MS: [M + H]+ = 432.14 | |
| 19 | ¹H NMR (400 MHz, DMSO-d₆) δ 8.82 (s, 1H), 8.69 | Example 2 |
| | (t, J = 5.1 Hz, 1H), 8.24 (dd, J = 8.8, 5.7 Hz, 1H), 7.74 | |
| | (d, J = 8.3 Hz, 2H), 7.71-7.59 (m, 1H), 7.52 (s, 1H), | |
| | 6.96 (t, J = 9.5 Hz, 1H), 6.72 (dd, J = 8.6, 3.8 Hz, 1H), | |
| | 4.86 (dd, J = 14.6, 8.8 Hz, 1H), 4.76 (d, J = 4.5 Hz, | |
| | 2H), 4.56 (t, J = 8.7 Hz, 2H), 4.11 (dd, J = 14.7, 5.5 | |
| | Hz, 1H), 3.35 (t, J = 8.8 Hz, 2H). | |
| | LC-MS: [M + H]+ = 484.13 | |
| 20 | ¹H NMR (400 MHz, DMSO-d₆) δ 8.84 (s, 1H), 8.64 | Example 2 |
| | (s, 1H), 8.49 (t, J = 7.2 Hz, 1H), 7.49 (s, 1H), 7.46- | |
| | 7.38 (m, 1H), 7.35-7.18 (m, 2H), 6.95 (t, J = 9.4 Hz, | |
| | 1H), 6.71 (dd, J = 8.7, 3.8 Hz, 1H), 4.74 (s, 2H), 4.58 | |
| | (dt, J = 17.7, 7.8 Hz, 3H), 4.31 (dd, J = 15.1, 5.8 Hz, | |
| | 1H), 3.34 (t, J = 8.8 Hz, 2H). | |
| | LC-MS: [M + H]+ = 434.14 | |
| 21 | ¹H NMR (400 MHz, DMSO-d₆) δ 8.84 (s, 1H), 8.64 | Example 2 |
| | (d, J = 5.8 Hz, 1H), 8.40 (dd, J = 8.4, 6.0 Hz, 1H), | |
| | 7.93 (d, J = 8.0 Hz, 1H), 7.49-7.39 (m, 2H), 6.96 | |
| | (dd, J = 10.3, 8.7 Hz, 1H), 6.71 (dd, J = 8.6, 3.9 Hz, | |
| | 1H), 4.95 (dd, J = 14.4, 8.5 Hz, 1H), 4.75 (d, J = 4.3 | |
| | Hz, 2H), 4.56 (t, J = 8.8 Hz, 2H), 4.05 (dd, J = 14.4, | |
| | 5.9 Hz, 1H), 3.34 (t, J = 8.7 Hz, 2H), 2.56 (s, 3H). | |
| | LC-MS: [M + H]+ = 431.15 | |
| 22 | ¹H NMR (400 MHz, DMSO-d₆) δ 8.83 (s, 2H), 8.76 | Example 2 |
| | (t, J = 5.2 Hz, 1H), 8.23 (dd, J = 8.8, 5.7 Hz, 1H), 7.77 | |
| | (s, 1H), 7.65 (s, 1H), 6.96 (t, J = 9.5 Hz, 1H), 6.72 | |
| | (dd, J = 8.7, 3.8 Hz, 1H), 4.93 (dd, J = 14.5, 8.8 Hz, | |
| | 1H), 4.77 (d, J = 4.8 Hz, 2H), 4.57 (t, J = 8.7 Hz, 2H), | |
| | 4.17 (dd, J = 14.6, 5.6 Hz, 1H), 3.35 (t, J = 8.7 Hz, | |
| | 2H). | |
| | LC-MS: [M + H]+ = 485.13 | |
| 23 | ¹H NMR (400 MHz, DMSO-d₆) δ 8.79 (s, 1H), 8.59 | Example 2 |
| | (t, J = 5.1 Hz, 1H), 8.21 (dd, J = 8.9, 5.6 Hz, 1H), 7.49 | |
| | (s, 1H), 7.35-7.27 (m, 2H), 7.26-7.18 (m, 1H), | |
| | 6.96 (dd, J = 10.3, 8.6 Hz, 1H), 6.71 (dd, J = 8.7, 3.9 | |
| | Hz, 1H), 4.75 (q, J = 5.0 Hz, 3H), 4.56 (t, J = 8.8 Hz, | |
| | 2H), 3.97 (dd, J = 14.7, 5.5 Hz, 1H), 3.35 (t, J = 8.7 | |
| | Hz, 2H). | |
| | LC-MS: [M + H]+ = 434.14 | |
| 24 | ¹H NMR (400 MHz, DMSO-d₆) δ 8.90 (s, 1H), 8.85 | Example 1 |
| | (s, 1H), 7.88 (d, J = 1.7 Hz, 1H), 7.86-7.74 (m, 3H), | |
| | 6.96 (dd, J = 10.3, 8.7 Hz, 1H), 6.72 (dd, J = 8.6, 3.9 | |
| | Hz, 1H), 5.86 (d, J = 12.5 Hz, 1H), 5.15 (d, J = 12.4 | |
| | Hz, 1H), 4.77 (d, J = 3.8 Hz, 2H), 4.57 (t, J = 8.8 Hz, | |
| | 2H), 3.37 (dt, J = 9.0, 5.1 Hz, 2H). | |
| | LC-MS: [M + H]+ = 485.12 | |
| 25 | ¹H NMR (400 MHz, DMSO-d₆) δ 8.84 (s, 1H), 8.77 | Example 2 |
| | (t, J = 5.1 Hz, 1H), 8.57-8.45 (m, 1H), 8.18 (d, J = | |
| | 8.1 Hz, 1H), 7.94 (d, J = 8.1 Hz, 1H), 7.55 (s, 1H), | |
| | 6.96 (dd, J = 10.3, 8.7 Hz, 1H), 6.72 (dd, J = 8.6, 3.9 | |
| | Hz, 1H), 5.07 (dd, J = 14.4, 8.3 Hz, 1H), 4.77 (d, J = | |
| | 4.8 Hz, 2H), 4.57 (t, J = 8.8 Hz, 2H), 4.10 (dd, J = | |
| | 14.4, 6.0 Hz, 1H), 3.35 (t, J = 8.7 Hz, 2H). | |
| | LC-MS: [M + H]+ = 485.12 | |
| 26 | ¹H NMR (400 MHz, DMSO-d₆) δ 8.81 (s, 1H), 8.65 | Example 2 |
| | (t, J = 5.1 Hz, 1H), 8.28 (t, J = 7.0 Hz, 1H), 7.76 (dq, J = | |
| | 3.7, 1.9 Hz, 2H), 7.60-7.43 (m, 2H), 6.96 (dd, J = | |
| | 10.3, 8.7 Hz, 1H), 6.71 (dd, J = 8.6, 3.9 Hz, 1H), 4.85 | |
| | (dd, J = 14.5, 8.8 Hz, 1H), 4.75 (d, J = 4.8 Hz, 2H), | |
| | 4.57 (t, J = 8.8 Hz, 2H), 4.07 (dd, J = 14.6, 5.5 Hz, | |
| | 1H), 3.36 (dd, J = 9.6, 7.5 Hz, 2H). | |
| | LC-MS: [M + H]+ = 484.13 | |
| 27 | ¹H NMR (400 MHz, DMSO-d₆) δ 9.02 (t, J = 5.5 Hz, | Example 1 |
| | 1H), 8.90 (s, 1H), 7.98 (d, J = 2.0 Hz, 1H), 7.86 (dd, J = | |
| | 8.6, 2.0 Hz, 1H), 7.78 (d, J = 8.2 Hz, 1H), 7.74 (s, | |
| | 1H), 6.96 (dd, J = 7.5, 1.6 Hz, 1H), 6.92-6.79 (m, | |
| | 2H), 6.07 (s, 2H), 5.87 (d, J = 12.7 Hz, 1H), 5.20 (d, J = | |
| | 12.6 Hz, 1H), 4.79 (d, J = 5.3 Hz, 2H). | |
| | LC-MS: [M + H]+ = 469.10 | |
| 28 | ¹H NMR (400 MHz, DMSO-d₆) δ 8.92 (s, 1H), 8.53 | Example 1 |
| | (t, J = 4.3 Hz, 1H), 7.98 (s, 1H), 7.94-7.83 (m, 1H), | |
| | 7.83-7.71 (m, 2H), 7.50-7.33 (m, 1H), 6.96 (d, J = | |
| | 8.4 Hz, 1H), 6.89 (t, J = 8.9 Hz, 1H), 5.87 (d, J = 12.5 | |
| | Hz, 1H), 5.20 (d, J = 12.4 Hz, 1H), 4.80 (s, 2H), 3.87 | |
| | (d, J = 1.0 Hz, 3H). | |
| | LC-MS: [M + H]+ = 473.11 | |
| 29 | ¹H NMR (400 MHz, DMSO-d₆) δ 9.05 (t, J = 5.2 Hz, | Example 1 |
| | 1H), 8.92 (s, 1H), 8.27 (d, J = 7.9 Hz, 1H), 8.07 (s, | |
| | 1H), 7.95 (d, J = 7.8 Hz, 1H), 6.97 (dd, J = 10.3, 8.7 | |
| | Hz, 1H), 6.72 (dd, J = 8.7, 3.8 Hz, 1H), 5.95 (s, 1H), | |
| | 5.23 (s, 1H), 4.81 (d, J = 5.0 Hz, 2H), 4.57 (t, J = 8.7 | |
| | Hz, 2H), 3.36 (dd, J = 9.6, 7.5 Hz, 2H). | |
| | LC-MS: [M + H]+ = 486.11 | |
| 30 | ¹H NMR (400 MHz, DMSO-d₆) δ 9.06-8.94 (m, | Example 1 |
| | 2H), 8.92 (s, 1H), 8.40 (dd, J = 2.1, 0.8 Hz, 1H), 7.87 | |
| | (s, 1H), 6.95 (dd, J = 10.3, 8.6 Hz, 1H), 6.70 (dd, J = | |
| | 8.6, 3.9 Hz, 1H), 6.05 (d, J = 12.2 Hz, 1H), 5.06 (d, J = | |
| | 12.0 Hz, 1H), 4.86-4.67 (m, 2H), 4.57 (t, J = 8.7 | |
| | Hz, 2H), 3.37 (t, J = 8.7 Hz, 2H). | |
| | LC-MS: [M + H]+ = 486.11 | |
| 31 | ¹H NMR (400 MHz, DMSO-d₆) δ 8.88 (s, 1H), 8.79 | Scheme 1 |
| | (t, J = 5.1 Hz, 1H), 7.71 (t, J = 4.3 Hz, 2H), 7.63 (dd, J = | |
| | 2.6, 1.1 Hz, 1H), 7.52 (ddd, J = 8.7, 2.6, 1.1 Hz, | |
| | 1H), 6.96 (dd, J = 10.3, 8.6 Hz, 1H), 6.72 (dd, J = 8.7, | |
| | 3.9 Hz, 1H), 5.80 (d, J = 12.5 Hz, 1H), 5.11 (d, J = | |
| | 12.5 Hz, 1H), 4.77 (d, J = 4.5 Hz, 2H), 4.57 (t, J = 8.7 | |
| | Hz, 2H), 3.36 (t, J = 8.8 Hz, 2H). | |
| | LC-MS: [M + H]+ = 501.11 | |
| 32 | ¹H NMR (400 MHz, DMSO-d₆) δ 9.08 (t, J = 5.1 Hz, | Example 1 |
| | 1H), 8.92 (s, 1H), 8.88 (s, 1H), 8.07 (s, 1H), 8.01 (s, | |
| | 1H), 7.03-6.90 (m, 1H), 6.72 (dd, J = 8.6, 3.9 Hz, | |
| | 1H), 5.95 (s, 1H), 5.26 (s, 1H), 4.79 (d, J = 4.9 Hz, | |
| | 2H), 4.58 (t, J = 8.7 Hz, 2H), 3.37 (t, J = 8.8 Hz, 2H). | |
| | LC-MS: [M + H]+ = 486.11 | |
| 33 | ¹H NMR (400 MHz, CDCl₃) δ 9.35 (s, 1H), 7.89 (d, J = | Example 4 |
| | 8.1 Hz, 1H), 7.78 (d, J = 8.1 Hz, 1H), 7.56 (s, 1H), | |
| | 6.66 (d, J = 9.2 Hz, 1H), 6.59-6.45 (m, 1H), 5.43 (d, | |
| | J = 14.5 Hz, 1H), 4.81 (d, J = 6.1 Hz, 2H), 4.50 (t, J = | |
| | 8.4 Hz, 2H), 4.24 (d, J = 14.4 Hz, 1H), 3.33 (t, J = 8.4 | |
| | Hz, 2H), 3.16 (s, 3H). | |
| | LC-MS: [M + H]+ = 499.14 | |
| 34 | ¹H NMR (400 MHz, methanol-d₄) δ 8.84 (s, 1H), 8.74 | Example 4 |
| | (s, 1H), 7.96 (s, 1H), 7.69 (s, 1H), 6.87 (t, J = 9.5 Hz, | |
| | 1H), 6.66 (dd, J = 8.8, 3.7 Hz, 1H), 5.54 (d, J = 14.8 | |
| | Hz, 1H), 4.81 (d, J = 6.1 Hz, 2H), 4.61 (t, J = 8.8 Hz, | |
| | 2H), 4.28 (d, J = 14.8 Hz, 1H), 3.43 (t, J = 8.8 Hz, | |
| | 2H), 3.16 (s, 3H). | |
| | LC-MS: [M + H]+ = 499.14 | |
| 35 | ¹H NMR (400 MHz, methanol-d₄) δ 8.78 (s, 1H), 8.14 | Example 4 |
| | (d, J = 8.1 Hz, 1H), 7.88 (d, J = 8.1 Hz, 1H), 7.65 (s, | |
| | 1H), 6.87 (t, J = 9.4 Hz, 1H), 6.66 (d, J = 8.6 Hz, 1H), | |
| | 5.54 (d, J = 16.3 Hz, 1H), 4.81 (d, J = 6.1 Hz, 2H), | |
| | 4.61-4.53 (m, 3H), 4.01 (s, 1H), 3.74 (dq, J = 48.4, | |
| | 11.0, 10.5 Hz, 5H), 3.60-3.45 (m, 2H), 3.42 (t, J = | |
| | 9.1 Hz, 2H), 3.33-3.30 (m, 1H). | |
| | LC-MS: [M + H]+ = 485.17 | |
| 37 | ¹H NMR (400 MHz, methanol-d₄) δ 8.82 (t, J = 4.7 | Example 4 |
| | Hz, 2H), 7.95 (d, J = 4.3 Hz, 1H), 7.69 (s, 1H), 6.86 | |
| | (t, J = 9.5 Hz, 1H), 6.71-6.58 (m, 1H), 5.47 (dd, J = | |
| | 15.0, 9.3 Hz, 1H), 4.81 (d, J = 6.1 Hz, 2H), 4.68- | |
| | 4.49 (m, 3H), 4.09-3.76 (m, 4H), 3.76-3.47 (m, | |
| | 4H), 3.41 (q, J = 8.6 Hz, 3H). | |
| | LC-MS: [M + H]+ = 585.17 | |
| 38 | ¹H NMR (400 MHz, methanol-d₄) δ 8.85 (s, 1H), 8.75 | Example 4 |
| | (s, 1H), 8.00 (s, 1H), 7.69 (s, 1H), 6.87 (t, J = 9.5 Hz, | |
| | 1H), 6.66 (d, J = 8.9 Hz, 1H), 5.46 (d, J = 14.9 Hz, | |
| | 1H), 4.81 (d, J = 6.1 Hz, 2H), 4.61 (t, J = 8.7 Hz, 2H), | |
| | 4.32 (d, J = 15.0 Hz, 1H), 3.92 (dd, J = 13.7, 8.9 Hz, | |
| | 1H), 3.58 (t, J = 11.7 Hz, 2H), 3.43 (t, J = 8.7 Hz, | |
| | 2H), 3.11 (dd, J = 13.7, 5.7 Hz, 1H), 2.99 (dd, J = | |
| | 29.9, 11.6 Hz, 2H), 2.90 (s, 3H), 2.23 (s, 1H), 2.07 (d, | |
| | J = 14.4 Hz, 1H), 1.98 (d, J = 14.6 Hz, 1H), 1.64 (d, J = | |
| | 14.1 Hz, 2H). | |
| | LC-MS: [M + H]+ = 596.23 | |
| 39 | ¹H NMR (400 MHz, DMSO-d₆) δ 8.87 (t, J = 4.8 Hz, | Example 4 |
| | 1H), 8.83 (s, 1H), 8.16 (d, J = 7.8 Hz, 1H), 7.86 (d, J = | |
| | 7.8 Hz, 1H), 7.82 (s, 1H), 6.95 (dd, J = 10.3, 8.6 Hz, | |
| | 1H), 6.70 (dd, J = 8.7, 3.9 Hz, 1H), 5.39 (d, J = 15.0 | |
| | Hz, 1H), 4.77 (d, J = 4.9 Hz, 2H), 4.55 (t, J = 8.8 Hz, | |
| | 2H), 4.20 (d, J = 15.0 Hz, 1H), 3.33 (td, J = 8.5, 4.5 | |
| | Hz, 2H), 2.98 (s, 3H). | |
| | LC-MS: [M + H]+ = 499.14 | |
| 40 | ¹H NMR (400 MHz, DMSO-d₆) δ 8.89 (s, 1H), 8.85 | Example 4 |
| | (s, 1H), 8.17 (d, J = 7.8 Hz, 1H), 7.86-7.79 (m, 2H), | |
| | 6.94 (dd, J = 10.3, 8.7 Hz, 1H), 6.70 (dd, J = 8.6, 3.9 | |
| | Hz, 1H), 5.34 (dd, J = 15.2, 6.4 Hz, 1H), 4.84-4.69 | |
| | (m, 2H), 4.54 (t, J = 8.7 Hz, 2H), 4.35 (dd, J = 15.2, | |
| | 10.1 Hz, 1H), 3.96-3.83 (m, 1H), 3.81-3.41 (m, | |
| | 6H), 3.39-3.21 (m, 3H), 3.11-2.90 (m, 1H). | |
| | LC-MS: [M + H]+ = 585.17 | |
| 41 | ¹H NMR (400 MHz, DMSO-d₆) δ 8.80 (s, 1H), 8.76 | Example 4 |
| | (t, J = 5.1 Hz, 1H), 8.20 (d, J = 8.1 Hz, 1H), 7.95 (d, J | |
| | = 8.1 Hz, 1H), 7.57 (s, 1H), 7.01-6.92 (m, 1H), 6.72 | |
| | (dd, J = 8.7, 3.9 Hz, 1H), 5.38 (d, J = 14.8 Hz, 1H), | |
| | 4.77 (d, J = 4.9 Hz, 2H), 4.56 (t, J = 8.8 Hz, 2H), 4.24 | |
| | (d, J = 14.7 Hz, 1H), 3.42 (t, J = 8.7 Hz, 2H), 2.63 (tt, | |
| | J = 7.4, 4.2 Hz, 1H), 0.99 (ddd, J = 10.9, 7.0, 5.1 Hz, | |
| | 1H), 0.92 (ddt, J = 9.4, 7.1, 3.5 Hz, 1H), 0.81-0.74 | |
| | (m, 1H), 0.72-0.64 (m, 1H). | |
| | LC-MS: [M + H]+ = 525.15 | |
| 42 | ¹H NMR (400 MHz, methanol-d₄) δ 8.76 (s, 1H), 8.17 | Example 4 |
| | (d, J = 8.1 Hz, 1H), 7.90 (d, J = 8.1 Hz, 1H), 7.67 (s, | |
| | 1H), 6.86 (t, J = 9.5 Hz, 1H), 6.65 (dd, J = 8.5, 3.6 Hz, | |
| | 1H), 5.57 (d, J = 14.7 Hz, 1H), 4.81 (d, J = 6.1 Hz, | |
| | 2H), 4.59 (t, J = 8.7 Hz, 2H), 4.35 (d, J = 14.7 Hz, | |
| | 1H), 3.81 (dd, J = 13.5, 8.5 Hz, 1H), 3.57 (d, J = 12.5 | |
| | Hz, 2H), 3.52-3.37 (m, 3H), 2.97 (dd, J = 24.7, 12.5 | |
| | Hz, 2H), 2.88 (s, 3H), 2.28 (brs, 1H), 1.98 (t, J = 13.7 | |
| | Hz, 2H), 1.65 (t, J = 13.5 Hz, 2H). | |
| | LC-MS: [M + H]+ = 596.23 | |
| 43 | ¹H NMR (400 MHz, methanol-d₄) δ 8.82 (s, 1H), 8.76 | Example 4 |
| | (s, 1H), 7.99 (s, 1H), 7.67 (s, 1H), 6.89-6.79 (m, | |
| | 1H), 6.63 (dd, J = 8.7, 3.8 Hz, 1H), 5.46 (d, J = 15.0 | |
| | Hz, 1H), 4.86 (s, 2H), 4.82 (d, J = 14.9 Hz, 1H), 4.58 | |
| | (td, J = 8.7, 1.2 Hz, 2H), 3.95 (d, J = 14.4 Hz, 1H), | |
| | 3.49 (d, J = 14.5 Hz, 1H), 3.41 (t, J = 8.7 Hz, 2H), | |
| | 0.91 (t, J = 2.4 Hz, 1H), 0.86-0.72 (m, 3H). | |
| | LC-MS: [M + H]+ = 555.16 | |
| 44 | ¹H NMR (400 MHz, methanol-d₄) δ 8.75 (s, 1H), 8.15 | Example 4 |
| | (d, J = 8.1 Hz, 1H), 7.88 (d, J = 8.1 Hz, 1H), 7.66 (s, | |
| | 1H), 6.87 (t, J = 9.5 Hz, 1H), 6.66 (dd, J = 8.8, 3.8 Hz, | |
| | 1H), 5.54 (d, J = 14.8 Hz, 1H), 4.86 (s, 2H), 4.82 (d, J = | |
| | 14.9 Hz, 1H), 4.60 (t, J = 8.7 Hz, 2H), 4.15 (d, J = | |
| | 14.1 Hz, 1H), 3.58 (d, J = 14.1 Hz, 1H), 3.42 (t, J = | |
| | 8.7 Hz, 2H), 0.92 (s, 1H), 0.80 (dd, J = 17.3, 9.1 Hz, | |
| | 3H). | |
| | LC-MS: [M + H]+ = 555.16 | |
| 45 | ¹H NMR (400 MHz, methanol-d₄) δ 8.83 (s, 1H), 8.77 | Example 4 |
| | (brs, 1H), 8.02 (s, 1H), 7.70 (s, 1H), 6.95-6.80 (m, | |
| | 1H), 6.66 (dd, J = 8.7, 3.9 Hz, 1H), 5.45 (d, J = 14.8 | |
| | Hz, 1H), 4.60 (t, J = 8.8 Hz, 2H), 4.39 (d, J = 15.0 Hz, | |
| | 1H), 3.96 (s, 2H), 3.85 (s, 1H), 3.56-3.35 (m, 4H), | |
| | 3.42 (m, 2H), 3.15-3.10 (m, 1H), 2.32-2.22 (m, 1H), | |
| | 1.57 (dd, J = 24.7, 12.9 Hz, 2H), 1.49-1.26 (m, 2H). | |
| | LC-MS: [M + H]+ = 583.20 | |
| 46 | ¹H NMR (400 MHz, methanol-d₄) δ 8.74 (s, 1H), 8.15 | Example 4 |
| | (d, J = 8.1 Hz, 1H), 7.89 (d, J = 8.1 Hz, 1H), 7.65 (s, | |
| | 1H), 6.86 (t, J = 9.5 Hz, 1H), 6.66 (d, J = 8.6 Hz, 1H), | |
| | 5.56 (d, J = 14.7 Hz, 1H), 4.60 (t, J = 8.7 Hz, 2H), | |
| | 4.37 (d, J = 14.9 Hz, 1H), 3.94 (d, J = 11.1 Hz, 2H), | |
| | 3.69 (d, J = 13.7 Hz, 1H), 3.56-3.35 (m, 4H), 3.42 (t, | |
| | J = 8.7 Hz, 2H), 3.34-3.30 (m, 1H), 2.32-2.22 (m, | |
| | 1H), 1.57 (dd, J = 24.7, 12.9 Hz, 2H), 1.49-1.26 (m, | |
| | 2H). | |
| | LC-MS: [M + H]+ = 583.20 | |
| 47 | ¹H NMR (400 MHz, methanol-d₄) δ 8.74 (s, 1H), 8.15 | Example 4 |
| | (d, J = 8.1 Hz, 1H), 7.88 (d, J = 8.1 Hz, 1H), 7.63 (s, | |
| | 1H), 6.87 (dd, J = 10.3, 8.7 Hz, 1H), 6.66 (dd, J = 8.7, | |
| | 3.9 Hz, 1H), 5.52 (d, J = 14.6 Hz, 1H), 4.85 (s, 2H), | |
| | 4.67-4.46 (m, 2H), 4.35 (d, J = 14.6 Hz, 1H), 3.91 | |
| | (dd, J = 13.4, 6.9 Hz, 1H), 3.57 (dd, J = 13.4, 7.4 Hz, | |
| | 1H), 3.42 (t, J = 8.7 Hz, 2H), 2.44-2.21 (m, 1H), | |
| | 2.11 (ddt, J = 11.9, 7.6, 4.0 Hz, 2H), 1.98 (q, J = 11.4 | |
| | Hz, 2H), 1.32 (s, 3H). | |
| | LC-MS: [M + H]+ = 583.20 | |
| 48 | ¹H NMR (400 MHz, methanol-d₄) δ 8.83 (s, 1H), 8.75 | Example 4 |
| | (s, 1H), 8.05 (s, 1H), 7.69 (s, 1H), 6.93-6.81 (m, | |
| | 1H), 6.66 (dd, J = 8.7, 3.9 Hz, 1H), 5.42 (d, J = 14.9 | |
| | Hz, 1H), 4.84 (d, J = 1.0 Hz, 2H), 4.65-4.52 (m, | |
| | 2H), 4.08 (d, J = 14.2 Hz, 1H), 3.42 (t, J = 8.7 Hz, | |
| | 2H), 3.08 (d, J = 14.2 Hz, 1H), 2.68 (s, 1H), 1.42- | |
| | 1.18 (m, 6H). | |
| | LC-MS: [M + H]+ = 557.18 | |
| 49 | ¹H NMR (400 MHz, methanol-d₄) δ 8.83 (s, 1H), 8.76 | Example 4 |
| | (q, J = 1.5 Hz, 1H), 7.94 (s, 1H), 7.66 (s, 1H), 6.86 (t, | |
| | J = 9.7 Hz, 1H), 6.65 (d, J = 8.5 Hz, 1H), 5.41 (d, J = | |
| | 14.9 Hz, 1H), 4.84 (d, J = 1.0 Hz, 2H), 4.59 (t, J = 8.8 | |
| | Hz, 2H), 4.34 (d, J = 14.9 Hz, 1H), 4.00 (dd, J = 13.6, | |
| | 7.3 Hz, 1H), 3.41 (t, J = 8.7 Hz, 2H), 3.26 (dd, J = | |
| | 13.6, 6.8 Hz, 1H), 2.31 (q, J = 8.1 Hz, 1H), 2.14 (ddt, | |
| | J = 17.7, 12.2, 5.1 Hz, 2H), 2.05-1.87 (m, 2H), 1.33 | |
| | (s, 3H). | |
| | LC-MS: [M + H]+ = 583.20 | |
| 50 | ¹H NMR (400 MHz, methanol-d₄) δ 8.77 (s, 1H), 8.13 | Example 4 |
| | (d, J = 8.0 Hz, 1H), 7.67-7.51 (m, 2H), 6.85 (dd, J = | |
| | 10.3, 8.7 Hz, 1H), 6.64 (dd, J = 8.7, 3.8 Hz, 1H), 5.49 | |
| | (d, J = 15.1 Hz, 1H), 4.82 (s, 2H), 4.65-4.50 (m, | |
| | 2H), 4.35 (d, J = 15.1 Hz, 1H), 3.98 (dd, J = 13.6, 7.6 | |
| | Hz, 1H), 3.49 (dd, J = 13.5, 6.4 Hz, 1H), 3.40 (t, J = | |
| | 8.7 Hz, 2H), 2.72 (s, 3H), 2.33 (q, J = 8.1 Hz, 1H), | |
| | 2.22-2.04 (m, 2H), 1.99 (td, J = 10.2, 3.8 Hz, 2H), | |
| | 1.33 (s, 3H). | |
| | LC-MS: [M + H]+ = 529.22 | |
| 51 | ¹H NMR (400 MHz, methanol-d₄) δ 8.81 (s, 1H), 8.17 | Example 4 |
| | (d, J = 8.1 Hz, 1H), 7.88 (d, J = 8.2 Hz, 1H), 7.66 (s, | |
| | 1H), 6.94-6.82 (m, 1H), 6.66 (dd, J = 8.7, 3.9 Hz, | |
| | 1H), 5.56-5.45 (m, 1H), 4.88 (s, 2H), 4.65-4.50 | |
| | (m, 2H), 4.45-4.41 (m, 1H), 4.12-3.93 (m, 2H), 3.55 | |
| | -3.39 (m, 4H), 3.39-3.36 (m, 2H), 2.58-2.50 (m, | |
| | 1H), 2.30-2.25 (m, 1H), 1.65-1.60 (m, 1H), 1.38-1.34 | |
| | (m, 1H). | |
| | LC-MS: [M + H]+ = 569.18 | |
| 52 | ¹H NMR (400 MHz, methanol-d₄) δ 8.76 (s, 1H), 8.15 | Example 4 |
| | (d, J = 8.1 Hz, 1H), 7.89 (d, J = 8.1 Hz, 1H), 7.65 (s, | |
| | 1H), 6.85 (t, J = 9.4 Hz, 1H), 6.64 (dd, J = 8.6, 3.8 Hz, | |
| | 1H), 5.54 (dd, J = 14.8, 6.6 Hz, 1H), 4.85 (s, 2H), 4.59 | |
| | (t, J = 8.8 Hz, 2H), 4.39 (t, J = 14.0 Hz, 1H), 4.00- | |
| | 3.84 (m, 2H), 3.79-3.75 (m, 2H), 3.64-3.48 (m, 1H), | |
| | 3.41 (t, J = 8.7 Hz, 2H), 3.24 (q, J = 7.3 Hz, 2H), 2.88 | |
| | (m, 1H), 2.11-1.92 (m, 1H), 1.84-1.61 (m, 1H). | |
| | LC-MS: [M + H]+ = 569.18 | |
| 53 | ¹H NMR (400 MHz, DMSO-d₆) δ 8.74 (s, 1H), 8.45 | Example 2 |
| | (t, J = 7.2 Hz, 1H), 8.37 (t, J = 5.2 Hz, 1H), 7.64 (s, | |
| | 1H), 7.44 (s, 1H), 6.93 (dd, J = 10.4, 8.7 Hz, 1H), 6.69 | |
| | (dd, J = 8.6, 3.9 Hz, 1H), 4.78-4.60 (m, 3H), 4.54 (t, | |
| | J = 8.7 Hz, 2H), 4.18 (dd, J = 16.0, 5.7 Hz, 1H), 3.88 | |
| | (s, 3H), 3.30 (t, J = 8.7 Hz, 2H). | |
| | LC-MS: [M + H]+ = 420.15 | |
| 54 | ¹H NMR (400 MHz, d₄-MeOD) δ 8.62 (s, 1H), 7.49- | Example 1 |
| | 7.52 (m, 2H), 6.85 (t, J = 9.6 Hz, 1H), 6.63 (m, 1H), | |
| | 4.80 (s, 2H), 4.57 (t, J = 8.4 Hz, 2H), 4.44 (s, 2H), | |
| | 3.90 (s, 3H), 3.37 (t, J = 8.4 Hz, 2H). | |
| | LC-MS: [M + H]+ = 421.15 | |
| 55 | ¹H NMR (400 MHz, DMSO-d₆) δ 8.80 (s, 1H), 8.21 | Example 4 |
| | (d, J = 8.1 Hz, 1H), 7.96 (d, J = 8.0 Hz, 1H), 7.59 (s, | |
| | 1H), 6.97 (t, J = 9.4 Hz, 1H), 6.72 (dd, J = 8.8, 3.7 Hz, | |
| | 1H), 5.44 (s, 1H), 4.76 (s, 2H), 4.57 (t, J = 8.7 Hz, | |
| | 2H), 4.41 (brs, 1H), 3.73 (brs, 1H), 3.38 (t, J = 8.7 Hz, | |
| | 2H), 3.10 (brs, 1H), 1.28-1.16 (m, 2H), 0.49 (d, J = | |
| | 39.7 Hz, 2H), 0.24 (s, 1H). | |
| | LC-MS: [M + H]+ = 539.17 | |
| 56 | ¹H NMR (400 MHz, DMSO-d₆) δ 8.73 (s, 1H), 8.33 | Example 4 |
| | (s, 1H), 7.33 (s, 1H), 6.96 (t, J = 9.5 Hz, 1H), 6.71 | |
| | (dd, J = 8.7, 3.8 Hz, 1H), 4.94 (d, J = 16.2 Hz, 1H), | |
| | 4.72 (s, 2H), 4.55 (t, J = 8.7 Hz, 2H), 4.34 (d, J = 16.2 | |
| | Hz, 1H), 3.92 (s, 3H), 3.32 (td, J = 8.9, 2.9 Hz, 2H), | |
| | 2.5-2.45 (m, 1H), 2.28 (s, 3H), 1.04 (d, J = 7.0 Hz, | |
| | 1H), 0.82 (dd, J = 9.9, 6.3 Hz, 2H), 0.72 (dd, J = 8.3, | |
| | 4.4 Hz, 1H). | |
| | LC-MS: [M + H]+ = 474.20 | |
| 57 | ¹H NMR (400 MHz, methanol-d₄) δ 8.76 (s, 1H), 8.15 | Example 4 |
| | (d, J = 8.1 Hz, 1H), 7.88 (d, J = 8.2 Hz, 1H), 7.65 (s, | |
| | 1H), 6.94-6.82 (m, 1H), 6.66 (dd, J = 8.7, 3.8 Hz, | |
| | 1H), 5.54 (d, J = 14.7 Hz, 1H), 4.87 (s, 2H), 4.64- | |
| | 4.57 (m, 2H), 4.52 (d, J = 14.7 Hz, 1H), 4.00-3.89 | |
| | (m, 1H), 3.75-3.68 (m, 3H), 3.43 (d, J = 8.7 Hz, 2H), | |
| | 3.40 (s, 3H). | |
| | LC-MS: [M + H]+ = 543.16 | |
| 58 | ¹H NMR (400 MHz, DMSO-d₆) δ 8.84 (s, 1H), 8.80 | Example 4 |
| | (t, J = 5.1 Hz, 1H), 8.20 (d, J = 8.1 Hz, 1H), 7.96 (d, J = | |
| | 8.1 Hz, 1H), 7.58 (s, 1H), 6.96 (t, J = 9.5 Hz, 1H), | |
| | 6.72 (dd, J = 8.9, 3.8 Hz, 1H), 5.38 (d, J = 14.6 Hz, | |
| | 1H), 4.77 (d, J = 4.8 Hz, 2H), 4.68 (d, J = 14.6 Hz, | |
| | 1H), 4.56 (t, J = 8.7 Hz, 2H), 3.79 (d, J = 13.5 Hz, | |
| | 1H), 3.34 (t, J = 8.7 Hz, 2H), 3.27 (d, J = 13.5 Hz, | |
| | 1H), 1.18 (s, 3H), 1.13 (s, 3H). | |
| | LC-MS: [M + H]+ = 557.18 | |
| 59 | ¹H NMR (400 MHz, DMSO-d₆) δ 8.85 (brs, 2H), 8.22 | Example 4 |
| | (d, J = 8.1 Hz, 1H), 7.94 (d, J = 8.1 Hz, 1H), 7.56 (s, | |
| | 1H), 6.95 (t, J = 9.5 Hz, 1H), 6.71 (dd, J = 8.8, 3.8 Hz, | |
| | 1H), 5.37 (d, J = 15.0 Hz, 1H), 4.76 (d, J = 3.7 Hz, | |
| | 2H), 4.53 (dt, J = 20.8, 7.8 Hz, 3H), 4.37 (d, J = 15.0 | |
| | Hz, 1H), 3.34 (t, J = 8.7 Hz, 2H), 1.27 (d, J = 6.7 Hz, | |
| | 3H), 1.07 (d, J = 6.5 Hz, 3H). | |
| | LC-MS: [M + H]+ = 527.17 | |
| 60 | ¹H NMR (400 MHz, DMSO-d₆) δ 8.79 (s, 1H), 8.66 | Example 4 |
| | (t, J = 5.1 Hz, 1H), 7.98 (d, J = 1.8 Hz, 1H), 7.81- | |
| | 7.67 (m, 2H), 7.47 (s, 1H), 6.96 (dd, J = 10.3, 8.6 Hz, | |
| | 1H), 6.71 (dd, J = 8.6, 3.9 Hz, 1H), 5.26 (d, J = 15.1 | |
| | Hz, 1H), 4.76 (d, J = 4.9 Hz, 2H), 4.56 (t, J = 8.8 Hz, | |
| | 2H), 4.38 (d, J = 15.1 Hz, 1H), 4.03 (s, 1H), 3.88 (s, | |
| | 2H), 3.35-3.30 (m, 3H), 2.47-2.21 (m, 3H), 1.59 (d, | |
| | J = 11.9 Hz, 1H), 0.95 (d, J = 12.2 Hz, 1H). | |
| | LC-MS: [M + H]+ = 568.54 | |
| 61 | LC-MS: [M + H]+ = 530.47 | Example 4 |
| 62 | ¹H NMR (400 MHz, DMSO-d₆) δ 8.84 (s, 1H), 8.70 | Example 4 |
| | (t, J = 5.1 Hz, 1H), 7.85-7.69 (m, 3H), 7.52 (s, 1H), | |
| | 6.96 (dd, J = 10.3, 8.6 Hz, 1H), 6.72 (dd, J = 8.7, 3.9 | |
| | Hz, 1H), 5.33 (d, J = 15.1 Hz, 1H), 4.77 (d, J = 4.8 | |
| | Hz, 2H), 4.56 (t, J = 8.7 Hz, 2H), 4.36 (d, J = 15.1 Hz, | |
| | 1H), 4.10 (dd, J = 33.2, 14.7 Hz, 1H), 3.34 (t, J = 8.7 | |
| | Hz, 2H), 3.21 (dd, J = 14.7, 12.3 Hz, 1H), 1.39 (dd, J = | |
| | 40.0, 21.9 Hz, 6H). | |
| | LC-MS: [M + H]+ = 558.52 | |
| 63 | ¹H NMR (400 MHz, DMSO-d₆) δ 8.85 (s, 1H), 8.72 | Example 4 |
| | (t, J = 5.1 Hz, 1H), 7.96 (d, J = 1.9 Hz, 1H), 7.80 (dd, | |
| | J = 8.3, 1.9 Hz, 1H), 7.73 (d, J = 8.2 Hz, 1H), 7.53 (s, | |
| | 1H), 6.96 (dd, J = 10.3, 8.7 Hz, 1H), 6.71 (dd, J = 8.7, | |
| | 3.9 Hz, 1H), 5.36 (d, J = 15.2 Hz, 1H), 4.77 (d, J = 4.0 | |
| | Hz, 2H), 4.56 (t, J = 8.7 Hz, 2H), 4.33 (dd, J = 15.3, | |
| | 3.0 Hz, 2H), 3.53 (td, J = 14.2, 9.4 Hz, 1H), 3.34 (t, J = | |
| | 8.7 Hz, 2H), 1.66 (t, J = 19.3 Hz, 3H). | |
| | LC-MS: [M + H]+ = 562.48 | |
| 64 | LC-MS: [M + H]+ = 474.48 | Example 4 |
| 65 | ¹H NMR (400 MHz, DMSO-d₆) δ 8.84 (s, 1H), 8.56 | Example 4 |
| | (d, J = 6.1 Hz, 1H), 7.53 (ddd, J = 19.8, 9.1, 4.3 Hz, | |
| | 2H), 7.38 (s, 1H), 7.27 (td, J = 8.5, 2.8 Hz, 1H), 6.95 | |
| | (dd, J = 10.3, 8.7 Hz, 1H), 6.71 (dd, J = 8.6, 3.9 Hz, | |
| | 1H), 5.15 (d, J = 15.0 Hz, 2H), 4.77-4.71 (m, 2H), | |
| | 4.55 (t, J = 8.7 Hz, 2H), 4.25 (d, J = 15.0 Hz, 1H), | |
| | 4.03 (ddt, J = 12.2, 7.9, 3.9 Hz, 1H), 3.90 (td, J = 12.2, | |
| | 4.3 Hz, 2H), 3.39-3.28 (m, 4H), 2.30 (qd, J = 12.0, | |
| | 4.4 Hz, 1H), 1.61 (d, J = 12.3 Hz, 1H), 1.11 (d, J = | |
| | 12.4 Hz, 1H). | |
| | LC-MS: [M + H]+ = 518.53 | |
| 66 | ¹H NMR (400 MHz, DMSO-d₆) δ 8.81 (s, 1H), 8.67 | Example 4 |
| | (d, J = 6.4 Hz, 1H), 7.99 (d, J = 1.9 Hz, 1H), 7.77 (dd, | |
| | J = 8.3, 1.9 Hz, 1H), 7.70 (d, J = 8.2 Hz, 1H), 7.48 (s, | |
| | 1H), 6.95 (dd, J = 10.2, 8.7 Hz, 1H), 6.70 (dd, J = 8.6, | |
| | 3.8 Hz, 1H), 5.23 (d, J = 14.8 Hz, 1H), 4.74 (d, J = 4.7 | |
| | Hz, 2H), 4.55 (t, J = 8.8 Hz, 2H), 4.24 (d, J = 15.0 Hz, | |
| | 1H), 3.85 (d, J = 11.4 Hz, 2H), 3.66 (dd, J = 12.9, 8.1 | |
| | Hz, 1H), 3.33 (t, J = 8.7 Hz, 2H), 3.28-3.15 (m, 2H), | |
| | 2.96 (s, 1H), 2.07 (s, 1H), 1.56-1.08 (m, 4H). | |
| | LC-MS: [M + H]+ = 582.20 | |
| 67 | ¹H NMR (400 MHz, DMSO-d₆) δ 8.78 (s, 1H), 8.67 | Example 4 |
| | (t, J = 5.1 Hz, 1H), 7.82-7.67 (m, 3H), 7.53 (s, 1H), | |
| | 6.95 (dd, J = 10.3, 8.7 Hz, 1H), 6.70 (dd, J = 8.6, 3.9 | |
| | Hz, 1H), 5.21 (d, J = 15.0 Hz, 1H), 4.75 (d, J = 4.5 | |
| | Hz, 2H), 4.55 (t, J = 8.8 Hz, 2H), 4.21 (d, J = 15.0 Hz, | |
| | 1H), 3.33 (d, J = 17.5 Hz, 2H), 2.31-2.20 (m, 1H), | |
| | 1.02 (tq, J = 6.8, 4.2, 3.4 Hz, 1H), 0.90-0.69 (m, | |
| | 3H). | |
| | LC-MS: [M + H]+ = 524.49 | |
| 68 | ¹H NMR (400 MHz, DMSO-d₆) δ 8.84 (s, 1H), 8.76- | Example 4 |
| | 8.62 (m, 1H), 7.89 (dd, J = 8.1, 1.9 Hz, 1H), 7.78 (dd, | |
| | J = 8.4, 2.1 Hz, 1H), 7.76-7.63 (m, 1H), 7.50 (s, | |
| | 1H), 6.95 (dd, J = 10.3, 8.7 Hz, 1H), 6.70 (dd, J = 8.7, | |
| | 3.9 Hz, 1H), 5.27 (d, J = 15.0 Hz, 1H), 4.75 (d, J = 4.3 | |
| | Hz, 2H), 4.55 (t, J = 8.8 Hz, 2H), 4.32 (dd, J = 15.1, | |
| | 9.3 Hz, 1H), 3.91-3.38 (m, 7H), 3.38-3.25 (m, 3H), | |
| | 3.09 (ddd, J = 53.1, 13.6, 5.9 Hz, 1H). | |
| | LC-MS: [M + H]+ = 584.54 | |
| 69 | ¹H NMR (400 MHz, DMSO-d₆) δ 8.83 (s, 1H), 8.43 | Example 4 |
| | (t, J = 4.4 Hz, 1H), 8.21 (d, J = 8.1 Hz, 1H), 7.96 (d, J = | |
| | 8.1 Hz, 1H), 7.58 (s, 1H), 7.40 (td, J = 8.4, 6.9 Hz, | |
| | 1H), 6.96 (d, J = 8.4 Hz, 1H), 6.88 (ddd, J = 9.3, 8.4, | |
| | 0.9 Hz, 1H), 5.38 (d, J = 14.7 Hz, 1H), 4.77 (d, J = 4.2 | |
| | Hz, 2H), 4.24 (d, J = 14.7 Hz, 1H), 3.86 (s, 3H), 2.62 | |
| | (tt, J = 7.5, 4.2 Hz, 1H), 1.04-0.87 (m, 2H), 0.77 (tt, | |
| | J = 9.4, 6.1 Hz, 1H), 0.72-0.62 (m, 1H). | |
| | LC-MS: [M + H]+ = 513.46 | |
| 70 | ¹H NMR (400 MHz, DMSO-d₆) δ 9.07 (s, 2H), 8.88 | Example 4 |
| | (s, 1H), 8.68 (s, 1H), 8.54 (t, J = 4.4 Hz, 1H), 8.16 (s, | |
| | 1H), 7.54 (s, 1H), 7.42 (td, J = 8.4, 6.8 Hz, 1H), 6.97 | |
| | (d, J = 8.4 Hz, 1H), 6.90 (t, J = 8.9 Hz, 1H), 4.86- | |
| | 4.68 (m, 2H), 3.87 (s, 3H), 2.62 (tt, J = 7.5, 4.2 Hz, | |
| | 1H), 0.82-0.48 (m, 4H). | |
| | LC-MS: [M + H]+ = 513.46 | |
| 71 | ¹H NMR (400 MHz, DMSO-d₆) δ 8.85 (s, 1H), 8.44 | Example 4 |
| | (s, 1H), 8.22 (d, J = 8.1 Hz, 1H), 7.95 (d, J = 8.2 Hz, | |
| | 1H), 7.56 (s, 1H), 7.40 (td, J = 8.4, 6.9 Hz, 1H), 6.96 | |
| | (d, J = 8.4 Hz, 1H), 6.88 (t, J = 8.9 Hz, 1H), 5.38 (d, J = | |
| | 15.1 Hz, 1H), 4.83-4.71 (m, 2H), 4.49 (p, J = 6.9 | |
| | Hz, 2H), 4.36 (d, J = 15.1 Hz, 1H), 3.35 (q, J = 8.5 | |
| | Hz, 2H), 1.26 (d, J = 6.9 Hz, 3H), 1.06 (d, J = 6.7 Hz, | |
| | 3H). | |
| | LC-MS: [M + H]+ = 515.48 | |
| 72 | LC-MS: [M + H]+ = 531.45 | Example 4 |
| 73 | ¹H NMR (400 MHz, DMSO-d₆) δ 9.02-8.67 (m, | Example 4 |
| | 2H), 8.11 (d, J = 7.3 Hz, 1H), 7.62 (d, J = 7.4 Hz, 1H), | |
| | 6.96 (q, J = 9.1 Hz, 1H), 6.72 (dq, J = 8.4, 3.7 Hz, | |
| | 1H), 5.26 (dd, J = 15.2, 7.2 Hz, 2H), 4.78 (d, J = 6.4 | |
| | Hz, 2H), 4.56 (q, J = 8.5 Hz, 2H), 4.39 (dd, J = 15.4, | |
| | 7.0 Hz, 1H), 4.27 (q, J = 7.3 Hz, 1H), 3.35 (q, J = 8.5 | |
| | Hz, 2H), 1.41-1.23 (m, 3H), 1.23-1.06 (m, 3H). | |
| | LC-MS: [M + H]+ = 527.49 | |
| 74 | LC-MS: [M + H]+ = 581.22 | Example 4 |
| 75 | ¹H NMR (400 MHz, DMSO-d₆) δ 9.45 (s, 1H), 8.78 | Example 4 |
| | (s, 1H), 8.60 (t, J = 5.0 Hz, 1H), 7.57 (dd, J = 8.7, 5.7 | |
| | Hz, 1H), 7.45 (dd, J = 9.3, 2.8 Hz, 1H), 7.38 (s, 1H), | |
| | 7.29 (td, J = 8.6, 2.8 Hz, 1H), 6.95 (dd, J = 10.3, 8.7 | |
| | Hz, 1H), 6.70 (dd, J = 8.6, 3.8 Hz, 1H), 5.19 (d, J = | |
| | 15.2 Hz, 1H), 4.73 (d, J = 5.0 Hz, 2H), 4.55 (t, J = 8.7 | |
| | Hz, 2H), 4.19 (d, J = 15.2 Hz, 1H), 4.05-3.88 (m, | |
| | 1H), 3.33 (d, J = 8.8 Hz, 4H), 3.04 (m, 4H), 2.81- | |
| | 2.64 (m, 4H), 1.91 (d, J = 13.4 Hz, 1H), 1.35-1.15 | |
| | (m, 3H). | |
| | LC-MS: [M + H]+ = 530.22 | |
| 76 | ¹H NMR (400 MHz, DMSO-d₆) δ 8.82 (d, J = 2.3 Hz, | Example 4 |
| | 1H), 8.59 (t, J = 5.2 Hz, 1H), 7.54 (td, J = 8.9, 4.2 Hz, | |
| | 2H), 7.42 (d, J = 1.3 Hz, 1H), 7.31 (td, J = 8.5, 2.7 Hz, | |
| | 1H), 7.03-6.89 (m, 1H), 6.71 (dd, J = 8.7, 3.8 Hz, | |
| | 1H), 5.24 (d, J = 15.2 Hz, 1H), 4.74 (d, J = 4.7 Hz, | |
| | 2H), 4.56 (t, J = 8.7 Hz, 2H), 4.35 (q, J = 15.5 Hz, | |
| | 2H), 4.18 (d, J = 15.2 Hz, 1H), 3.33 (t, J = 8.7 Hz, | |
| | 2H), 1.67 (t, J = 19.2 Hz, 3H). | |
| | LC-MS: [M + H]+ = 512.48 | |
| 77 | ¹H NMR (400 MHz, DMSO-d₆) δ 8.85 (d, J = 10.4 | Example 4 |
| | Hz, 2H), 8.23 (d, J = 8.1 Hz, 1H), 8.00 (d, J = 8.2 Hz, | |
| | 1H), 7.63 (s, 1H), 7.01-6.92 (m, 1H), 6.72 (dd, J = | |
| | 8.7, 3.9 Hz, 1H), 5.54 (d, J = 14.9 Hz, 1H), 4.77 (d, J = | |
| | 4.9 Hz, 2H), 4.56 (t, J = 8.8 Hz, 2H), 4.30 (dd, J = | |
| | 13.9, 7.1 Hz, 3H), 3.35 (q, J = 8.5 Hz, 2H), 1.66 (t, J = | |
| | 19.4 Hz, 3H). | |
| | LC-MS: [M + H]+ = 563.48 | |
| 78 | ¹H NMR (400 MHz, DMSO-d₆) δ 8.82 (s, 1H), 8.57 | Example 4 |
| | (d, J = 5.6 Hz, 1H), 7.55 (dd, J = 8.7, 5.7 Hz, 1H), | |
| | 7.45-7.33 (m, 2H), 7.30 (td, J = 8.5, 2.7 Hz, 1H), | |
| | 6.95 (dd, J = 10.3, 8.6 Hz, 1H), 6.70 (dd, J = 8.7, 3.8 | |
| | Hz, 1H), 5.21 (d, J = 15.0 Hz, 1H), 4.82-4.67 (m, | |
| | 2H), 4.55 (t, J = 8.7 Hz, 2H), 4.24 (d, J = 15.0 Hz, | |
| | 1H), 4.08 (dd, J = 33.6, 15.1 Hz, 1H), 3.33 (t, J = 8.9 | |
| | Hz, 3H), 1.44 (d, J = 21.5 Hz, 3H), 1.31 (d, J = 22.2 | |
| | Hz, 3H). | |
| | LC-MS: [M + H]+ = 508.52 | |
| 79 | ¹H NMR (400 MHz, DMSO-d₆) δ 8.78 (s, 1H), 8.56 | Example 4 |
| | (t, J = 5.1 Hz, 1H), 7.52 (ddd, J = 12.1, 9.0, 4.2 Hz, | |
| | 2H), 7.39 (s, 1H), 7.28 (td, J = 8.6, 2.8 Hz, 1H), 6.95 | |
| | (dd, J = 10.3, 8.7 Hz, 1H), 6.70 (dd, J = 8.6, 3.8 Hz, | |
| | 1H), 5.19 (d, J = 15.0 Hz, 1H), 4.71 (dd, J = 15.6, 4.8 | |
| | Hz, 2H), 4.65-4.46 (m, 3H), 4.18 (d, J = 15.0 Hz, | |
| | 1H), 4.12-3.93 (m, 1H), 3.47 (td, J = 15.0, 7.5 Hz, | |
| | 2H), 3.33 (t, J = 8.7 Hz, 2H). | |
| | LC-MS: [M + H]+ = 480.46 | |
| 80 | ¹H NMR (400 MHz, DMSO-d₆) δ 8.80 (s, 1H), 8.58 | Example 4 |
| | (t, J = 4.8 Hz, 1H), 7.60 (d, J = 9.2 Hz, 1H), 7.41(d, J = | |
| | 2.8 Hz, 1H), 7.41 (s, 1H), 7.26-7.31 (m, 1H), 6.95 | |
| | (t, J = 9.6 Hz, 1H), 6.70 (dd, J = 8.6, 3.9 Hz, 1H), 6.26 | |
| | (t, J = 56.4 Hz, 1H), 5.24 (d, J = 14.9 Hz, 1H), 4.73 | |
| | (d, J = 4.9 Hz, 2H), 4.55 (t, J = 8.9 Hz, 2H), 4.19 (d, J | |
| | = 15.2 Hz, 1H), 4.04-4.14 (m, 1H), 3.58-3.69 (m, 2H), | |
| | 3.31-3.35 (m, 2H). | |
| | LC-MS: [M + H]+ = 548.46 | |
| 81 | ¹H NMR (400 MHz, DMSO-d₆) δ 8.83 (s, 1H), 8.72 | Example 4 |
| | (t, J = 4.8 Hz, 1H), 8.07 (d, J = 1.2 Hz, 1H), 7.79-7.70 | |
| | (m, 2H), 7.52 (s, 1H), 6.95 (dd, J = 10.3, 8.6 Hz, 1H), | |
| | 6.71 (dd, J = 8.6, 3.9 Hz, 1H), 6.24 (t, J = 56.4 Hz, | |
| | 1H), 5.34 (d, J = 15.2 Hz, 1H), 4.75 (d, J = 4.8 Hz, | |
| | 2H), 4.56 (t, J = 8.9 Hz, 2H), 4.34 (d, J = 15.1 Hz, | |
| | 1H), 4.03-4.14 (m, 1H), 3.60-3.71 (m, 2H), 3.31-3.36 | |
| | (m, 2H). | |
| | LC-MS: [M + H]+ = 548.46 | |
| 82 | ¹H NMR (400 MHz, DMSO-d₆) δ 8.87 (d, J = 2.0 Hz, | Example 4 |
| | 2H), 8.83 (s, 1H), 8.18 (s, 1H), 7.67 (s, 1H), 6.96 (dd, | |
| | J = 10.3, 8.7 Hz, 1H), 6.71 (dd, J = 8.7, 3.9 Hz, 1H), | |
| | 5.48-5.28 (m, 1H), 4.77 (d, J = 4.2 Hz, 2H), 4.56 (t, | |
| | J = 8.8 Hz, 2H), 4.41-4.20 (m, 2H), 3.62 (td, J = | |
| | 14.3, 10.0 Hz, 1H), 3.34 (t, J = 8.7 Hz, 2H), 1.66 (t, J | |
| | = 19.3 Hz, 3H). | |
| | LC-MS: [M + H]+ = 563.15 | |
| 83 | ¹H NMR (400 MHz, DMSO-d₆) δ 8.84 (s, 1H), 8.74 | Example 4 |
| | (t, J = 5.1 Hz, 1H), 8.17 (d, J = 1.9 Hz, 1H), 7.80 (dd, | |
| | J = 8.5, 1.9 Hz, 1H), 7.72 (d, J = 8.2 Hz, 1H), 7.54 (s, | |
| | 1H), 6.96 (dd, J = 10.3, 8.7 Hz, 1H), 6.72 (dd, J = 8.6, | |
| | 3.9 Hz, 1H), 5.38 (d, J = 15.3 Hz, 1H), 4.76 (d, J = 5.0 | |
| | Hz, 2H), 4.67-4.58 (m, 1H), 4.56 (t, J = 8.8 Hz, 2H), | |
| | 4.35 (d, J = 15.3 Hz, 1H), 4.17-4.03 (m, 1H), 3.34 (t, | |
| | J = 8.8 Hz, 2H). | |
| | LC-MS: [M + H]+ = 566.13 | |
| 84 | ¹H NMR (400 MHz, DMSO-d₆) δ 8.85 (dd, J = 14.5, | Example 4 |
| | 5.1 Hz, 3H), 8.36 (s, 1H), 7.69 (s, 1H), 7.02-6.92 | |
| | (m, 1H), 6.72 (dd, J = 8.6, 3.9 Hz, 1H), 5.43 (d, J = | |
| | 15.1 Hz, 1H), 4.77 (d, J = 5.0 Hz, 2H), 4.69-4.51 | |
| | (m, 3H), 4.38 (d, J = 15.1 Hz, 1H), 4.21-4.11 (m, | |
| | 1H), 3.34 (t, J = 8.8 Hz, 2H). | |
| | LC-MS: [M + H]+ = 567.13 | |
| 85 | ¹H NMR (400 MHz, DMSO-d₆) δ 8.87 (s, 1H), 8.81 | Example 4 |
| | (d, J = 7.0 Hz, 2H), 8.02 (s, 1H), 7.66 (s, 1H), 6.96 (t, | |
| | J = 9.5 Hz, 1H), 6.72 (dd, J = 8.6, 3.8 Hz, 1H), 5.34 | |
| | (d, J = 15.2 Hz, 1H), 4.77 (d, J = 4.8 Hz, 2H), 4.56 (t, | |
| | J = 8.8 Hz, 2H), 4.36 (d, J = 15.1 Hz, 1H), 4.21 (td, J | |
| | = 8.3, 3.7 Hz, 1H), 3.34 (t, J = 8.8 Hz, 2H), 3.19- | |
| | 3.02 (m, 2H), 2.92 (d, J = 19.6 Hz, 2H). | |
| | LC-MS: [M + H]+ = 575.15 | |
| 86 | ¹H NMR (400 MHz, DMSO-d₆) δ 8.77 (s, 1H), 8.64 | Example 4 |
| | (t, J = 5.1 Hz, 1H), 7.42 (s, 1H), 6.95 (dd, J = 10.3, 8.7 | |
| | Hz, 1H), 6.70 (dd, J = 8.6, 3.9 Hz, 1H), 5.03 (d, J = | |
| | 15.4 Hz, 1H), 4.72 (d, J = 4.9 Hz, 2H), 4.55 (t, J = 8.8 | |
| | Hz, 2H), 4.19 (d, J = 15.4 Hz, 1H), 3.30 (t, J = 8.7 Hz, | |
| | 2H), 2.78 (tt, J = 7.6, 4.1 Hz, 1H), 2.66 (s, 3H), 1.02 | |
| | (ddt, J = 9.2, 6.8, 4.3 Hz, 1H), 0.89 (dtd, J = 9.6, 7.0, | |
| | 5.3 Hz, 1H), 0.77 (dtd, J = 9.6, 7.0, 5.1 Hz, 1H), 0.64 | |
| | (ddt, J = 9.2, 6.9, 4.8 Hz, 1H). | |
| | LC-MS: [M + H]+ = 477.14 | |
| 87 | ¹H NMR (400 MHz, DMSO-d₆) δ 8.76 (s, 1H), 8.58 | Example 4 |
| | (t, J = 5.1 Hz, 1H), 7.51 (d, J = 5.1 Hz, 1H), 7.46 (s, | |
| | 1H), 7.16 (d, J = 5.1 Hz, 1H), 6.95 (dd, J = 10.3, 8.6 | |
| | Hz, 1H), 6.71 (dd, J = 8.7, 3.9 Hz, 1H), 5.01 (d, J = | |
| | 15.2 Hz, 1H), 4.72 (d, J = 4.9 Hz, 2H), 4.55 (t, J = 8.7 | |
| | Hz, 2H), 4.10 (d, J = 15.2 Hz, 1H), 3.32 (t, J = 8.7 Hz, | |
| | 2H), 2.50 (m, 1H), 1.04-0.92 (m, 1H), 0.88-0.65 | |
| | (m, 3H). | |
| | LC-MS: [M + H]+ = 462.13 | |
| 88 | ¹H NMR (400 MHz, DMSO-d₆) δ 8.75 (s, 1H), 8.50 | Example 4 |
| | (t, J = 5.1 Hz, 1H), 7.63-7.50 (m, 2H), 7.32 (d, J = | |
| | 5.3 Hz, 1H), 6.95 (dd, J = 10.3, 8.7 Hz, 1H), 6.70 (dd, | |
| | J = 8.6, 3.9 Hz, 1H), 5.31 (d, J = 16.1 Hz, 1H), 4.80- | |
| | 4.65 (m, 2H), 4.55 (t, J = 8.7 Hz, 2H), 4.17 (d, J = | |
| | 16.1 Hz, 1H), 3.32 (t, J = 8.7 Hz, 2H), 2.72-2.59 (m, | |
| | 1H), 1.04-0.93 (m, 1H), 0.77 (dddd, J = 16.2, 10.0, | |
| | 7.6, 4.4 Hz, 3H). | |
| | LC-MS: [M + H]+ = 462.13 | |
| 89 | ¹H NMR (400 MHz, DMSO-d₆) δ 8.86-8.80 (m, 2H), | Example 4 |
| | 8.27 (s, 1H), 7.66 (s, 1H), 6.96 (t, J = 8.8 Hz, 1H), | |
| | 6.71 (dd, J = 8.6, 3.8 Hz, 1H), 6.24 (t, J = 56.4 Hz, | |
| | 1H), 5.40 (d, J = 14.8 Hz, 1H), 4.76 (d, J = 4.0 Hz, | |
| | 1H), 4.56 (d, J = 8.8 Hz, 1H), 4.37 (d, J = 14.8 Hz, | |
| | 1H), 4.13-4.03 (m, 1H), 3.77-3.68 (m, 2H), 3.36-3.32 | |
| | (m, 2H). | |
| | LC-MS: [M + H]+ = 549.13 | |
| 90 | ¹H NMR (400 MHz, DMSO-d₆) δ 8.86 (s, 1H), 8.85- | Example 4 |
| | 8.74 (m, 2H), 8.15 (s, 1H), 7.63 (s, 1H), 6.96 (dd, J = | |
| | 10.3, 8.7 Hz, 1H), 6.72 (dd, J = 8.7, 3.9 Hz, 1H), 5.25 | |
| | (d, J = 14.8 Hz, 1H), 4.77 (d, J = 4.8 Hz, 2H), 4.56 (t, | |
| | J = 8.8 Hz, 2H), 4.32 (d, J = 14.8 Hz, 1H), 3.78 (dq, J | |
| | = 13.8, 6.9 Hz, 1H), 3.34 (t, J = 8.9 Hz, 2H), 3.12 (dq, | |
| | J = 13.6, 6.7 Hz, 1H), 1.10 (t, J = 7.0 Hz, 3H). | |
| | LC-MS: [M + H]+ = 513.15 | |
| 91 | ¹H NMR (400 MHz, DMSO-d₆) δ 8.89 (t, J = 5.1 Hz, | Example 4 |
| | 1H), 8.82 (s, 1H), 7.68 (s, 1H), 6.95 (dd, J = 10.3, 8.7 | |
| | Hz, 1H), 6.71 (dd, J = 8.7, 3.9 Hz, 1H), 5.22 (d, J = | |
| | 15.6 Hz, 1H), 4.74 (d, J = 4.8 Hz, 2H), 4.55 (t, J = 8.7 | |
| | Hz, 2H), 4.35 (d, J = 15.6 Hz, 1H), 3.32 (t, J = 8.7 Hz, | |
| | 2H), 2.76 (tt, J = 7.4, 4.1 Hz, 1H), 1.03-0.73 (m, | |
| | 3H), 0.71-0.60 (m, 1H). | |
| | LC-MS: [M + H]+ = 531.11 | |
| 92 | LC-MS: [M + H]+ = 488.15 | Example 4 |
| 93 | ¹H NMR (400 MHz, DMSO-d₆) δ 8.81 (s, 1H), 8.68 | Example 4 |
| | (t, J = 5.0 Hz, 1H), 7.97 (d, J = 1.9 Hz, 1H), 7.85- | |
| | 7.66 (m, 2H), 7.47 (d, J = 3.8 Hz, 1H), 6.95 (dd, J = | |
| | 10.3, 8.7 Hz, 1H), 6.71 (dd, J = 8.6, 3.9 Hz, 1H), 5.30 | |
| | (d, J = 15.0 Hz, 1H), 4.75 (d, J = 4.5 Hz, 2H), 4.55 (t, | |
| | J = 8.8 Hz, 2H), 4.39 (d, J = 15.0 Hz, 1H), 4.07-3.88 | |
| | (m, 3H), 3.33 (t, J = 8.7 Hz, 2H), 2.42-2.19 (m, 2H), | |
| | 1.59 (dt, J = 13.0, 7.7 Hz, 1H), 1.12 (d, J = 6.2 Hz, | |
| | 3H), 1.08 (dd, J = 15.7, 6.1 Hz, 1H), 1.02 (d, J = 6.3 | |
| | Hz, 3H), 0.84 (dt, J = 12.2, 5.7 Hz, 1H). | |
| | LC-MS: [M + H]+ = 596.22 | |
| 94 | ¹H NMR (400 MHz, DMSO-d₆) δ 9.04 (t, J = 4.8 Hz, | Example 6 |
| | 1H), 8.88 (s, 1H), 8.61 (dd, J = 3.6, 1.4 Hz, 1H), 8.25 | |
| | (dd, J = 8.0, 1.6 Hz, 1H), 8.14 (s, 1H), 7.53 (dd, J = | |
| | 8.0, 4.4 Hz, 1H), 6.96 (t, J = 8.8 Hz, 1H), 6.71 (dd, J = | |
| | 8.8, 4.0 Hz, 1H), 5.05 (d, J = 14.0 Hz, 1H), 4.79 (t, J = | |
| | 6.4 Hz, 2H), 4.53-4.58 (m, 3H), 3.34 (t, J = 8.8 Hz, | |
| | 2H). | |
| | LC-MS: [M + H]+ = 438.09 | |
| 95 | ¹H NMR (400 MHz, DMSO-d₆) δ 8.98 (t, J = 4.8 Hz, | Example 6 |
| | 1H), 8.86 (s, 1H), 8.15 (d, J = 8.4 Hz, 1H), 8.10 (s, | |
| | 1H), 7.40 (d, J = 8.4 Hz, 1H), 6.96 (t, J = 8.8 Hz, 1H), | |
| | 6.71 (dd, J = 8.4, 4.0 Hz, 1H), 5.01 (d, J = 14.0 Hz, | |
| | 1H), 4.78 (d, J = 5.6 Hz, 2H), 4.57-4.53 (m, 3H), 3.33 | |
| | (t, J = 8.8 Hz, 2H), 2.55 (s, 3H). | |
| | LC-MS: [M + H]+ = 452.11 | |
| 96 | LC-MS: [M + H]+ = 469.10 | Example 6 |
| 97 | LC-MS: [M + H]+ = 466.12 | Example 6 |
| 98 | LC-MS: [M + H]+ = 597.21 | Example 4 |
| E 1 | ¹H NMR (400 MHz, DMSO-d⁶) δ 8.85 (s, 1H), 8.62 | |
| | (t, J = 4.9 Hz, 1H), 7.87 (d, J = 8.0 Hz, 1H), 7.44 (s, | |
| | 1H), 7.36 (d, J = 8.0 Hz, 1H), 6.96 (dd, J = 10.3, 8.6 | |
| | Hz, 1H), 6.71 (dd, J = 8.6, 3.9 Hz, 1H), 5.23 (d, J = | |
| | 14.9 Hz, 1H), 4.74 (d, J = 4.5 Hz, 2H), 4.55 (t, J = 8.8 | |
| | Hz, 2H), 4.46 (p, J = 6.9 Hz, 1H), 4.24 (d, J = 14.9 | |
| | Hz, 1H), 3.33 (t, J = 8.7 Hz, 2H), 2.53 (s, 3H), 1.27 | |
| | (d, J = 6.8 Hz, 3H), 1.13 (d, J = 6.7 Hz, 3H). | |
| | LC-MS: [M + H]+ = 473.20 | |
| E 2 | ¹H NMR (400 MHz, DMSO-d⁶) δ 8.79 (d, J = 14.9 | |
| | Hz, 3H), 7.80 (s, 1H), 7.52 (s, 1H), 6.95 (dd, J = 10.3, | |
| | 8.7 Hz, 1H), 6.70 (dd, J = 8.6, 3.9 Hz, 1H), 5.22 (d, J = | |
| | 14.9 Hz, 1H), 4.75 (d, J = 4.7 Hz, 2H), 4.55 (t, J = | |
| | 8.8 Hz, 2H), 4.27 (d, J = 14.9 Hz, 1H), 4.19 (p, J = 6.8 | |
| | Hz, 1H), 3.32 (t, J = 8.7 Hz, 2H), 2.64 (s, 3H), 1.24 | |
| | (dd, J = 35.6, 6.8 Hz, 6H). | |
| | LC-MS: [M + H]+ = 473.20 | |
| E 3 | ¹H NMR (400 MHz, DMSO-d⁶) δ 8.85 (s, 1H), 8.71 | |
| | (t, J = 5.1 Hz, 1H), 7.93 (d, J = 8.0 Hz, 1H), 7.54 (s, | |
| | 1H), 7.44 (d, J = 8.1 Hz, 1H), 6.96 (dd, J = 10.3, 8.6 | |
| | Hz, 1H), 6.71 (dd, J = 8.7, 3.9 Hz, 1H), 5.46 (d, J = | |
| | 15.0 Hz, 1H), 4.75 (d, J = 4.5 Hz, 2H), 4.71-4.61 | |
| | (m, 1H), 4.56 (t, J = 8.8 Hz, 2H), 4.19 (d, J = 15.0 Hz, | |
| | 1H), 4.08 (dq, J = 15.2, 9.0 Hz, 1H), 3.34 (t, J = 8.7 | |
| | Hz, 2H), 2.57 (s, 3H). | |
| | LC-MS: [M + H]+ = 513.15 | |
| E 4 | ¹H NMR (400 MHz, DMSO-d⁶) δ 8.79 (s, 1H), 8.71 | |
| | (t, J = 5.1 Hz, 1H), 8.04 (d, J = 1.8 Hz, 1H), 7.88 (dd, | |
| | J = 8.1, 1.8 Hz, 1H), 7.71 (d, J = 8.1 Hz, 1H), 7.46 (s, | |
| | 1H), 6.96 (dd, J = 10.3, 8.7 Hz, 1H), 6.71 (dd, J = 8.6, | |
| | 3.9 Hz, 1H), 5.19 (d, J = 15.1 Hz, 1H), 4.80-4.69 (m, | |
| | 2H), 4.55 (t, J = 8.8 Hz, 2H), 4.23 (d, J = 15.3 Hz, | |
| | 1H), 4.17 (q, J = 6.8 Hz, 1H), 3.33 (t, J = 8.7 Hz, 2H), | |
| | 1.29 (d, J = 6.7 Hz, 3H), 1.17 (d, J = 6.8 Hz, 3H). | |
| | LC-MS: [M + H]+ = 483.19 | |
| E 5 | ¹H NMR (400 MHz, DMSO-d⁶) δ 8.86 (s, 1H), 8.84- | |
| | 8.70 (m, 2H), 7.86 (s, 1H), 7.62 (s, 1H), 6.96 (dd, J = | |
| | 10.3, 8.6 Hz, 1H), 6.71 (dd, J = 8.6, 3.9 Hz, 1H), 5.22 | |
| | (d, J = 15.0 Hz, 1H), 4.77 (d, J = 4.9 Hz, 2H), 4.56 (t, | |
| | J = 8.8 Hz, 2H), 4.40 (d, J = 15.1 Hz, 1H), 4.35-4.17 | |
| | (m, 1H), 3.33 (t, J = 8.7 Hz, 2H), 2.62-2.54 (m, 1H), | |
| | 2.34-2.27 (m, 1H), 2.27-2.09 (m, 2H), 1.70 (ddt, J = | |
| | 25.8, 10.6, 7.5 Hz, 2H). | |
| | LC-MS: [M + H]+ = 539.17 | |
| E 6 | ¹H NMR (400 MHz, DMSO-d⁶) δ 8.86 (d, J = 4.0 Hz, | |
| | 1H), 8.74 (d, J = 6.5 Hz, 1H), 8.03 (d, J = 8.2 Hz, 1H), | |
| | 7.58-7.42 (m, 2H), 6.94 (dd, J = 10.2, 8.7 Hz, 1H), | |
| | 6.70 (dd, J = 8.6, 3.8 Hz, 1H), 6.47-6.09 (m, 1H), | |
| | 5.46 (d, J = 15.1 Hz, 1H), 4.74 (d, J = 3.5 Hz, 2H), | |
| | 4.55 (t, J = 8.7 Hz, 2H), 4.26 (dt, J = 15.3, 5.5 Hz, | |
| | 1H), 4.12 (dddd, J = 23.0, 13.9, 10.0, 3.2 Hz, 1H), | |
| | 3.80 (qd, J = 12.9, 4.7 Hz, 1H), 3.33 (t, J = 8.7 Hz, | |
| | 2H), 2.59 (t, J = 2.9 Hz, 3H). | |
| | LC-MS: [M + H]+ = 495.47 | |
| E 7 | ¹H NMR (400 MHz, DMSO-d⁶) δ 8.87 (d, J = 4.6 Hz, | |
| | 1H), 8.70 (d, J = 5.9 Hz, 1H), 7.99 (d, J = 7.9 Hz, | |
| | 1H), 7.63-7.43 (m, 2H), 6.96 (t, J = 9.4 Hz, 1H), | |
| | 6.71 (dd, J = 8.5, 3.9 Hz, 1H), 5.42 (d, J = 15.1 Hz, | |
| | 1H), 4.76 (d, J = 4.1 Hz, 2H), 4.56 (t, J = 8.7 Hz, 2H), | |
| | 4.36-4.17 (m, 2H), 3.80 (td, J = 14.1, 7.1 Hz, 1H), | |
| | 3.34 (t, J = 8.6 Hz, 2H), 2.59 (s, 3H), 1.66 (dd, J = | |
| | 21.1, 17.6 Hz, 3H). | |
| | LC-MS: [M + H]+ = 509.18 | |
| E 8 | ¹H NMR (400 MHz, DMSO-d⁶) δ 8.84 (d, J = 3.6 Hz, | |
| | 2H), 8.22 (d, J = 8.1 Hz, 1H), 7.98 (d, J = 8.1 Hz, 1H), | |
| | 7.62 (s, 1H), 6.95 (dd, J = 10.3, 8.7 Hz, 1H), 6.71 (dd, | |
| | J = 8.6, 3.8 Hz, 1H), 6.54 (brs, 1H), 5.56 (d, J = 14.9 | |
| | Hz, 1H), 4.76 (d, J = 5.0 Hz, 2H), 4.55 (t, J = 8.8 Hz, | |
| | 2H), 4.28 (d, J = 14.9 Hz, 1H), 4.10 (dt, J = 22.2, 11.0 | |
| | Hz, 1H), 3.65 (qd, J = 13.1, 5.2 Hz, 1H), 3.34 (t, J = | |
| | 8.6 Hz, 2H). | |
| | LC-MS: [M + H]+ = 549.13 | |
| E 9 | ¹H NMR (400 MHz, DMSO-d⁶) δ 8.83 (d, J = 10.8 | |
| | Hz, 2H), 8.73 (s, 1H), 7.86 (s, 1H), 7.57 (s, 1H), 6.95 | |
| | (dd, J = 10.3, 8.7 Hz, 1H), 6.70 (dd, J = 8.6, 3.9 Hz, | |
| | 1H), 6.47-6.09 (m, 1H), 5.36 (d, J = 14.9 Hz, 1H), | |
| | 4.75 (d, J = 4.2 Hz, 2H), 4.55 (t, J = 8.8 Hz, 2H), 4.25 | |
| | (d, J = 14.9 Hz, 1H), 4.10 (dddd, J = 22.8, 13.5, 9.2, | |
| | 3.3 Hz, 1H), 3.68-3.45 (m, 1H), 3.33 (t, J = 8.7 Hz, | |
| | 2H), 2.63 (s, 3H). | |
| | LC-MS: [M + H]+ = 495.16 | |
| E 10 | ¹H NMR (400 MHz, DMSO-d⁶) δ 9.08 (s, 1H), 9.01 | |
| | (s, 1H), 8.85 (s, 1H), 8.06 (s, 1H), 7.80 (s, 1H), 7.47 | |
| | (d, J = 1.3 Hz, 1H), 6.98 (dd, J = 10.3, 8.7 Hz, 1H), | |
| | 6.74 (dd, J = 8.6, 3.9 Hz, 1H), 5.79 (d, J = 14.7 Hz, | |
| | 1H), 5.57 (d, J = 14.8 Hz, 1H), 4.88-4.72 (m, 2H), | |
| | 4.59 (t, J = 8.9 Hz, 3H), 3.34 (t, J = 8.6 Hz, 2H), 2.56- | |
| | 2.53 (m, 3H). | |
| | LC-MS: [M + H]+ = 522.16 | |
| E 11 | ¹H NMR (400 MHz, DMSO-d⁶) δ 8.79 (d, J = 8.1 Hz, | |
| | 2H), 8.70 (t, J = 5.1 Hz, 1H), 7.86 (s, 1H), 7.56 (s, | |
| | 1H), 7.08 (d, J = 55.0 Hz, 1H), 6.99-6.85 (m, 1H), | |
| | 6.70 (dd, J = 8.7, 3.9 Hz, 1H), 5.20 (d, J = 14.9 Hz, | |
| | 1H), 4.75 (d, J = 4.7 Hz, 2H), 4.55 (t, J = 8.8 Hz, 2H), | |
| | 4.36 (d, J = 15.0 Hz, 1H), 4.25 (q, J = 6.8 Hz, 1H), | |
| | 3.33 (t, J = 8.7 Hz, 2H), 1.27 (d, J = 6.7 Hz, 3H), 1.17 | |
| | (d, J = 6.8 Hz, 3H). | |
| | LC-MS: [M + H]+ = 509.18 | |
| E 12 | ¹H NMR (400 MHz, DMSO-d⁶) δ 8.83 (s, 1H), 8.66 | |
| | (brs, 1H), 7.88 (d, J = 8.0 Hz, 1H), 7.54 (s, 1H), 7.40 | |
| | (d, J = 8.1 Hz, 1H), 6.96 (dd, J = 10.3, 8.7 Hz, 1H), | |
| | 6.71 (dd, J = 8.6, 3.8 Hz, 1H), 5.46 (d, J = 14.9 Hz, | |
| | 1H), 4.75 (d, J = 4.9 Hz, 2H), 4.67 (dd, J = 15.2, 9.5 | |
| | Hz, 1H), 4.56 (t, J = 8.8 Hz, 2H), 4.17 (d, J = 14.9 Hz, | |
| | 1H), 4.14-3.99 (m, 1H), 3.34 (t, J = 8.6 Hz, 2H), | |
| | 2.82 (q, J = 7.6 Hz, 2H), 1.28 (td, J = 7.6, 1.3 Hz, | |
| | 3H). | |
| | LC-MS: [M + H]+ = 527.49 | |
| E 13 | ¹H NMR (400 MHz, DMSO-d⁶) δ 8.82 (s, 1H), 8.64 | |
| | (t, J = 5.2 Hz, 1H), 7.87 (d, J = 8.1 Hz, 1H), 7.51 (s, | |
| | 1H), 7.38 (d, J = 8.1 Hz, 1H), 6.96 (dd, J = 10.3, 8.7 | |
| | Hz, 1H), 6.71 (dd, J = 8.6, 3.9 Hz, 1H), 5.41 (d, J = | |
| | 14.7 Hz, 2H), 4.75 (d, J = 4.9 Hz, 2H), 4.56 (t, J = 8.8 | |
| | Hz, 2H), 4.20-4.14 (m, 2H), 3.80-3.76 (m, 1H), 3.34 | |
| | (t, J = 8.6 Hz, 2H), 2.82 (q, J = 7.6 Hz, 2H), 1.28 (t, J = | |
| | 7.6 Hz, 3H). | |
| | LC-MS: [M + H]+ = 509.50 | |
| E 14 | ¹H NMR (400 MHz, DMSO-d⁶) δ 8.83 (s, 1H), 8.65 | |
| | (t, J = 5.1 Hz, 1H), 7.80 (d, J = 8.1 Hz, 1H), 7.51 (s, | |
| | 1H), 7.41 (d, J = 8.1 Hz, 1H), 6.95 (dd, J = 10.3, 8.7 | |
| | Hz, 1H), 6.71 (dd, J = 8.7, 3.9 Hz, 1H), 5.41 (d, J = | |
| | 14.8 Hz, 1H), 4.75 (d, J = 4.8 Hz, 2H), 4.68 (dd, J = | |
| | 15.2, 9.6 Hz, 1H), 4.56 (t, J = 8.8 Hz, 2H), 4.08 (d, J = | |
| | 14.8 Hz, 1H), 3.96 (dt, J = 15.3, 9.2 Hz, 1H), 3.34 | |
| | (t, J = 8.7 Hz, 2H), 2.28-2.21 (m, 1H), 1.32-1.15 (m, | |
| | 1H), 1.16-0.99 (m, 3H). | |
| | LC-MS: [M + H]+ = 539.17 | |
| E 15 | ¹H NMR (400 MHz, DMSO-d⁶) δ 8.83 (s, 1H), 8.66 | |
| | (t, J = 5.1 Hz, 1H), 7.88 (d, J = 8.1 Hz, 1H), 7.54 (s, | |
| | 1H), 7.41 (d, J = 8.1 Hz, 1H), 6.96 (dd, J = 10.3, 8.7 | |
| | Hz, 1H), 6.71 (dd, J = 8.7, 3.9 Hz, 1H), 5.47 (d, J = | |
| | 14.9 Hz, 1H), 4.75 (d, J = 4.9 Hz, 2H), 4.67 (dd, J = | |
| | 15.0, 9.5 Hz, 1H), 4.56 (t, J = 8.8 Hz, 2H), 4.17 (d, J = | |
| | 14.8 Hz, 1H), 4.13-3.98 (m, 1H), 3.34 (t, J = 8.7 | |
| | Hz, 2H), 3.09 (p, J = 6.9 Hz, 1H), 1.29 (dd, J = 6.9, | |
| | 5.4 Hz, 6H). | |
| | LC-MS: [M + H]+ = 541.18 | |
| E 16 | ¹H NMR (400 MHz, DMSO-d⁶) δ 8.85 (s, 1H), 8.65 | |
| | (d, J = 5.4 Hz, 1H), 7.91 (d, J = 8.0 Hz, 1H), 7.52 (s, | |
| | 1H), 7.43 (d, J = 8.1 Hz, 1H), 6.96 (dd, J = 10.3, 8.7 | |
| | Hz, 1H), 6.71 (dd, J = 8.6, 3.9 Hz, 1H), 5.40 (d, J = | |
| | 14.8 Hz, 1H), 4.75 (d, J = 4.4 Hz, 2H), 4.56 (t, J = 8.8 | |
| | Hz, 2H), 4.27-4.12 (m, 3H), 3.34 (t, J = 8.7 Hz, 2H), | |
| | 2.84 (q, J = 7.6 Hz, 2H), 1.67 (t, J = 19.3 Hz, 3H), | |
| | 1.29 (t, J =7.6 Hz, 3H). | |
| | LC-MS: [M + H]+ = 523.19 | |
| E 17 | ¹H NMR (400 MHz, DMSO-d⁶) δ 9.06 (s, 1H), 8.88 | |
| | (t, J = 5.1 Hz, 1H), 8.60 (dd, J = 4.8, 1.6 Hz, 1H), | |
| | 7.92 (dd, J = 8.0, 1.6 Hz, 1H), 7.67 (s, 1H), 7.57 (dd, | |
| | J = 7.9, 4.8 Hz, 1H), 7.45 (d, J = 1.3 Hz, 1H), 6.98 (t, | |
| | J = 9.5 Hz, 1H), 6.73 (dd, J = 8.6, 3.9 Hz, 1H), 5.82 | |
| | (d, J = 14.4 Hz, 1H), 5.34 (d, J = 14.3 Hz, 1H), 4.78 | |
| | (t, J = 4.6 Hz, 2H), 4.59 (t, J = 8.8 Hz, 2H), 3.34 (t, J = | |
| | 8.7 Hz, 2H), 2.60 (s, 3H). | |
| | LC-MS: [M + H]+ = 454.17 | |
| E 18 | ¹H NMR (400 MHz, DMSO-d⁶) δ 9.04 (s, 1H), 8.85 | |
| | (s, 1H), 7.78 (d, J = 8.0 Hz, 1H), 7.62 (s, 1H), 7.42 (d, | |
| | J = 7.8 Hz, 2H), 7.03-6.91 (m, 1H), 6.72 (dd, J = | |
| | 8.7, 3.9 Hz, 1H), 5.76 (d, J = 14.3 Hz, 1H), 5.27 (d, J = | |
| | 14.2 Hz, 1H), 4.76 (d, J = 3.9 Hz, 2H), 4.58 (t, J = | |
| | 8.8 Hz, 2H), 3.34 (t, J = 8.7 Hz, 2H), 2.61 (s, 3H), | |
| | 2.53 (s, 3H). | |
| | LC-MS: [M + H]+ = 468.18 | |
| E 19 | ¹H NMR (400 MHz, DMSO-d⁶) δ 8.85 (s, 1H), 8.71 | |
| | (t, J = 5.1 Hz, 1H), 7.93 (d, J = 8.0 Hz, 1H), 7.54 (s, | |
| | 1H), 7.44 (d, J = 8.1 Hz, 1H), 6.96 (dd, J = 10.3, 8.6 | |
| | Hz, 1H), 6.71 (dd, J = 8.7, 3.9 Hz, 1H), 5.46 (d, J = | |
| | 15.0 Hz, 1H), 4.75 (d, J = 4.5 Hz, 2H), 4.71-4.61 | |
| | (m, 1H), 4.56 (t, J = 8.8 Hz, 2H), 4.19 (d, J = 15.0 Hz, | |
| | 1H), 4.08 (dq, J = 15.2, 9.0 Hz, 1H), 3.34 (t, J = 8.7 | |
| | Hz, 2H), 2.57 (s, 3H). | |
| | LC-MS: [M + H]+ = 516.15 | |
| E 20 | LC-MS: [M + H]+ = 515.18 | |
| E 21 | LC-MS: [M + H]+ = 518.18 | |
| E 22 | LC-MS: [M + H]+ = 517.18 | |
| E 23 | LC-MS: [M + H]+ = 520.20 | |
| E 24 | LC-MS: [M + H]+ = 571.19 | |
| E 25 | LC-MS: [M + H]+ = 492.14 | |
| E 26 | LC-MS: [M + H]+ = 504.14 | |
| E 27 | LC-MS: [M + H]+ = 478.12 | |
| E 28 | ¹H NMR (400 MHz, DMSO-d⁶) δ 8.74 (s, 1H), 8.44 | |
| | (t, J = 5.1 Hz, 1H), 7.48 (t, J = 6.7 Hz, 1H), 7.38 (td, J = | |
| | 7.4, 1.6 Hz, 1H), 7.29 (td, J = 7.4, 1.4 Hz, 1H), 7.22 | |
| | (td, J = 7.5, 1.4 Hz, 2H), 7.10 (s, 1H), 6.96 (dd, J = | |
| | 10.3, 8.6 Hz, 1H), 6.72 (dd, J = 8.6, 3.9 Hz, 1H), 4.85- | |
| | 4.62 (m, 2H), 4.56 (t, J = 8.8 Hz, 2H), 3.38-3.20 | |
| | (m, 4H), 2.82 (dd, J = 8.9, 6.2 Hz, 2H). | |
| | LC-MS: [M + H]+ = 430.16 | |
| E 29 | LC-MS: [M + H]+ = 445.17 | |
| E 30 | LC-MS: [M + H]+ = 527.17 | |
| E 31 | LC-MS: [M + H]+ = 541.18 | |
| E 32 | LC-MS: [M + H]+ = 508.14 | |
| E 33 | LC-MS: [M + H]+ = 508.14 | |
| E 34 | LC-MS: [M + H]+ = 454.17 | |
| E 35 | LC-MS: [M + H]+ = 523.14 | |
| E 36 | LC-MS: [M + H]+ = 455.15 | |
| E 37 | LC-MS: [M + H]+ = 536.17 | |
| E 38 | LC-MS: [M + H]+ = 482.20 | |
| E 39 | LC-MS: [M + H]+ = 527.17 | |
| E 40 | ¹H NMR (400 MHz, DMSO-d⁶) δ 9.02-8.67 (m, | |
| | 2H), 8.11 (d, J = 7.3 Hz, 1H), 7.62 (d, J = 7.4 Hz, 1H), | |
| | 6.96 (q, J = 9.1 Hz, 1H), 6.72 (dq, J = 8.4, 3.7 Hz, | |
| | 1H), 5.26 (dd, J = 15.2, 7.2 Hz, 2H), 4.78 (d, J = 6.4 | |
| | Hz, 2H), 4.56 (q, J = 8.5 Hz, 2H), 4.39 (dd, J = 15.4, | |
| | 7.0 Hz, 1H), 4.27 (q, J = 7.3 Hz, 1H), 3.35 (q, J = 8.5 | |
| | Hz, 2H), 1.41-1.23 (m, 3H), 1.23-1.06 (m, 3H). | |
| | LC-MS: [M + H]+ = 527.17 | |
| E 41 | ¹H NMR (400 MHz, DMSO-d⁶) δ 8.86 (s, 1H), 8.68 | |
| | (t, J = 5.1 Hz, 1H), 7.95 (d, J = 8.0 Hz, 1H), 7.52 (s, | |
| | 1H), 7.46 (d, J = 8.1 Hz, 1H), 6.95 (dd, J = 10.3, 8.7 | |
| | Hz, 1H), 6.70 (dd, J = 8.6, 3.9 Hz, 1H), 5.40 (d, J = | |
| | 14.9 Hz, 1H), 4.75 (d, J = 4.2 Hz, 2H), 4.55 (t, J = 8.7 | |
| | Hz, 2H), 4.27 (dd, J = 17.6, 13.4 Hz, 2H), 3.78 (td, J = | |
| | 14.0, 6.8 Hz, 1H), 3.33 (t, J = 8.7 Hz, 2H), 2.57 (s, | |
| | 3H), 1.65 (t, J = 19.3 Hz, 3H). | |
| | LC-MS: [M + H]+ = 509.18 | |
| E 42 | ¹H NMR (400 MHz, DMSO-d⁶) δ 8.85 (s, 1H), 8.78 | |
| | (d, J = 8.3 Hz, 2H), 8.10 (s, 1H), 7.64 (s, 1H), 7.16- | |
| | 6.83 (m, 2H), 6.71 (dd, J = 8.6, 3.9 Hz, 1H), 5.39 (d, J = | |
| | 15.2 Hz, 1H), 4.76 (d, J = 4.8 Hz, 2H), 4.69-4.48 | |
| | (m, 3H), 4.35 (d, J = 15.1 Hz, 1H), 4.06 (dt, J = 15.1, | |
| | 9.1 Hz, 1H), 3.34 (t, J = 8.7 Hz, 2H). | |
| | LC-MS: [M + H]+ = 549.13 | |
| E 43 | LC-MS: [M + H]+ = 545.16 | |
| E 44 | ¹H NMR (400 MHz, DMSO-d⁶) δ 8.81 (d, J = 25.3 | |
| | Hz, 3H), 8.02 (s, 1H), 7.63 (s, 1H), 7.19-6.81 (m, | |
| | 2H), 6.71 (dd, J = 8.6, 3.8 Hz, 1H), 6.44-6.03 (m, | |
| | 1H), 5.36 (d, J = 15.0 Hz, 1H), 4.76 (d, J = 4.8 Hz, | |
| | 2H), 4.55 (t, J = 8.8 Hz, 2H), 4.35 (d, J = 14.9 Hz, | |
| | 1H), 4.06 (dt, J = 15.1, 9.1 Hz, 1H), 3.66 (td, J = 13.0, | |
| | 10.9, 4.6 Hz, 1H), 3.34 (t, J = 8.8 Hz, 2H). | |
| | LC-MS: [M + H]+ = 531.14 | |
| E 48 | LC-MS: [M + H]+ = 585.18 | Example 10 |
| E 49 | LC-MS: [M + H]+ = 599.20 | Example 10 |
| E 50 | LC-MS: [M + H]+ = 555.21 | Example 10 |
| E 51 | LC-MS: [M + H]+ = 555.21 | Example 10 |
| E 52 | LC-MS: [M + H]+ = 553.19 | Example 10 |

### Biological Assays

### Example 14: EED Alpha Screen Binding Assay and Anti-Proliferative Activities (IC₅₀ Values) Assay

### Analysis of Cell Proliferation:

The human B cell lymphoma cell KARPAS422 was purchased from the American Type Culture Collection (ATCC), and was cultured using standard cell culture conditions in RPMI-1640 (Invitrogen, cat #11875) supplemented with 10% FBS (Invitrogen, cat #10099-141) in humidified incubator at 37° C., 5% CO₂. To assess the effect of PRC2 inhibition on cell growth, cells were seeded in 96-well cell culture plates at a density of 2 000-3 000 cells/well in 200 μL of culture medium, and treated with serially diluted compounds for 7 days at 37° C. in an atmosphere of 5% CO₂. Cell growth was evaluated by a lactate dehydrogenase-based WST-8 assay (Dojindo Molecular Technologies) using a Tecan Infinite M1000 multimode microplate reader (Tecan, Morrisville, NC). The WST-8 reagent was added to the plate, incubated for 1-4 h, and read at 450 nm. The readings were normalized to the DMSO-treated cells, and the IC₅₀ was calculated by nonlinear regression analysis using GraphPad Prism 6 software

### EED-H3K27Me3 Peptide Competition Binding Assay by AlphaScreen (α-Screen):

To assess the potency in the EED-H3K27Me3 competition binding assay, representative Compounds of the Disclosure were serially diluted 3-fold in DMSO to obtain a total of twelve concentrations. The compounds at each concentration (2.5 μl of each) were transferred into a 384-well Perkin Elmer OptiPlate-384 white plates. 5 μl of solutions containing 20 nM EED (1-441)-His protein in the buffer (25 mM HEPES, pH 8, 0.02% Tween-20, 0.5% BSA) were added to the wells and then incubated with compound for 15 min. 2.5 μl of solutions containing 20 nM biotin-H3K27Me3 (19-33) peptide in the buffer (25 mM HEPES, pH 8, 0.02% Tween-20, 0.5% BSA) were added to the wells and then incubated with compound for 30 min. AlphaScreen detection beads mix was prepared immediately before use by mixing nickel chelate acceptor beads and streptavidin donor beads in a 1:1 ratio (Perkin Elmer, Product No. 6760619C/M/R) into the buffer described above. Then 10 μl of the detection beads mix was added to the plate and incubated in the dark at RT for 1 h. The final concentration of donor and acceptor beads was 10 μg/ml for each. Plates were read on CLARIOStar plate reader (BMG Labtech) using the AlphaScreen setting adapted for optimal signal detection with a 615 nm filter, after sample excitation at 680 nm. The emission signal at 615 nm was used to quantify compound inhibition. AlphaScreen signals were normalized based on the reading coming from the positive (maximum signal control) and negative controls (minimum signal control) to give percentage of activities left. The data were then fit to a dose response equation to get the IC₅₀ values.

The results are presented in Tables 4A and 4B.

TABLE 4A

| Cpd. | Pfeiffer Cell | Karpas Cell | Alpha |
|---|---|---|---|
| No. | line (nM) | line (nM) | screen (nM) |
| 1 | >100 | >100 | >10 |
| 2 | >100 | >100 | >10 |
| 3 | 83.95 | N/A | 6.88 |
| 4 | >100 | >100 | >10 |
| 5 | 18.05 | 32.23 | 0.26 |
| 6 | 0.22 | 1.98 | 0.19 |
| 7 | 0.68 | 3.35 | 0.61 |
| 8 | 0.57 | 2.91 | 1.97 |
| 9 | 1.81 | 5.60 | 0.84 |
| 10 | 2.93 | 15.82 | 1.11 |
| 11 | 1.22 | 4.61 | 0.75 |
| 12 | 0.65 | 3.84 | 0.34 |
| 13 | 1.99 | 9.03 | 1.09 |
| 14 | 0.36 | 3.49 | 3.35 |
| 15 | 1.25 | 4.36 | 0.52 |
| 16 | 3.39 | 14.67 | 0.70 |
| 17 | 4.17 | 20.51 | 0.66 |
| 18 | 0.73 | 15.28 | 0.70 |
| 19 | 0.79 | 9.60 | 1.13 |
| 20 | 0.90 | 13.88 | 0.61 |
| 21 | 0.61 | 14.95 | 0.55 |
| 22 | 4.88 | 45.57 | 0.67 |
| 23 | 9.95 | 89.21 | 1.18 |
| 24 | 38.38 | >100 | 4.75 |
| 25 | 0.29 | 16.46 | 3.31 |
| 26 | 32.91 | 41.4 | 9.84 |
| 27 | >100 | >100 | 5.87 |
| 28 | >100 | >100 | 2.58 |
| 29 | N/A | N/A | 20.73 |
| 30 | N/A | N/A | 2.03 |
| 31 | N/A | 3.68 | 0.6041 |
| 32 | 11.6 | 84.41 | 1.84 |
| 33 | 0.22 | 1.11 | 1.06 |
| 34 | 0.35 | 2.64 | 0.67 |
| 35 | 0.12 | 1.23 | 1.16 |
| 36 | 1.04 | 5.66 | 1.44 |
| 37 | 0.25 | 1.79 | 0.94 |
| 38 | 3.22 | 15.71 | 1.25 |
| 39 | 22.67 | >100 | 26.14 |
| 40 | 38.78 | >100 | 10.48 |
| 41 | 1.03 | 1.69 | 0.60 |
| 42 | 4.19 | 4.01 | 0.84 |
| 43 | 1.05 | 5.46 | 1.05 |
| 44 | 1.03 | 2.44 | 1.34 |
| 45 | 0.61 | 6.15 | 3.39 |
| 46 | N/A | 0.57 | 1.07 |
| 47 | N/A | 0.65 | 0.91 |
| 48 | N/A | 0.74 | 1.65 |
| 49 | N/A | 2.78 | 1.81 |
| 50 | N/A | 0.71 | 1.16 |
| 51 | N/A | 2.08 | 0.70 |
| 52 | N/A | 0.54 | 0.70 |
| 53 | N/A | >100 | 3.96 |
| 54 | N/A | >100 | 0.95 |
| 55 | N/A | 8.08 | 2.14 |
| 56 | N/A | 21.01 | 4.49 |
| 57 | N/A | 2.63 | 4.20 |
| 58 | N/A | N/A | 2.74 |
| 59 | N/A | 39.86 | 2.14 |
| 60 | N/A | 1.18 | N/A |
| 61 | N/A | 1.63 | 1.19 |
| 62 | N/A | 4.86 | 1.44 |
| 63 | N/A | 3.69 | 1.89 |
| 64 | N/A | 2.44 | N/A |
| 65 | N/A | 0.69 | N/A |
| 66 | N/A | 0.74 | 3.61 |
| 67 | N/A | 7.78 | 2.87 |
| 68 | N/A | 1.35 | 3.86 |
| 69 | N/A | >100 | 19.83 |
| 70 | N/A | >100 | >100 |
| 71 | N/A | >100 | 8.94 |
| 72 | N/A | 3.86 | 2.69 |
| 73 | N/A | 11.33 | 0.50 |
| 74 | N/A | 46.98 | 1.67 |
| 75 | N/A | 11.06 | 1.72 |
| 76 | N/A | 14.53 | 0.97 |
| 77 | N/A | 22.46 | 1.90 |
| 78 | N/A | 34.46 | 3.23 |
| 79 | N/A | 15.28 | 1.61 |
| 80 | N/A | 2.42 | 1.61 |
| 81 | N/A | 1.43 | 1.70 |
| 82 | 0.074 | 3.51 | 10.49 |
| 83 | 0.304 | 6.32 | 9.95 |
| 84 | 0.048 | 1.48 | 3.07 |
| 85 | 0.13 | 2.71 | 9.82 |
| 86 | 0.021 | 2.29 | 2.56 |
| 87 | 0.018 | 8.67 | 11.15 |
| 88 | 0.035 | 25.68 | 12.32 |
| 89 | N/A | N/A | N/A |
| 90 | 0.044 | 0.078 | N/A |
| 91 | 0.043 | 5.23 | N/A |
| 92 | N/A | N/A | N/A |
| 93 | 0.55 | 3.71 | N/A |
| 94 | 0.20 | 13.9 | 14.00 |
| 95 | 0.023 | 0.89 | 13.12 |
| 96 | 0.056 | 21.19 | 21.88 |
| 97 | 0.058 | 1.847 | N/A |
| 98 | N/A | N/A | N/A |

TABLE 4B

| Cpd. | Pfeiffer Cell | Karpas Cell | Alpha |
|---|---|---|---|
| No. | line (nM) | line (nM) | screen (nM) |
| E 1 | A | A | A |
| E 2 | A | A | A |
| E 3 | A | A | A |
| E 4 | A | A | A |
| E 5 | A | A | A |
| E 6 | A | A | A |
| E 7 | A | A | A |
| E 8 | A | A | A |
| E 9 | A | A | A |
| E 10 | B | B | A |
| E 11 | A | A | A |
| E 12 | A | A | A |
| E 13 | A | A | A |
| E 14 | A | A | A |
| E 15 | A | A | A |
| E 16 | A | A | A |
| E 17 | B | B | A |
| E 18 | A | B | A |
| E 19 | A | A | A |
| E 20 | A | A | A |
| E 21 | A | A | A |
| E 22 | A | A | A |
| E 23 | A | A | A |
| E 24 | B | B | B |
| E 25 | B | B | A |
| E 26 | A | B | B |
| E 27 | A | B | B |
| E 28 | B | C | B |
| E 29 | B | B | B |
| E 30 | B | B | B |
| E 31 | B | B | B |
| E 32 | A | B | B |
| E 33 | B | B | B |
| E 34 | A | B | B |
| E 35 | B | B | B |
| E 36 | B | B | B |
| E 37 | B | B | B |
| E 38 | B | B | B |
| E 39 | A | B | B |
| E 40 | A | A | A |
| E 45 | A | A | A |
| E 46 | A | A | A |
| E 47 | A | A | A |
| E 48 | A | A | A |
| E 49 | A | A | A |
| E 50 | A | A | A |
| E 51 | A | A | A |
| E 52 | A | A | A |

IC₅₀ definition:
A = <10 nM;
B = >10 nM-<100 nM; and
C = >100 nM Cell

### Examples 15-27

### Evaluation Method for In Vivo Pharmacological Experiment

A subcutaneous xenograft tumor model of human tumor immunodeficient mice was established by cell inoculation: tumor cells in logarithmic growth phase were collected, counted, resuspended in 1×PBS, and the cell suspension concentration was adjusted to 2.5-5×10⁷/mL. The tumor cells were inoculated subcutaneously in the right side of immunodeficient mice with a 1 ml syringe (4 gauge needle), 5-10×10⁶/0.2 mL/mouse. All animal experiments were strictly in accordance with the specifications for the use and management of experimental animals in GenePharma Co., Ltd. and Suzhou Ascentage Pharma Co., Ltd. The calculation of relevant parameters refers to the Chinese NMPA “Guidelines for Non-Clinical Research Techniques of Cytotoxic Anti-tumor Drugs”.

Animal body weight and tumor size were measured twice weekly during the experiment. The state of the animal and the presence or absence of death were observed every day. The growth of tumor and the effects of treatment on normal behavior of animals were monitored routinely, specifically involving experimental animal activity, feeding and drinking, weight gain or loss, eyes, clothing hair and other abnormalities. The deaths and clinical symptoms observed during the experiment were recorded in the raw data. All operations for administration and measurement of mouse body weight and tumor volume were performed in a clean bench. According to the requirements of the experimental protocol, after the end of the last administration, plasma and tumor tissues were collected, weighed and photographed. The plasma and tumor samples were frozen at −80° C. for ready-to-use.

Tumor volume (TV) is calculated as: TV=a×b²/2, wherein a and b represent the length and width of the tumor to be measured, respectively.

The relative tumor volume (RTV) is calculated as: RTV=Vt/V1, wherein V1 is the tumor volume at the start of grouping and administration, and Vt is the tumor volume measured on the t day after administration.

The evaluation index of anti-tumor activity is the relative tumor proliferation rate T/C (%), and the calculation formula thereof is: relative tumor proliferation rate T/C (%)=(T_{RTV}/C_{RTV})×100%, T_{RTV} is the RTV of treatment group, C_{RTV} is the RTV of solvent control group.

Tumor regression rate (%) is calculated as: the number of tumor-bearing mice which exhibit SD (stable disease), PR (partial regression) and CR (complete regression) after treatment/the total number of the mice in this group×100%.

Change of body weight (%)=(measured body weight-body weight at the start of grouping)/body weight at the start of grouping×100%.

Evaluation criteria for therapeutic efficiency: According to the Chinese NMPA “Guidelines for Non-Clinical Research Techniques of Cytotoxic Anti-tumor Drugs” (November 2006), when T/C (%) value is ≤40% and statistical analysis shows p<0.05, efficiency is confirmed. A dose of drug is considered to be severely toxic if the body weight of mouse is reduced by more than 20% or the number of drug-related deaths exceeds 20%.

According to the description by Clarke R., Issues in experimental design and endpoint analysis in the study of experimental cytotoxic agents in vivo in breast cancer and other models [J]. Breast Cancer Research & Treatment, 1997, 46(2-3): 255-278, synergy analysis was evaluated using the following formula: synergy factor=((A/C)×(B/C))/(AB/C); A=RTV value of drug A alone group; B=RTV value of drug B alone group; C=RTV value of the solvent control group, and AB=RTV value of the A and B combination group. Synergistic factor >1 indicates that synergy is achieved; synergy factor=1 indicates that additive effect is achieved; and synergy factor <1 indicates that antagonistic effect is achieved.

Use of mRECIST (Gao et al., 2015) measured tumor responses included stable disease (SD), partial tumor regression (PR), and complete regression (CR), determined by comparing tumor volume change at day t to its baseline: tumor volume change (%)=(Vt−V1)/V1. The BestResponse was the minimum value of tumor volume change (%) for t≥10. For each time t, the average of tumor volume changes from t=1 to t was also calculated. BestAvgResponse was defined as the minimum value of this average for t≥10. The criteria for response (mRECIST) were adapted from RECIST criteria (Gao et al., 2015; Therasse et al., 2000) and defined as follows: mCR, BestResponse<−95% and BestAvg Response<−40%; mPR, BestResponse<−50% and BestAvgResponse<−20%; mSD, BestResponse<35% and BestAvgResponse<30%; mPD, not otherwise categorized. SD, PR, and CR were considered responders and used to calculate response rate (%). Disease control rate (DCR) is calculated with the proportion of animals demonstrating CR, PR, or SD based on mRECIST; Overall response rate (ORR) is calculated with the proportion of animals demonstrating CR or PR based on mRECIST. Body weight of animals were monitored simultaneously. The change in body weight was calculated based on the animal weight of the first day of dosing (day 1). Tumor volume and changes in body weight (%) were represented as the mean±standard error of the mean (SEM).

### Cell Viability Assays (CTG)

Cell viability was determined using CellTiter-Glo® luminescent cell viability assay (Promega) or WST assay (Cell counting Kit-8, Shanghai life iLab, China) by following manufacturer's instruction. Cell viability was calculated as cell viability=(mean RLU sample−mean RLU blank)/(RLU cell control−RLU blank)×100. IC₅₀ value was calculated using GraphPad Prism. Combination index (CI) value was calculated by CalcuSyn software (BIOSOFT, UK). CI<0.9 indicate a synergistic combination effect. CI<0.1 scored as 5+ indicates very strong synergistic combination effect, CI between 0.1 and 0.3 scored as 4+ indicates strong synergistic combination effect, CI between 0.3 and 0.7 scored as 3+ indicates medium synergistic combination effect.

The evaluation method as described is used in following Examples 15-27.

### Example 15. Treatment with Compound 73 in BAP1 Mut Mesothelioma PDX Model

In this experiment, a BAP1 mut mesothelioma PDX model (Lide Biotech) was established to evaluate the anti-tumor effect of Compound 73. The dosing regimen was as follows:
Compound 73: 50 mg/kg, orally, once per day, for a total of 28 days;
Compound 73: 100 mg/kg, orally, once per day, for a total of 28 days;

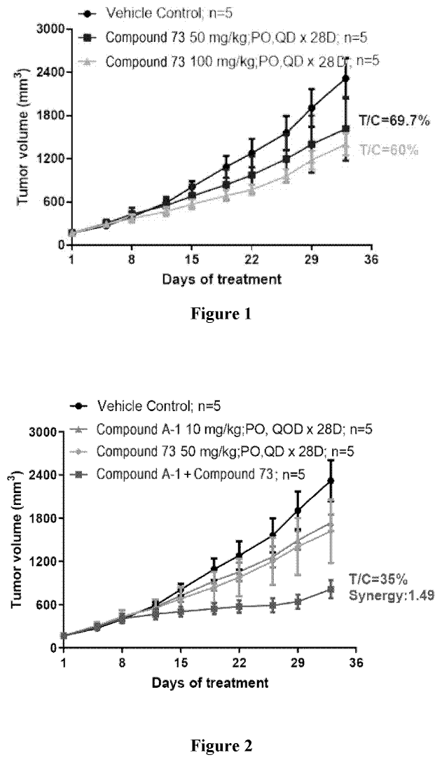

As shown in FIG. 1, after 28 days of administration, the T/C (%) values of Compound 73 (50 mg/kg) and Compound 73 (100 mg/kg) were 69.7% and 60% respectively.

In conclusion, Compound 73 single agent showed dose-dependent antitumor activity. The T/C values were 69.7% and 60.0% when Compound 73 were dosed at 50 mg/kg and 100 mg/kg respectively.

TABLE 5
Genetic information of the BAP1 mutant PDX model

| PDX | Genetic Information | | | | |
|---|---|---|---|---|---|
| Model | BAP1 | CDKN2A | TP53 | NF2 | others |
| LD1- | Mut | WT | p.R72R | — | FGFR1(TPM) = |
| 0045- | (stopgain) | | | | 33.17, |
| 361820 | | | | | FGFR1CNV = 1 |
| | | | | | FGFR2(TPM) = 8.69, |
| | | | | | FGFR1CNV = 1 |
| | | | | | FGFR3(TPM) = |
| | | | | | 11.08, |
| | | | | | FGFR1CNV = 2 |

### Example 16. Combination Treatment with Compound 73 and Compound A-1 in BAP1 Mut Mesothelioma PDX Model

In this experiment, a BAP1 mut mesothelioma PDX model (Lide Biotech) was established to evaluate the anti-tumor effect of Compound 73 in combination with Compound A-1. The dosing regimen was as follows:
Compound A-1: 10 mg/kg, orally, every other day, for a total of 28 days;
Compound 73: 50 mg/kg, orally, once per day, for a total of 28 days;

The dosing regimen of each drug in the dosing regimen for the combination is the same as the dosing regimen for the single drug.

As shown in FIG. 2, the single agent of Compound 73 and Compound A-1 showed moderate antitumor activity, with T/C values of 69.7% and 74.8% respectively at the end of the study. The combination treatment further reduced the T/C value to 35%, with a synergistic factor of 1.49, indicating synergistic effects.

### Example 17. Combination Treatment with Compound 73 and Compound 2-5 in BAP1 Mut Mesothelioma PDX Model

In this experiment, a BAP1 mut mesothelioma PDX model (Lide Biotech) was established to evaluate the anti-tumor effect of Compound 73 in combination with Compound 2-5. The dosing regimen was as follows:
Compound 2-5: 50 mg/kg, orally, once per day, for a total of 28 days;
Compound 73: 50 mg/kg, orally, once per day, for a total of 28 days;

The dosing regimen of each drug in the dosing regimen for the combination is the same as the dosing regimen for the single drug.

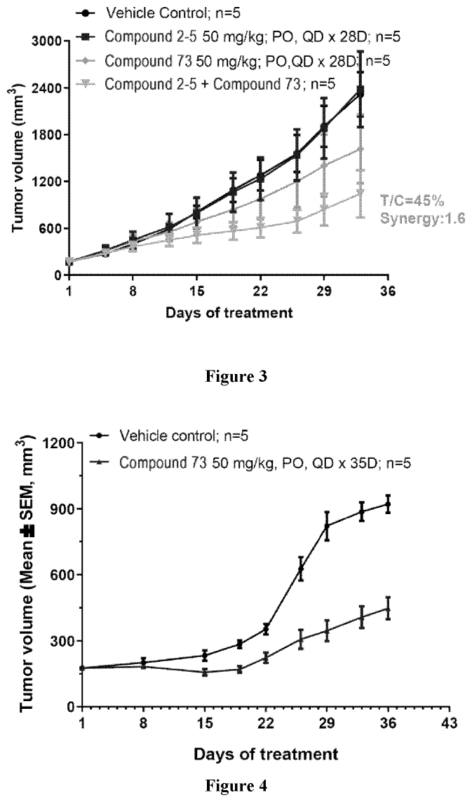

As shown in FIG. 3, Compound 2-5 was not effective in inhibiting tumor growth, and Compound 73 showed moderate antitumor activity, with T/C value of 69.7% at the end of the study. The combination treatment further reduced the T/C value to 45%, with a synergistic factor of 1.6, indicating synergistic effects.

### Example 18. Treatment with Compound 73 in NCI-H226 (BAP1mut, CDKN2Adel, FGFR High) Xenograft Model

In this experiment, NCI-H226 (BAP1^{mut}, CDKN2^{Adel}, FGFR^{high}) xenograft model was established to evaluate the anti-tumor effect of Compound 73. The dosing regimen was as follows:
Compound 73: 50 mg/kg, orally, once per day, for a total of 35 days.

As shown in FIG. 4, after 35 days of administration, the T/C (%) values of Compound 73 was 48.6%, with all the mice achieving stable disease (SD).

### Example 19. Combination Treatment with Compound 73 and Compound 2-5 in in NCI-H226 (BAP1mut, CDKN2Adel, FGFR High) Xenograft Model

In this experiment, NCI-H226 (BAP1^{mut}, CDKN2^{Adel}, FGFR^{high}) xenograft model was established to evaluate the anti-tumor effect of Compound 73 in combination with Compound 2-5. The dosing regimen was as follows:
Compound 2-5: 50 mg/kg, orally, once per day, for a total of 35 days;
Compound 73: 50 mg/kg, orally, once per day, for a total of 35 days.

The dosing regimen of each drug in the dosing regimen for the combination is the same as the dosing regimen for the single drug.

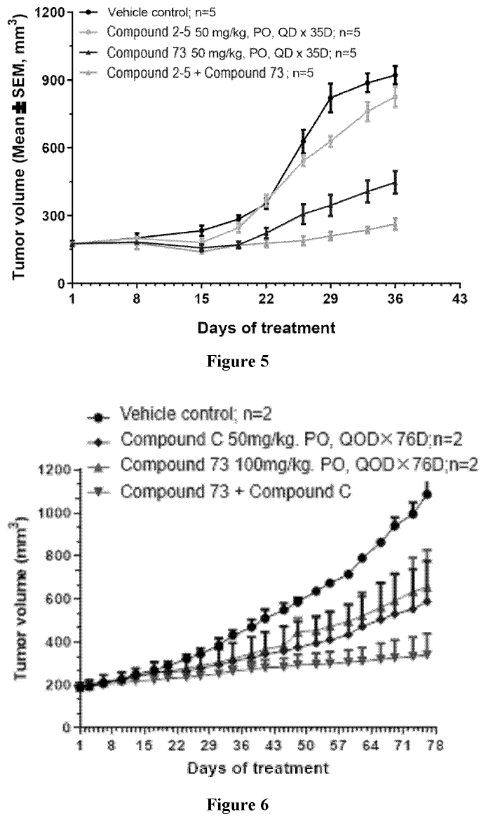

As shown in FIG. 5, Compound 2-5 was almost not effective in inhibiting tumor growth, and Compound 73 showed moderate antitumor activity, with T/C value of 48.6% at the end of the study. The combination treatment further reduced the T/C value to 28.5%, with a synergistic factor of 1.4, indicating synergistic effects.

### Example 20. Combination Treatment with Compound 73 and Compound C in Subcutaneous Malignant Pleural Mesothelioma (MPM) PDX Model (FGFR1^{high}; BAP1^{mut})

In this experiment, subcutaneous malignant pleural mesothelioma (MPM) PDX model (FGFR1^{high}; BAP1^{mut}) was established to evaluate the anti-tumor effect of Compound 73 in combination with Compound C. The dosing regimen was as follows:
Compound C: 50 mg/kg, orally, every other day, for a total of 76 days;
Compound 73: 100 mg/kg, orally, every other day, for a total of 76 days.

The dosing regimen of each drug in the dosing regimen for the combination is the same as the dosing regimen for the single drug.

As shown in FIG. 6 and Table 6, single agent of Compound 73 and Compound C showed moderate antitumor activity, with T/C value of 53% and 60% at the end of the study. The combination treatment further reduced the T/C value to 31%, with a synergistic factor of 1.02, indicating synergistic effects.

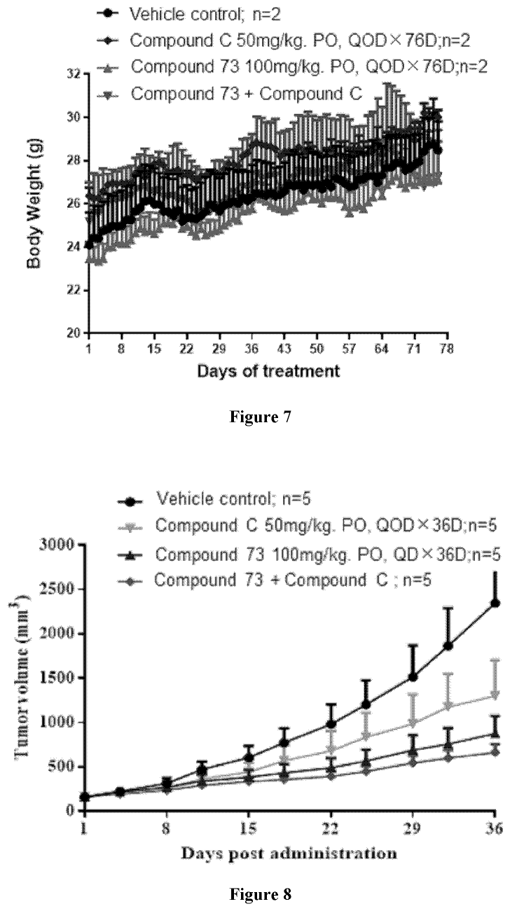

As shown in FIG. 7, combination treatment did not lead to body weight loss during all treatments.

TABLE 6
Antitumor effects of compound 73 in combination with
compound C in subcutaneous malignant pleural mesothelioma (MPM)
PDX model(FGFR1^{high}; BAP 1^{mut})

| | RTV @ | T/C (%) | Synergy |
|---|---|---|---|
| Treatment | D 76 | @ D 76 | ratio @ D 76 |
| Vehicle controls | 5.82 | — | — |
| Compound 73 100 mg/kg | 3.06 | 53 | — |
| Compound C 50 mg/kg | 3.47 | 60 | — |
| Compound 73 + | 1.78 | 31 | 1.02 |
| Compound C | | | |

Synergy: Ratio >1, synergistic; Ratio = 1, additive; Ratio <1, antagonistic.

### Example 21. Combination Treatment with Compound 73 and Compound C in Subcutaneous BAP1 Mut Mesothelioma PDX Model

In this experiment, subcutaneous BAP1 mut mesothelioma PDX model was established (Lide Biotech) to evaluate the anti-tumor effect of Compound 73 in combination with Compound C. The dosing regimen was as follows:
Compound C: 50 mg/kg, orally, every other day, for a total of 36 days;
Compound 73: 100 mg/kg, orally, once per day, for a total of 36 days.

The dosing regimen of each drug in the dosing regimen for the combination is the same as the dosing regimen for the single drug.

As shown in FIG. 8 and Table 7, single agent of Compound C showed moderate antitumor activity, with a T/C value of 55.17% on day 36. Single agent of Compound 73 exerted potent antitumor activity, with a T/C value of 37.22% on day 36. Combination treatment significantly enhance tumor repression, with T/C value of 28.07%.

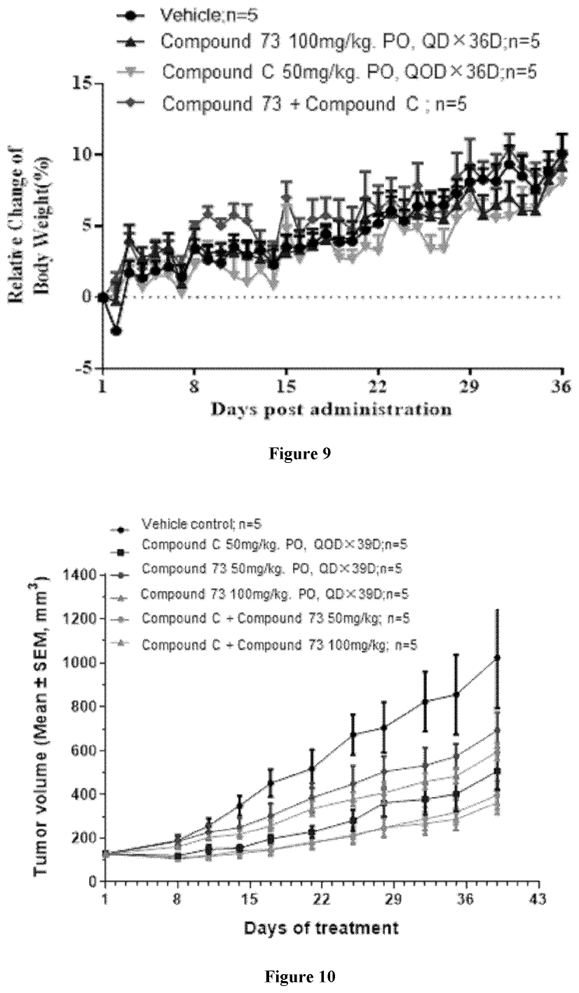

As shown in FIG. 9, combination treatment did not lead to body weight loss during all treatments.

TABLE 7
Antitumor effects of compound 73 in combination with compound
C in subcutaneous BAP1 mut mesothelioma PDX model

| Treatment | RTV @ D 36 | T/C (%) @ D 36 |
|---|---|---|
| Vehicle controls | 14.58 | — |
| Compound 73 100 mg/kg | 5.43 | 37.22 |
| Compound C 50 mg/kg | 8.04 | 55.17 |
| Compound 73 + Compound C | 4.09 | 28.07 |

### Example 22. In Vivo Antitumor Activity of Compound 73 and Compound C in H226 (BAP1mut, UHRF1high, TP53WT, CDKN2Adel, FGFRhigh) Subcutaneous Mesothelioma Xenograft Model

In this experiment, H226 (BAP1mut, UHRF1high, TP53WT, CDKN2Adel, FGFRhigh) subcutaneous mesothelioma xenograft model was established to evaluate the anti-tumor effect of Compound 73 in combination with Compound C. The dosing regimen was as follows:
Compound C: 50 mg/kg, orally, every other day, for a total of 39 days;
Compound 73: 50 mg/kg, orally, once per day, for a total of 39 days;
Compound 73: 100 mg/kg, orally, once per day, for a total of 39 days.

As shown in FIG. 10, single agent of Compound C and Compound 73 showed moderate antitumor activity.

As shown in Table 8, T/C (%) value of the combination group was 41.05% (Compound C 50 mg/kg+Compound 73 50 mg/kg) and 34.79% (Compound C 50 mg/kg+Compound 73 100 mg/kg) on day 39, compared to T/C values of 49.45% for Compound C (50 mg/kg), 67.7% for Compound 73 (50 mg/kg) and 59.73% for Compound 73 (100 mg/kg) single agent groups.

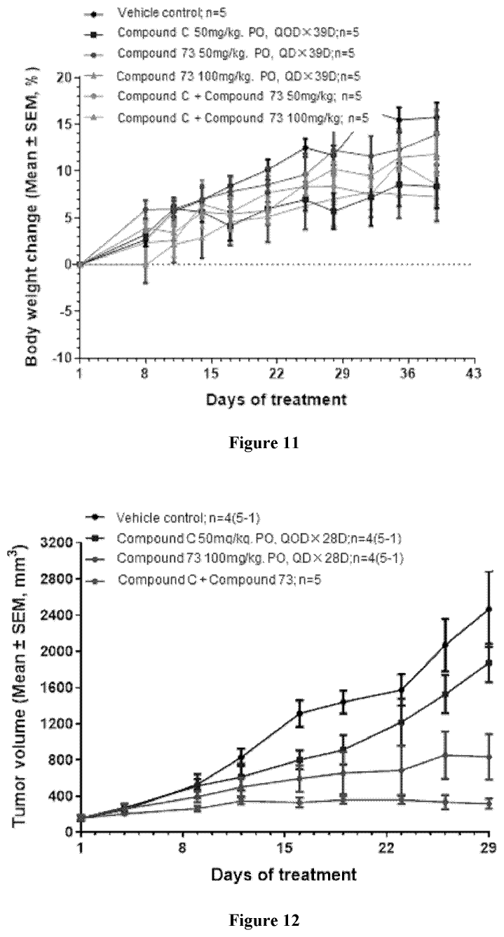

As shown in FIG. 11, combination treatment did not lead to body weight loss during all treatments.

TABLE 8
Antitumor effects of compound 73 in combination with compound
C in H226 (BAP1mut, UHRF1high, TP53WT, CDKN2Adel, FGFRhigh)
subcutaneous mesothelioma xenograft model

| Treatment | RTV @ D 39 | T/C (%) @ D 39 |
|---|---|---|
| Vehicle control | 7.97 ± 1.92 | — |
| Compound C 50 mg/kg | 3.94 ± 0.69* | 49.45 |
| Compound 73 50 mg/kg | 5.39 ± 0.56 | 67.70 |
| Compound 73 100 mg/kg | 4.76 ± 0.61 | 59.73 |
| Compound C 50 mg/kg + | 3.27 ± 0.69** | 41.05 |
| Compound 73 50 mg/kg | | |
| Compound C 50 mg/kg + | 2.17 ± 0.30** | 34.79 |
| Compound 73 100 mg/kg | | |

*p < 0.05,
**p < 0.01 vs. vehicle;

### Example 23. In Vivo Antitumor Activity of Compound C and Compound 73 in NCI-H28(X1) (BAP1^{mut}, UHRF1^{high}, TP53^{WT}, CDKN2A^{del}, FGFR^{low}, NF2^{WT}) Subcutaneous Mesothelioma Xenograft Model

In this experiment, NCI-H28(X1) (BAP1^{mut}, UHRF1^{high}, TP53^{WT}, CDKN2A^{del}, FGFR^{low}, NF2^{WT}) subcutaneous mesothelioma xenograft model was established to evaluate the anti-tumor effect of Compound 73 in combination with Compound C. The dosing regimen was as follows:
Compound C: 50 mg/kg, orally, every other day, for a total of 28 days;
Compound 73: 100 mg/kg, orally, once per day, for a total of 28 days;

As shown in FIG. 12 and Table 9, single agent of Compound C showed moderate antitumor activity, with a T/C value of 92.16% on day 29. Single agent of Compound 73 exerted potent antitumor activity, with a T/C value of 33.06% on day 29. Combination treatment significantly enhance tumor repression, with T/C value of 14.77% and a synergistic factor of 2.06, indicating synergistic effects.

TABLE 9
Antitumor effects of compound 73 in combination
with compound C in NCI-H28(X1) (BAP1^{mut}, UHRF1^{high}, TP53^{WT},
CDKN2A^{del}, FGFR^{low}, NF2^{WT}) subcutaneous mesothelioma
xenograft model

| | | T/C (%) | Synergy |
|---|---|---|---|
| Treatment | RTV @ D 29 | @ D 29 | ratio @ D 29 |
| Vehicle | 15.26 ± 1.39 | — | — |
| control | | | |
| Compound C | 14.06 ± 1.22 | 92.16 | — |
| 50 mg/kg | | | |
| Compound | 5.05 ± 0.41*** | 33.06 | — |
| 73 100 mg/kg | | | |
| Compound C + | 2.25 ± 0.58 ***††† | 14.77 | 2.06 |
| Compound 73 | | | |

***p < 0.001, vs. vehicle;,
†††p < 0.001, vs. Compound C; Synergy: Ratio >1, synergistic; Ratio = 1, additive; Ratio <1, antagonistic.

### Example 24. In Vivo Antitumor Activity of Compound C and Compound 73 in Subcutaneous 22RV1 Prostate Xenograft Model

In this experiment, subcutaneous 22RV1 prostate xenograft model was established to evaluate the anti-tumor effect of Compound 73 in combination with Compound C. The dosing regimen was as follows:
Compound C: 100 mg/kg, orally, every other day, for a total of 28 days;
Compound 73: 100 mg/kg, orally, once per day, for a total of 28 days;

The dosing regimen of each drug in the dosing regimen for the combination is the same as the dosing regimen for the single drug, for a total of 28 days.

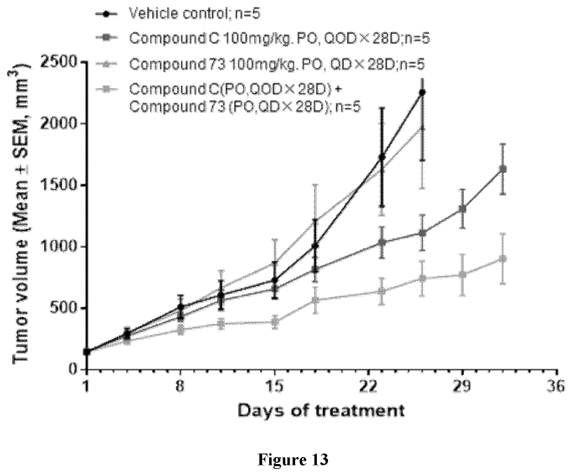

As shown in FIG. 13 and Table 10, single agent of Compound C and Compound 73 showed moderate antitumor activity, with a T/C value of 93.39% and 49.89% on day 26. Combination treatment significantly enhance tumor repression, with T/C value of 33.22% and a synergistic factor of 1.40, indicating synergistic effects.

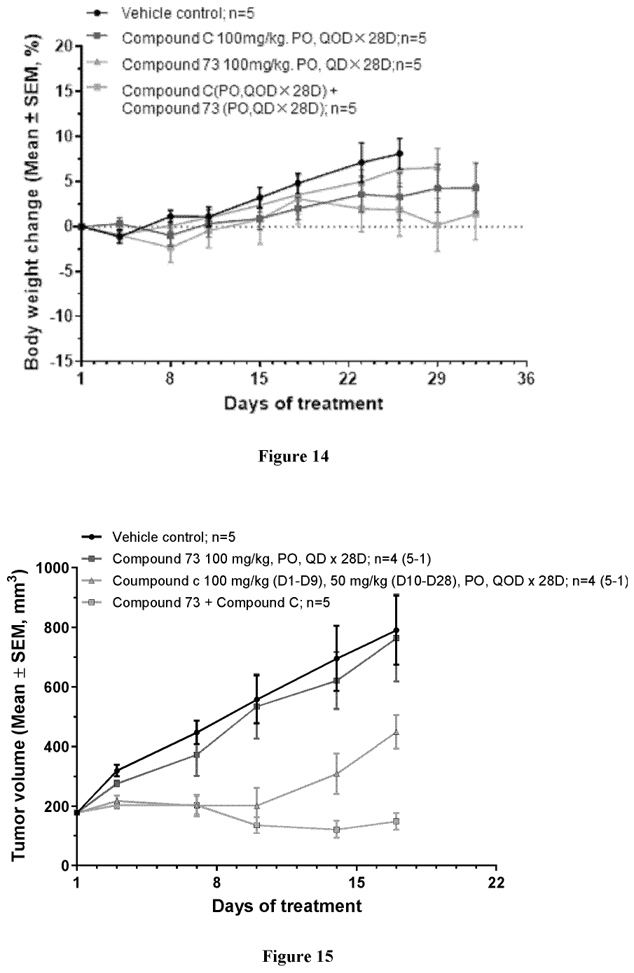

As shown in FIG. 14, combination treatment did not lead to body weight loss during all treatments.

TABLE 10
Antitumor effects of compound 73 in combination with compound
C in subcutaneous 22RV1 prostate xenograft model

| | | T/C(%) | Synergy | | |
|---|---|---|---|---|---|
| Treatment | RTV@ D 26 | @ D 26 | ratio @ D 26 | RTV@ D 29 | RTV@ D 32 |
| Vehicle | 15.86 ± 3.92 | — | — | — | — |
| Compound 73 100 mg/kg | 7.91 ± 0.74 | 49.89 | — | 9.28 ± 0.61 | 11.53 ± 0.68 |
| Compound C 50 mg/kg | 14.81 ± 4.35 | 93.39 | — | 18.54 ± 7.96 | — |
| Compound 73 + Compound C | 5.27 ± 0.97 | 33.22 | 1.40 | 5.48 ± 1.15 | 6.39 ± 1.38 |

Synergy: Ratio >1, synergistic; Ratio = 1, additive; Ratio <1, antagonistic.

### Example 25. In Vivo Antitumor Activity of Compound C and Compound 73 in Subcutaneous LnCap Prostate Xenograft Model

In this experiment, subcutaneous LnCap prostate xenograft model was established to evaluate the anti-tumor effect of Compound 73 in combination with Compound C. The dosing regimen was as follows:
Compound C: 100 mg/kg, orally, every other day, for day 1-day 9; 50 mg/kg, orally, every other day, for day 10-day 28;
Compound 73: 100 mg/kg, orally, once per day, for a total of 28 days;

The dosing regimen of each drug in the dosing regimen for the combination is the same as the dosing regimen for the single drug, for a total of 28 days.

As shown in FIG. 15 and Table 11, single agent of Compound C and Compound 73 showed moderate antitumor activity, with a T/C value of 57.46% and 91.30% on day 17. Combination treatment significantly enhance tumor repression, with T/C value of 18.86% and a synergistic factor of 2.81, indicating synergistic effects.

TABLE 11
Antitumor effects of compound 73 in combination with compound
C in subcutaneous LnCap prostate xenograft model

| | | T/C(%) | Synergy |
|---|---|---|---|
| Treatment | RTV@ D 17 | @ D 17 | ratio @ D 17 |
| Vehicle | 4.48 ± 0.72 | — | — |
| Compound 73 100 mg/kg | 4.09 ± 0.75 | 91.30 | — |
| Compound C 100 | 2.57 ± 0.24 | 57.46 | — |
| mg/kg/50 mg/kg | | | |
| Compound 73 + | 0.84 ± 0.14 | 18.68 | 2.81 |
| Compound C | | | |

### Example 26. In Vitro Anti-Proliferative Activity of Compound 73 and Compound 4 in EZH2 Mut BCL2 Translocation DLBCL Cells Lines

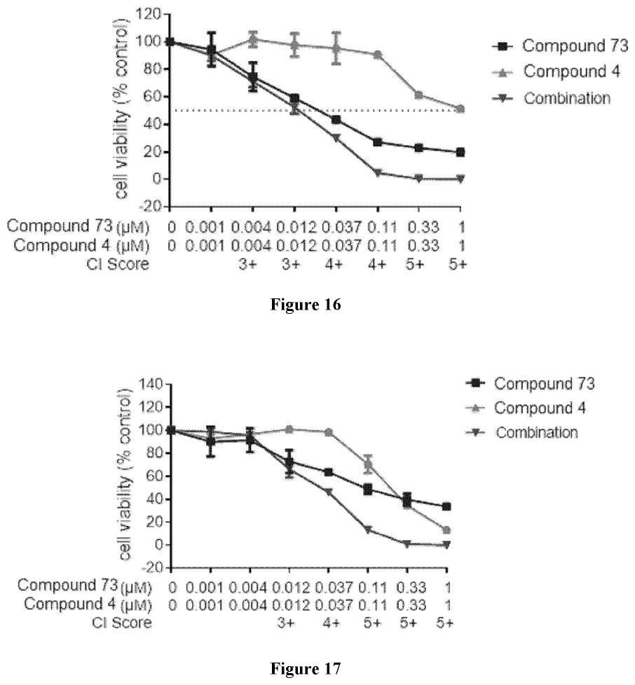

DLBCL cells were incubated with compound 73, compound 4 and their combination for 6 days, and the cell viability was determined by CTG assay. As seen in FIG. 16 and FIG. 17, the combination of compound 73 and compound 4 showed more stronger anti-proliferative activity than their single agent in KARPAS-422 and SU-DHL-6 cells, both carrying mutated EZH2 and translocated BCL-2. The combination index (CI) were calculated and scored to define the level of synergy. At most of the concentration combinations, the CI scores were 3+, 4+ and 5+, suggesting medium, strong and very strong synergy respectively.

### Example 27. In Vivo Antitumor Activity of Compound 73 and Compound 4 in KARPAS-422 Subcutaneous DLBCL Xenograft Model

In this experiment, KARPAS-422 subcutaneous DLBCL xenograft model was established to evaluate the anti-tumor effect of Compound 73 in combination with Compound 4. The dosing regimen was as follows:
Compound 73: 30 mg/kg, orally, once per day, for a total of 28 days;
Compound 4: 50 mg/kg, orally, once per day, for a total of 28 days;

The dosing regimen of each drug in the dosing regimen for the combination is the same as the dosing regimen for the single drug, for a total of 28 days.

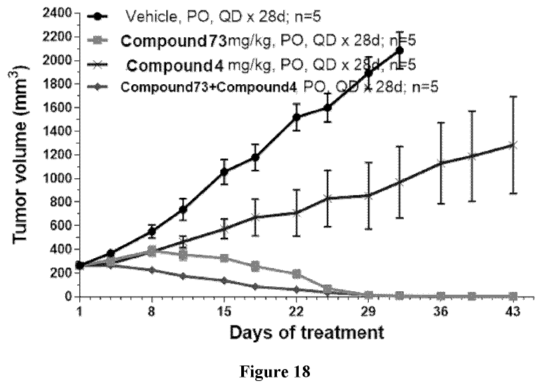

As seen in FIG. 18, the combination of compound 73 and compound 4 showed stronger anti-tumor activity than their single agents in KARPAS-422 xenograft model. The synergistic anti-tumor activity was observed after 8 days of treatment with the synergy ratio of 1.18. The synergy ratio reached 1.47 after 22 days of treatment.

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof.

All patents, patent applications, and publications cited herein are fully incorporated by reference herein in their entirety.

1. A pharmaceutical combination comprising an embryonic ectoderm development (EED) inhibitor and one or more anticancer reagents.

2. The pharmaceutical combination according to claim 1, wherein the one or more anticancer reagents are selected from the group consisting of an ALK inhibitor, a BCR-ABL inhibitor, a MDM2 inhibitor, a Bcl-2 inhibitor and other inhibitors.

3. The pharmaceutical combination according to claim 1 or 2, wherein the EED inhibitor is a compound of Formula I: 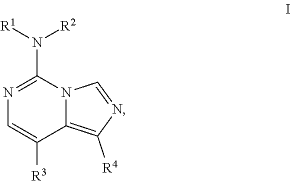
wherein:
R¹ is aralkyl;
R² is selected from the group consisting of hydrogen and C₁-C₄ alkyl;
R³ and R⁴ taken together with the carbon atoms to which they are attached form a radical of Formula I-A, I-B, or I-C: 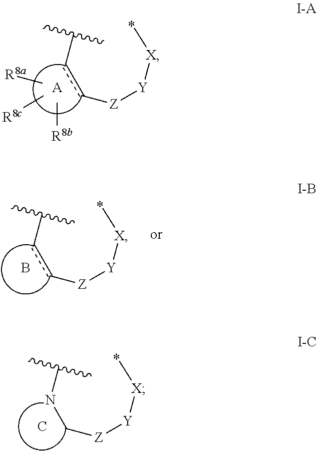
X is selected from the group consisting of —C(R^{5a})(R^{5b})—, —C(═O)—, and —S(═O)₂—;
R^{5a} and R^{5b} are independently selected from the group consisting of hydrogen and C₁-C₄ alkyl;
Y is selected from the group consisting of —C(R^{6a})(R^{6b})—, —S—, —O—, and —N(R⁷)—;
Z is —C(R^{6c})(R^{6d})ₘ—;
R^{6a} and R^{6b} are independently selected from the group consisting of hydrogen and C₁-C₄ alkyl;
each R^{6c} and R^{6d} is independently selected from the group consisting of hydrogen and C₁-C₄ alkyl;
m is 0, 1, or 2;
R⁷ is selected from the group consisting of hydrogen, C₁-C₆ alkyl, C₁-C₆ haloalkyl, optionally substituted C₃-C₈ cycloalkyl, optionally substituted C₄-C₈ heterocyclo, hydroxyalkyl, (alkoxy)alkyl, (cycloalkyl)alkyl, and (heterocyclo)alkyl;
R^{8a}, R^{8b}, and R^{8c} are independently selected from the group consisting of hydrogen, halo, C₁-C₄ alkyl, C₁-C₄ haloalkyl, C₁-C₄ alkoxy, and alkylsulfonyl; 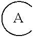
is a fused phenyl, fused 5-membered heteroaryl, or fused 6-membered heteroaryl; 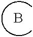
is an optionally substituted fused 3- to 8-membered cycloalkyl or optionally substituted fused 4- to 8-membered heterocyclo; 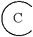
is an optionally substituted fused 4- to 8-membered heterocyclo;
the bond designated with a “ [symbol] ” is attached at the R³ position of Formula I and the bond designated with an “*” is attached at the R⁴ position of Formula I; and [symbol]
is a single or double bond,
or a pharmaceutically acceptable salt or solvate thereof.

4. The pharmaceutical combination according to claim 3, wherein the EED inhibitor is a compound of Formula II: 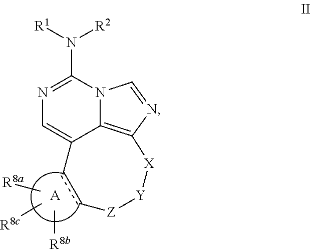
or a pharmaceutically acceptable salt or solvate thereof.

5. The pharmaceutical combination according to any one of claims 3-4, wherein Z is —CH₂—, or a pharmaceutically acceptable salt or solvate thereof.

6. The pharmaceutical combination according to any one of claims 3-5, wherein X is —C(═O)—, or a pharmaceutically acceptable salt or solvate thereof.

7. The pharmaceutical combination according to any one of claims 3-6, wherein Y is —N(R⁷)—, and preferably R⁷ is selected from the group consisting of C₁-C₆ alkyl, C₁-C₆ haloalkyl, and optionally substituted C₃-C₈ cycloalkyl, or a pharmaceutically acceptable salt or solvate thereof.

8. The pharmaceutical combination according to claim 3, wherein the EED inhibitor is selected from the group consisting of:
4-ethyl-12-(((5-fluoro-2,3-dihydrobenzofuran-4-yl)methyl)amino)-7-(trifluoromethyl)-4,5-dihydro-3H-2,4,8,11,12a-pentaazabenzo[4,5]cycloocta[1,2,3-cd]inden-3-one;
12-(((5-fluoro-2,3-dihydrobenzofuran-4-yl)methyl)amino)-4-(2,2,2-trifluoroethyl)-7-(trifluoromethyl)-4,5-dihydro-3H-2,4,8,11,12a-pentaazabenzo[4,5]cycloocta[1,2,3-cd]inden-3-one;
4-cyclopropyl-12-(((5-fluoro-2,3-dihydrobenzofuran-4-yl)methyl)amino)-7-(trifluoro methyl)-4,5-dihydro-3H-2,4,8,11,12a-pentaazabenzo[4,5]cycloocta[1,2,3-cd]inden-3-one;
12-(((5-fluoro-2,3-dihydrobenzofuran-4-yl)methyl)amino)-4-isopropyl-7-(trifluoromethyl)-4,5-dihydro-3H-2,4,8,11,12a-pentaazabenzo[4,5]cycloocta[1,2,3-cd]inden-3-one; and
11-(((5-fluoro-2,3-dihydrobenzofuran-4-yl)methyl)amino)-6-methyl-4H-3-thia-2,5,10,11a-tetraazadibenzo[cd,f]azulene-3,3-dioxide,
or a pharmaceutically acceptable salt or solvate thereof.

9. The pharmaceutical combination according to claim 3, wherein the EED inhibitor is: 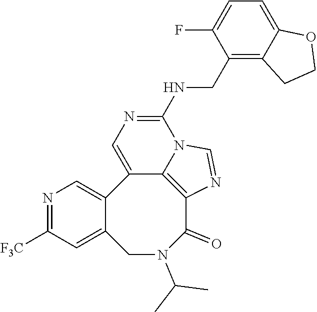 [SMILES: CC(C)N1Cc2cc(C(F)(F)F)ncc2-c2cnc(NCc3c(F)ccc4c3CCO4)n3cnc(c23)C1=O]
12-(((5-fluoro-2,3-dihydrobenzofuran-4-yl)methyl)amino)-4-isopropyl-7-(trifluoromethyl)-4,5-dihydro-3H-2,4,8,11,12a-pentaazabenzo[4,5]cycloocta[1,2,3-cd]inden-3-one, or a pharmaceutically acceptable salt or solvate thereof.

10. The pharmaceutical combination according to any one of claims 2 to 9, wherein the ALK inhibitor is:
5-chloro-N2-(2-isopropoxy-5-methyl-4-(1-(tetrahydro-2H-pyran-4-yl)-1,2,3,6-tetrahydropyridin-4-yl)phenyl)-N-(2-(isopropylsulfonyl)phenyl)pyrimidine-2,4-diamine, or a pharmaceutically acceptable salt or hydrate thereof.

11. The pharmaceutical combination according to any one of claims 2 to 10, wherein the BCR-ABL inhibitor is a compound of the formula (A) or a pharmaceutically acceptable salt or solvate thereof: 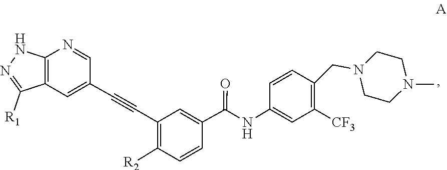
wherein,
R¹ is hydrogen, C₁-C₄ alkyl, C₃-C₆ cycloalkyl, C₁-C₄ alkyloxy, or phenyl; and R² is hydrogen, C₁-C₄ alkyl, C₃-C₆ cycloalkyl, or halogen.

12. The pharmaceutical combination according to claim 11, wherein the BCR-ABL inhibitor is the compound with the following structure or a pharmaceutically acceptable salt or solvate thereof:

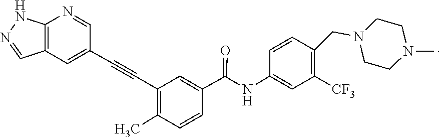 [SMILES: Cc1ccc(C(=O)Nc2ccc(CN3CCN(C)CC3)c(C(F)(F)F)c2)cc1C#Cc1cnc2[nH]ncc2c1]

13. The pharmaceutical combination according to any one of claims 2-12, wherein the MDM2 inhibitor is the compound with the following structure or a pharmaceutically acceptable salt or solvate thereof:

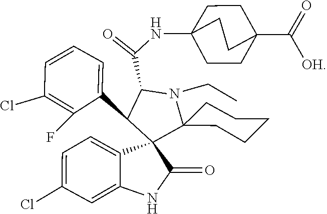 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

14. The pharmaceutical combination according to any one of claims 2-13, wherein the Bcl-2 inhibitor is the compound with the following structure or a pharmaceutically acceptable salt or solvate thereof:

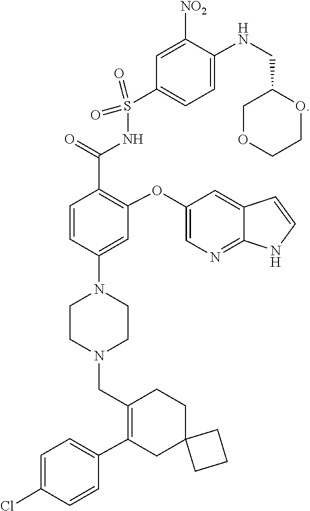 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

15. The pharmaceutical combination according to any one of claims 1 to 14 for use in treating or suppressing a cancer, reducing its severity, lowering its risk or inhibiting its metastasis in an individual, wherein the cancer is preferably selected from the group consisting of bladder cancer, breast cancer, cervical cancer, colon cancer (including colorectal cancer), esophageal cancer, esophageal squamous cell carcinoma, head and neck cancer, liver cancer, lung cancer (including small cell lung cancer, non-small cell lung cancer and lung squamous cell carcinoma), mesothelial tumor, melanoma, myeloma, rhabdomyosarcoma, inflammatory myofibroblastic tumor, neuroturbo chargeoma, pancreatic cancer, prostate cancer, kidney cancer, renal cell carcinoma, sarcoma (including osteosarcoma), skin cancer, squamous cell carcinoma, spindle cell carcinoma, gastric cancer, testicular cancer, thyroid cancer, uterine cancer, mesothelioma, neuroblastoma, cholangiocarcinoma, leiomyosarcoma, liposarcoma, nasopharyngeal carcinoma, neuroendocrine carcinoma, ovarian cancer, salivary gland cancer, metastasis caused by spindle cell carcinoma, anaplastic large cell lymphoma, thyroid undifferentiated carcinoma, non-Hodgkin's lymphoma, Hodgkin's lymphoma and hematological malignancies, such as acute myeloid leukemia (AML), acute lymphoblastic leukemia (ALL), diffuse large B-cell lymphoma (DLBCL), follicular lymphoma (FL), chronic lymphocytic leukemia (CLL), chronic myeloid leukemia (CML), uveal melanoma, pleural mesothelioma, peritoneal mesothelioma;
Preferably, the cancer is mesothelioma, neuroblastoma, non-small cell lung cancer, lung adenocarcinoma (LUAD), lung squamous cell carcinoma, ovarian cancer, uveal melanoma, colon cancer, and liver cancer;
Further preferably, the cancer is mesothelioma (including pleural mesothelioma, peritoneal mesothelioma, mesothelioma with wild type BAP1 and mesothelioma with mutant BAP1), prostate cancer and diffuse large B-cell lymphoma (DLBCL)(including EZH2 mut DLBCL and EZH2 mut Bcl-2 translocation DLBCL).

16. The pharmaceutical combination according to any one of claims 1 to 15, wherein the weight ratio between the EED inhibitor and the one or more anticancer reagents is 0.005-5000:0.005-5000, for example, 0.05-1500:0.005-5000, 0.1-6:0.005-4, 100:0.5-400, 100:1-350, 100:2-300, 100:5-200, 100:10-150, 100:10-100, 100:10-90, or 100: 20-80.

17. The pharmaceutical combination according to any one of claims 1 to 15, wherein the molar ratio between the EED inhibitor and the one or more anticancer reagents is 10-1: 1-10, for example, 10:1, 9:1, 8:1, 7:1, 6:1, 5:1, 4:1, 3:1, 2:1, 1:1:1:2, 1:3, 1:4, 1:5, 1:6, 1:7, 1:8, 1:9, 1:10, and the ranges between any of the aforementioned values are also included.

18. The pharmaceutical combination according to any one of claims 2 to 17 wherein the EED inhibitor is:
12-(((5-fluoro-2,3-dihydrobenzofuran-4-yl)methyl)amino)-4-isopropyl-7-(trifluoromethyl)-4,5-dihydro-3H-2,4,8,11,12a-pentaazabenzo[4,5]cycloocta[1,2,3-cd]inden-3-one, or a pharmaceutically acceptable salt or solvate thereof;
the ALK inhibitor is:
5-chloro-N2-(2-isopropoxy-5-methyl-4-(1-(tetrahydro-2H-pyran-4-yl)-1,2,3,6-tetrahydropyridin-4-yl)phenyl)-N⁴-(2-(isopropylsulfonyl)phenyl)pyrimidine-2,4-diamine, or a pharmaceutically acceptable salt or hydrate thereof; and/or
the BCR-ABL inhibitor is the compound with the following structure or a pharmaceutically acceptable salt or solvate thereof: 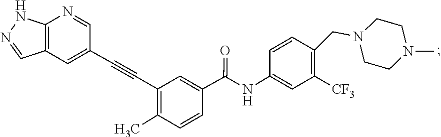 [SMILES: Cc1ccc(C(=O)Nc2ccc(CN3CCN(C)CC3)c(C(F)(F)F)c2)cc1C#Cc1cnc2[nH]ncc2c1]
the MIDM2 inhibitor is the compound with the following structure or a pharmaceutically acceptable salt or solvate thereof: 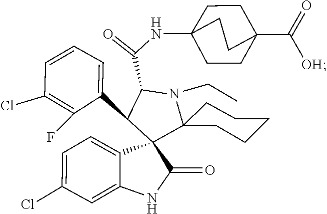 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]
the Bcl-2 inhibitor is the compound with the following structure or a pharmaceutically acceptable salt or solvate thereof:

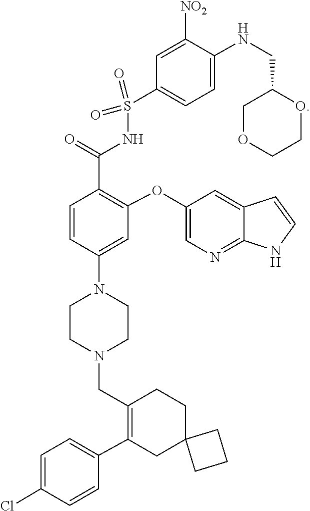 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

19. A pharmaceutical composition comprising the pharmaceutical combination according to any one of claims 1 to 18, and optionally a pharmaceutically acceptable carrier.

20. The pharmaceutical composition according to claim 19, which is in the form of a tablet, a capsule, a granule, a syrup, a powder, a lozenge, a sachet, a cachet, an elixir, a suspension, an emulsion, a solution, an aerosol, an ointment, a cream and an injection.

21. A method for treating or suppressing a cancer, reducing its severity, lowering its risk or inhibiting its metastasis in an individual, comprising administering to the individual a therapeutically effective amount of an EED inhibitor, and optionally a therapeutically effective amount of one or more anticancer reagents;
preferably, the EED inhibitor is as defined in any one of claims 3 to 9 and the anticancer reagent is selected from the group consisting of an ALK inhibitor, a BCR-ABL inhibitor, a MDM2 inhibitor, a Bcl-2 inhibitor and other inhibitors, such as those defined in any one of claims 8 to 14, and
preferably, the cancer is as defined in claim 15.

22. The method according to claim 21, wherein the EED inhibitor is administrated in an amount of from about 0.005 mg/day to about 5000 mg/day, such as an amount of about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

23. The method according to claim 21 or 22, wherein the EED inhibitor is administrated in an amount of from about 1 ng/kg to about 200 mg/kg, about 1 μg/kg to about 100 mg/kg, or about 1 mg/kg to about 50 mg/kg per unit dose, for example, administrated in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg per unit dose, and administrated with one or more (e.g., 1, 2, 3, 4, 5, 6, 7, 8, 9 or 10) unit doses per day.

24. The method according to any one of claims 21 to 23, wherein the one or more anticancer reagents are administrated in an amount of from 0.005 mg/day to about 5000 mg/day, for example, about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

25. The method according to any one of claims 21 to 23, wherein the one or more anticancer reagents are administrated in an amount of from about 1 ng/kg to about 200 mg/kg, from about 1 μg/kg to about 100 mg/kg, or from about 1 mg/kg to about 50 mg/kg per unit dose, for example, administrated in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg per unit dose, and administered with one or more (e.g., 1, 2, 3, 4, 5, 6, 7, 8, 9, or 10) unit doses per day.

26. The method according to any one of claims 21 to 25, wherein the EED inhibitor, and the one or more anticancer reagents are administered together, simultaneously, sequentially or alternately.

27. The method according to any one of claims 21 to 26, wherein the EED inhibitor, and the one or more anticancer reagents are administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 28 days, at least 30 days, at least 35 days, at least 40 days, at least 45 days, or at least 50 days.

28. The method according to any one of claims 21 to 27, wherein the EED inhibitor, and the one or more anticancer reagents are administered for one or more (e.g., 1, 2, 3, 4, 5, 6, 7, 8, 9 or 10) courses of treatment, in which each of the courses lasts at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days; and there is an interval of 0, 1, 2, 3, 4, 5, 6, 7, 8, 9, 10 days, two weeks, three weeks or four weeks between every two courses of treatment.

29. The method according to any one of claims 21 to 28, wherein the EED inhibitor, and the one or more anticancer reagents are administrated via the same (e.g., oral) or different routes (e.g., oral and parenteral (e.g., injection), respectively).

30. Use of an EED inhibitor alone or in combination with one or more anticancer reagents for treating or suppressing a cancer, reducing its severity, lowering its risk or inhibiting its metastasis in an individual,
preferably, the EED inhibitor is as defined in any one of claims 3 to 9 and the anticancer reagent is selected from the group consisting of an ALK inhibitor, a BCR-ABL inhibitor, a MDM2 inhibitor, a Bcl-2 inhibitor and other inhibitors, such as those defined in any one of claims 8 to 14, and
preferably, the cancer is as defined in claim 15.

31. Use of an EED inhibitor alone or in combination with one or more anticancer reagents in the manufacture of a medicament for treating or suppressing a cancer, reducing its severity, lowering its risk or inhibiting its metastasis in an individual,
preferably, the EED inhibitor is as defined in any one of claims 3 to 9 and the anticancer reagent is selected from the group consisting of an ALK inhibitor, a BCR-ABL inhibitor, a MDM2 inhibitor, a Bcl-2 inhibitor and other inhibitors, such as those defined in any one of claims 8 to 14, and
preferably, the cancer is as defined in claim 15.

32. A kit, comprising:
(a) a first component in a first container, the first component comprising an EED inhibitor (preferably an EED inhibitor as defined in any one of claims 3 to 9), and optionally a pharmaceutically acceptable carrier;
(b) a second component in a second container, the second component comprising an anticancer reagent (preferably an anticancer reagent as defined in any one of claims 8 to 14), and optionally a pharmaceutically acceptable carrier; and
(c) an optional specification.
